SEQUENCE LISTING (128 sequences)

SEQ ID NO: 1 - protein, 585 aa, Artificial Sequence [sequence omitted]

SEQ ID NO: 2 - protein, 585 aa, Artificial Sequence
  HSA variant
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 3 - protein, 585 aa, Artificial Sequence
  HSA variant
DAHKSEVAHRFKDLGEENFKALVLIAFAQYLQQAPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 4 - protein, 20 aa, Artificial sequence
  Linker
GGGGSGGGGSGGGGSGGGGS

SEQ ID NO: 5 - protein, 719 aa, Artificial Sequence
  fusion protein
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGSGGGGSGGGGSGGGGSGGGGSARNGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 6 - protein, 112 aa, Artificial Sequence
  GDF15 variant
ARNGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 7 - protein, 109 aa, Artificial Sequence
  GDF15 variant
GDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 8 - protein, 108 aa, Artificial Sequence
  GDF15 variant
DHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 9 - protein, 107 aa, Artificial Sequence
  GDF15 variant
HCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 10 - protein, 106 aa, Artificial Sequence
  GDF15 variant
CPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 11 - protein, 98 aa, Artificial Sequence
  GDF15 variant
CRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 12 - protein, 42 aa, Artificial sequence
  Linker
GSGGGGSGGGGSGGGGSGGGGSGGGGSGGGGSGGGGSGGGGS

SEQ ID NO: 13 - protein, 42 aa, Artificial sequence
  Linker
GAGGGGAGGGGAGGGGAGGGGAGGGGAGGGGAGGGGAGGGGA

SEQ ID NO: 14 - protein, 20 aa, Artificial sequence
  linker
APAPAPAPAPAPAPAPAPAP

SEQ ID NO: 15 - protein, 24 aa, Artificial sequence
  linker
APAPAPAPAPAPAPAPAPAPAPAP

SEQ ID NO: 16 - protein, 48 aa, Artificial sequence
  linker
GGSEGKSSGSGSESKSTEGKSSGSGSESKSTEGKSSGSGSESKSTGGS

SEQ ID NO: 17 - protein, 42 aa, Artificial sequence
  linker
GSPGGGSPGGGSPGGGSPGGGSPGGGSPGGGSPGGGSPGGGS

SEQ ID NO: 18 - protein, 42 aa, Artificial sequence
  linker
GSAGGGSAGGGSAGGGSAGGGSAGGGSAGGGSAGGGSAGGGS

SEQ ID NO: 19 - protein, 34 aa, Artificial sequence
  linker
GGSEGKSSGSGSESKSTEGKSSGSGSESKSTGGS

SEQ ID NO: 20 - protein, 14 aa, Artificial sequence
  linker
ASGGGGSGGGGSGT

SEQ ID NO: 21 - protein, 44 aa, Artificial sequence
  linker
ASGGGGSGGGGSGGGGSGGGGSGGGGSGGGGSGGGGSGGGGSGT

SEQ ID NO: 22 - protein, 14 aa, Artificial sequence
  linker
ASAPAPAPAPAPGT

SEQ ID NO: 23 - protein, 24 aa, Artificial sequence
  linker
ASAPAPAPAPAPAPAPAPAPAPGT

SEQ ID NO: 24 - protein, 44 aa, Artificial sequence
  linker
ASAPAPAPAPAPAPAPAPAPAPAPAPAPAPAPAPAPAPAPAPGT

SEQ ID NO: 25 - protein, 711 aa, Artificial Sequence
  fusion protein
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLASGGGGSGGGGSGTARNGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 26 - protein, 741 aa, Artificial Sequence
  fusion protein
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLASGGGGSGGGGSGGGGSGGGGSGGGGSGGGGSGGGGSGGGGSGTARNGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 27 - protein, 711 aa, Artificial Sequence
  fusion protein
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLASAPAPAPAPAPGTARNGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 28 - protein, 721 aa, Artificial Sequence
  fusion protein
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLASAPAPAPAPAPAPAPAPAPAPGTARNGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 29 - protein, 741 aa, Artificial Sequence
  fusion protein
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLASAPAPAPAPAPAPAPAPAPAPAPAPAPAPAPAPAPAPAPAPGTARNGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 30 - protein, 721 aa, Artificial Sequence
  fusion protein
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLASEAAAKEAAAKEAAAKEAAAKGTARNGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 31 - protein, 741 aa, Artificial Sequence
  fusion protein
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLASEAAAKEAAAKEAAAKEAAAKEAAAKEAAAKEAAAKEAAAKGTARNGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 32 - protein, 24 aa, Artificial sequence
  linker
ASEAAAKEAAAKEAAAKEAAAKGT

SEQ ID NO: 33 - protein, 44 aa, Artificial sequence
  linker
ASEAAAKEAAAKEAAAKEAAAKEAAAKEAAAKEAAAKEAAAKGT

SEQ ID NO: 34 - protein, 10 aa, Artificial sequence
  linker
GGGGSGGGGS

SEQ ID NO: 35 - protein, 15 aa, Artificial sequence
  linker
GGGGSGGGGSGGGGS

SEQ ID NO: 36 - protein, 706 aa, Artificial Sequence
  fusion protein
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGSGGGGSGGGGSGGGGSGGGGSCSRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 37 - protein, 705 aa, Artificial Sequence
  fusion protein
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGSGGGGSGGGGSGGGGSGGGGSCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 38 - protein, 20 aa, Artificial sequence
  linker
GGGGSGGGGSGGGGSGGGGS

SEQ ID NO: 39 - protein, 25 aa, Artificial sequence
  linker
GGGGSGGGGSGGGGSGGGGSGGGGS

SEQ ID NO: 40 - protein, 719 aa, Artificial Sequence; identical to SEQ ID NO: 5
  fusion protein

SEQ ID NO: 41 - protein, 30 aa, Artificial sequence
  linker
GGGGSGGGGSGGGGSGGGGSGGGGSGGGGS

SEQ ID NO: 42 - protein, 35 aa, Artificial sequence
  linker
GGGGSGGGGSGGGGSGGGGSGGGGSGGGGSGGGGS

SEQ ID NO: 43 - protein, 40 aa, Artificial sequence
  linker
GGGGSGGGGSGGGGSGGGGSGGGGSGGGGSGGGGSGGGGS

SEQ ID NO: 44 - protein, 45 aa, Artificial sequence
  linker
GGGGSGGGGSGGGGSGGGGSGGGGSGGGGSGGGGSGGGGSGGGGS

SEQ ID NO: 45 - protein, 50 aa, Artificial sequence
  linker
GGGGSGGGGSGGGGSGGGGSGGGGSGGGGSGGGGSGGGGSGGGGSGGGGS

SEQ ID NO: 46 - protein, 55 aa, Artificial sequence
  linker
GGGGSGGGGSGGGGSGGGGSGGGGSGGGGSGGGGSGGGGSGGGGSGGGGSGGGGS

SEQ ID NO: 47 - protein, 60 aa, Artificial sequence
  linker
GGGGSGGGGSGGGGSGGGGSGGGGSGGGGSGGGGSGGGGSGGGGSGGGGSGGGGSGGGGS

SEQ ID NO: 48 - protein, 719 aa, Artificial Sequence
  fusion protein
DAHKSEVAHRFKDLGEENFKALVLIAFAQYLQQAPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGSGGGGSGGGGSGGGGSGGGGSARNGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 49 - protein, 65 aa, Artificial sequence
  linker
GGGGSGGGGSGGGGSGGGGSGGGGSGGGGSGGGGSGGGGSGGGGSGGGGSGGGGSGGGGSGGGGS

SEQ ID NO: 50 - protein, 70 aa, Artificial sequence
  linker
GGGGSGGGGSGGGGSGGGGSGGGGSGGGGSGGGGSGGGGSGGGGSGGGGSGGGGSGGGGSGGGGSGGGGS

SEQ ID NO: 51 - protein, 75 aa, Artificial sequence
  linker
GGGGSGGGGSGGGGSGGGGSGGGGSGGGGSGGGGSGGGGSGGGGSGGGGSGGGGSGGGGSGGGGSGGGGSGGGGS

SEQ ID NO: 52 - protein, 80 aa, Artificial sequence
  linker
GGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 53 - protein, 85 aa, Artificial sequence
  linker
GGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 54 - protein, 90 aa, Artificial sequence
  linker
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 55 - protein, 739 aa, Artificial Sequence
  fusion protein
DAHKSEVAHRFKDLGEENFKALVLIAFAQYLQQAPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGSGGGGSGGGGSGGGGSGGGGSGGGGSGGGGSGGGGSGGGGSARNGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 56 - protein, 717 aa, Artificial Sequence
  fusion protein
DAHKSEVAHRFKDLGEENFKALVLIAFAQYLQQAPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLAPAPAPAPAPAPAPAPAPAPARNGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 57 - protein, 719 aa, Artificial Sequence; identical to SEQ ID NO: 5
  fusion protein

SEQ ID NO: 58 - protein, 719 aa, Artificial Sequence; identical to SEQ ID NO: 48
  fusion protein

SEQ ID NO: 59 - protein, 717 aa, Artificial Sequence
  fusion protein
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLAPAPAPAPAPAPAPAPAPAPARNGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 60 - protein, 739 aa, Artificial Sequence
  fusion protein
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGSGGGGSGGGGSGGGGSGGGGSGGGGSGGGGSGGGGSGGGGSARNGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 61 - protein, 95 aa, Artificial sequence
  linker
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 62 - protein, 100 aa, Artificial sequence
  linker
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 63 - protein, 10 aa, Artificial sequence
  linker
EAAAKEAAAK

SEQ ID NO: 64 - protein, 719 aa, Artificial Sequence
  fusion protein
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGSGGGGSGGGGSGGGGSGGGGSARNGDHCPLGPGRCCRLHTVRASLEDLGWADWVLSPREVQVTMCIGACPSQFRAANMHAQIKTSLHRLKPDTVPAPCCVPASYNPMVLRQKTDTGVSLQTYDDLLAKDCHCI

SEQ ID NO: 65 - protein, 719 aa, Artificial Sequence
  fusion protein
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGSGGGGSGGGGSGGGGSGGGGSARNGDHCPLGPGRCCRLHTVRASLEDLGWADWVLSPREVQVTMCIGACPSQFRAANMHAQIKTSLHRLKPDTVPAPCCVPASYNPMVLWQKTDTGVSLQTYDDLLAKDCHCI

SEQ ID NO: 66 - protein, 719 aa, Artificial Sequence
  fusion protein
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGSGGGGSGGGGSGGGGSGGGGSARNGDHCPLGPGRCCRLHTVRASLEDLGWADWVAAPAEVQVTMCIGACPSQFRAANMHAQIKTSLHRLKPDTVPAPCCVPASYNPMVLIQKTDTGVSLQTYDDLLAKDCHCI

SEQ ID NO: 67 - protein, 719 aa, Artificial Sequence
  fusion protein
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGSGGGGSGGGGSGGGGSGGGGSARNGDHCPLGPGRCCRLHTVRASLEDLGWADWVLSPREVQVTMCIGACPSQFRAANMHAQIKTSLHRLKPDTVPAPCCVPASYNPMALAQKTDTGVSAQTYDDLLAKDCHCI

SEQ ID NO: 68 - protein, 719 aa, Artificial Sequence
  fusion protein
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGSGGGGSGGGGSGGGGSGGGGSARNGDHCPLGPGRCCRLHTVRASLEDLGWADWVAAPREVQVTMCIGACPSQFRAANMHAQIKTSLHRLKPDTVPAPCCVPASYNPMVLAQKTDTGVSLQTYDDLLAKDCHCI

SEQ ID NO: 69 - protein, 719 aa, Artificial Sequence
  fusion protein
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGSGGGGSGGGGSGGGGSGGGGSARNGDHCPLGPGRCCRLHTVRASLEDLGWADWVLSPREVQVTMCIGACPSQFRAANMHAQIKTSLHRLKPDTVPAPCCVPASYNPMALAQKTDTGVSLQTYDDLLAKDCHCI

SEQ ID NO: 70 - protein, 719 aa, Artificial Sequence
  fusion protein
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGSGGGGSGGGGSGGGGSGGGGSARNGDHCPLGPGRCCRLHTVRASLEDLGWADWVLSPREVQVTMCIGACPSQFRAANMHAWIKTSLHRLKPDTVPAPCCVPASYNPMVLIQKTDTGVSLQTYDDLLAKDCHCI

SEQ ID NO: 71 - protein, 719 aa, Artificial Sequence
  fusion protein
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGSGGGGSGGGGSGGGGSGGGGSARNGDHCPLGPGRCCRLHTVRASLEDLGWADAVLSPREVQVTMCIGACPSQFRAANMHAQIKTSLHRLKPDTVPAPCCVPASYNPMVLIQKTDTGVSLQTYDDLLAKDCHCI

SEQ ID NO: 72 - protein, 719 aa, Artificial Sequence
  fusion protein
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGSGGGGSGGGGSGGGGSGGGGSARNGDHCPLGPGRCCRLHTVRASLEDLGAADWVLSPREVQVTMCIGACPSQFRAANMHAQIKTSLHRLKPDTVPAPCCVPASYNPMVLIQKTDTGVSLQTYDDLLAKDCHCI

SEQ ID NO: 73 - protein, 719 aa, Artificial Sequence
  fusion protein
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGSGGGGSGGGGSGGGGSGGGGSARNGDHCPLGPGRCCRLHTVRASLEDLGWADWVLSPREVQVTMCIGACPSQFRAANMHAAIKTALHALKPDTVPAPCCVPASYNPMVLIQKTDTGVSLQTYDDLLAKDCHCI

SEQ ID NO: 74 - protein, 719 aa, Artificial Sequence
  fusion protein
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGSGGGGSGGGGSGGGGSGGGGSARNGDHCPLGPGRCCRLHTVRASLEDLGAADWVLSPREVQVTMCIGACPSQFRAANMHAAAKTSLHRLKPDTVPAPCCVPASYNPMVLIQKTDTGVSLQTYDDLLAKDCHCI

SEQ ID NO: 75 - protein, 719 aa, Artificial Sequence
  fusion protein
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGSGGGGSGGGGSGGGGSGGGGSARNGDHCPLGPGRCCRLHTVRASLEDLGAADAVLSPREVQVTMCIGACPSQFRAANMHAQIKTSLHRLKPDTVPAPCCVPASYNPMVLIQKTDTGVSLQTYDDLLAKDCHCI

SEQ ID NO: 76 - DNA, 2157 bp, Artificial Sequence
  fusion protein coding sequence
gacgcccacaagagcgaggtggcccaccggttcaaggacctgggcgaggagaacttcaaggccctggtgctgatcgccttcgcccagtacctgcagcagtcccccttcgaggaccacgtgaagctggtgaacgaggtgaccgagttcgccaagacctgcgtggccgacgagagcgccgagaactgcgacaagagcctgcacaccctgttcggcgacaagctgtgcaccgtggccaccctgcgggagacctacggcgagatggccgactgctgcgccaagcaggagcccgagcggaacgagtgcttcctgcagcacaaggacgacaaccccaacctgccccggctggtgcggcccgaggtggacgtgatgtgcaccgccttccacgacaacgaggagaccttcctgaagaagtacctgtacgagatcgcccggcggcacccctacttctacgcccccgagctgctgttcttcgccaagcggtacaaggccgccttcaccgagtgctgccaggccgccgacaaggccgcctgcctgctgcccaagctggacgagctgcgggacgagggcaaggccagcagcgccaagcagcggctgaagtgcgccagcctgcagaagttcggcgagcgggccttcaaggcctgggccgtggcccggctgagccagcggttccccaaggccgagttcgccgaggtgagcaagctggtgaccgacctgaccaaggtgcacaccgagtgctgccacggcgacctgctggagtgcgccgacgaccgggccgacctggccaagtacatctgcgagaaccaggacagcatcagcagcaagctgaaggagtgctgcgagaagcccctgctggagaagagccactgcatcgccgaggtggagaacgacgagatgcccgccgacctgcccagcctggccgccgacttcgtggagagcaaggacgtgtgcaagaactacgccgaggccaaggacgtgttcctgggcatgttcctgtacgagtacgcccggcggcaccccgactacagcgtggtgctgctgctgcggctggccaagacctacgagaccaccctggagaagtgctgcgccgccgccgacccccacgagtgctacgccaaggtgttcgacgagttcaagcccctggtggaggagccccagaacctgatcaagcagaactgcgagctgttcgagcagctgggcgagtacaagttccagaacgccctgctggtgcggtacaccaagaaggtgccccaggtgagcacccccaccctggtggaggtgagccggaacctgggcaaggtgggcagcaagtgctgcaagcaccccgaggccaagcggatgccctgcgccgaggactacctgagcgtggtgctgaaccagctgtgcgtgctgcacgagaagacccccgtgagcgaccgggtgaccaagtgctgcaccgagagcctggtgaaccggcggccctgcttcagcgccctggaggtggacgagacctacgtgcccaaggagttcaacgccgagaccttcaccttccacgccgacatctgcaccctgagcgagaaggagcggcagatcaagaagcagaccgccctggtggagctggtgaagcacaagcccaaggccaccaaggagcagctgaaggccgtgatggacgacttcgccgccttcgtggagaagtgctgcaaggccgacgacaaggagacctgcttcgccgaggagggcaagaagctggtggccgccagccaggccgccctgggcctgggcagcggcggcggcggcagcggcggcggcggatctggtggaggtggcagtggaggagggggatccgctcgcaacggtgaccactgccctctgggtcctggtcgctgctgccgcctgcacaccgttcgcgcttctctggaagacctgggttgggctgactgggttctgtctcctcgcgaagttcaggttaccatgtgcatcggtgcttgcccttctcagttccgcgctgctaacatgcacgctcagatcaaaacctctctgcaccgcctgaaacctgacaccgttcctgctccttgctgcgttcctgcttcttacaaccctatggttctgatccagaaaaccgacaccggtgtttctctgcagacctacgacgacctgctggctaaagactgccactgcatc

SEQ ID NO: 77 - DNA, 2133 bp, Artificial Sequence
  fusion protein coding sequence
gatgctcataagtccgaagtcgcccacagattcaaggacctcggagaagaaaattttaaggccctcgtgcttatcgccttcgcccaatacctccagcagtccccgttcgaggaccacgtgaagctcgtgaacgaagtgaccgagtttgccaagacttgtgtggcggatgaatccgccgagaactgcgacaagagcctccacacgctgttcggcgacaagctgtgcaccgtcgccacgctgagagaaacttacggagagatggccgactgctgcgcaaagcaggagccggaacggaacgaatgcttcctgcaacataaggacgataaccctaacttgcctcgcctggtccgccctgaggtcgacgtgatgtgcaccgcgttccacgacaacgaggaaacctttcttaagaagtacctgtacgagattgcgcggaggcacccttatttctacgcccccgaactgttgttcttcgccaagcggtacaaggctgcctttaccgaatgctgccaggccgccgataaggcggcttgcctgctgccgaagctcgacgagttgcgcgatgaggggaaggcgtcctccgctaagcagcggctgaaatgtgcgagcctccagaagttcggggagcgcgccttcaaggcctgggccgtggcgcgcctgtctcaacggttcccgaaggccgagttcgccgaagtgtcgaagctggtcaccgacctgacgaaagtgcacaccgaatgttgtcacggcgatctgctggaatgcgccgatgacagagccgatttggccaagtacatctgcgaaaaccaggacagcatttcgtcaaagctgaaggaatgctgcgaaaagcccttgctggaaaagtcccactgcatcgcggaagtggagaacgacgagatgcccgccgacctcccgtccctggccgccgatttcgtggagtcgaaggatgtgtgcaagaactacgcagaagccaaggacgtgttcctgggaatgtttctgtatgagtacgcccgccgccacccggactactcggtcgtgctcctgctgcgactggcaaagacctacgaaaccactctggagaagtgctgcgccgccgcggacccgcacgagtgctacgcaaaggtgttcgacgagttcaagccacttgtcgaggagcctcagaacctgatcaagcagaactgcgaactgttcgagcagctgggagagtacaaattccagaacgcgcttctcgtgcgctacaccaagaaggtcccccaggtgtccactccgaccctggtggaagtgtccaggaacctgggaaaggtcggctccaagtgttgcaagcatcccgaggctaagcgcatgccctgcgccgaggactacttgtccgtggtgctgaatcagctgtgcgtgctccatgaaaagaccccagtgtccgacagagtgaccaagtgctgtaccgaatcgctcgtgaaccggcggccgtgcttttccgcactggaggtggacgaaacctacgtgccgaaggagttcaacgcagaaaccttcactttccacgccgacatctgcactctgtccgagaaggagcggcagattaagaagcagactgccctggtggagcttgtgaaacacaagcctaaggccaccaaagagcagctgaaggccgtcatggatgatttcgcggccttcgtggaaaagtgttgtaaagcggacgacaaggagacttgcttcgccgaagaaggaaagaagctcgtggcagcgtcacaggccgctctgggcctcgctagcggtggagggggcagcggtggtggaggatccggtaccgcgcgcaacggggaccactgtccgctcgggcccgggcgttgctgccgtctgcacacggtccgcgcgtcgctggaagacctgggctgggccgattgggtgctgtcgccacgggaggtgcaagtgaccatgtgcatcggcgcgtgcccgagccagttccgggcggcaaacatgcacgcgcagatcaagacgagcctgcaccgcctgaagcccgacacggtgccagcgccctgctgcgtgcccgccagctacaatcccatggtgctcattcaaaagaccgacaccggggtgtcgctccagacctatgatgacttgttagccaaagactgccactgcata

SEQ ID NO: 78 - DNA, 2223 bp, Artificial Sequence
  fusion protein coding sequence
gatgctcataagtccgaagtcgcccacagattcaaggacctcggagaagaaaattttaaggccctcgtgcttatcgccttcgcccaatacctccagcagtccccgttcgaggaccacgtgaagctcgtgaacgaagtgaccgagtttgccaagacttgtgtggcggatgaatccgccgagaactgcgacaagagcctccacacgctgttcggcgacaagctgtgcaccgtcgccacgctgagagaaacttacggagagatggccgactgctgcgcaaagcaggagccggaacggaacgaatgcttcctgcaacataaggacgataaccctaacttgcctcgcctggtccgccctgaggtcgacgtgatgtgcaccgcgttccacgacaacgaggaaacctttcttaagaagtacctgtacgagattgcgcggaggcacccttatttctacgcccccgaactgttgttcttcgccaagcggtacaaggctgcctttaccgaatgctgccaggccgccgataaggcggcttgcctgctgccgaagctcgacgagttgcgcgatgaggggaaggcgtcctccgctaagcagcggctgaaatgtgcgagcctccagaagttcggggagcgcgccttcaaggcctgggccgtggcgcgcctgtctcaacggttcccgaaggccgagttcgccgaagtgtcgaagctggtcaccgacctgacgaaagtgcacaccgaatgttgtcacggcgatctgctggaatgcgccgatgacagagccgatttggccaagtacatctgcgaaaaccaggacagcatttcgtcaaagctgaaggaatgctgcgaaaagcccttgctggaaaagtcccactgcatcgcggaagtggagaacgacgagatgcccgccgacctcccgtccctggccgccgatttcgtggagtcgaaggatgtgtgcaagaactacgcagaagccaaggacgtgttcctgggaatgtttctgtatgagtacgcccgccgccacccggactactcggtcgtgctcctgctgcgactggcaaagacctacgaaaccactctggagaagtgctgcgccgccgcggacccgcacgagtgctacgcaaaggtgttcgacgagttcaagccacttgtcgaggagcctcagaacctgatcaagcagaactgcgaactgttcgagcagctgggagagtacaaattccagaacgcgcttctcgtgcgctacaccaagaaggtcccccaggtgtccactccgaccctggtggaagtgtccaggaacctgggaaaggtcggctccaagtgttgcaagcatcccgaggctaagcgcatgccctgcgccgaggactacttgtccgtggtgctgaatcagctgtgcgtgctccatgaaaagaccccagtgtccgacagagtgaccaagtgctgtaccgaatcgctcgtgaaccggcggccgtgcttttccgcactggaggtggacgaaacctacgtgccgaaggagttcaacgcagaaaccttcactttccacgccgacatctgcactctgtccgagaaggagcggcagattaagaagcagactgccctggtggagcttgtgaaacacaagcctaaggccaccaaagagcagctgaaggccgtcatggatgatttcgcggccttcgtggaaaagtgttgtaaagcggacgacaaggagacttgcttcgccgaagaaggaaagaagctcgtggcagcgtcacaggccgctctgggcctcgctagcggaggtggcggatcaggtggcggaggtagcggtggaggcggctctggcggaggtggatcaggcggaggaggttccggtggaggaggctcaggaggaggaggaagtggaggagggggatccggtaccgcgcgcaacggggaccactgtccgctcgggcccgggcgttgctgccgtctgcacacggtccgcgcgtcgctggaagacctgggctgggccgattgggtgctgtcgccacgggaggtgcaagtgaccatgtgcatcggcgcgtgcccgagccagttccgggcggcaaacatgcacgcgcagatcaagacgagcctgcaccgcctgaagcccgacacggtgccagcgccctgctgcgtgcccgccagctacaatcccatggtgctcattcaaaagaccgacaccggggtgtcgctccagacctatgatgacttgttagccaaagactgccactgcata

SEQ ID NO: 79 - DNA, 2133 bp, Artificial Sequence
  fusion protein coding sequence
gatgctcataagtccgaagtcgcccacagattcaaggacctcggagaagaaaattttaaggccctcgtgcttatcgccttcgcccaatacctccagcagtccccgttcgaggaccacgtgaagctcgtgaacgaagtgaccgagtttgccaagacttgtgtggcggatgaatccgccgagaactgcgacaagagcctccacacgctgttcggcgacaagctgtgcaccgtcgccacgctgagagaaacttacggagagatggccgactgctgcgcaaagcaggagccggaacggaacgaatgcttcctgcaacataaggacgataaccctaacttgcctcgcctggtccgccctgaggtcgacgtgatgtgcaccgcgttccacgacaacgaggaaacctttcttaagaagtacctgtacgagattgcgcggaggcacccttatttctacgcccccgaactgttgttcttcgccaagcggtacaaggctgcctttaccgaatgctgccaggccgccgataaggcggcttgcctgctgccgaagctcgacgagttgcgcgatgaggggaaggcgtcctccgctaagcagcggctgaaatgtgcgagcctccagaagttcggggagcgcgccttcaaggcctgggccgtggcgcgcctgtctcaacggttcccgaaggccgagttcgccgaagtgtcgaagctggtcaccgacctgacgaaagtgcacaccgaatgttgtcacggcgatctgctggaatgcgccgatgacagagccgatttggccaagtacatctgcgaaaaccaggacagcatttcgtcaaagctgaaggaatgctgcgaaaagcccttgctggaaaagtcccactgcatcgcggaagtggagaacgacgagatgcccgccgacctcccgtccctggccgccgatttcgtggagtcgaaggatgtgtgcaagaactacgcagaagccaaggacgtgttcctgggaatgtttctgtatgagtacgcccgccgccacccggactactcggtcgtgctcctgctgcgactggcaaagacctacgaaaccactctggagaagtgctgcgccgccgcggacccgcacgagtgctacgcaaaggtgttcgacgagttcaagccacttgtcgaggagcctcagaacctgatcaagcagaactgcgaactgttcgagcagctgggagagtacaaattccagaacgcgcttctcgtgcgctacaccaagaaggtcccccaggtgtccactccgaccctggtggaagtgtccaggaacctgggaaaggtcggctccaagtgttgcaagcatcccgaggctaagcgcatgccctgcgccgaggactacttgtccgtggtgctgaatcagctgtgcgtgctccatgaaaagaccccagtgtccgacagagtgaccaagtgctgtaccgaatcgctcgtgaaccggcggccgtgcttttccgcactggaggtggacgaaacctacgtgccgaaggagttcaacgcagaaaccttcactttccacgccgacatctgcactctgtccgagaaggagcggcagattaagaagcagactgccctggtggagcttgtgaaacacaagcctaaggccaccaaagagcagctgaaggccgtcatggatgatttcgcggccttcgtggaaaagtgttgtaaagcggacgacaaggagacttgcttcgccgaagaaggaaagaagctcgtggcagcgtcacaggccgctctgggcctcgctagcgcacctgcccccgctccagctcctgcaccaggtaccgcgcgcaacggggaccactgtccgctcgggcccgggcgttgctgccgtctgcacacggtccgcgcgtcgctggaagacctgggctgggccgattgggtgctgtcgccacgggaggtgcaagtgaccatgtgcatcggcgcgtgcccgagccagttccgggcggcaaacatgcacgcgcagatcaagacgagcctgcaccgcctgaagcccgacacggtgccagcgccctgctgcgtgcccgccagctacaatcccatggtgctcattcaaaagaccgacaccggggtgtcgctccagacctatgatgacttgttagccaaagactgccactgcata

SEQ ID NO: 80 - DNA, 2163 bp, Artificial Sequence
  fusion protein coding sequence
gatgctcataagtccgaagtcgcccacagattcaaggacctcggagaagaaaattttaaggccctcgtgcttatcgccttcgcccaatacctccagcagtccccgttcgaggaccacgtgaagctcgtgaacgaagtgaccgagtttgccaagacttgtgtggcggatgaatccgccgagaactgcgacaagagcctccacacgctgttcggcgacaagctgtgcaccgtcgccacgctgagagaaacttacggagagatggccgactgctgcgcaaagcaggagccggaacggaacgaatgcttcctgcaacataaggacgataaccctaacttgcctcgcctggtccgccctgaggtcgacgtgatgtgcaccgcgttccacgacaacgaggaaacctttcttaagaagtacctgtacgagattgcgcggaggcacccttatttctacgcccccgaactgttgttcttcgccaagcggtacaaggctgcctttaccgaatgctgccaggccgccgataaggcggcttgcctgctgccgaagctcgacgagttgcgcgatgaggggaaggcgtcctccgctaagcagcggctgaaatgtgcgagcctccagaagttcggggagcgcgccttcaaggcctgggccgtggcgcgcctgtctcaacggttcccgaaggccgagttcgccgaagtgtcgaagctggtcaccgacctgacgaaagtgcacaccgaatgttgtcacggcgatctgctggaatgcgccgatgacagagccgatttggccaagtacatctgcgaaaaccaggacagcatttcgtcaaagctgaaggaatgctgcgaaaagcccttgctggaaaagtcccactgcatcgcggaagtggagaacgacgagatgcccgccgacctcccgtccctggccgccgatttcgtggagtcgaaggatgtgtgcaagaactacgcagaagccaaggacgtgttcctgggaatgtttctgtatgagtacgcccgccgccacccggactactcggtcgtgctcctgctgcgactggcaaagacctacgaaaccactctggagaagtgctgcgccgccgcggacccgcacgagtgctacgcaaaggtgttcgacgagttcaagccacttgtcgaggagcctcagaacctgatcaagcagaactgcgaactgttcgagcagctgggagagtacaaattccagaacgcgcttctcgtgcgctacaccaagaaggtcccccaggtgtccactccgaccctggtggaagtgtccaggaacctgggaaaggtcggctccaagtgttgcaagcatcccgaggctaagcgcatgccctgcgccgaggactacttgtccgtggtgctgaatcagctgtgcgtgctccatgaaaagaccccagtgtccgacagagtgaccaagtgctgtaccgaatcgctcgtgaaccggcggccgtgcttttccgcactggaggtggacgaaacctacgtgccgaaggagttcaacgcagaaaccttcactttccacgccgacatctgcactctgtccgagaaggagcggcagattaagaagcagactgccctggtggagcttgtgaaacacaagcctaaggccaccaaagagcagctgaaggccgtcatggatgatttcgcggccttcgtggaaaagtgttgtaaagcggacgacaaggagacttgcttcgccgaagaaggaaagaagctcgtggcagcgtcacaggccgctctgggcctcgctagcgcacctgcccccgctccagcacccgccccagcccctgctcccgcaccagctcctgcaccaggtaccgctcgcaacggtgaccactgccctctgggtcctggtcgctgctgccgcctgcacaccgttcgcgcttctctggaagacctgggttgggctgactgggttctgtctcctcgcgaagttcaggttaccatgtgcatcggtgcttgcccttctcagttccgcgctgctaacatgcacgctcagatcaaaacctctctgcaccgcctgaaacctgacaccgttcctgctccttgctgcgttcctgcttcttacaaccctatggttctgatccagaaaaccgacaccggtgtttctctgcagacctacgacgacctgctggctaaagactgccactgcatc

SEQ ID NO: 81 - DNA, 2223 bp, Artificial Sequence
  fusion protein coding sequence
gatgctcataagtccgaagtcgcccacagattcaaggacctcggagaagaaaattttaaggccctcgtgcttatcgccttcgcccaatacctccagcagtccccgttcgaggaccacgtgaagctcgtgaacgaagtgaccgagtttgccaagacttgtgtggcggatgaatccgccgagaactgcgacaagagcctccacacgctgttcggcgacaagctgtgcaccgtcgccacgctgagagaaacttacggagagatggccgactgctgcgcaaagcaggagccggaacggaacgaatgcttcctgcaacataaggacgataaccctaacttgcctcgcctggtccgccctgaggtcgacgtgatgtgcaccgcgttccacgacaacgaggaaacctttcttaagaagtacctgtacgagattgcgcggaggcacccttatttctacgcccccgaactgttgttcttcgccaagcggtacaaggctgcctttaccgaatgctgccaggccgccgataaggcggcttgcctgctgccgaagctcgacgagttgcgcgatgaggggaaggcgtcctccgctaagcagcggctgaaatgtgcgagcctccagaagttcggggagcgcgccttcaaggcctgggccgtggcgcgcctgtctcaacggttcccgaaggccgagttcgccgaagtgtcgaagctggtcaccgacctgacgaaagtgcacaccgaatgttgtcacggcgatctgctggaatgcgccgatgacagagccgatttggccaagtacatctgcgaaaaccaggacagcatttcgtcaaagctgaaggaatgctgcgaaaagcccttgctggaaaagtcccactgcatcgcggaagtggagaacgacgagatgcccgccgacctcccgtccctggccgccgatttcgtggagtcgaaggatgtgtgcaagaactacgcagaagccaaggacgtgttcctgggaatgtttctgtatgagtacgcccgccgccacccggactactcggtcgtgctcctgctgcgactggcaaagacctacgaaaccactctggagaagtgctgcgccgccgcggacccgcacgagtgctacgcaaaggtgttcgacgagttcaagccacttgtcgaggagcctcagaacctgatcaagcagaactgcgaactgttcgagcagctgggagagtacaaattccagaacgcgcttctcgtgcgctacaccaagaaggtcccccaggtgtccactccgaccctggtggaagtgtccaggaacctgggaaaggtcggctccaagtgttgcaagcatcccgaggctaagcgcatgccctgcgccgaggactacttgtccgtggtgctgaatcagctgtgcgtgctccatgaaaagaccccagtgtccgacagagtgaccaagtgctgtaccgaatcgctcgtgaaccggcggccgtgcttttccgcactggaggtggacgaaacctacgtgccgaaggagttcaacgcagaaaccttcactttccacgccgacatctgcactctgtccgagaaggagcggcagattaagaagcagactgccctggtggagcttgtgaaacacaagcctaaggccaccaaagagcagctgaaggccgtcatggatgatttcgcggccttcgtggaaaagtgttgtaaagcggacgacaaggagacttgcttcgccgaagaaggaaagaagctcgtggcagcgtcacaggccgctctgggcctcgctagcgcacctgcccccgctccagccccagctcctgcacctgctccagcaccagctcctgcaccagctccagcccctgcacctgcacccgctccagccccagctcctgcacctgctccagcaccaggtaccgcgcgcaacggggaccactgtccgctcgggcccgggcgttgctgccgtctgcacacggtccgcgcgtcgctggaagacctgggctgggccgattgggtgctgtcgccacgggaggtgcaagtgaccatgtgcatcggcgcgtgcccgagccagttccgggcggcaaacatgcacgcgcagatcaagacgagcctgcaccgcctgaagcccgacacggtgccagcgccctgctgcgtgcccgccagctacaatcccatggtgctcattcaaaagaccgacaccggggtgtcgctccagacctatgatgacttgttagccaaagactgccactgcata

SEQ ID NO: 82 - DNA, 2163 bp, Artificial Sequence
  fusion protein coding sequence
gatgctcataagtccgaagtcgcccacagattcaaggacctcggagaagaaaattttaaggccctcgtgcttatcgccttcgcccaatacctccagcagtccccgttcgaggaccacgtgaagctcgtgaacgaagtgaccgagtttgccaagacttgtgtggcggatgaatccgccgagaactgcgacaagagcctccacacgctgttcggcgacaagctgtgcaccgtcgccacgctgagagaaacttacggagagatggccgactgctgcgcaaagcaggagccggaacggaacgaatgcttcctgcaacataaggacgataaccctaacttgcctcgcctggtccgccctgaggtcgacgtgatgtgcaccgcgttccacgacaacgaggaaacctttcttaagaagtacctgtacgagattgcgcggaggcacccttatttctacgcccccgaactgttgttcttcgccaagcggtacaaggctgcctttaccgaatgctgccaggccgccgataaggcggcttgcctgctgccgaagctcgacgagttgcgcgatgaggggaaggcgtcctccgctaagcagcggctgaaatgtgcgagcctccagaagttcggggagcgcgccttcaaggcctgggccgtggcgcgcctgtctcaacggttcccgaaggccgagttcgccgaagtgtcgaagctggtcaccgacctgacgaaagtgcacaccgaatgttgtcacggcgatctgctggaatgcgccgatgacagagccgatttggccaagtacatctgcgaaaaccaggacagcatttcgtcaaagctgaaggaatgctgcgaaaagcccttgctggaaaagtcccactgcatcgcggaagtggagaacgacgagatgcccgccgacctcccgtccctggccgccgatttcgtggagtcgaaggatgtgtgcaagaactacgcagaagccaaggacgtgttcctgggaatgtttctgtatgagtacgcccgccgccacccggactactcggtcgtgctcctgctgcgactggcaaagacctacgaaaccactctggagaagtgctgcgccgccgcggacccgcacgagtgctacgcaaaggtgttcgacgagttcaagccacttgtcgaggagcctcagaacctgatcaagcagaactgcgaactgttcgagcagctgggagagtacaaattccagaacgcgcttctcgtgcgctacaccaagaaggtcccccaggtgtccactccgaccctggtggaagtgtccaggaacctgggaaaggtcggctccaagtgttgcaagcatcccgaggctaagcgcatgccctgcgccgaggactacttgtccgtggtgctgaatcagctgtgcgtgctccatgaaaagaccccagtgtccgacagagtgaccaagtgctgtaccgaatcgctcgtgaaccggcggccgtgcttttccgcactggaggtggacgaaacctacgtgccgaaggagttcaacgcagaaaccttcactttccacgccgacatctgcactctgtccgagaaggagcggcagattaagaagcagactgccctggtggagcttgtgaaacacaagcctaaggccaccaaagagcagctgaaggccgtcatggatgatttcgcggccttcgtggaaaagtgttgtaaagcggacgacaaggagacttgcttcgccgaagaaggaaagaagctcgtggcagcgtcacaggccgctctgggcctcgctagcgaagcagcagccaaagaagcagccgcaaaagaagcagccgctaaggaggccgcagcaaagggtaccgcgcgcaacggggaccactgtccgctcgggcccgggcgttgctgccgtctgcacacggtccgcgcgtcgctggaagacctgggctgggccgattgggtgctgtcgccacgggaggtgcaagtgaccatgtgcatcggcgcgtgcccgagccagttccgggcggcaaacatgcacgcgcagatcaagacgagcctgcaccgcctgaagcccgacacggtgccagcgccctgctgcgtgcccgccagctacaatcccatggtgctcattcaaaagaccgacaccggggtgtcgctccagacctatgatgacttgttagccaaagactgccactgcata

SEQ ID NO: 83 - DNA, 2157 bp, Artificial Sequence
  fusion protein coding sequence
gacgcccacaagagcgaggtggcccaccggttcaaggacctgggcgaggagaacttcaaggccctggtgctgatcgccttcgcccagtacctgcagcagtcccccttcgaggaccacgtgaagctggtgaacgaggtgaccgagttcgccaagacctgcgtggccgacgagagcgccgagaactgcgacaagagcctgcacaccctgttcggcgacaagctgtgcaccgtggccaccctgcgggagacctacggcgagatggccgactgctgcgccaagcaggagcccgagcggaacgagtgcttcctgcagcacaaggacgacaaccccaacctgccccggctggtgcggcccgaggtggacgtgatgtgcaccgccttccacgacaacgaggagaccttcctgaagaagtacctgtacgagatcgcccggcggcacccctacttctacgcccccgagctgctgttcttcgccaagcggtacaaggccgccttcaccgagtgctgccaggccgccgacaaggccgcctgcctgctgcccaagctggacgagctgcgggacgagggcaaggccagcagcgccaagcagcggctgaagtgcgccagcctgcagaagttcggcgagcgggccttcaaggcctgggccgtggcccggctgagccagcggttccccaaggccgagttcgccgaggtgagcaagctggtgaccgacctgaccaaggtgcacaccgagtgctgccacggcgacctgctggagtgcgccgacgaccgggccgacctggccaagtacatctgcgagaaccaggacagcatcagcagcaagctgaaggagtgctgcgagaagcccctgctggagaagagccactgcatcgccgaggtggagaacgacgagatgcccgccgacctgcccagcctggccgccgacttcgtggagagcaaggacgtgtgcaagaactacgccgaggccaaggacgtgttcctgggcatgttcctgtacgagtacgcccggcggcaccccgactacagcgtggtgctgctgctgcggctggccaagacctacgagaccaccctggagaagtgctgcgccgccgccgacccccacgagtgctacgccaaggtgttcgacgagttcaagcccctggtggaggagccccagaacctgatcaagcagaactgcgagctgttcgagcagctgggcgagtacaagttccagaacgccctgctggtgcggtacaccaagaaggtgccccaggtgagcacccccaccctggtggaggtgagccggaacctgggcaaggtgggcagcaagtgctgcaagcaccccgaggccaagcggatgccctgcgccgaggactacctgagcgtggtgctgaaccagctgtgcgtgctgcacgagaagacccccgtgagcgaccgggtgaccaagtgctgcaccgagagcctggtgaaccggcggccctgcttcagcgccctggaggtggacgagacctacgtgcccaaggagttcaacgccgagaccttcaccttccacgccgacatctgcaccctgagcgagaaggagcggcagatcaagaagcagaccgccctggtggagctggtgaagcacaagcccaaggccaccaaggagcagctgaaggccgtgatggacgacttcgccgccttcgtggagaagtgctgcaaggccgacgacaaggagacctgcttcgccgaggagggcaagaagctggtggccgccagccaggccgccctgggcctgggcagcggcggcggcggcagcggcggcggcggatctggtggaggtggcagtggaggagggggatccgcgcgcaacggggaccactgtccgctcgggcccgggcgttgctgccgtctgcacacggtccgcgcgtcgctggaagacctgggctgggccgattgggtgctgtcgccacgggaggtgcaagtgaccatgtgcatcggcgcgtgcccgagccagttccgggcggcaaacatgcacgcgcagatcaagacgagcctgcaccgcctgaagcccgacacggtgccagcgccctgctgcgtgcccgccagctacaatcccatggtgctcattcaaaagaccgacaccggggtgtcgctccagacctatgatgacttgttagccaaagactgccactgcata

SEQ ID NO: 84 - DNA, 2217 bp, Artificial Sequence
  fusion protein coding sequence
gatgcacacaagagtgaggttgctcatcggtttaaagatttgggagaagaaaatttcaaagccttggtgttgattgcctttgctcagtatcttcagcaggccccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagctcgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttaggcagcggcggcggcggcagcggcggcggcggatctggtggaggtggcagtggaggagggggatccggcggcggcggcagcggcggcggcggatctggtggaggtggcagtggaggagggggatccgcgcgcaacggggaccactgtccgctcgggcccgggcgttgctgccgtctgcacacggtccgcgcgtcgctggaagacctgggctgggccgattgggtgctgtcgccacgggaggtgcaagtgaccatgtgcatcggcgcgtgcccgagccagttccgggcggcaaacatgcacgcgcagatcaagacgagcctgcaccgcctgaagcccgacacggtgccagcgccctgctgcgtgcccgccagctacaatcccatggtgctcattcaaaagaccgacaccggggtgtcgctccagacctatgatgacttgttagccaaagactgccactgcata

SEQ ID NO: 85 - DNA, 2151 bp, Artificial Sequence
  fusion protein coding sequence
gatgcacacaagagtgaggttgctcatcggtttaaagatttgggagaagaaaatttcaaagccttggtgttgattgcctttgctcagtatcttcagcaggccccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagctcgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttagcacctgcccccgctccagcacccgccccagcccctgctcccgcaccagctcctgcaccagcgcgcaacggggaccactgtccgctcgggcccgggcgttgctgccgtctgcacacggtccgcgcgtcgctggaagacctgggctgggccgattgggtgctgtcgccacgggaggtgcaagtgaccatgtgcatcggcgcgtgcccgagccagttccgggcggcaaacatgcacgcgcagatcaagacgagcctgcaccgcctgaagcccgacacggtgccagcgccctgctgcgtgcccgccagctacaatcccatggtgctcattcaaaagaccgacaccggggtgtcgctccagacctatgatgacttgttagccaaagactgccactgcata

SEQ ID NO: 86 - DNA, 2157 bp, Artificial Sequence
  fusion protein coding sequence
gatgcacacaagagtgaggttgctcatcggtttaaagatttgggagaagaaaatttcaaagccttggtgttgattgcctttgctcagtatcttcagcagtccccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagctcgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttaggcagcggcggcggcggcagcggcggcggcggatctggtggaggtggcagtggaggagggggatccgcgcgcaacggggaccactgtccgctcgggcccgggcgttgctgccgtctgcacacggtccgcgcgtcgctggaagacctgggctgggccgattgggtgctgtcgccacgggaggtgcaagtgaccatgtgcatcggcgcgtgcccgagccagttccgggcggcaaacatgcacgcgcagatcaagacgagcctgcaccgcctgaagcccgacacggtgccagcgccctgctgcgtgcccgccagctacaatcccatggtgctcattcaaaagaccgacaccggggtgtcgctccagacctatgatgacttgttagccaaagactgccactgcata

SEQ ID NO: 87 - DNA, 2157 bp, Artificial Sequence
  fusion protein coding sequence
gacgcccacaagagcgaggtggcccacagattcaaggacctgggcgaggaaaacttcaaggccctggtgctgatcgccttcgcccagtacctgcagcagagccccttcgaggaccacgtgaagctggtcaacgaagtgaccgagttcgccaagacctgcgtggccgacgagagcgccgagaactgcgacaagagcctgcacaccctgttcggcgacaagctgtgcaccgtggccaccctgcgggaaacctacggcgagatggccgactgctgcgccaagcaggaacccgagcggaacgagtgcttcctgcagcacaaggacgacaaccccaacctgcccagactcgtgcggcccgaggtggacgtgatgtgcaccgccttccacgacaacgaggaaaccttcctgaagaagtacctgtacgagatcgccagacggcacccctacttctacgcccccgagctgctgttcttcgccaagcggtacaaggccgccttcaccgagtgctgccaggccgccgataaggccgcctgcctgctgcccaagctggacgagctgagagatgagggcaaggccagctccgccaagcagcggctgaagtgcgccagcctgcagaagttcggcgagcgggcctttaaggcttgggctgtggcccggctgagccagagattccccaaggccgagtttgccgaggtgtccaagctggtcaccgacctgaccaaggtgcacaccgagtgttgtcacggcgacctgctggaatgcgccgacgacagagccgacctggccaagtacatctgcgagaaccaggacagcatcagcagcaagctgaaagagtgctgcgagaagcccctgctggaaaagagccactgtatcgccgaggtggaaaacgacgagatgcccgctgacctgcccagcctggccgccgacttcgtggaaagcaaggacgtgtgcaagaactacgccgaggccaaggatgtgttcctgggcatgttcctgtatgagtacgcccgcagacaccccgactacagcgtggtgctgctgctgcggctggccaagacctacgagacaaccctggaaaagtgctgcgccgctgccgacccccacgagtgctacgccaaggtgttcgacgagttcaagcctctggtggaagaaccccagaacctgatcaagcagaactgcgagctgttcgagcagctgggcgagtacaagttccagaacgccctgctcgtgcggtacaccaagaaagtgccccaggtgtccacccccaccctggtcgaagtgtcccggaacctgggcaaagtgggcagcaagtgctgcaagcaccctgaggccaagcggatgccctgcgccgaggactacctgtccgtggtgctgaaccagctgtgcgtgctgcacgagaaaacccccgtgtccgacagagtgaccaagtgctgtaccgagagcctggtcaacagacggccctgcttcagcgccctggaagtggacgagacatacgtgcccaaagagttcaacgccgagacattcaccttccacgccgacatctgcaccctgagcgagaaagagcggcagatcaagaagcagaccgccctggtcgagctggtcaagcacaagcccaaggccaccaaagaacagctgaaggccgtgatggacgacttcgccgccttcgtcgagaagtgttgcaaggccgacgacaaagagacatgcttcgccgaagagggcaagaaactggtggccgcctctcaggccgccctgggactgggatctggcggcggaggaagcggaggcggaggatctgggggaggcggctctggcggagggggatccgccagaaatggcgaccactgtcccctgggccctggccggtgttgcagactgcacacagtgcgggccagcctggaagatctgggctgggccgattgggtgctgagccccagagaagtgcaggtcacaatgtgcatcggcgcctgccccagccagttcagagccgccaacatgcacgcccagatcaagaccagcctgcaccggctgaagcccgacaccgtgcctgccccttgttgcgtgcccgccagctacaaccccatggtgctgattcagaaaaccgacaccggcgtgtccctgcagacctacgacgatctgctggccaaggactgccactgcatc

SEQ ID NO: 88 - DNA, 2157 bp, Artificial Sequence
  fusion protein coding sequence
gatgcacacaagagtgaggttgctcatcggtttaaagatttgggagaagaaaatttcaaagccttggtgttgattgcctttgctcagtatcttcagcaggccccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagctcgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttaggcagcggcggcggcggcagcggcggcggcggatctggtggaggtggcagtggaggagggggatccgcgcgcaacggggaccactgtccgctcgggcccgggcgttgctgccgtctgcacacggtccgcgcgtcgctggaagacctgggctgggccgattgggtgctgtcgccacgggaggtgcaagtgaccatgtgcatcggcgcgtgcccgagccagttccgggcggcaaacatgcacgcgcagatcaagacgagcctgcaccgcctgaagcccgacacggtgccagcgccctgctgcgtgcccgccagctacaatcccatggtgctcattcaaaagaccgacaccggggtgtcgctccagacctatgatgacttgttagccaaagactgccactgcata

SEQ ID NO: 89 - DNA, 2151 bp, Artificial Sequence
  fusion protein coding sequence
gatgcacacaagagtgaggttgctcatcggtttaaagatttgggagaagaaaatttcaaagccttggtgttgattgcctttgctcagtatcttcagcagtccccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagctcgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttagcacctgcccccgctccagcacccgccccagcccctgctcccgcaccagctcctgcaccagcgcgcaacggggaccactgtccgctcgggcccgggcgttgctgccgtctgcacacggtccgcgcgtcgctggaagacctgggctgggccgattgggtgctgtcgccacgggaggtgcaagtgaccatgtgcatcggcgcgtgcccgagccagttccgggcggcaaacatgcacgcgcagatcaagacgagcctgcaccgcctgaagcccgacacggtgccagcgccctgctgcgtgcccgccagctacaatcccatggtgctcattcaaaagaccgacaccggggtgtcgctccagacctatgatgacttgttagccaaagactgccactgcata

SEQ ID NO: 90 - DNA, 2217 bp, Artificial Sequence
  fusion protein coding sequence
gatgcacacaagagtgaggttgctcatcggtttaaagatttgggagaagaaaatttcaaagccttggtgttgattgcctttgctcagtatcttcagcagtccccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagctcgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttaggcagcggcggcggcggcagcggcggcggcggatctggtggaggtggcagtggaggagggggatccggcggcggcggcagcggcggcggcggatctggtggaggtggcagtggaggagggggatccgcgcgcaacggggaccactgtccgctcgggcccgggcgttgctgccgtctgcacacggtccgcgcgtcgctggaagacctgggctgggccgattgggtgctgtcgccacgggaggtgcaagtgaccatgtgcatcggcgcgtgcccgagccagttccgggcggcaaacatgcacgcgcagatcaagacgagcctgcaccgcctgaagcccgacacggtgccagcgccctgctgcgtgcccgccagctacaatcccatggtgctcattcaaaagaccgacaccggggtgtcgctccagacctatgatgacttgttagccaaagactgccactgcata

SEQ ID NO: 91 - DNA, 2157 bp, Artificial Sequence
  fusion protein coding sequence
gacgcccacaagagcgaggtggcccaccggttcaaggacctgggcgaggagaacttcaaggccctggtgctgatcgccttcgcccagtacctgcagcagtcccccttcgaggaccacgtgaagctggtgaacgaggtgaccgagttcgccaagacctgcgtggccgacgagagcgccgagaactgcgacaagagcctgcacaccctgttcggcgacaagctgtgcaccgtggccaccctgcgggagacctacggcgagatggccgactgctgcgccaagcaggagcccgagcggaacgagtgcttcctgcagcacaaggacgacaaccccaacctgccccggctggtgcggcccgaggtggacgtgatgtgcaccgccttccacgacaacgaggagaccttcctgaagaagtacctgtacgagatcgcccggcggcacccctacttctacgcccccgagctgctgttcttcgccaagcggtacaaggccgccttcaccgagtgctgccaggccgccgacaaggccgcctgcctgctgcccaagctggacgagctgcgggacgagggcaaggccagcagcgccaagcagcggctgaagtgcgccagcctgcagaagttcggcgagcgggccttcaaggcctgggccgtggcccggctgagccagcggttccccaaggccgagttcgccgaggtgagcaagctggtgaccgacctgaccaaggtgcacaccgagtgctgccacggcgacctgctggagtgcgccgacgaccgggccgacctggccaagtacatctgcgagaaccaggacagcatcagcagcaagctgaaggagtgctgcgagaagcccctgctggagaagagccactgcatcgccgaggtggagaacgacgagatgcccgccgacctgcccagcctggccgccgacttcgtggagagcaaggacgtgtgcaagaactacgccgaggccaaggacgtgttcctgggcatgttcctgtacgagtacgcccggcggcaccccgactacagcgtggtgctgctgctgcggctggccaagacctacgagaccaccctggagaagtgctgcgccgccgccgacccccacgagtgctacgccaaggtgttcgacgagttcaagcccctggtggaggagccccagaacctgatcaagcagaactgcgagctgttcgagcagctgggcgagtacaagttccagaacgccctgctggtgcggtacaccaagaaggtgccccaggtgagcacccccaccctggtggaggtgagccggaacctgggcaaggtgggcagcaagtgctgcaagcaccccgaggccaagcggatgccctgcgccgaggactacctgagcgtggtgctgaaccagctgtgcgtgctgcacgagaagacccccgtgagcgaccgggtgaccaagtgctgcaccgagagcctggtgaaccggcggccctgcttcagcgccctggaggtggacgagacctacgtgcccaaggagttcaacgccgagaccttcaccttccacgccgacatctgcaccctgagcgagaaggagcggcagatcaagaagcagaccgccctggtggagctggtgaagcacaagcccaaggccaccaaggagcagctgaaggccgtgatggacgacttcgccgccttcgtggagaagtgctgcaaggccgacgacaaggagacctgcttcgccgaggagggcaagaagctggtggccgccagccaggccgccctgggcctgggcagcggcggcggcggcagcggcggcggcggatctggtggaggtggcagtggaggagggggatccgctcgcaacggtgaccactgccctctgggtcctggtcgctgctgccgcctgcacaccgttcgcgcttctctggaagacctgggttgggctgactgggttctgtctcctcgcgaagttcaggttaccatgtgcatcggtgcttgcccttctcagttccgcgctgctaacatgcacgcttggatcaaaacctctctgcaccgcctgaaacctgacaccgttcctgctccttgctgcgttcctgcttcttacaaccctatggttctgatccagaaaaccgacaccggtgtttctctgcagacctacgacgacctgctggctaaagactgccactgcatc

SEQ ID NO: 92 - protein, 735 aa, Artificial Sequence
  fusion protein coding sequence
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGSGGGGSGGGGSGGGGSGGGGSGGGGSGGGGSGGGGSGGGGSDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 93 - protein, 734 aa, Artificial Sequence
  fusion protein coding sequence
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGSGGGGSGGGGSGGGGSGGGGSGGGGSGGGGSGGGGSGGGGSHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 94 - protein, 735 aa, Artificial Sequence
  fusion protein coding sequence
EFHHHHHHDAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGSGGGGSGGGGSGGGGSGGGGSLEVLFQGPARNGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 95 - DNA, 2205 bp, Artificial Sequence
  fusion protein coding sequence
gatgcacacaagagtgaggttgctcatcggtttaaagatttgggagaagaaaatttcaaagccttggtgttgattgcctttgctcagtatcttcagcagtccccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagctcgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttagggcttggaagcggcggaggggggagtggcggcggtggctccggggggggcggatccggcggagggggcagcgggggtggagggagtggcgggggaggatcagggggaggaggatcaggagggggcggaagtgatcattgccctctcgggcccggacggtgttgccgcctccacactgtgagggcttcacttgaagaccttggatgggccgactgggtgctgtccccaagagaggtacaagtcacaatgtgtattggcgcctgccccagccagtttcgcgccgctaacatgcacgcccagataaaaaccagcctgcaccgcctgaagcccgacacggtgccagcgccctgctgcgtgcccgccagctacaatcccatggtgctcattcaaaagaccgacaccggggtgtcgctccagacctatgatgacttgttagccaaagactgccactgcata

SEQ ID NO: 96 - DNA, 327 bp, Artificial Sequence
  GDF15 variant
ggggaccactgtccgctcgggcccgggcgttgctgccgtctgcacacggtccgcgcgtcgctggaagacctgggctgggccgattgggtgctgtcgccacgggaggtgcaagtgaccatgtgcatcggcgcgtgcccgagccagttccgggcggcaaacatgcacgcgcagatcaagacgagcctgcaccgcctgaagcccgacacggtgccagcgccctgctgcgtgcccgccagctacaatcccatggtgctcattcaaaagaccgacaccggggtgtcgctccagacctatgatgacttgttagccaaagactgccactgcata

SEQ ID NO: 97 - DNA, 324 bp, Artificial Sequence
  GDF15 variant
gaccactgtccgctcgggcccgggcgttgctgccgtctgcacacggtccgcgcgtcgctggaagacctgggctgggccgattgggtgctgtcgccacgggaggtgcaagtgaccatgtgcatcggcgcgtgcccgagccagttccgggcggcaaacatgcacgcgcagatcaagacgagcctgcaccgcctgaagcccgacacggtgccagcgccctgctgcgtgcccgccagctacaatcccatggtgctcattcaaaagaccgacaccggggtgtcgctccagacctatgatgacttgttagccaaagactgccactgcata

SEQ ID NO: 98 - DNA, 324 bp, Artificial Sequence
  GDF15 variant
gatcattgccctctcgggcccggacggtgttgccgcctccacactgtgagggcttcacttgaagaccttggatgggccgactgggtgctgtccccaagagaggtacaagtcacaatgtgtattggcgcctgccccagccagtttcgcgccgctaacatgcacgcccagataaaaaccagcctgcaccgcctgaagcccgacacggtgccagcgccctgctgcgtgcccgccagctacaatcccatggtgctcattcaaaagaccgacaccggggtgtcgctccagacctatgatgacttgttagccaaagactgccactgcata

SEQ ID NO: 99 - DNA, 324 bp, Artificial Sequence
  GDF15 variant
gatcattgtccccttggaccgggtagatgctgtcgcctgcacactgtgcgggcttcactggaggacctcggctgggctgactgggtgctgtccccacgggaggtgcaagtgaccatgtgcatcggcgcctgtccttcgcaattccgggccgcgaatatgcacgcccagatcaagacctccctgcatcgcctcaagcccgacactgtgcctgctccatgctgtgtgccggcctcctataaccccatggtgctgatccagaaaaccgataccggcgtcagcctgcagacgtatgatgatctgctggccaaggactgccattgcatc

SEQ ID NO: 100 - DNA, 321 bp, Artificial Sequence
  GDF15 variant
cactgtccgctcgggcccgggcgttgctgccgtctgcacacggtccgcgcgtcgctggaagacctgggctgggccgattgggtgctgtcgccacgggaggtgcaagtgaccatgtgcatcggcgcgtgcccgagccagttccgggcggcaaacatgcacgcgcagatcaagacgagcctgcaccgcctgaagcccgacacggtgccagcgccctgctgcgtgcccgccagctacaatcccatggtgctcattcaaaagaccgacaccggggtgtcgctccagacctatgatgacttgttagccaaagactgccactgcata

SEQ ID NO: 101 - DNA, 321 bp, Artificial Sequence
  GDF15 variant
cattgccctctcgggcccggacggtgttgccgcctccacactgtgagggcttcacttgaagaccttggatgggccgactgggtgctgtccccaagagaggtacaagtcacaatgtgtattggcgcctgccccagccagtttcgcgccgctaacatgcacgcccagataaaaaccagcctgcaccgcctgaagcccgacacggtgccagcgccctgctgcgtgcccgccagctacaatcccatggtgctcattcaaaagaccgacaccggggtgtcgctccagacctatgatgacttgttagccaaagactgccactgcata

SEQ ID NO: 102 - DNA, 321 bp, Artificial Sequence
  GDF15 variant
cattgtccccttggaccgggtagatgctgtcgcctgcacactgtgcgggcttcactggaggacctcggctgggctgactgggtgctgtccccacgggaggtgcaagtgaccatgtgcatcggcgcctgtccttcgcaattccgggccgcgaatatgcacgcccagatcaagacctccctgcatcgcctcaagcccgacactgtgcctgctccatgctgtgtgccggcctcctataaccccatggtgctgatccagaaaaccgataccggcgtcagcctgcagacgtatgatgatctgctggccaaggactgccattgcatc

SEQ ID NO: 103 - DNA, 318 bp, Artificial Sequence
  GDF15 variant
tgtccgctcgggcccgggcgttgctgccgtctgcacacggtccgcgcgtcgctggaagacctgggctgggccgattgggtgctgtcgccacgggaggtgcaagtgaccatgtgcatcggcgcgtgcccgagccagttccgggcggcaaacatgcacgcgcagatcaagacgagcctgcaccgcctgaagcccgacacggtgccagcgccctgctgcgtgcccgccagctacaatcccatggtgctcattcaaaagaccgacaccggggtgtcgctccagacctatgatgacttgttagccaaagactgccactgcata

SEQ ID NO: 104 - DNA, 318 bp, Artificial Sequence
  GDF15 variant
tgccctctcgggcccggacggtgttgccgcctccacactgtgagggcttcacttgaagaccttggatgggccgactgggtgctgtccccaagagaggtacaagtcacaatgtgtattggcgcctgccccagccagtttcgcgccgctaacatgcacgcccagataaaaaccagcctgcaccgcctgaagcccgacacggtgccagcgccctgctgcgtgcccgccagctacaatcccatggtgctcattcaaaagaccgacaccggggtgtcgctccagacctatgatgacttgttagccaaagactgccactgcata

SEQ ID NO: 105 - DNA, 318 bp, Artificial Sequence
  GDF15 variant
tgtccccttggaccgggtagatgctgtcgcctgcacactgtgcgggcttcactggaggacctcggctgggctgactgggtgctgtccccacgggaggtgcaagtgaccatgtgcatcggcgcctgtccttcgcaattccgggccgcgaatatgcacgcccagatcaagacctccctgcatcgcctcaagcccgacactgtgcctgctccatgctgtgtgccggcctcctataaccccatggtgctgatccagaaaaccgataccggcgtcagcctgcagacgtatgatgatctgctggccaaggactgccattgcatc

SEQ ID NO: 106 - DNA, 294 bp, Artificial Sequence
  GDF15 variant
tgccgtctgcacacggtccgcgcgtcgctggaagacctgggctgggccgattgggtgctgtcgccacgggaggtgcaagtgaccatgtgcatcggcgcgtgcccgagccagttccgggcggcaaacatgcacgcgcagatcaagacgagcctgcaccgcctgaagcccgacacggtgccagcgccctgctgcgtgcccgccagctacaatcccatggtgctcattcaaaagaccgacaccggggtgtcgctccagacctatgatgacttgttagccaaagactgccactgcata

SEQ ID NO: 107 - DNA, 294 bp, Artificial Sequence
  GDF15 variant
tgccgcctccacactgtgagggcttcacttgaagaccttggatgggccgactgggtgctgtccccaagagaggtacaagtcacaatgtgtattggcgcctgccccagccagtttcgcgccgctaacatgcacgcccagataaaaaccagcctgcaccgcctgaagcccgacacggtgccagcgccctgctgcgtgcccgccagctacaatcccatggtgctcattcaaaagaccgacaccggggtgtcgctccagacctatgatgacttgttagccaaagactgccactgcata

SEQ ID NO: 108 - DNA, 294 bp, Artificial Sequence
  GDF15 variant
tgtcgcctgcacactgtgcgggcttcactggaggacctcggctgggctgactgggtgctgtccccacgggaggtgcaagtgaccatgtgcatcggcgcctgtccttcgcaattccgggccgcgaatatgcacgcccagatcaagacctccctgcatcgcctcaagcccgacactgtgcctgctccatgctgtgtgccggcctcctataaccccatggtgctgatccagaaaaccgataccggcgtcagcctgcagacgtatgatgatctgctggccaaggactgccattgcatc

SEQ ID NO: 109 - DNA, 2217 bp, Artificial Sequence
  fusion protein coding sequence
gatgcgcacaagtcggaagtggcccatcgctttaaggacctgggagaagagaacttcaaggccctggtcctgatcgcgttcgcccagtacctccagcagtccccgtttgaggaccacgtcaagcttgtgaacgaagtgaccgagttcgcaaagacttgtgtggccgatgagtccgccgaaaactgcgacaagtccctgcacaccttgttcggagacaagctgtgcaccgtcgcgactttgcgggagacttacggcgaaatggcggactgctgcgcaaagcaggagcccgaaaggaacgagtgcttcctgcaacacaaggacgacaacccgaaccttccgagactcgtgcggcctgaggtcgacgtgatgtgcactgcattccatgataacgaagaaacattcctgaagaagtacctgtatgaaattgccagacgccacccgtacttctacgcccccgaactgctgttcttcgccaagagatacaaggccgcctttaccgaatgttgtcaagccgccgataaggcagcgtgcctgctgccgaagttggacgagctcagggacgaaggaaaggcctcgtccgccaagcagaggctgaagtgcgcgtcgctccagaagtttggagagcgggcttttaaggcctgggcagtggctaggttgagccagaggttccccaaggcggagtttgccgaagtgtccaagctcgtgactgacctgactaaagtccataccgaatgctgccacggcgatctgctcgaatgcgcagatgaccgggcggatttggccaagtacatttgcgaaaaccaagactccataagctccaagctgaaggagtgctgtgaaaagcctctgctcgagaagtcccactgtatcgccgaggtggagaacgacgaaatgccggcagacctccctagcctggcagccgacttcgtcgaatccaaggacgtgtgcaagaactacgccgaagcgaaggacgtgttcctgggaatgttcctgtacgagtacgccagacggcatccagactactccgtggtgcttctcttgcggctggccaagacttatgaaacgaccctggagaaatgttgcgctgctgctgacccacacgagtgctacgccaaagtgttcgacgagtttaagcctctcgtggaggaaccccagaacctcatcaagcagaactgcgaacttttcgagcagctcggggagtacaagttccaaaacgcgctgcttgtccgctacaccaagaaagtgccgcaagtgtccacaccgaccctcgtggaagtgtccaggaacctgggcaaagtcggaagcaaatgttgcaagcaccccgaagccaagcgcatgccgtgcgcagaggactacctttcggtggtgttgaaccagctctgcgtcctgcacgaaaagaccccggtgtcagaccgcgtgaccaagtgctgtaccgaaagcctcgtgaatcggcgcccctgcttctcggccctggaggtggacgaaacttacgtgccgaaagagttcaacgcggaaaccttcacctttcatgccgatatctgcaccctgtccgagaaggagcggcagatcaagaagcagaccgccctggtggagcttgtgaaacacaagccgaaggccactaaggaacagctgaaggccgtcatggacgatttcgctgccttcgtcgagaagtgctgcaaggccgacgacaaggagacttgcttcgctgaagaagggaagaagcttgtggccgctagccaggctgcactgggactgggtagcggtggagggggatcagggggtggtggatcgggaggaggaggatcaggaggtggcggctcaggaggaggcggatcaggcggtggaggatccggaggcggaggatcgggtggaggaggctcagcgaggaacggggatcattgtccccttggaccgggtagatgctgtcgcctgcacactgtgcgggcttcactggaggacctcggctgggctgactgggtgctgtccccacgggaggtgcaagtgaccatgtgcatcggcgcctgtccttcgcaattccgggccgcgaatatgcacgcccagatcaagacctccctgcatcgcctcaagcccgacactgtgcctgctccatgctgtgtgccggcctcctataaccccatggtgctgatccagaaaaccgataccggcgtcagcctgcagacgtatgatgatctgctggccaaggactgccattgcatc

SEQ ID NO: 110 - DNA, 2205 bp, Artificial Sequence
  fusion protein coding sequence
gatgcgcacaagtcggaagtggcccatcgctttaaggacctgggagaagagaacttcaaggccctggtcctgatcgcgttcgcccagtacctccagcagtccccgtttgaggaccacgtcaagcttgtgaacgaagtgaccgagttcgcaaagacttgtgtggccgatgagtccgccgaaaactgcgacaagtccctgcacaccttgttcggagacaagctgtgcaccgtcgcgactttgcgggagacttacggcgaaatggcggactgctgcgcaaagcaggagcccgaaaggaacgagtgcttcctgcaacacaaggacgacaacccgaaccttccgagactcgtgcggcctgaggtcgacgtgatgtgcactgcattccatgataacgaagaaacattcctgaagaagtacctgtatgaaattgccagacgccacccgtacttctacgcccccgaactgctgttcttcgccaagagatacaaggccgcctttaccgaatgttgtcaagccgccgataaggcagcgtgcctgctgccgaagttggacgagctcagggacgaaggaaaggcctcgtccgccaagcagaggctgaagtgcgcgtcgctccagaagtttggagagcgggcttttaaggcctgggcagtggctaggttgagccagaggttccccaaggcggagtttgccgaagtgtccaagctcgtgactgacctgactaaagtccataccgaatgctgccacggcgatctgctcgaatgcgcagatgaccgggcggatttggccaagtacatttgcgaaaaccaagactccataagctccaagctgaaggagtgctgtgaaaagcctctgctcgagaagtcccactgtatcgccgaggtggagaacgacgaaatgccggcagacctccctagcctggcagccgacttcgtcgaatccaaggacgtgtgcaagaactacgccgaagcgaaggacgtgttcctgggaatgttcctgtacgagtacgccagacggcatccagactactccgtggtgcttctcttgcggctggccaagacttatgaaacgaccctggagaaatgttgcgctgctgctgacccacacgagtgctacgccaaagtgttcgacgagtttaagcctctcgtggaggaaccccagaacctcatcaagcagaactgcgaacttttcgagcagctcggggagtacaagttccaaaacgcgctgcttgtccgctacaccaagaaagtgccgcaagtgtccacaccgaccctcgtggaagtgtccaggaacctgggcaaagtcggaagcaaatgttgcaagcaccccgaagccaagcgcatgccgtgcgcagaggactacctttcggtggtgttgaaccagctctgcgtcctgcacgaaaagaccccggtgtcagaccgcgtgaccaagtgctgtaccgaaagcctcgtgaatcggcgcccctgcttctcggccctggaggtggacgaaacttacgtgccgaaagagttcaacgcggaaaccttcacctttcatgccgatatctgcaccctgtccgagaaggagcggcagatcaagaagcagaccgccctggtggagcttgtgaaacacaagccgaaggccactaaggaacagctgaaggccgtcatggacgatttcgctgccttcgtcgagaagtgctgcaaggccgacgacaaggagacttgcttcgctgaagaagggaagaagcttgtggccgctagccaggctgcactgggactgggtagcggtggagggggatcagggggtggtggatcgggaggaggaggatcaggaggtggcggctcaggaggaggcggatcaggcggtggaggatccggaggcggaggatcgggtggaggaggctcagatcattgtccccttggaccgggtagatgctgtcgcctgcacactgtgcgggcttcactggaggacctcggctgggctgactgggtgctgtccccacgggaggtgcaagtgaccatgtgcatcggcgcctgtccttcgcaattccgggccgcgaatatgcacgcccagatcaagacctccctgcatcgcctcaagcccgacactgtgcctgctccatgctgtgtgccggcctcctataaccccatggtgctgatccagaaaaccgataccggcgtcagcctgcagacgtatgatgatctgctggccaaggactgccattgcatc

SEQ ID NO: 111 - protein, 734 aa, Artificial Sequence; identical to SEQ ID NO: 93
  fusion protein

SEQ ID NO: 112 - protein, 725 aa, Artificial Sequence
  fusion protein
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGSGGGGSGGGGSGGGGSGGGGSGGGGSGGGGSGGGGSGGGGSCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 113 - protein, 733 aa, Artificial Sequence
  fusion protein
HKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGSGGGGSGGGGSGGGGSGGGGSGGGGSGGGGSGGGGSGGGGSDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 114 - DNA, 2199 bp, Artificial Sequence
  fusion protein coding sequence
cacaagagtgaggttgctcatcggtttaaagatttgggagaagaaaatttcaaagccttggtgttgattgcctttgctcagtatcttcagcagtccccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagctcgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttagggcttggaagcggcggaggggggagtggcggcggtggctccggggggggcggatccggcggagggggcagcgggggtggagggagtggcgggggaggatcagggggaggaggatcaggagggggcggaagtgatcattgccctctcgggcccggacggtgttgccgcctccacactgtgagggcttcacttgaagaccttggatgggccgactgggtgctgtccccaagagaggtacaagtcacaatgtgtattggcgcctgccccagccagtttcgcgccgctaacatgcacgcccagataaaaaccagcctgcaccgcctgaagcccgacacggtgccagcgccctgctgcgtgcccgccagctacaatcccatggtgctcattcaaaagaccgacaccggggtgtcgctccagacctatgatgacttgttagccaaagactgccactgcata

SEQ ID NO: 115 - protein, 735 aa, Artificial Sequence
  fusion protein
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGAGGGGAGGGGAGGGGAGGGGAGGGGAGGGGAGGGGAGGGGADHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 116 - DNA, 2205 bp, Artificial Sequence
  fusion protein coding sequence
gatgcacacaagagtgaggttgctcatcggtttaaagatttgggagaagaaaatttcaaagccttggtgttgattgcctttgctcagtatcttcagcagtccccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagctcgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttagggcttggtgctggaggaggcggggcgggcggcgggggtgccggtgggggtggcgcagggggaggtggtgcgggtggtggtggggctggtgggggaggtgcaggcggtggcggtgccggggggggtggcgcggatcattgccctctcgggcccggacggtgttgccgcctccacactgtgagggcttcacttgaagaccttggatgggccgactgggtgctgtccccaagagaggtacaagtcacaatgtgtattggcgcctgccccagccagtttcgcgccgctaacatgcacgcccagataaaaaccagcctgcaccgcctgaagcccgacacggtgccagcgccctgctgcgtgcccgccagctacaatcccatggtgctcattcaaaagaccgacaccggggtgtcgctccagacctatgatgacttgttagccaaagactgccactgcata

SEQ ID NO: 117 - protein, 713 aa, Artificial Sequence
  fusion protein
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLAPAPAPAPAPAPAPAPAPAPDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 118 - DNA, 2139 bp, Artificial Sequence
  fusion protein coding sequence
gatgcacacaagagtgaggttgctcatcggtttaaagatttgggagaagaaaatttcaaagccttggtgttgattgcctttgctcagtatcttcagcagtccccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagctcgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttagggcttgcaccagcccctgcccctgcacctgcacctgctcccgcaccggctccagccccagctccggatcattgccctctcgggcccggacggtgttgccgcctccacactgtgagggcttcacttgaagaccttggatgggccgactgggtgctgtccccaagagaggtacaagtcacaatgtgtattggcgcctgccccagccagtttcgcgccgctaacatgcacgcccagataaaaaccagcctgcaccgcctgaagcccgacacggtgccagcgccctgctgcgtgcccgccagctacaatcccatggtgctcattcaaaagaccgacaccggggtgtcgctccagacctatgatgacttgttagccaaagactgccactgcata

SEQ ID NO: 119 - protein, 717 aa, Artificial Sequence
  fusion protein
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLAPAPAPAPAPAPAPAPAPAPAPAPDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 120 - DNA, 2151 bp, Artificial Sequence
  fusion protein coding sequence
gatgcacacaagagtgaggttgctcatcggtttaaagatttgggagaagaaaatttcaaagccttggtgttgattgcctttgctcagtatcttcagcagtccccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagctcgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttagggcttgcaccagcccctgcccctgcacctgcacctgctcccgcaccggctccagccccagctccggctccagctcctgatcattgccctctcgggcccggacggtgttgccgcctccacactgtgagggcttcacttgaagaccttggatgggccgactgggtgctgtccccaagagaggtacaagtcacaatgtgtattggcgcctgccccagccagtttcgcgccgctaacatgcacgcccagataaaaaccagcctgcaccgcctgaagcccgacacggtgccagcgccctgctgcgtgcccgccagctacaatcccatggtgctcattcaaaagaccgacaccggggtgtcgctccagacctatgatgacttgttagccaaagactgccactgcata

SEQ ID NO: 121 - protein, 741 aa, Artificial Sequence
  fusion protein
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGSEGKSSGSGSESKSTEGKSSGSGSESKSTEGKSSGSGSESKSTGGSDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 122 - DNA, 2223 bp, Artificial Sequence
  fusion protein coding sequence
gatgcacacaagagtgaggttgctcatcggtttaaagatttgggagaagaaaatttcaaagccttggtgttgattgcctttgctcagtatcttcagcagtccccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcacaaaatgctgtactgagagcttggtcaacaggcggccgtgcttcagcgccctcgaggtggatgagacttatgtcccaaaggagtttaatgcggaaacttttactttccacgcagacatttgcaccttgtctgaaaaggaaagacagattaagaaacagactgctcttgtggaactggtaaaacataaaccaaaagctacgaaggagcagcttaaggctgttatggatgatttcgccgcgtttgtcgagaagtgctgcaaagcggacgataaggaaacttgctttgcagaggaaggtaagaaactcgtagcggcaagtcaggctgcgcttggccttggaggcagtgaaggcaaatcctctgggagtggctctgaaagtaaatccaccgagggcaaatccagtggatctgggtctgaatctaagtctaccgaggggaagtcttctggcagtgggtcagaatctaaatctacaggcggctctgaccattgcccgttgggaccaggacgctgctgtcgccttcatacagtgcgagcgagtttggaagacctgggctgggctgactgggtgcttagccctcgggaggtccaggtcacaatgtgcattggcgcgtgtcccagtcaatttagagcagcaaatatgcacgcccaaataaaaacctccctgcataggcttaagccagatactgtccccgcaccatgctgtgtgcctgcttcttacaatcctatggtactcatccagaagaccgacacgggagttagcctccagacttatgacgacctcttggctaaagattgccattgtatt

SEQ ID NO: 123 - protein, 735 aa, Artificial Sequence
  fusion protein
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGSPGGGSPGGGSPGGGSPGGGSPGGGSPGGGSPGGGSPGGGSDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 124 - DNA, 2205 bp, Artificial Sequence
  fusion protein coding sequence
gatgcacacaagagtgaggttgctcatcggtttaaagatttgggagaagaaaatttcaaagccttggtgttgattgcctttgctcagtatcttcagcagtccccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcacgaaatgttgcacagagtcactggtcaacaggagaccttgcttctccgctcttgaggttgacgaaacgtatgtcccaaaagagttcaacgccgaaacgtttacgtttcatgcggacatatgcactctcagtgagaaggagcgacaaatcaaaaaacagactgctcttgtagagttggtaaaacacaaacctaaagcaacaaaagagcaattgaaagctgtgatggacgattttgcagctttcgtagaaaagtgctgcaaggccgacgataaagaaacctgtttcgctgaagaaggcaaaaaacttgttgcggcatctcaggccgctcttggacttgggagcccgggtggcgggtctccaggcggaggctctccgggcggaggtagtcccggagggggtagtccgggcggcggttctccaggtggaggttctcctggtggtggcagtcctggcggaggatctgatcactgtccccttgggcccgggaggtgctgccgacttcatacagttcgcgccagccttgaagatttggggtgggccgactgggtgttgagcccgagagaggtccaagtcacgatgtgtattggagcctgtccctctcaattccgagccgcaaatatgcatgcgcaaataaagacgagtctccatcggttgaagcctgatactgtcccagctccgtgctgcgtccccgcgagttataatcccatggtccttatacagaaaacagacactggtgtcagccttcagacgtatgacgatttgcttgctaaagactgtcattgtatt

SEQ ID NO: 125 - protein, 735 aa, Artificial Sequence
  fusion protein
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGSAGGGSAGGGSAGGGSAGGGSAGGGSAGGGSAGGGSAGGGSDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 126 - DNA, 2205 bp, Artificial Sequence
  fusion protein coding sequence
gatgcacacaagagtgaggttgctcatcggtttaaagatttgggagaagaaaatttcaaagccttggtgttgattgcctttgctcagtatcttcagcagtccccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcactaaatgttgtaccgagtctcttgttaataggcggccatgcttcagtgcattggaagtcgacgaaacctatgtaccaaaggagttcaacgcagaaacatttacattccatgctgatatctgcacattgagcgagaaagagagacagattaagaaacagacagcgcttgttgaactggttaaacacaaaccaaaagctaccaaggagcagcttaaggcagtaatggatgacttcgcggcctttgtcgagaaatgttgtaaagcggatgataaagagacatgcttcgccgaagagggcaaaaaacttgtagcggcaagccaggccgcactgggtctcggtagtgcgggcggtggttcagcggggggaggatctgcaggtggtggctcagcgggtggcggtagcgctggggggggctccgcaggtgggggatcagcaggcggcggatcagccggcggtggatccgaccactgtcctctcgggcctggtcggtgttgccgcctccatactgtgcgcgcgtctcttgaggatctggggtgggctgattgggttctctctccccgcgaagtgcaggtgaccatgtgtattggtgcttgcccaagtcaattccgagcagctaacatgcacgcccagatcaagactagcctgcatcggcttaagcccgacactgttcctgccccttgctgtgttcctgcatcttataatccaatggtcctgatccagaaaaccgatacgggtgtatcattgcaaacatacgacgacttgcttgccaaagattgccattgcatt

SEQ ID NO: 127 - protein, 727 aa, Artificial Sequence
  fusion protein
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGSEGKSSGSGSESKSTEGKSSGSGSESKSTGGSDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 128 - DNA, 2181 bp, Artificial Sequence
  fusion protein coding sequence
gatgcacacaagagtgaggttgctcatcggtttaaagatttgggagaagaaaatttcaaagccttggtgttgattgcctttgctcagtatcttcagcagtccccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcacgaaatgctgtacagaatccctcgtgaatagaaggccctgcttctctgcccttgaggtggacgagacttacgtccctaaggagtttaacgccgagacctttacttttcatgctgatatttgcaccctttccgaaaaggagcggcagatcaagaaacaaacagccttggtggaactcgtaaaacataaacccaaagccaccaaggaacaacttaaagctgttatggatgacttcgcagccttcgtcgagaaatgttgcaaggcggatgataaggaaacgtgttttgctgaggaagggaagaagttggttgctgcctctcaagcggctctggggcttggcggatcagaggggaagtcctccgggtccggtagcgagtccaaatctacggaagggaagtcatccggttctgggtcagagtccaaatccacaggaggatcagaccattgcccattgggaccaggacgatgttgtcgcctgcatacggtaagagcgtctctggaggatctcggctgggccgattgggttctctcaccacgagaagtacaggtcacaatgtgcataggagcttgtccgagccaattccgggcggctaatatgcacgcacagatcaagacctctttgcaccgcttgaagcccgataccgtgccagcaccgtgttgcgtcccagcatcttacaaccctatggttttgatacagaaaactgacacaggtgtgagcctccagacatatgatgatttgctggctaaggattgccactgtata

(19) United States
(12) Patent Application Publication
(10) Pub. No.: US 2022/0315633 A1
(43) Pub. Date: Oct. 6, 2022

(54) GDF15 ANALOGS AND METHODS FOR USE IN DECREASING BODY WEIGHT AND/OR REDUCING FOOD INTAKE

(71) Applicant: Janssen Pharmaceutica NV, Beerse (BE)

(72) Inventors: Holly KIMKO, Spring House, PA (US); Robert HERMANN, Zug (CH); Elisa FABBRINI, Spring House, PA (US); Vedrana STOJANOVIC-SUSULIC, Spring House, PA (US); Paul ROTHENBERG, Madison, NJ (US); Songmao ZHENG, Spring House, PA (US)

(21) Appl. No.: 17/309,328

(22) PCT Filed: Nov. 19, 2019
(86) PCT No.: PCT/IB2019/059945

§ 371 Date: May 19, 2021

Related U.S. Application Data
(60) Provisional application No. 62/769,675, filed on Nov. 20, 2018.

(51) Int. Cl. C07K 14/495; A61P 3/04; C07K 14/765; A61K 38/18; A61K 9/00

(52) U.S. Cl. CPC C07K 14/495; A61P 3/04; C07K 14/765; A61K 38/18; A61K 9/0019

(57) ABSTRACT

The present invention is related to the fusion proteins containing a half-life extension protein, a linker, and a GDF15 protein which function as GDF15 agonists. These GDF15 agonists may be useful in the treatment of obesity, reduction of body weight, decrease in food intake, or decrease of appetite.

### CROSS-REFERENCE TO RELATED APPLICATIONS

This application is a Section 371 of International Application No. PCT/IB2019/059945, filed on Nov. 19, 2019, which published in the English language on May 28, 2020 under International Publication No. WO 2020/104948 A1, which claims priority to U.S. Provisional Application No. 62/769,675, filed on Nov. 20, 2018, the disclosures of which are incorporated herein by reference in their entirety.

### REFERENCE TO SEQUENCE LISTING SUBMITTED ELECTRONICALLY

This application contains a sequence listing, which is submitted electronically via EFS-Web as an ASCII formatted sequence listing with a file name “004852_183US1_Sequence_Listing” and a creation date of May 17, 2021 and having a size of 388 kb. The sequence listing submitted via EFS-Web is part of the specification and is herein incorporated by reference in its entirety.

### FIELD OF THE INVENTION

The invention relates to GDF15 fusion proteins. In particular, the invention relates to a fusion protein comprising a half-life extension protein, a linker and a GDF15 protein, nucleic acids and expression vectors encoding the fusion proteins, recombinant cells thereof, and pharmaceutical compositions comprising the fusion proteins. Methods of using the fusion proteins to decrease body weight and/or reduce food intake are provided.

### BACKGROUND OF THE INVENTION

GDF15, a member of the TGFβ family, is a secreted protein that circulates in plasma as a 25 kDa homodimer. Plasma levels of GDF15 range between 150 and 1150 pg/ml in most individuals (Tsai et al., J Cachexia Sarcopenia Muscle. 2012, 3: 239-243). High plasma levels of GDF15 are associated with weight loss due to anorexia and cachexia in cancer, and in renal and heart failure. In a clinical trial, GDF15 levels were an independent predictor of insulin resistance in obese, non-diabetic subjects (Kempf et al., Eur. J. Endo. 2012, 167: 671-678). A study in twins showed that the differences in levels of GDF15 within twin pairs correlated to the differences in BMI within that pair, suggesting that GDF15 serves as a long-term regulator of energy homeostasis (Tsai et al., PLoS One. 2015, 10(7):e0133362).

Numerous reports have demonstrated the improvement of glucose tolerance and insulin sensitivity in mouse models upon treatment with GDF15 protein. Two independent strains of transgenic mice overexpressing GDF15 have decreased body weight and fat mass, as well as improved glucose tolerance (Johnen et al., Nat. Med. 2007, 13:1333-1340; Macia et al., PLoS One. 2012, 7:e34868; Chrysovergis et al., Int. J. Obesity. 2014, 38: 1555-1564). Increases in whole-body energy expenditure and oxidative metabolism were reported in GDF15 transgenic mice (Chrysovergis et al., 2014, Id.). These were accompanied by an increase in thermogenic gene expression in brown adipose tissue and an increase in lipolytic gene expression in white adipose tissue. Mice lacking the GDF15 gene have increased body weight and fat mass (Tsai et al., PLoS One. 2013, 8(2):e55174). An Fc-fusion of GDF15 was shown to decrease body weight and improve glucose tolerance as well as insulin sensitivity in an obese cynomolgus monkey model when administered weekly over a period of six weeks (WO 2013/113008).

The effects of GDF15 on body weight are thought to be mediated via the reduction of food intake and possibly increased energy expenditure. GDF15 may improve glycemic control via body weight-dependent and possibly independent mechanisms.

Together, these observations suggest that increasing levels of GDF15 can be beneficial as a therapy for metabolic diseases. There is a need in the art for GDF15-based compositions that can be used to treat or prevent metabolic diseases, disorders, or conditions.

Current obesity treatments include dietary and behavioral interventions, pharmacotherapy, and bariatric surgery. Lifestyle interventions, including diet and increased physical activity, form the foundation to any weight loss efforts and can be effective in achieving weight loss in the short term (3 to 6 months). However, in most cases the weight loss achieved with lifestyle interventions is not sustained in the long term, and only 5-10% of individuals are able to sustain significant weight reduction over time (Fisher B L and Schauer P., Am J Surg. 2002; 184:9S-16S, Rueda-Claussen C F et al., Annu. Rev. Nutr. 2015; 35:475-516). Pharmacotherapy is recommended as second-line therapy when lifestyle changes are ineffective in achieving significant weight loss. Medications approved in the US and EU for chronic weight management include orlistat (gastrointestinal lipase inhibitor), naltrexone/bupropion (combination of an opioid antagonist and a dopamine and norepinephrine-reuptake-inhibitor), and liraglutide (glucagon-like peptide-1 receptor agonist); in the US, lorcaserin (a selective 5-HT₂C receptor agonist) and phentermine/topiramate (combination of a sympathomimetic amine and an anti-epileptic) are also available. In addition, phentermine, as well as some other anorectic agents (including diethylpropion, benzphetamine, and phendimetrazine), are registered in the US for short-term use (up to 12 weeks). In combination with behavioral interventions, these pharmacologic agents have variable efficacy, resulting in an additional weight loss ranging between 2% and 10% of initial body weight. Furthermore, the use of pharmacologic agents may be limited by side effects, including gastrointestinal effects (ie, nausea, vomiting, bloating, diarrhea), neuropsychiatric effects (ie, cognitive impairment, disordered sleep), and elevations in heart rate (depending on the specific agent). Because of these inherent limitations of the available pharmacological approaches (limited efficacy, safety profile, and proportion of non-responders that ranges from 30-65%), there is still a clear unmet medical need for more effective, well tolerated, and safe pharmacological therapies for obesity that can also improve obesity-related comorbidities such as cardio-vascular disease, type 2 diabetes mellitus, and hypertension. Although bariatric surgery (gastric banding, sleeve gastrectomy, and Roux-en-Y gastric bypass) can lead to greater and more durable weight loss than medical therapy, ranging from approximately 15% to 30% loss after 10 years, and can produce remarkable health improvement and reduce mortality for patients with severe obesity, peri-operative (eg, venous thromboembolism) and post-operative (eg, nausea, dumping syndrome, fat-soluble vitamin malabsorption) complications can occur. In addition, given the limitations of both cost and the surgical capacity in most health systems, bariatric surgery can accommodate only a small fraction of eligible patients (Rueda-Claussen C F et al., Annu. Rev. Nutr. 2015; 35:475-516). Therefore, there is a need for more effective and well-tolerated chronic weight management therapies that may also positively affect obesity-related comorbidities such as hypertension, dyslipidemia and type 2 diabetes mellitus.

### BRIEF SUMMARY OF THE INVENTION

The invention satisfies this need by providing a GDF15 agonist, FP2, which represents a novel mechanism of action for the reduction of food intake and the achievement of weight loss. In nonclinical pharmacology and safety studies, FP2 has been shown to exert favorable pharmacological effects and promising safety characteristics to qualify as a candidate for transition into clinical development.

In one aspect, the invention provides a method of decreasing body weight in a subject, comprising administering a composition comprising a fusion protein comprising SEQ ID NO: 92, and at least one pharmaceutically acceptable carrier or diluent, wherein said fusion protein is administered at a dose in the range of about 0.8 mg to about 90 mg, and wherein the subject weight is about 80 kg or more. In one aspect of the invention, the subject is overweight. In one aspect of the invention, the subject has a BMI of about 25 kg/m² or more. In one aspect of the invention, the subject has a BMI in the range of 25 kg/m² to 29.9 kg/m². In one aspect of the invention, the fusion protein is administered at a dose selected from the group consisting of: about 0.8 mg, about 2.5 mg, about 7.5 mg, about 15 mg, about 30 mg, about 60 mg, and about 90 mg. In one aspect of the invention, the fusion protein is administered at a dose range of about 0.01 mg/kg to about 1.08 mg/kg. In one aspect of the invention, the fusion protein is administered at a dose selected from the group consisting of: about 0.01 mg/kg, about 0.03 mg/kg, about 0.09 mg/kg, about 0.18 mg/kg, about 0.36 mg/kg, about 0.72 mg/kg, and about 1.08 mg/kg.

In one aspect of the invention, the fusion protein is administered via subcutaneous injection.

In one aspect of the invention the fusion protein is administered once weekly to the subject.

In one aspect, the invention provides a method of decreasing food intake in a subject, comprising administering a composition comprising a fusion protein comprising SEQ ID NO: 92, and at least one pharmaceutically acceptable carrier or diluent, wherein said fusion protein is administered at a dose in the range of about 0.8 mg to about 90 mg, and wherein the subject weight is 80 kg or more. In one aspect of the invention, the subject is overweight. In one aspect of the invention, the subject has a BMI of 25 kg/m² or more. In one aspect of the invention, the subject has a BMI in the range of 25 kg/m² to 29.9 kg/m². In one aspect of the invention, the fusion protein is administered at a dose selected from the group consisting of: about 0.8 mg, about 2.5 mg, about 7.5 mg, about 15 mg, about 30 mg, about 60 mg, and about 90 mg. In one aspect of the invention, the fusion protein is administered at a dose range of about 0.01 mg/kg to about 1.08 mg/kg. In one aspect of the invention, the fusion protein is administered at a dose selected from the group consisting of: about 0.01 mg/kg, about 0.03 mg/kg, about 0.09 mg/kg, about 0.18 mg/kg, about 0.36 mg/kg, about 0.72 mg/kg, and about 1.08 mg/kg.

In one aspect of the invention, the fusion protein is administered via subcutaneous injection.

In one aspect of the invention, the fusion protein is administered once weekly to the subject.

Other aspects, features and advantages of the invention will be apparent from the following disclosure, including the detailed description of the invention and its preferred embodiments and the appended claims.

### BRIEF DESCRIPTION OF THE DRAWINGS

The foregoing summary, as well as the following detailed description of the invention, will be better understood when read in conjunction with the appended drawings. It should be understood that the invention is not limited to the precise embodiments shown in the drawings.

In the drawings:

FIGS. 1A and 1B show the crystal structure of GDF15, where the disulfide pairing of the first and second Cysteine residues (C1-C2) formed a loop at the N terminus of the protein.

FIG. 2 shows the effects of subcutaneous administration of fusion proteins according to embodiments of the invention, e.g., fusion proteins FP1 (SEQ ID NO: 60) and 6×His-FP1 (SEQ ID NO: 26 with a 6×His tag attached at the N-terminus), on food intake in C57BL/6 mice, the cumulative food intake at 24 hours post-administration is depicted. Values shown within the bars are % reduction compared to vehicle (PBS) group±SEM; N=8 animals per group for all groups, except N=9 in FP1 16 nmol/kg group. *-p<0.05, as compared to vehicle; p values were calculated using One-way ANOVA and Tukey's test for multiple comparisons.

FIG. 3 shows the effects of subcutaneous administration of FP1 and 6×His-FP1 (SEQ ID NO: 26 with a 6×His tag attached at the N-terminus) on food intake in Sprague-Dawley rats, the cumulative food intake at 48 hours post-administration is depicted. Values shown within the bars are % reduction compared to vehicle (PBS) group±SEM; N=8 animals per group. *-p<0.05, as compared to vehicle; p values were calculated using One-way ANOVA and Tukey's test for multiple comparisons.

FIG. 4 shows the change in body weight of diet induced obese (DIO) mice during treatment with FP1. Arrows indicate time (days) of subcutaneous administration post initial dose (Day 0); N=8 animals per group. *-p<0.05, for FP1 1 nmol/kg group as compared to vehicle; #-p<0.05, for FP1 10 nmol/kg group as compared to vehicle; p values were calculated using Two-way RM ANOVA and Tukey's test for multiple comparisons.

FIGS. 5A and 5B show the blood glucose levels in DIO mice during an oral glucose tolerance test (OGTT) after 14 days of dosing of FP1 every 3 days (q3d), the levels are expressed as the area under the curve. N=8 animals per group. *-p<0.05, for FP1 1 nmol/kg group as compared to vehicle; p values were calculated using One-way ANOVA and Tukey's test for multiple comparisons.

FIG. 6 shows the fed blood glucose levels in DIO mice during treatment with FP1. N=8 animals per group. *-p<0.05, as compared to vehicle; p values were calculated using Two-way RM ANOVA and Tukey's test for multiple comparisons.

FIG. 7 shows the 4 hour fasting homeostatic model assessment of insulin resistance (HOMA-IR) in DIO mice after 14 days of treatment with FP1. N=8 animals per group. *-p<0.05, as compared to vehicle; p values were calculated using One-way ANOVA and Tukey's test for multiple comparisons.

FIG. 8 shows the change in body weight in ob/ob mice during treatment with FP1 every 3 days (qd3). Arrows indicate time (days) of subcutaneous administration post initial dose (Day 0); N=9 animals per group. *-p<0.05, for FP1 10 nmol/kg group as compared to vehicle; #-p<0.05, for FP1 1 nmol/kg group as compared to vehicle; p values were calculated using Two-way RM ANOVA and Tukey's test for multiple comparisons.

FIG. 9 shows the blood glucose levels in ob/ob mice during treatment with FP1. Arrows indicate time (days) of subcutaneous administration post initial dose (Day 0); N=9 animals per group. *-p<0.05, for FP1 10 nmol/kg group as compared to vehicle; #-p<0.05, for FP1 1 nmol/kg group as compared to vehicle; p values were calculated using Two-way RM ANOVA and Tukey's test for multiple comparisons.

FIG. 10 shows the mean (±standard deviation, SD) of the serum drug concentration-time profile of FP1 following 2 mg/kg intravenous (IV) and subcutaneous (SC) administration in C57BI/6 mice.

FIG. 11 shows the mean (±SD) of the serum drug concentration-time profile of FP1 following 2 mg/kg IV and SC administration in Sprague-Dawley rats.

FIG. 12 shows the mean (±SD) of the serum drug concentration-time profile of FP1 following 1 mg/kg IV and SC administration in cynomolgus monkeys, as determined by immunoassays.

FIG. 13 shows the serum concentration (ng/mL) of FP1 as an intact dimer over time following a single IV administration in cynomolgus monkeys, as determined by immuno-affinity (IA) capture-LCMS analysis.

FIG. 14 shows the serum concentration (ng/mL) of FP1 as an intact dimer over time following a single SC administration in cynomolgus monkeys, as determined by immuno-affinity capture-LCMS analysis.

FIG. 15 shows the concentration of FP1, represented as a % of the starting concentration, after 0, 4, 24 and 48 hours of ex vivo incubation in plasma obtained from two human subjects (Sub), as determined by immunoassay.

FIG. 16 shows the average concentration of FP1, represented as a % of time 0, as an intact dimer after 0, 4, 24 and 48 hours of ex vivo incubation in plasma obtained from two human subjects (Sub), as determined by intact mass immuno-affinity capture-LCMS analysis.

FIG. 17 shows acute food intake in lean C57BL6N male mice before and after the administration of various N-terminal deletion variants of GDF15. (SEQ ID NOs: 92, 111, and 112, compared to wild type fusion with no deletion (SEQ ID 26 with a 6×His tag attached at the N-terminus). N=8 animals per group; *-p<0.05, as compared to vehicle; p values were calculated using Two-way RM ANOVA and Tukey's test for multiple comparisons.

FIG. 18 shows the effect of a single dose of FP2 on food intake in C57BL/6 mice; specifically, cumulative food intake at 24 hours post administration is shown. Values shown within the bars are % reduction compared to PBS group (mean±SEM); N=8 animals per group for all groups, except N=6 in 6×His-FP1. **-p<0.01, ***-p<0.001, ****-p<0.0001; p values were calculated using Two-way ANOVA and Dunnetts's test for multiple comparisons.

FIG. 19 shows cumulative food intake measured in Sprague-Dawley rats at 24 hours post administration of a single dose of FP2. Values shown within the bars are % reduction compared to PBS group (mean±SEM); N=8 animals per group. **-p<0.01, p values were calculated using Two-way ANOVA and Tukey's test for multiple comparisons.

FIG. 20 shows the percent change in body weight during treatment with FP2 q3d in DIO mice. Arrows indicate the time of subcutaneous injections of FP2; N=6 animals per group; *-p, 0.05, as compared with the vehicle, using Two Way ANOVA and Tukey's test for multiple comparisons;

FIGS. 21A and 21B show the area under the curve (AUC) for the blood glucose concentration levels during an OGTT test after 14 days of q3d dosing of FP2 in DIO mice. *-p<0.05, using One Way ANOVA and Tukey's multiple comparisons test, using n=8 animals per group.

FIG. 22A shows the plasma insulin levels during an OGTT after 8 days of q3d dosing of FP2 in DIO mice. *-p<0.05, Vehicle vs. FP2 (0.3 nmol/kg); FP2 (10 nmol/kg); and Rosiglitazone. #-p<0.05, as compared to Rosiglitazone (10 mg/kg), using Two Way RM ANOVA and Tukey's multiple comparisons test.

FIG. 22B shows the AUC for the plasma insulin levels during an OGTT after 8 days of q3d dosing of FP2 in DIO mice. *-p<0.05, as compared to Vehicle; #-p<0.05, as compared to Rosiglitazone.

FIG. 23 shows the fed blood glucose levels after 8 days of q3d dosing of FP2 in DIO mice. *-p<0.05, as compared to Vehicle, using Two Way RM ANOVA and Tukey's multiple comparisons test, n=8 animals per group.

FIG. 24 shows fasting HOMA-IR after 14 days of treatment with FP2 q3d, followed by 5-hour fast on Day 14, in DIO mice. *-p<0.05, as compared to Vehicle, using One Way ANOVA and Tukey's multiple comparisons test, for n=8 animals per group.

FIG. 25 shows serum concentrations of FP2 following 2 mg/kg intravenous (IV) and 2 mg/kg subcutaneous (SC) administration in C57Bl/6 mice. Values represent mean±SD (n=5 samples per timepoint).

FIG. 26 shows serum concentrations of FP2 following 2 mg/kg intravenous (IV) and 2 mg/kg subcutaneous (SC) administration in Sprague Dawley rats. N=5 samples per time point.

FIG. 27 shows plasma concentrations of FP2 in cynomolgus monkeys analyzed by immunoassay. Values represent mean±SD of n=3, except n=2 for IV at day 22 (528 hr). IV—intravenous, SC—subcutaneous.

FIG. 28 shows plasma concentrations of FP2 as intact dimer in cynomolgus monkeys analyzed by LCMS. Values represent mean±SEM of n=3, except n=2 for subcutaneous (SC) at 168 hours, n=1 for SC at 120 hours and 432 hours, and n=1 for IV—intravenous at 168 hours and 432 hours.

FIG. 29 shows ex vivo stability of FP2 (Normalized Percent Recovery) over 48 hours in human plasma measured by immunoassay.

FIG. 30 shows ex vivo stability of FP2 (Normalized Percent Recovery) over 48 hours in human plasma measured by intact LC/MS.

FIG. 31 shows daily food intake (g) prior to and following a single dose of FP1 in cynomolgus monkeys. *-p<0.05 for 10 mg/kg of FP1 as compared to vehicle.

FIG. 32 shows percent body weight change prior to and following a single dose of FP1 in cynomolgus monkeys. *-p<0.05 for 10 mg/kg FP1 as compared to vehicle; #-p<0.05 for 3 mg/kg as compared to vehicle using Two Way RM ANOVA and Tukey's multiple comparisons test, for n=8 animals per group.

FIG. 33 shows daily food intake (g) prior to and following a single dose of FP2 in cynomolgus monkeys. *-p<0.05, as compared to Vehicle, using Two Way RM ANOVA and Tukey's multiple comparisons test, for n=8 animals per group.

FIG. 34 shows percent body weight change prior to and following a single dose of FP2 in cynomolgus monkeys. *-p<0.05, for 10 nmol/kg of FP2 as compared to Vehicle, #-p<0.05, for 3 nmol/kg of FP2 as compared to Vehicle, & -p<0.05, for 1 nmol/kg of FP2 as compared to Vehicle, using Two Way RM ANOVA and Tukey's multiple comparisons test, for n=8 animals per group.

FIG. 35 shows food intake in spontaneously obese cynomolgus monkeys during 12-week long period of once-weekly subcutaneous administration of FP2, normalized as percent reduction from baseline, calculated as average daily food intake during week prior to dosing. Data represented mean±SEM, with N number of animals below the graph; error bars were ±range for N=2.

FIG. 36 shows body weight (% change from baseline) in spontaneously obese cynomolgus monkeys during 12-week long period of once-weekly subcutaneous administration of FP2. Data represented mean±SEM, with N number of animals below the graph; error bars were ±range for N=2.

FIG. 37 shows serum concentration (nM) of FP2 measured by immunoassay in spontaneously obese cynomolgus monkeys during 12-week long period of once-weekly subcutaneous administration of FP2. Data represented mean±SEM, with N number of animals with detectable exposure below the graph; error bars were ±range for N=2.

FIG. 38 shows schematic overview of the study. DG—dosing group.

### DETAILED DESCRIPTION OF THE INVENTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning commonly understood to one of ordinary skill in the art to which this invention pertains. Otherwise, certain terms used herein have the meanings as set in the specification. All patents, published patent applications and publications cited herein are incorporated by reference as if set forth fully herein. It must be noted that as used herein and in the appended claims, the singular forms “a,” “an,” and “the” include plural reference unless the context clearly dictates otherwise.

The invention relates to a fusion protein comprising (a) a half life-extension protein, (b) a linker, and (c) a GDF15 protein, wherein the fusion protein is arranged from N-terminus to C-terminus in the order (a)-(b)-(c).

It is found that a fusion protein according to an embodiment of the invention, comprising a half life-extension protein, a linker, and a GDF15 protein, results in an increased half life of the GDF15 protein, and fusion proteins of the invention exhibit metabolic effects that demonstrate their suitability as therapeutics for treating and preventing metabolic diseases, disorders or conditions. Such effects include, but are not limited to, decreasing body weight, increasing glucose tolerance, and improving insulin sensitivity of animals administered with the fusion proteins.

As used herein, the term “fusion protein” refers to a protein having two or more portions covalently linked together, where each of the portions is derived from different proteins.

Fusion proteins according to embodiments of the invention can include any GDF15 protein. As used herein, the term “GDF15 protein” refers to any naturally-occurring wild-type growth differentiation factor 15 protein or a functional variant thereof. The GDF15 protein can be from any mammal, such as a human or another suitable mammal, such as a mouse, rabbit, rat, pig, dog, or a primate. In particular embodiments, the GDF15 protein is a human GDF15 protein or a functional variant thereof. In preferred embodiments, the GDF15 protein is a mature GDF15 protein or a functional variant thereof.

As used herein, the term “mature GDF15 protein” refers to the portion of the pre-pro-protein of GDF15 that is released from the full-length protein following intracellular cleavage at the RXXR furin-like cleavage site. Mature GDF15 proteins are secreted as homodimers linked by disulfide bonds. In one embodiment of the invention, a mature GDF15 protein, shorthand GDF15(197-308) (SEQ ID NO: 6), contains amino acids 197-308 of a full-length human GDF15 protein.

As used herein, “functional variant” refers to a variant of a parent protein having substantial or significant sequence identity to the parent protein and retains at least one of the biological activities of the parent protein. A functional variant of a parent protein can be prepared by means known in the art in view of the present disclosure. A functional variant can include one or more modifications to the amino acid sequence of the parent protein. The modifications can change the physico-chemical properties of the polypeptide, for example, by improving the thermal stability of the polypeptide, altering the substrate specificity, changing the pH optimum, and the like. The modifications can also alter the biological activities of the parent protein, as long as they do not destroy or abolish all of the biological activities of the parent protein. The modifications can also be deletions or insertions of one or more amino acids.

According to other embodiments of the invention, a functional variant of a parent protein comprises a deletion and/or insertion of one or more amino acids to the parent protein. For example, a functional variant of a mature GDF15 protein can include a deletion and/or insertion of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more amino acids to the mature GDF15 protein, preferably, a deletion of 1 to 30 amino acids at the N-terminus of the mature GDF15 protein.

According to embodiments of the invention, a fusion protein of the invention comprises a GDF15 protein that has an amino acid sequence at least 90% identical to the amino acid sequence of a mature GDF15, such as GDF15(197-308) (SEQ ID NO: 6); or an amino acid sequence at least 90% identical to the amino acid sequence of a mature GDF15 truncated at the N-terminus, such as GDF15(200-308) (SEQ ID NO: 7), GDF15(201-308) (SEQ ID NO: 8), GDF15(202-308) (SEQ ID NO: 9), GDF15(203-308) (SEQ ID NO: 10), or GDF15(211-308) (SEQ ID NO: 11). The GDF15 protein can have at least one of substitutions, insertions and deletions to SEQ ID NO: 6, 7, 8, 9, 10 or 11, as long as it maintains at least one of the biological activities of the GDF15 protein, such as its effects on food intake, blood glucose levels, insulin resistance, and body weight, etc.

In particular embodiments, a fusion protein of the invention comprises a GDF15 protein having the amino acid sequence of SEQ ID NO: 11, including but not limited to, the amino acid sequence of SEQ ID NO: 6, 7, 8, 9, 10 or 11.

Any suitable half life extension protein can be used in fusion proteins according to embodiments of the invention. As used herein, the term “half life extension protein” can be any protein or fragment thereof that is known to extend the half life of proteins to which it is fused. Examples of such half life extension proteins include, but are not limited to, human serum albumin (HSA), the constant fragment domain (Fc) of an immunoglobulin (Ig), or transferrin (Tf). In embodiments of the invention, the half life extension protein comprises HSA or a functional variant thereof. In particular embodiments of the invention, the half life extension protein comprises an amino acid sequence that is at least 90% identity to SEQ ID NO: 1. In preferred embodiments of the invention, the half life extension protein comprises HSA or functional variant thereof wherein the cysteine residue at position 34 of the HSA has been replaced by serine or alanine.

In particular embodiments, a fusion protein of the invention comprises a half life extension protein having an amino acid sequence selected from the group consisting of SEQ ID NOs: 1-3.

Any suitable linker can be used in fusion proteins according to embodiments of the invention. As used herein, the term “linker” refers to a linking moiety comprising a peptide linker. Preferably, the linker helps insure correct folding, minimizes steric hindrance and does not interfere significantly with the structure of each functional component within the fusion protein. In some embodiments of the invention, the peptide linker comprises 2 to 120 amino acids. For example, the peptide linker comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119 or 120 amino acids.

In embodiments of the invention, the linker increases the flexibility of the fusion protein components. In particular embodiments of the invention, the linker can be a flexible linker comprising the sequence (GGGGS)n, including but not limited to, GS-(GGGGS)n or AS-(GGGGS)n-GT, wherein n is 2 to 20, such as 2, 3, 4, 5, 6, 7, 8, 9, 10, 11, 12, 13, 14 or 15.

In other embodiments of the invention, the linker is structured. In particular embodiments of the invention, the linker can be a structured linker comprising the sequence (AP)n or (EAAAK)n, including but not limited to, AS-(AP)n-GT or AS-(EAAAK)n-GT, wherein n is 2 to 20, such as 2, 3, 4, 5, 6, 7, 8, 9, 10, 11, 12, 13, 14 or 15. In other embodiments of the invention, the linker comprises the sequences (GGGGA)ₙ, (PGGGS)ₙ, (AGGGS)ₙ or GGS-(EGKSSGSGSESKST)ₙ-GGS wherein n is 2 to 20.

In embodiments of the invention, the fusion protein comprises an amino acid sequence having at least 90% sequence identity to SEQ ID NOs: 5, 25-30, 36-37, 40, 48, 55-56, 59-60 or 64-75. In particular embodiments of the invention, the fusion protein comprises an amino acid sequence selected from the group consisting of SEQ ID NOs: 5, 25-30, 36-37, 40, 48, 55-56, 59-60 and 64-75. In more particular embodiments of the invention, the fusion protein comprises an amino acid sequence selected from the group consisting of SEQ ID NOs: 5, 25-30, 40, 55-56, 55-56, 59-60, and 70. In further more particular embodiments of the invention, the fusion protein comprises the amino acid sequence of SEQ ID NO: 92, SEQ ID NO: 60 or SEQ ID NO: 26. The fusion protein can also include small extension(s) at the amino- or carboxyl-terminal end of the protein, such as a tag that facilitates purification, such as a poly-histidine tag, an antigenic epitope or a binding domain.

The fusion proteins disclosed herein can be characterized or assessed for GDF15 biological activities including, but not limited to effects on food intake, oral glucose tolerance tests, measurements of blood glucose levels, insulin resistance analysis, changes in body weight, pharmacokinetic analysis, toxicokinetic analysis, immunoassays and mass spec analysis of the level and stability of full-length fusion proteins, and human plasma ex vivo stability analysis.

The invention also provides an isolated nucleic acid molecule encoding a fusion protein of the invention. In embodiments of the invention, the isolated nucleic acid molecule encodes a fusion protein comprising an amino acid sequence having at least 90% sequence identity to SEQ ID NOs: 5, 25-30, 36-37, 40, 48, 55-56, 59-60, 64-75 or 92. In particular embodiments, the isolated nucleic acid molecule encodes a fusion protein comprising an amino acid sequence selected from the group consisting of SEQ ID NOs: 5, 25-31, 36-37, 40, 48, 55-56, 59-60, 64-75, and 92. In more particular embodiments, the isolated nucleic acid molecule encodes a fusion protein comprising an amino acid sequence selected from the group consisting of SEQ ID NOs: 5, 25-30, 40, 55-56, 59-60, 70, and 92. In more particular embodiments, the isolated nucleic acid molecule comprises the nucleotide sequence of SEQ ID NOs: 76-91, 95, and 110.

According to other embodiments of the invention, the nucleic acid molecule encoding the fusion protein can be in an expression vector. Expression vectors include, but are not limited to, vectors for recombinant protein expression and vectors for delivery of nucleic acids into a subject for expression in a tissue of the subject, such as viral vectors. Examples of viral vectors suitable for use with the invention include, but are not limited to adenoviral vectors, adeno-associated virus vectors, lentiviral vectors, etc. The vector can also be a non-viral vector. Examples of non-viral vectors include, but are not limited to plasmids, bacterial artificial chromosomes, yeast artificial chromosomes, bacteriophages, etc. The vector can include any element to establish a conventional function of an expression vector, for example, a promoter, ribosome binding element, terminator, enhancer, selection marker, or an origin of replication.

According to other embodiments of the invention, the nucleic acid molecule encoding the fusion protein can be codon optimized for improved recombinant expression from a desired host cell, such as Human Embryonic Kidney (HEK) or Chinese hamster ovary (CHO) cells, using methods known in the art in view of the present disclosure.

The invention also provides a host cell comprising a nucleic acid molecule encoding a fusion protein of the invention. Host cells include, but are not limited to, host cells for recombinant protein expression and host cells for delivery of the nucleic acid into a subject for expression in a tissue of the subject. Examples of host cells suitable for use with the invention include, but are not limited to HEK or CHO cells.

In another general aspect, the invention relates to a method of obtaining a fusion protein of the invention. In a general aspect, the method comprises: (1) culturing a host cell comprising a nucleic acid molecule encoding a fusion protein under a condition that the fusion protein is produced, and (2) recovering the fusion protein produced by the host cell. The fusion protein can be purified further using methods known in the art.

In some embodiments, the fusion protein is expressed in host cells and purified therefrom using a combination of one or more standard purification techniques, including, but not limited to, affinity chromatography, size exclusion chromatography, ultrafiltration, and dialysis. Preferably, the fusion protein is purified to be free of any proteases.

The invention also provides a pharmaceutical composition comprising a fusion protein of the invention and a pharmaceutically acceptable carrier.

The invention further provides a composition comprising a nucleic acid molecule encoding a fusion protein of the invention and a pharmaceutically acceptable carrier. Compositions comprising a nucleic acid molecule encoding a fusion protein of the invention can comprise a delivery vehicle for introduction of the nucleic acid molecule into a cell for expression of the fusion protein. Examples of nucleic acid delivery vehicles include liposomes, biocompatible polymers, including natural polymers and synthetic polymers, lipoproteins, polypeptides, polysaccharides, lipopolysaccharides, artificial viral envelopes, metal particles, and bacteria, viruses, such as baculoviruses, adenoviruses and retroviruses, bacteriophages, cosmids, plasmids, fungal vectors and other recombination vehicles typically used in the art which have been described for expression in a variety of eukaryotic hosts.

The invention also relates to kits comprising a pharmaceutical composition of the invention. The kits can contain a first container having a dried fusion protein of the invention and a second container having an aqueous solution to be mixed with the dried fusion protein prior to administration to a subject, or a single container containing a liquid pharmaceutical composition of the invention. The kit can contain a single-dose administration unit or multiple dose administration units of a pharmaceutical composition of the invention. The kit can also include one or more pre-filled syringes (e.g., liquid syringes and lyosyringes). A kit can also comprise instructions for the use thereof. The instructions can describe the use and nature of the materials provided in the kit, and can be tailored to the precise metabolic disorder being treated.

The invention also relates to use of the pharmaceutical compositions described herein to treat or prevent a metabolic disease, disorder or condition, such as type 2 diabetes, elevated glucose levels, elevated insulin levels, obesity, dyslipidemia, diabetic nephropathy, myocardial ischemic injury, congestive heart failure, or rheumatoid arthritis. According to embodiments of the invention, a method of treating or preventing a metabolic disease, disorder or condition in a subject in need of the treatment comprises administering to the subject a therapeutically or prophylactically effective amount of a pharmaceutical composition of the invention. Any of the pharmaceutical compositions described herein can be used in a method of the invention, including pharmaceutical compositions comprising a fusion protein of the invention or pharmaceutical compositions comprising a nucleic acid encoding the fusion protein.

Provided herein are methods of decreasing body weight in a subject, comprising administering a composition comprising a fusion protein comprising SEQ ID NO: 92, and at least one pharmaceutically acceptable carrier or diluent, wherein said fusion protein is administered at a dose in the range of about 0.8 mg to about 90 mg, and wherein the subject weight is 80 kg or more.

According to certain embodiments, the subject is overweight. In certain embodiments of the invention, the subject has a BMI of 25 kg/m² or more and in certain embodiments, the subject has a BMI in the range of 25 kg/m² to 29.9 kg/m².

According to certain embodiments, the fusion protein is administered at a dose selected from the group consisting of: about 0.8 mg, about 2.5 mg, about 7.5 mg, about 15 mg, about 30 mg, about 60 mg, and about 90 mg. In certain embodiments, the fusion protein is administered at a dose of about 0.8 mg. In other embodiments, the fusion protein is administered at a dose of about 2.5 mg. In other embodiments, the fusion protein is administered at a dose of about 7.5 mg. In other embodiments, the fusion protein is administered at a dose of about 15 mg. In other embodiments, the fusion protein is administered at a dose of about 30 mg. In other embodiments, the fusion protein is administered at a dose of about 60 mg. In other embodiments, the fusion protein is administered at a dose of about 90 mg.

According to certain embodiments, the fusion protein is administered at a dose range of about 0.01 mg/kg to about 1.08 mg/kg. In certain of such embodiments, the fusion protein is administered at a dose selected from the group consisting of: about 0.01 mg/kg, about 0.03 mg/kg, about 0.09 mg/kg, about 0.18 mg/kg, about 0.36 mg/kg, about 0.72 mg/kg, and about 1.08 mg/kg. In certain embodiments, the fusion protein is administered at a dose of about 0.01 mg/kg. In other embodiments, the fusion protein is administered at a dose of about 0.03 mg/kg. In other embodiments, the fusion protein is administered at a dose of about 0.09 mg/kg. In other embodiments, the fusion protein is administered at a dose of about 0.18 mg/kg. In other embodiments, the fusion protein is administered at a dose of about 0.36 mg/kg. In other embodiments, the fusion protein is administered at a dose of about 0.72 mg/kg. In other embodiments, the fusion protein is administered at a dose of about 1.08 mg/kg.

According to certain embodiments of the present invention, the fusion protein is administered via subcutaneous injection.

According to certain embodiments of the present invention, the fusion protein is administered once weekly to the subject.

Provided herein are methods of decreasing food intake in a subject, comprising administering a composition comprising a fusion protein comprising SEQ ID NO: 92, and at least one pharmaceutically acceptable carrier or diluent, wherein said composition is administered at a dose in the range of about 0.8 mg to about 90 mg, and wherein the subject weight is 80 kg or more.

According to certain embodiments, the subject is overweight. In certain embodiments of the invention, the subject has a BMI of 25 kg/m² or more and in certain embodiments, the subject has a BMI in the range of 25 kg/m² to 29.9 kg/m².

According to certain embodiments, the fusion protein is administered at a dose selected from the group consisting of: about 0.8 mg, about 2.5 mg, about 7.5 mg, about 15 mg, about 30 mg, about 60 mg, and about 90 mg. In certain embodiments, the fusion protein is administered at a dose of about 0.8 mg. In other embodiments, the fusion protein is administered at a dose of about 2.5 mg. In other embodiments, the fusion protein is administered at a dose of about 7.5 mg. In other embodiments, the fusion protein is administered at a dose of about 15 mg. In other embodiments, the fusion protein is administered at a dose of about 30 mg. In other embodiments, the fusion protein is administered at a dose of about 60 mg. In other embodiments, the fusion protein is administered at a dose of about 90 mg.

According to certain embodiments, the fusion protein is administered at a dose range of about 0.01 mg/kg to about 1.08 mg/kg. In certain of such embodiments, the fusion protein is administered at a dose selected from the group consisting of: about 0.01 mg/kg, about 0.03 mg/kg, about 0.09 mg/kg, about 0.18 mg/kg, about 0.36 mg/kg, about 0.72 mg/kg, and about 1.08 mg/kg. In certain embodiments, the fusion protein is administered at a dose of about 0.01 mg/kg. In other embodiments, the fusion protein is administered at a dose of about 0.03 mg/kg. In other embodiments, the fusion protein is administered at a dose of about 0.09 mg/kg. In other embodiments, the fusion protein is administered at a dose of about 0.18 mg/kg. In other embodiments, the fusion protein is administered at a dose of about 0.36 mg/kg. In other embodiments, the fusion protein is administered at a dose of about 0.72 mg/kg. In other embodiments, the fusion protein is administered at a dose of about 1.08 mg/kg.

According to certain embodiments of the present invention, the fusion protein is administered via subcutaneous injection.

According to certain embodiments of the present invention, the fusion protein is administered once weekly to the subject.

The term “about” when used in reference to numerical ranges, cutoffs, or specific values is used to indicate that the recited values may vary by up to as much as 10% from the listed value. Thus, the term “about” is used to encompass variations of ±10% or less, variations of 5% or less, variations of ±1% or less, variations of ±0.5% or less, variations of ±5% or less, or variations of ±0.1% or less from the specified value.

As used herein, “subject” means any animal, particularly a mammal, most particularly a human, who will be or has been treated by a method according to an embodiment of the invention. The term “mammal” as used herein, encompasses any mammal. Examples of mammals include, but are not limited to, cows, horses, sheep, pigs, cats, dogs, mice, rats, rabbits, guinea pigs, non-human primates (NHPs) such as monkeys or apes, humans, etc., more particularly a human.

As used herein, “overweight” refers to excessive body weight. Various parameters are used to determine whether a subject is overweight compared to a reference healthy individual, including the subject's age, height, sex and health status. For example, a subject may be considered overweight or obese by assessment of the subject's Body Mass Index (BMI), which is calculated by dividing a subject's weight in kilograms by the square of subject's height in meters. An adult having a BMI in the range of 18.5 to 24.9 kg/m² is considered to have a normal weight; an adult having a BMI between 25 and 29.9 kg/m² may be considered overweight (pre-obese); an adult having a BMI of 30 kg/m² or higher may be considered obese. Enhanced appetite frequently contributes to excessive body weight.

A “metabolic disease, disorder or condition” refers to any disorder related to abnormal metabolism. Examples of metabolic diseases, disorders or conditions that can be treated according to a method of the invention include, but are not limited to, type 2 diabetes, elevated glucose levels, elevated insulin levels, obesity, being overweight, dyslipidemia, diabetic nephropathy, myocardial ischemic injury, congestive heart failure, or rheumatoid arthritis.

The terms “treat,” “treating,” and “treatment” as used herein refer to administering a composition to a subject to achieve a desired therapeutic or clinical outcome in the subject. In one embodiment, the terms “treat,” “treating,” and “treatment” refer to administering a pharmaceutical composition of the invention to reduce, alleviate or slow the progression or development of a metabolic disorder, such as type 2 diabetes, elevated glucose levels, elevated insulin levels, obesity, dyslipidemia, diabetic nephropathy, myocardial ischemic injury, congestive heart failure, or rheumatoid arthritis.

According to embodiments of the invention, a pharmaceutical composition of the invention can be administered to a subject by any method known to those skilled in the art in view of the present disclosure, such as by intramuscular, subcutaneous, oral, intravenous, cutaneous, intramucosal (e.g., gut), intranasal or intraperitoneal route of administration. In particular embodiments, a pharmaceutical composition of the invention is administered to a subject by intravenous injection or subcutaneous injection.

As used herein, the “once weekly” administration is performed within a single day. Preferably, the “once weekly” administration is performed in a single step, such as a single injection.

In certain embodiments, the present invention provides a clinically proven safe and clinically proven effective dose of a GDF15 fusion protein having a sequence comprising SEQ ID NO:92 for use in a method of decreasing body weight in a subject, wherein said clinically proven safe and clinically proven effective dose is a single subcutaneous (SC) injection administered at a dose in the range of 0.8 mg to 90 mg to a subject weighing 80 kg or more.

In certain embodiments, the present invention provides a clinically proven safe and clinically proven effective dose of a GDF15 fusion protein having a sequence comprising SEQ ID NO:92 for use in a method of decreasing food intake in a subject, wherein said clinically proven safe and clinically proven effective dose is a single subcutaneous (SC) injection administered at a dose in the range of 0.8 mg to 90 mg to a subject weighing 80 kg or more.

According to the invention as defined herein, the term “clinically proven safe”, as it relates to a dose or treatment with the GDF15 fusion protein having a sequence comprising SEQ ID NO:92, refers to a favorable risk:benefit ratio with a relatively low or reduced frequency and/or low or reduced severity of adverse events, including adverse vital signs (heart rate, systolic and diastolic blood pressure, body temperature), adverse standard clinical laboratory tests (hematology, clinical chemistry, urinalysis, lipids, coagulation), allergic reactions/hypersensitivity, adverse local injection site reactions, or adverse EKG.

According to the invention as defined herein, the terms “clinically proven effective” or “clinically proven efficacy”, as they relate to terms such as dose, dosage regimen, or treatment with the GDF15 fusion protein having a sequence comprising SEQ ID NO:92, refer to decreased food intake, decreased appetite ratings, decreased food palatability assessed by using questionnaires, or decreased body weight.

As used herein, a decrease in body weight is a decrease of at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, or any number in between.

As used herein, a decrease in food intake is a decrease of at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, or any number in between. Food intake may be measured by measuring calories consumed estimated based on the grams consumed of each food item, and its nutritional content.

As used herein, unless otherwise noted, the term “clinically proven” (used independently or to modify the terms “safe” and/or “effective”) shall mean that it has been proven by a clinical trial wherein the clinical trial has met the standards of U.S. Food and Drug Administration, EMEA or a corresponding national regulatory agency. For example, the clinical study may be an adequately sized, randomized, double blinded study used to clinically prove the effects of the drug. In some embodiments, “clinically proven” indicates that it has been proven by a clinical trial that has met the standards of the U.S. Food and Drug Administration, EMEA or a corresponding national regulatory agency for a Phase I clinical trial.

### EMBODIMENTS

1. A method of decreasing body weight in a subject, comprising administering a composition comprising a fusion protein comprising SEQ ID NO: 92, and at least one pharmaceutically acceptable carrier or diluent, wherein said fusion protein is administered at a dose in the range of 0.8 mg to 90 mg, and wherein the subject's weight is 80 kg or more.

2. The method of embodiment 1, wherein the subject is overweight.

3. The method of embodiment 2, wherein the subject has a BMI of 25 kg/m2 or more.

4. The method of embodiment 3, wherein the subject has the BMI in the range of 25 kg/m2 to 29.9 kg/m2.

5. The method of embodiment 1, wherein the fusion protein is administered at a dose selected from the group consisting of: 0.8 mg, 2.5 mg, 7.5 mg, 15 mg, 30 mg, 60 mg, and 90 mg.

6. The method of embodiment 5, wherein fusion protein is administered at a dose of 0.8 mg.

7. The method of embodiment 5, wherein fusion protein is administered at a dose of 2.5 mg.

8. The method of embodiment 5, wherein fusion protein is administered at a dose of 7.5 mg.

9. The method of embodiment 5, wherein fusion protein is administered at a dose of 15 mg.

10. The method of embodiment 5, wherein fusion protein is administered at a dose of 30 mg.

11. The method of embodiment 5, wherein fusion protein is administered at a dose of 60 mg.

12. The method of embodiment 5, wherein fusion protein is administered at a dose of 90 mg.

13. The method of embodiment 1, wherein the fusion protein is administered via subcutaneous injection.

14. A method of decreasing body weight in a subject, comprising administering a composition comprising a fusion protein comprising SEQ ID NO: 92, and at least one pharmaceutically acceptable carrier or diluent, wherein said fusion protein is administered at a dose in a range of 0.01 mg/kg to 1.08 mg/kg.

15. The method of embodiment 14, wherein said fusion protein is administered at a dose selected from the group consisting of: 0.01 mg/kg, 0.03 mg/kg, 0.09 mg/kg, 0.18 mg/kg, 0.36 mg/kg, 0.72 mg/kg, and 1.08 mg/kg.

16. The method of embodiment 15, wherein fusion protein is administered at a dose of 0.01 mg/kg.

17. The method of embodiment 15, wherein fusion protein is administered at a dose of 0.03 mg/kg.

18. The method of embodiment 15, wherein fusion protein is administered at a dose of 0.09 mg/kg.

19. The method of embodiment 15, wherein fusion protein is administered at a dose of 0.18 mg/kg.

20. The method of embodiment 15, wherein fusion protein is administered at a dose of 0.36 mg/kg.

21. The method of embodiment 15, wherein fusion protein is administered at a dose of 0.72 mg/kg.

22. The method of embodiment 15, wherein fusion protein is administered at a dose of 1.08 mg/kg.

23. The method of embodiment 14, wherein the fusion protein is administered via subcutaneous injection.

24. The method of embodiment 14 wherein the composition is administered once weekly to the subject.

25. The method of embodiment 1 wherein the composition is administered once weekly to the subject.

1A. A method of decreasing food intake in a subject, comprising administering a composition comprising a fusion protein comprising SEQ ID NO: 92, and at least one pharmaceutically acceptable carrier or diluent, wherein said fusion protein is administered at a dose in the range of 0.8 mg to 90 mg, and wherein the subject weight is 80 kg or more.

2A. The method of embodiment 1A, wherein the subject is overweight.

3A. The method of embodiment 2A, wherein the subject has a BMI of 25 kg/m2 or more.

4A. The method of embodiment 3A, wherein the subject has the BMI in the range of 25 kg/m2 to 29.9 kg/m2.

5A. The method of embodiment 1A, wherein the fusion protein is administered at a dose selected from the group consisting of: 0.8 mg, 2.5 mg, 7.5 mg, 15 mg, 30 mg, 60 mg, and 90 mg.

6A. The method of embodiment 5A, wherein fusion protein is administered at a dose of 0.8 mg.

7A. The method of embodiment 5A, wherein fusion protein is administered at a dose of 2.5 mg.

8A. The method of embodiment 5A, wherein fusion protein is administered at a dose of 7.5 mg.

9A. The method of embodiment 5A, wherein fusion protein is administered at a dose of 15 mg.

10A. The method of embodiment 5A, wherein fusion protein is administered at a dose of 30 mg.

11A. The method of embodiment 5A, wherein fusion protein is administered at a dose of 60 mg.

12A. The method of embodiment 5A, wherein fusion protein is administered at a dose of 90 mg.

13A. The method of embodiment 1A, wherein the fusion protein is administered via subcutaneous injection.

14A. A method of decreasing food intake in a subject, comprising administering a composition comprising a fusion protein comprising SEQ ID NO: 92, and at least one pharmaceutically acceptable carrier or diluent, wherein said fusion protein is administered at a dose in a range of 0.01 mg/kg to 1.08 mg/kg.

15A. The method of embodiment 14A, wherein said fusion protein is administered at a dose selected from the group consisting of: 0.01 mg/kg, 0.03 mg/kg, 0.09 mg/kg, 0.18 mg/kg, 0.36 mg/kg, 0.72 mg/kg, and 1.08 mg/kg.

16A. The method of embodiment 15A, wherein fusion protein is administered at a dose of 0.01 mg/kg.

17A. The method of embodiment 15A, wherein fusion protein is administered at a dose of 0.03 mg/kg.

18A. The method of embodiment 15A, wherein fusion protein is administered at a dose of 0.09 mg/kg.

19A. The method of embodiment 15A, wherein fusion protein is administered at a dose of 0.18 mg/kg.

20A. The method of embodiment 15A, wherein fusion protein is administered at a dose of 0.36 mg/kg.

21A. The method of embodiment 15A, wherein fusion protein is administered at a dose of 0.72 mg/kg.

22A. The method of embodiment 15A, wherein fusion protein is administered at a dose of 1.08 mg/kg.

23A. The method of embodiment 15A, wherein the fusion protein is administered via subcutaneous injection.

24A. The method of embodiment 14A wherein the composition is administered once weekly to the subject.

25A. The method of embodiment 1A wherein the composition is administered once weekly to the subject.

1B. A method of decreasing body weight in a subject, comprising administering once weekly to the subject a composition comprising a fusion protein comprising SEQ ID NO: 92, and at least one pharmaceutically acceptable carrier or diluent, wherein said fusion protein is administered at a dose in the range of 0.8 mg to 90 mg, and wherein the subject's weight is 80 kg or more.

2B. The method of embodiment 1B, wherein the subject is overweight.

3B. The method of embodiment 2B, wherein the subject has a BMI of 25 kg/m2 or more.

4B. The method of embodiment 3B, wherein the subject has the BMI in the range of 25 kg/m2 to 29.9 kg/m2.

5B. The method of embodiment 1B, wherein the fusion protein is administered at a dose selected from the group consisting of: 0.8 mg, 2.5 mg, 7.5 mg, 15 mg, 30 mg, 60 mg, and 90 mg.

6B. The method of embodiment 5B, wherein fusion protein is administered at a dose of 0.8 mg.

7B. The method of embodiment 5B, wherein fusion protein is administered at a dose of 2.5 mg.

8B. The method of embodiment 5B, wherein fusion protein is administered at a dose of 7.5 mg.

9B. The method of embodiment 5B, wherein fusion protein is administered at a dose of 15 mg.

10B. The method of embodiment 5B, wherein fusion protein is administered at a dose of 30 mg.

11B. The method of embodiment 5B, wherein fusion protein is administered at a dose of 60 mg.

12B. The method of embodiment 5B, wherein fusion protein is administered at a dose of 90 mg.

13B. The method of embodiment 1B, wherein the fusion protein is administered via subcutaneous injection.

14B. A method of decreasing body weight in a subject, comprising administering once weekly to the subject a composition comprising a fusion protein comprising SEQ ID NO: 92, and at least one pharmaceutically acceptable carrier or diluent, wherein said fusion protein is administered at a dose in a range of 0.01 mg/kg to 1.08 mg/kg.

15B. The method of embodiment 14B, wherein said fusion protein is administered at a dose selected from the group consisting of: 0.01 mg/kg, 0.03 mg/kg, 0.09 mg/kg, 0.18 mg/kg, 0.36 mg/kg, 0.72 mg/kg, and 1.08 mg/kg.

16B. The method of embodiment 15B, wherein fusion protein is administered at a dose of 0.01 mg/kg.

17B. The method of embodiment 15B, wherein fusion protein is administered at a dose of 0.03 mg/kg.

18B. The method of embodiment 15B, wherein fusion protein is administered at a dose of 0.09 mg/kg.

19B. The method of embodiment 15B, wherein fusion protein is administered at a dose of 0.18 mg/kg.

20B. The method of embodiment 15B, wherein fusion protein is administered at a dose of 0.36 mg/kg.

21B. The method of embodiment 15B, wherein fusion protein is administered at a dose of 0.72 mg/kg.

22B. The method of embodiment 15B, wherein fusion protein is administered at a dose of 1.08 mg/kg.

23B. The method of embodiment 22B, wherein the fusion protein is administered via subcutaneous injection.

1C. A method of decreasing food intake in a subject, comprising administering once weekly to the subject a composition comprising a fusion protein comprising SEQ ID NO: 92, and at least one pharmaceutically acceptable carrier or diluent, wherein said fusion protein is administered at a dose in the range of 0.8 mg to 90 mg, and wherein the subject weight is 80 kg or more.

2C. The method of embodiment 1C, wherein the subject is overweight.

3C. The method of embodiment 2C, wherein the subject has a BMI of 25 kg/m2 or more.

4C. The method of embodiment 3C, wherein the subject has the BMI in the range of 25 kg/m2 to 29.9 kg/m2.

5C. The method of embodiment 1C, wherein the fusion protein is administered at a dose selected from the group consisting of: 0.8 mg, 2.5 mg, 7.5 mg, 15 mg, 30 mg, 60 mg, and 90 mg.

6C. The method of embodiment 5C, wherein fusion protein is administered at a dose of 0.8 mg.

7C. The method of embodiment 5C, wherein fusion protein is administered at a dose of 2.5 mg.

8C. The method of embodiment 5C, wherein fusion protein is administered at a dose of 7.5 mg.

9C. The method of embodiment 5C, wherein fusion protein is administered at a dose of 15 mg.

10C. The method of embodiment 5C, wherein fusion protein is administered at a dose of 30 mg.

11C. The method of embodiment 5C, wherein fusion protein is administered at a dose of 60 mg.

12C. The method of embodiment 5C, wherein fusion protein is administered at a dose of 90 mg.

13C. The method of embodiment 1C, wherein the fusion protein is administered via subcutaneous injection.

14C. A method of decreasing food intake in a subject, comprising administering once weekly to a subject a composition comprising a fusion protein comprising SEQ ID NO: 92, and at least one pharmaceutically acceptable carrier or diluent, wherein said fusion protein is administered at a dose in a range of 0.01 mg/kg to 1.08 mg/kg.

15C. The method of embodiment 14C, wherein said fusion protein is administered at a dose selected from the group consisting of: 0.01 mg/kg, 0.03 mg/kg, 0.09 mg/kg, 0.18 mg/kg, 0.36 mg/kg, 0.72 mg/kg, and 1.08 mg/kg.

16C. The method of embodiment 15C, wherein fusion protein is administered at a dose of 0.01 mg/kg.

17C. The method of embodiment 15C, wherein fusion protein is administered at a dose of 0.03 mg/kg.

18C. The method of embodiment 15C, wherein fusion protein is administered at a dose of 0.09 mg/kg.

19C. The method of embodiment 15C, wherein fusion protein is administered at a dose of 0.18 mg/kg.

20C. The method of embodiment 15C, wherein fusion protein is administered at a dose of 0.36 mg/kg.

21C. The method of embodiment 15C, wherein fusion protein is administered at a dose of 0.72 mg/kg.

22C. The method of embodiment 15C, wherein fusion protein is administered at a dose of 1.08 mg/kg.

23C. The method of embodiment 22C, wherein the fusion protein is administered via subcutaneous injection.

### EXAMPLES

The following examples of the invention are to further illustrate the nature of the invention. It is believed that one of ordinary skill in the art can, using the preceding description and the following illustrative examples, make and utilize the present invention and practice the claimed methods. It should be understood that the following examples do not limit the invention and that the scope of the invention is to be determined by the appended claims.

### Example 1: Design of Fusion Molecules Comprising GDF15—Effect of GDF15 Truncations

Like other TGFβ family members, GDF15 is synthesized as a pre-pro-protein that forms a dimer in the endoplasmic reticulum and undergoes furin cleavage to produce secreted mature GDF15 (amino acids 197-308). The secreted mature GDF15 homodimer is about 25k Daltons, and each monomer has the potential to form up to 4 intramolecular disulfide bonds with a single intermolecular disulfide linking the homodimer components.

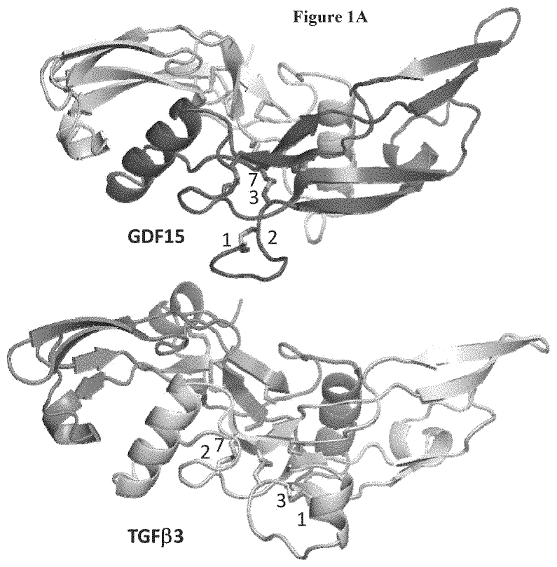

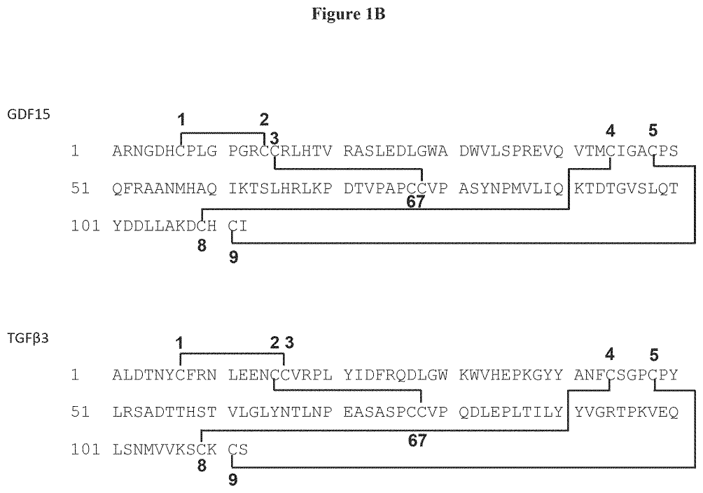

The crystal structure of GDF15 was determined in the invention and is depicted in FIGS. 1A and 1B. The crystal structure shows that the C-terminus of the mature GDF15 is buried in the dimer interface, while the N-terminus is exposed. This exposed terminus allows for the linkage of fusion proteins, such as half life extension proteins, to the N-terminus of GDF15.

The crystal structure also depicts the novel disulfide paring pattern of GDF15 cysteine residues. While TGFβ1 has C1-C3 and C2-C7 pairing (i.e., pairing between its first and third cysteine residues as well as between its second and seventh cysteine residues), GDF15 has C1-C2 and C3-C7 pairing (see FIGS. 1A and 1B). This unique disulfide pairing results in a loop formed by the C1-C2 pairing that is located at the N-terminus of the protein and away from the cysteine knot that contains other disulfide bonds. The structure predicts that the N-terminus of GDF15 may not be critical for dimer formation or overall protein folding, and that GDF15 and N-terminal fusion molecules thereof may be tolerable to N-terminal deletions that delete C1 and C2, residues within the C1-C2 loop, or even residues C-terminal to C2.

### Example 2: Design of Fusion Molecules Comprising GDF15—Effect of the Linker

Different linkers between the HSA molecule and the GDF15 molecule were evaluated. Both flexible linkers, containing the sequence (GGGGS)n, and structured linkers, containing the sequence (AP)n or (EAAAK)n, wherein n is 2 to 20, were evaluated.

Fusion proteins comprising the different linkers were compared for their biophysical properties, their effect on the efficacy of food intake in lean mice, their mouse pharmacokinetic (PK) values, and their ex vivo stability in human blood. The results of tested linker variants are shown in Table 1. The molecule comprising SEQ ID NO: 31, which contained the (EAAAK)₈ linker, showed aggregation by HPLC. The remaining seven linker variants in Table 1 demonstrated no aggregation.

TABLE 1
Summary of linker variant analysis

| SEQ | | Aggre- | Good Mouse | Ex vivo stability |
|---|---|---|---|---|
| ID NO* | Linker of | gation | PK (WT) | in human blood |
| 25 | AS(GGGGS)₂GT | No | Yes | Yes |
| 5 | GS(GGGGS)₄ | No | Yes | Yes |
| 26 | AS(GGGGS)₈GT | No | Yes | Yes |
| 27 | AS(AP)₅GT | No | Yes | Yes |
| 28 | AS(AP)₁₀GT | No | Yes | Yes |
| 29 | AS(AP)₂₀GT | No | Yes | Yes |
| 30 | AS(EAAAK)₄GT | No | Yes | Yes |
| 31 | AS(EAAAK)₈GT | YES | Not tested | Not tested |

*-6xHis tag was attached at the N-terminus for purification purpose

Linker stability was also evaluated for these variants by in vivo studies in mice and by ex vivo stability studies in human whole blood and plasma samples. Two forms of detection were used to analyze the results from these studies. An immunoassay with anti-GDF15 capture and anti-HSA detection antibody pairs was used to evaluate how intact the linker was by measuring the presence of both molecules on either side of the linker. A broader picture of the whole-molecule integrity was analyzed by liquid chromatography-mass spectrometry (LC-MS) analysis using different surrogate peptide sequences from both HSA and GDF15. The immunoassay demonstrated a stable PK profile for all of the linker variants and no loss of spiked plasma sample concentration for any of the linker variants observed over 48 hours. The LC-MS results were consistent with the immunoassay showing that the surrogate peptides from different parts of the HSA and GDF15 molecules were intact. The PK profile of the linker variants analyzed by LC-MS using surrogate peptides showed a similar trend for different linker variants, where they all had detectable levels at day 7. All the variants in Table 1 except for SEQ ID 31 had desirable biophysical properties and PK values.

The linker variants were evaluated for their in vivo activity by carrying out food intake studies in lean mice. Table 2 shows the influence of the linker variants on the efficacy of the fusion protein in decreasing food intake. There was a clear influence of the linker on the efficacy. With regard to the flexible (GGGGS)n linkers, an increase in the linker length from 2 to 4 to 8 dramatically increased the fusion protein efficacy. For the more rigid (AP)n linkers, the trend was less obvious, suggesting that the degree of freedom of the GDF15 molecule within the fusion protein plays a critical role in its efficacy.

TABLE 2
Effect of the linker on the in vivo efficacy of
HSA-GDF15 fusion proteins in lean mice

| SEQ | | % Decrease in |
|---|---|---|
| ID NO* | Linker | food intake (mean) |
| 25 | AS(GGGGS)₂GT | 28.8 |
| 5 | GS(GGGGS)₄ | 40.5 |
| 26 | AS(GGGGS)₈GT | 60.7 |
| 27 | AS(AP)₅GT | 48.2 |
| 28 | AS(AP)₁₀GT | 66.2 |
| 29 | AS(AP)₂₀GT | 55.1 |
| 30 | AS(EAAAK)₄GT | 51.9 |

*-6xHis tag was attached at the N-terminus for purification purpose

### Example 3: Design of Fusion Molecules Comprising GDF15—Effect of HSA Mutations

Recombinant proteins with the half life extension protein human serum albumin fused to the N-terminus of GDF15 through a linker were designed. This design should allow for the GDF15 dimerization interface to remain unperturbed and allow for the formation of the native inter-chain disulfide linkages, resulting in a GDF15 homodimer with HSA fusion extended from each GDF15 arm. With this approach, only a single gene is required to generate the HSA-GDF15 homodimer.

Native human serum albumin protein contains 35 cysteine (Cys, C) residues that form 17 disulfide bonds, with the Cys-34 residue being the only free cysteine in the molecule. This free Cys-34 has been shown to function as a free radical scavenger, by trapping multiple reactive oxygen species (ROS) and reactive nitrogen species (RNS). This free Cys was thus mutated to minimize the risk of heterogeneity due to oxidation.

The free cysteine at position 34 of HSA was mutated to either serine or alanine, and the GDF15 fusion molecules with either a HSA(C34S) or a HSA(C34A) mutation were analyzed. Both of the molecules were purified using a three-step purification method: (i) ion-exchange chromatography, (ii) hydrophobic interaction chromatography, and (iii) size-exclusion chromatography. When they were first generated, HPLC analysis showed that both molecules were pure and aggregation-free (Table 3).

However, two weeks after its generation, the fusion protein containing the HSA(C34A) mutation (comprising SEQ ID NO: 48) showed aggregation by HPLC, while the fusion protein containing the HSA(C34S) mutation (SEQ ID NO: 40) remained aggregation-free after four weeks.

TABLE 3
The influence of mutating HSA C34 on fusion protein aggregation

| SEQ | HSA | % aggregation when | % aggregation 2 weeks |
|---|---|---|---|
| ID NO | mutation | purified | post purification |
| 40 | C34S | 0 | 0 |
| 48 | C34A | 0 | 33.29 |

### Example 4: Protease Cleavage Propensity on GDF15

It was observed by the inventors that the arginine residue at amino acid position 198 of GDF15 (R198) is susceptible to protease degradation within the HSA-GDF15 fusion molecules. Such degradation results in a heterogeneous population and is undesirable for therapeutic compositions. The cleavage can be prevented by a protease inhibitor cocktail. Purification methods were investigated for the removal of the protease. Table 4 lists the two types of HSA affinity columns that were tested for purification of HSA-GDF15 fusion proteins, as measured by HPLC. At the time of purification, the HSA-GDF15 fusion proteins purified by both methods were 100% pure and intact. At low concentrations (2-5 mg/ml), proteins purified by both methods remained intact for the entire test period of 4 weeks. However, at high concentrations (40-50 mg/ml), only the antibody-based HSA resin (CaptureSelect) produced protease-free proteins that remain intact for the entire 4 week test period. The HSA-ligand-based resin (Albupure) generated proteins that were intact initially but demonstrated degradation over time when stored at high concentrations. Adding a protease inhibitor cocktail (PI) and EDTA completely arrested the degradation of the high concentration HSA-GDF15 fusion protein batch purified using the Albupure resin. Thus, the purification method plays a critical role in generating a stable therapeutic composition. Corresponding degradation was not observed in vivo or ex vivo, suggesting that once the therapeutic composition has been made protease-free, degradation of the fusion proteins is not an issue in vivo. Therefore, purification methods that can effectively remove potential proteases during production, such as those using the CaptureSelect resin, are key to successfully manufacturing GDF15 therapeutics that are homogenous, intact and stable.

TABLE 4
Protease cleavage of the HSA-GDF15 fusion proteins
can be eliminated by sample purification methods

| Condition | | | % degraded population of HSA-GDF15 (SEQ ID 60) | | | | |
|---|---|---|---|---|---|---|---|
| | | +PI/ | WEEK | WEEK | WEEK | WEEK | WEEK |
| Concentration | Resin | EDTA | 0 | 1 | 2 | 3 | 4 |
| Low | Capture | No | 0 | 0 | 0 | 0 | 0 |
| | Select | | | | | | |
| Low | Albupure | No | 0 | 0 | 0 | 0 | 0 |
| High | Capture | No | 0 | 0 | 0 | 0 | 0 |
| | Select | | | | | | |
| High | Albupure | No | 0 | 3.04 | 9.47 | 11.57 | 13.89 |
| High | Capture | Yes | 0 | 0 | 0 | 0 | 0 |
| | Select | | | | | | |
| High | Albupure | Yes | 0 | 0 | 0 | 0 | 0 |

### Example 5: N-Terminal Deletion Variants of GDF15

The GDF15 crystal structure depicted in FIGS. 1A and 1B predicts that the N-terminus of GDF15 involved in the deletion variants is not critical for dimer formation and overall protein folding. It also predicts that such N-terminal deletions should not affect any potential receptor interaction. HSA-GDF15 fusion proteins comprising various deletions of the N terminal of GDF15 were tested for in vivo activity.

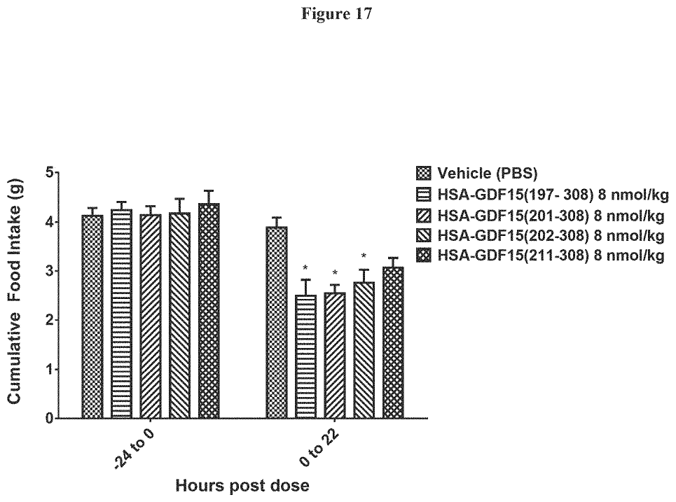

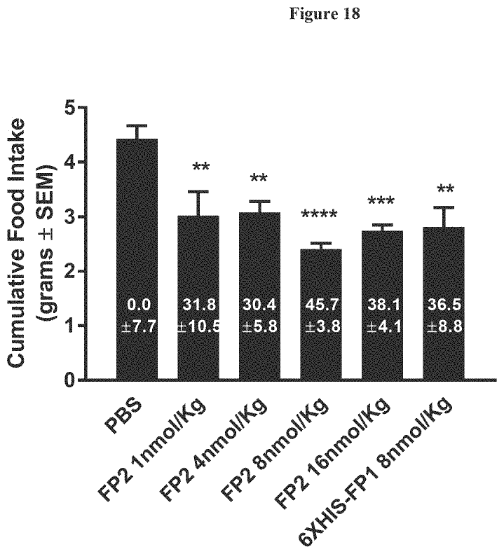

GDF15 N-terminal deletion variants were designed that removed the protease cleavage site at GDF15 (R198). Immediately following the R198 residue, there is a potential deamidation site at residues N199-G200, and substrate deamidation is also not favored in therapeutic compositions. GDF15 N-terminal deletions can remove both the proteolytic cleavage site and the deamidation sites simultaneously. The resulting GDF15 deletion variants that were incorporated into fusion proteins with HSA included GDF15 (201-308; SEQ ID NO: 8), GDF15 (202-308; SEQ ID NO: 9), and GDF15 (211-308; SEQ ID NO: 11). In vivo studies in mice showed that the N-terminal deletion variants of GDF15 are still active in reducing food intake (FIG. 17). The experimental results confirmed that such GDF15 N-terminal deletion variants express properly, form appropriate dimers, and are active in vivo.

### Example 6: Inactive Mutants of GDF15

Table 5 lists twelve mutants of GDF15 that were made to eliminate GDF15 in vivo activity and identify the functional epitope of GDF15. The mutants include five single mutants, two double mutants, and five triple mutants. HSA-GDF15 fusion proteins comprising these mutations were characterized for their biophysical properties and activities (Table 5). Out of the 12 mutants, one did not express and four formed aggregates over time, indicating that the mutations interrupt protein folding and biophysical properties. Of the remaining seven mutants, four of them contained a single mutation of GDF15, and these mutants were tested in mice for food intake reduction compared to wild type. Three of the single mutants (I89R, 189W and W32A) lost in vivo activity, while the remaining mutant (Q60W) is as active as the wild type. These results indicated that the I89R, I89W or W32A mutation interrupts the interaction of the receptor/co-receptor with GDF15, suggesting that the functional epitopes of GDF15 are around residues 189 and W32. The numbering of the mutation is based on the mature GDF15 present in fusion protein, e.g., “1” refers to the 1^{st} amino acid of the mature GDF15 (SEQ ID NO: 6) and “89” refers to the 89^{th} amino acid of the mature GDF15 protein.

TABLE 5
Summary of the biophysical properties and activities
of fusion proteins comprising GDF15 mutants

| SEQ | | | |
|---|---|---|---|
| ID NO | Mutations in GDF15 | Biophysical properties | Activities |
| 5 | Wild type | Expresses well, stable | Wild type |
| | | | activity |
| 64 | I89R | Expresses well, stable | Complete loss |
| | | | of activity |
| 65 | I89W | Expresses well, stable | Complete loss |
| | | | of activity |
| 66 | L34A, S35A, R37A | Expresses well, stable | |
| 67 | V87A, I89A, L98A | Expresses well, unstable | |
| 68 | L34A, S35A, I89A | Expresses well, stable | |
| 69 | V87A, I89A | Expresses well, stable | |
| 70 | Q60W | Expresses well, stable | As active as |
| | | | wild type |
| 71 | W32A | Expresses well, stable | Complete loss |
| | | | of activity |
| 72 | W29A | Expresses well, unstable | |
| 73 | Q60A, S64A, R67A | Expresses well, unstable | |
| 74 | W29A, Q60A, I61A | Does not express | |
| 75 | W29A, W32A | Expresses well, unstable | |

* 6xHis tag was attached at the N-terminus for purification purpose

### Example 7: Expression and Purification Methods

Expression

For expression of 20 ml and greater, the expression was done using HEK Expi293™ cells grown in Expi293™ Expression media. The cells were grown at 37° C. while shaking at 125 RPM with 8% C02. The cells were transfected at 2.5×106 cells per ml using the Expi293™ Expression Kit. For each liter of cells transfected, 1 mg of total DNA was diluted in 25 ml of Opti-MEM, and 2.6 ml of Expi293™ reagent was diluted in 25 ml of Opti-MEM and incubated for 5 minutes at room temperature. The diluted DNA and diluted Expi293 reagent were combined and incubated for 20 minutes at room temperature. The DNA complex was then added to the cells. The cells were placed in the shaking incubator overnight. The day after transfection, 5 ml of Enhancer 1 from the kit was diluted into 50 ml of Enhancer 2 from the kit, and the total volume of the two Enhancers was added to the cells. The transfected cells were placed back into the incubator for 4 days until they were harvested. The cells were concentrated by centrifugation at 6,000 g for 30 minutes and then filtered with a 0.2 um filter before the purification step.

The expression was also done in CHO cells. The plasmid was purified and characterized. Prior to transfection, 1 aliquot of 200 μg of plasmid DNA containing the coding region of HSA-GDF15 was linearized by restriction enzyme digestion with Acl I. The digestion with this restriction endonuclease ensures the removal of the ampicillin resistance gene. Two linearized 15 μg DNA aliquots were transfected into two 1×107 CHO cells (designated transfection pool A and B) using the BTX ECM 830 Electro Cell Manipulator (Harvard Apparatus, Holliston, Mass.). Cells were electroporated 3 times at 250 volts with 15 millisecond pulse lengths and 5 second pulse intervals in a 4 mm gap cuvette. Transfected cells were transferred to MACH-1+L-glutamine in a shake flask and incubated for 1 day. Transfection pool A and transfection pool B were centrifuged, resuspended in MACH-1+MSX, and transferred to shake flasks to incubate for 6 days. Transfected HSA-protein fusion-producing cells from transfection pool A and transfection pool B were pooled and plated in methylcellulose on day 8 post-electroporation.

Purification

Two-step purification using CaptureSelect resin and size exclusion chromatography was used. Cell supernatants from transiently transfected Expi293™ cells were loaded onto a pre-equilibrated (PBS, pH 7.2) HSA CaptureSelect column (CaptureSelect Human Albumin Affinity Matrix from ThermoFisher Scientific) at an approximate capacity of 10 mg protein per ml of resin. After loading, unbound proteins were removed by washing the column with 10 column volumes (CV) of PBS pH7.2. The HSA-GDF15 that was bound to the column was eluted with 10 CV of 2M MgCl₂ in 20 mM Tris, pH 7.0. Peak fractions were pooled, filtered (0.2μ), and dialyzed against PBS pH 7.2 at 4° C. After dialysis, the protein was filtered (0.2μ) again and concentrated to an appropriate volume before loading onto a 26/60 superdex 200 column (GE Healthcare). Protein fractions that eluted from the size exclusion chromatography (SEC) column with high purity (determined by SDS-PAGE) were pooled. The concentration of protein was determined by the absorbance at 280 nm on a BioTek Synergy HT™ spectrophotometer. The quality of the purified proteins was assessed by SDS-PAGE and analytical size exclusion HPLC (SE-HPLC, Dionex HPLC system). Endotoxin levels were measured using a LAL assay (Pyrotell®-T, Associates of Cape Cod).

Two-step purification using Albupure resin and SEC was also used. HSA-GDF15 fusion proteins were purified at room temperature using AlbuPure resin (ProMetic BioSciences Ltd) which utilizes an immobilized synthetic triazine ligand to selectively bind HSA. The expression supernatants were applied to the AlbuPure resin. The resin was then washed, first with 4 CV PBS pH 7.2 followed by 4 CV of 50 mM Tris pH 8.0, 150 mM NaCl buffer. The HSA-GDF15 that was bound to the column was eluted with 4 CV of PBS pH 7.2 buffer containing 100 mM Na Octanoate. The protein-containing fractions were concentrated to a 10 mL volume using a 30,000 kDa molecular weight cutoff spin concentrator (Amicon) and then applied to a 26/60 Superdex S200pg column (GE) that was equilibrated in PBS pH 7.2 buffer. SEC fractions containing HSA-GDF15 homodimer were identified via SDS-PAGE and pooled for analysis. The protein purities were assessed by SDSPAGE and SE-HPLC.

The Examples 8-14, and 19 involve characterization of an exemplary fusion protein of the invention, has the amino acid sequence of SEQ ID NO: 60. This fusion protein is a fully recombinant protein that exists as a homodimer of a fusion of HSA with the mature human GDF15 through a 42-amino acid linker consisting of glycine and serine residues, GS-(GGGGS)₈. The predicted molecular weight of this fusion protein is 162,696 Daltons, and the single native free cysteine at position 34 of HSA has been mutated to serine. This particular HSA-GDF15 fusion protein will be referred to simply as “FP1” in the following examples, for simplicity. A 6×His-tagged variant of FP1 (6×His-FP1, SEQ ID NO: 26), containing an AS-(GGGGS)×8-GT linker, was used for comparison in some of the following examples.

### Example 8: Effects of FP1 on the Food Intake of C57Bl/6 Mice

The purpose of this experiment was to demonstrate the dose-responsive effect of FP1 on the inhibition of food intake in C57Bl/6 mice.

Male C57Bl/6 mice were acclimated for a minimum of 72 hours in BioDAQ cages. Mice were then grouped based on food intake in the previous 24 hours into six groups of eight. Between 4:00 and 5:00 pm, animals were weighed and dosed with vehicle or a composition comprising FP1 via subcutaneous injection. The change in food weight for each cage was recorded continuously by the BioDAQ system for a period of 48 hours after the injections. 6×His-FP1 was used for comparison in this study.

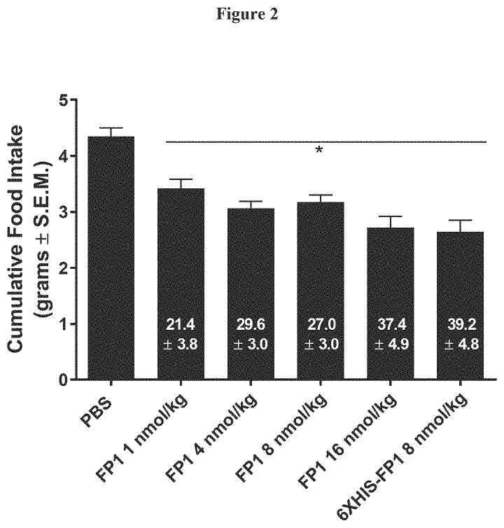

The results (FIG. 2 and Table 6) were expressed as an average of cumulative food intake for a given time interval. The results indicated that subcutaneous administration of FP1 to C57BL/6 mice significantly inhibited food intake relative to vehicle-treated animals at all doses and time points tested. 6×His-FP1 reduced food intake at the 8 nmol/kg dose.

TABLE 6
Effects of subcutaneous administration of FP1 on food
intake in C57BL/6 mice; Cumulative food intake at
12, 24 and 48 hours post administration is shown
Cumulative Food Intake (g)

| Treatment | 12 hours | 24 hours | 48 hours |
|---|---|---|---|
| PBS | 3.7 ± 0.2 | 4.3 ± 0.1 | 8.4 ± 0.3 |
| FP1, 1 nmol/kg | 2.9 ± 0.1* | 3.4 ± 0.2** | 7.0 ± 0.3** |
| FP1, 4 nmol/kg | 2.3 ± 0.2**** | 3.1 ± 0.1**** | 6.6 ± 0.3*** |
| FP1, 8 nmol/kg | 2.1 ± 0.2**** | 3.2 ± 0.1*** | 6.4 ± 0.2**** |
| FP1, 16 nmol/kg | 1.7 ± 0.2**** | 2.7 ± 0.2**** | 6.2 ± 0.3**** |
| 6xHis-FP1, 8 nmol/kg | 1.8 ± 0.2**** | 2.6 ± 0.2**** | 6.0 ± 0.2**** |

Data are expressed as Mean ± SEM.
*p ≤ 0.05, versus PBS;
**p ≤ 0.01, versus PBS;
***p ≤ 0.001, versus PBS;
****p ≤ 0.0001, versus PBS
One-Way ANOVA-Tukey's multiple comparisons test;
n = 8/group

### Example 9: Effects of FP1 on Food Intake in Sprague Dawley Rats

The purpose of this experiment was to demonstrate the dose-responsive effect of FP1 on the inhibition of food intake in Sprague Dawley rats.

Male Sprague-Dawley rats were acclimated for a minimum of 72 hours in the BioDAQ cages. Rats were then grouped based on food intake in the previous 24 hours into six groups of eight. Between 4:00 and 5:00 pm, animals were weighed and dosed with vehicle or a composition comprising the fusion protein via subcutaneous injection. The change in food weight for each cage was recorded continuously by the BioDAQ system, for a period of 48 hours after the injections. 6×His-FP1 was used for comparison in this study.

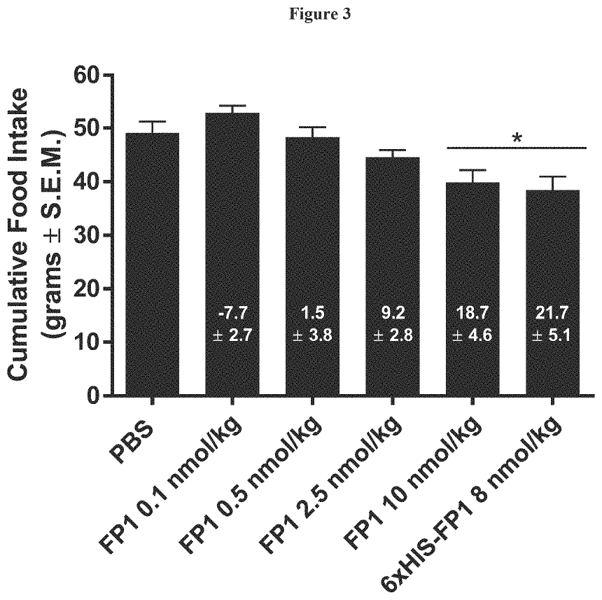

The results are shown in FIG. 3 and Table 7. Subcutaneous administration of FP1 inhibited food intake at doses of 2.5 nmol/kg and 10 nmol/kg compared to vehicle-treated animals. The inhibition reached statistical significance only with the highest dose tested (10 nmol/kg) at 24 and 48 hours post-administration. FP1 reduced food intake at the 8 nmol/kg dose, and the effect was significant at 24 and 48 hours.

TABLE 7
Effects of subcutaneous administration of FP1 on food
intake in Sprague-Dawley rats; cumulative food intake at
12, 24 and 48 hours post administration is shown
Cumulative Food Intake (g)

| Treatment | 12 hours | 24 hours | 48 hours |
|---|---|---|---|
| PBS | 20.6 ± 1.3 | 25.3 ± 1.3 | 49.1 ± 2.1 |
| FP1, 0.1 nmol/kg | 23.3 ± 1.4 | 26.9 ± 0.8 | 52.8 ± 1.3 |
| FP1, 0.5 nmol/kg | 22.6 ± 1.7 | 25.1 ± 1.0 | 48.3 ± 1.8 |
| FP1, 2.5 nmol/kg | 20.0 ± 1.4 | 22.0 ± 1.0 | 44.6 ± 1.4 |
| FP1, 10 nmol/kg | 18.7 ± 0.9 | 19.9 ± 1.0* | 39.9 ± 2.3* |
| 6xHis-FP1, 8 nmol/kg | 17.0 ± 1.5 | 18.8 ± 1.4** | 38.4 ± 2.5** |

Data are expressed as Mean ± SEM.
*p ≤ 0.05, versus PBS;
**p ≤ 0.01, versus PBS
One-Way ANOVA-Tukey's multiple comparisons test;
n = 8/group

### Example 10: Effects of FP1 on Glucose Homeostasis and Body Weight in Diet-Induced Obese (DIO) Mice

The purpose of this experiment was to evaluate the effects of FP1 on food intake, body weight, and glucose homeostasis throughout two weeks of treatment in DIO C57Bl/6 mice.

Male DIO mice were weighed, and FP1 was dosed subcutaneously at 2 ml/kg every three days (q3d) at Day 0, 3, 6, 9, and 12. The vehicle and rosiglitazone treatment groups were dosed with PBS on a similar regimen. The control rosiglitazone was provided in the diet at 0.015% ad libitum. Mouse and food weights were recorded daily. Glucose was measured using a glucometer (One Touch® Ultra®, Lifescan, Milpitas, Calif.). Fat and lean mass was quantitated in conscious mice by time-domain NMR (TD-NMR) using the Bruker Mini-Spec LF110. For an oral glucose tolerance test (OGTT), mice were fasted for 4 hours. Blood glucose was measured via tail snip at 0, 30, 60, 90, and 120 minutes post oral gavage administration of 2 g/kg glucose at 10 mL/kg. Insulin was measured at 0, 30, and 90 minutes post glucose administration.

At the end of the study, the mice were euthanized via CO2 inhalation, and a terminal blood sample was collected. Serum was placed into a 96 well plate on wet ice and then stored at −80° C. The liver was removed, and the fat content relative to the total mass of liver sections was assessed using TD-NMR with the Bruker MiniSpec mq60 according to the manufacturer's instructions.

The fasted homeostatic model assessment of insulin resistance (HOMA-IR) was calculated based on the product of fasted glucose (in mg/dL) and insulin (in mU/L) divided by a factor of 405.

Treatment of DIO mice with FP1 q3d at 1 nmol/kg and 10 nmol/kg reduced body weight (Table 8) and food intake (Table 9). The reductions reached statistical significance only at certain time points, as described below.

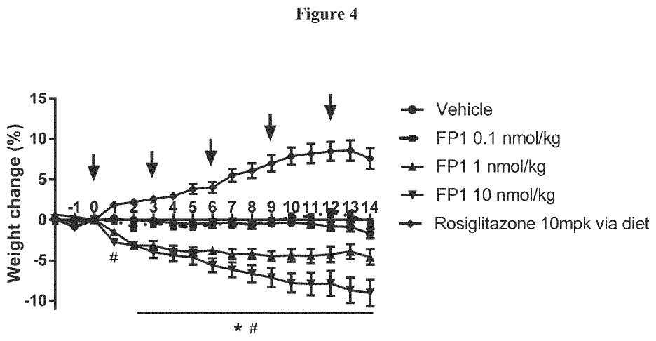

Fp1 decreased body weight at doses of 1 (from day 2 to 14) and 10 nmol/kg (from day 1 to 14) in DIO mice (Table 8 and FIG. 4). A significant reduction in food intake was seen at days 1 and 2 of the study at the dose of 1 nmol/kg and at days 1, 8 and 9 at the 10 nmol/kg dose (Table 9).

TABLE 8
Body weight change (% of starting) during treatment with FP1 in DIO mice

| | | | | | Rosiglit- |
|---|---|---|---|---|---|
| Treat- | | | | | azone |
| ment | Vehicle | FP1 (nmol/kg) | | | 10 |
| Day | n/a | 0.1 | 1 | 10 | mpk/day |
| −2 | 0.1 ± 0.2 | −0.1 ± 0.4 | 0.6 ± 0.4 | 0.2 ± 0.3 | 0.1 ± 0.2 |
| −1 | −0.8 ± 0.3 | −0.2 ± 0.3 | 0.4 ± 0.3 | −0.3 ± 0.4 | −0.9 ± 0.2 |
| 0 | 0.0 ± 0.0 | 0.0 ± 0.0 | 0.0 ± 0.0 | 0.0 ± 0.0 | 0.0 ± 0.0 |
| 1 | 0.1 ± 0.2 | −0.1 ± 0.4 | −1.5 ± 0.5 | −2.8 ± 0.4* | 1.9 ± 0.2 |
| 2 | 0.0 ± 0.3 | −0.8 ± 0.5 | −3.2 ± 0.4* | −3.1 ± 0.5* | 2.2 ± 0.5 |
| 3 | −0.2 ± 0.4 | −0.4 ± 0.4 | −3.2 ± 0.6* | −4.0 ± 0.7* | 2.6 ± 0.5 |
| 4 | −0.4 ± 0.5 | −0.7 ± 0.5 | −3.8 ± 0.6* | −4.4 ± 0.8* | 3.0 ± 0.5* |
| 5 | −0.5 ± 0.4 | −1.0 ± 0.3 | −4.0 ± 0.5* | −4.6 ± 0.9* | 3.8 ± 0.6* |
| 6 | −0.4 ± 0.6 | −0.5 ± 0.5 | −3.8 ± 0.4* | −5.6 ± 0.9* | 4.0 ± 0.7* |
| 7 | −0.3 ± 0.5 | −0.5 ± 0.5 | −4.3 ± 0.6* | −6.1 ± 0.9* | 5.5 ± 0.8* |
| 8 | −0.6 ± 0.5 | −0.5 ± 0.5 | −4.2 ± 0.6* | −6.7 ± 1.1* | 6.1 ± 0.9* |
| 9 | −0.4 ± 0.5 | −0.2 ± 0.5 | −4.5 ± 0.6* | −7.2 ± 1.2* | 7.0 ± 1.0* |
| 10 | −0.3 ± 0.5 | 0.3 ± 0.5 | −4.4 ± 0.8* | −7.8 ± 1.2* | 7.8 ± 1.1* |
| 11 | −0.5 ± 0.5 | 0.5 ± 0.4 | −4.4 ± 0.8* | −7.9 ± 1.4* | 8.2 ± 1.2* |
| 12 | −0.8 ± 0.6 | 0.8 ± 0.5 | −4.3 ± 1.0* | −7.9 ± 1.5* | 8.5 ± 1.2* |
| 13 | −0.8 ± 0.6 | 0.5 ± 0.5 | −3.9 ± 0.9* | −8.7 ± 1.6* | 8.6 ± 1.3* |
| 14 | −1.7 ± 0.6 | −0.4 ± 0.5 | −4.6 ± 1.0* | −9.0 ± 1.7* | 7.6 ± 1.3* |

Data are expressed as Mean ± SEM.
n = 8 per group.
*= p < 0.05, compared to that of the vehicle treated group.

TABLE 9
Daily food intake (gm) during treatment with FP1 in DIO mice

| | | | | | Rosiglit- |
|---|---|---|---|---|---|
| Treat- | | | | | azone |
| ment | Vehicle | FP1 (nmol/kg) | | | 10 |
| Day | n/a | 0.1 | 1 | 10 | mpk/day |
| −2 | 2.9 ± 0.1 | 3.0 ± 0.2 | 2.9 ± 0.1 | 2.8 ± 0.1 | 2.8 ± 0.1 |
| 0 | 3.0 ± 0.1 | 3.0 ± 0.1 | 2.8 ± 0.1 | 2.8 ± 0.1 | 3.0 ± 0.1 |
| 1 | 2.9 ± 0.1 | 3.1 ± 0.1 | 2.1 ± 0.2* | 1.5 ± 0.1* | 3.6 ± 0.1* |
| 2 | 3.0 ± 0.1 | 3.0 ± 0.1 | 2.3 ± 0.1* | 2.6 ± 0.2 | 3.9 ± 0.2* |
| 3 | 2.8 ± 0.1 | 3.0 ± 0.1 | 2.6 ± 0.1 | 2.4 ± 0.2 | 3.5 ± 0.1* |
| 4 | 2.3 ± 0.1 | 2.6 ± 0.1 | 2.2 ± 0.1 | 2.3 ± 0.1 | 3.3 ± 0.1* |
| 5 | 2.8 ± 0.1 | 3.0 ± 0.1 | 2.8 ± 0.1 | 2.6 ± 0.1 | 3.7 ± 0.2* |
| 6 | 2.8 ± 0.1 | 3.1 ± 0.1 | 2.9 ± 0.1 | 2.4 ± 0.2 | 3.7 ± 0.2* |
| 7 | 2.7 ± 0.1 | 2.9 ± 0.1 | 2.6 ± 0.1 | 2.4 ± 0.1 | 3.8 ± 0.2* |
| 8 | 2.7 ± 0.1 | 2.9 ± 0.1 | 2.6 ± 0.1 | 2.1 ± 0.1* | 3.5 ± 0.2* |
| 9 | 3.0 ± 0.1 | 3.3 ± 0.1 | 2.8 ± 0.1 | 2.5 ± 0.2* | 4.3 ± 0.2* |
| 10 | 2.6 ± 0.1 | 3.0 ± 0.1 | 2.6 ± 0.1 | 2.2 ± 0.1 | 3.6 ± 0.1* |
| 11 | 2.9 ± 0.1 | 3.1 ± 0.1 | 2.7 ± 0.2 | 2.6 ± 0.2 | 3.4 ± 0.1* |
| 12 | 2.7 ± 0.1 | 3.1 ± 0.1 | 3.0 ± 0.1 | 3.0 ± 0.2 | 3.6 ± 0.2* |
| 13 | 2.6 ± 0.1 | 2.8 ± 0.1 | 2.8 ± 0.1 | 2.2 ± 0.2 | 3.2 ± 0.1* |

Data are expressed as Mean ± SEM.
n = 8 per group.
*= p < 0.05, compared to that of the vehicle treated group.

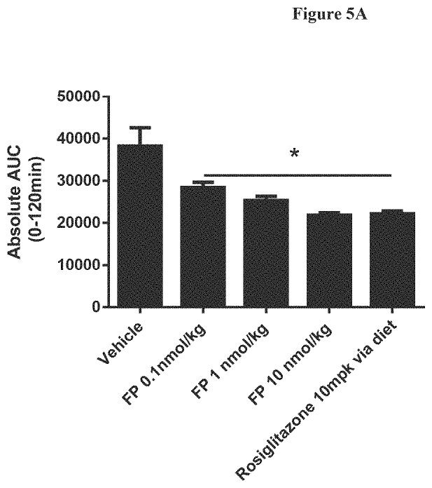

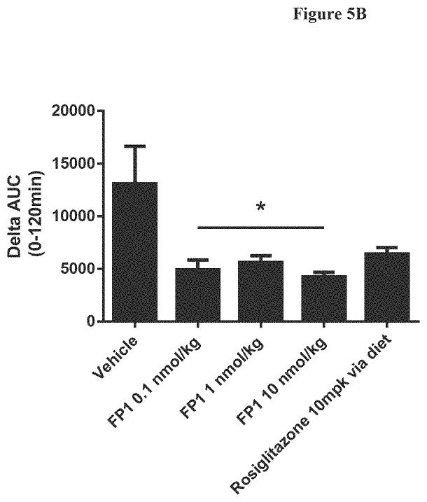

In an OGTT performed on day 14 of the study, FP1 significantly lowered glucose levels compared to vehicle-treated animals at all time points after time 0 at all three doses tested (Table 10). This was further quantitated as total area under the curve (AUC) and delta AUC, which were significantly lower compared to vehicle for all three doses tested (Table 10 and FIGS. 5A and 5B).

TABLE 10
Blood glucose (mg/dL) levels during an OGTT after fourteen days of q3d dosing of FP1 in DIO mice

| | Dose | Time after Glucose Challenge (min) | | | | | Total AUC | Delta AUC |
|---|---|---|---|---|---|---|---|---|
| Treatment | (nmol/kg) | 0 | 30 | 60 | 90 | 120 | (mg/dL/120 min) | (mg/dL/120 min) |
| Vehicle | NA | 209.6 ± | 399.4 ± | 338.5 ± | 297.5 ± | 269.3 ± | 38244.4 ± | 13089.4 ± |
| | | 16.5 | 46.4 | 46.3 | 38.0 | 36.6 | 4308.2 | 3573.0 |
| FP1 | 0.1 | 196.6 ± | 319.0 ± | 241.6 ± | 196.0 ± | 184.0 ± | 28408.1 ± | 4885.5 ± |
| | | 7.6 | 21.5* | 14.4* | 12.8* | 6.3* | 1236.1* | 955.1* |
| | 1 | 164.0 ± | 251.4 ± | 227.6 ± | 191.9 ± | 180.6 ± | 25295.6 ± | 5615.6 ± |
| | | 7.2 | 13.0* | 12.6* | 9.4* | 13.1* | 1026.7* | 647.6* |
| | 10 | 146.6 ± | 195.6 ± | 197.1 ± | 172.1 ± | 174.9 ± | 21768.8 ± | 4211.4 ± |
| | | 5.9 | 14.5* | 6.9* | 6.3* | 13.8* | 603.0* | 425.8* |
| Rosiglitazone | 10 | 130.8 ± | 199.1 ± | 204.4 ± | 184.1 ± | 169.1 ± | 22115.6 ± | 6425.6 ± |
| | mpk/day | 6.0 | 12.9* | 8.5* | 10.9* | 5.7* | 671.9* | 599.6 |

Data are expressed as Mean ± SEM.
n = 8 per group.
*= p < 0.05, compared to that of the vehicle treated group.

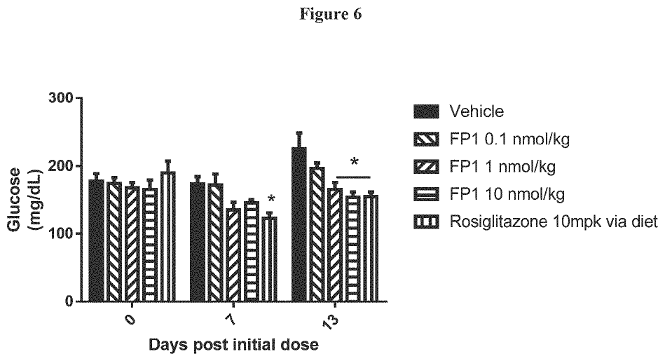

Fed blood glucose levels were measured at the start (day 0), at day 7 and at day 13 of the study (Table 11 and FIG. 6). FP1 decreased blood glucose in a statistically significant manner at doses of 1 nmol/kg and 10 nmol/kg on day 13 of the study.

TABLE 11
Fed blood glucose during treatment of
DIO mice with q3d treatment of FP1

| | Dose | Time after start of treatment (days) | | |
|---|---|---|---|---|
| Treatment | (nmol/kg) | 0 | 7 | 13 |
| Vehicle | NA | 177.6 ± 10.9 | 173.6 ± 10.6 | 225.3 ± 23.0 |
| FP1 | 0.1 | 174.1 ± 8.5 | 171.8 ± 16.3 | 196.4 ± 8.1 |
| | 1 | 167.5 ± 7.8 | 135.1 ± 11.2 | 165.3 ± 10.3* |
| | 10 | 165.3 ± 13.7 | 145.3 ± 4.9 | 153.8 ± 7.5* |
| Rosiglit- | 10 mpk/day | 189.6 ± 17.3 | 122.6 ± 8.1* | 154.8 ± 6.5* |
| azone | | | | |

Data are expressed as Mean ± SEM.
n = 8 per group.
*= p < 0.05, compared to that of the vehicle treated group.

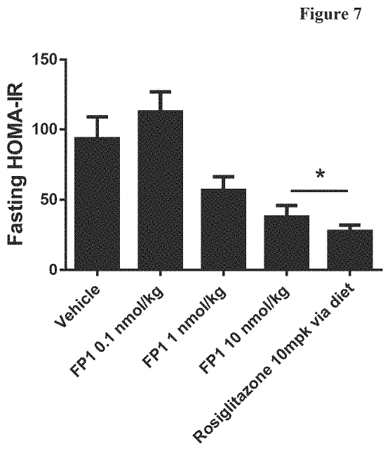

Plasma insulin levels during the OGTT were significantly higher for FP1 than for the corresponding vehicle group for a 0.1 nmol/kg dose at 30 minutes, and lower at the 1 and 10 nmol/kg doses at the same time point (Table 12). The insulin excursion during the OGTT, as measured by total AUC, was higher than the vehicle group for the 0.1 nmol/kg dose of FP1 (Table 12), and lower at the 1 and 10 nmol/kg dose. In both cases, statistical significance was reached only at the lowest dose. At the 90 minute time point, mice treated with 1 and 10 nmol/kg of FP1 had lower insulin levels; however, this effect did not achieve statistical significance. HOMA-IR, used as a measure of insulin sensitivity, was measured on day 14 of the study. At this time point, FP1 decreased HOMA-IR, or improved insulin sensitivity, at 10 nmol/kg (Table 13 and FIG. 7).

TABLE 12
Plasma insulin (pg/mL) levels during an OGTT after fourteen days of q3d dosing of FP1 in DIO mice

| | Dose | Time after Glucose Challenge (min) | | | Total AUC |
|---|---|---|---|---|---|
| Treatment | (nmol/kg) | 0 | 30 | 90 | (pg/mL/90 min) |
| Vehicle | NA | 6096.0 ± 774.3 | 14660.0 ± 3031.2 | 5034.4 ± 405.1 | 902151.9 ± 143123.2 |
| FP1 | 0.1 | 7861.0 ± 779.5 | 33825.8 ± 7902.0* | 6494.4 ± 797.0 | 1834808.8 ± 381276.1* |
| | 1 | 4808.1 ± 795.8 | 13061.8 ± 2226.3 | 3443.5 ± 342.5 | 763218.1 ± 119766.8 |
| | 10 | 3478.3 ± 634.6 | 7147.0 ± 823.8 | 2958.0 ± 414.0 | 462528.8 ± 44653.1 |
| Rosiglitazone | 10 mpk/day | 2965.6 ± 524.2 | 2203.1 ± 193.5* | 1180.0 ± 57.1 | 179025.0 ± 9970.7 |

Data are expressed as Mean ± SEM.
n = 8 per group.
*= p < 0.05, compared to that of the vehicle treated group.

TABLE 13
Fasted HOMA-IR in DIO mice after fourteen
days of q3d treatment of FP1

| Treatment | Dose (nmol/kg) | HOMA-IR |
|---|---|---|
| Vehicle | NA | 93.5 ± 15.3 |
| FP1 | 0.1 | 112.4 ± 14.6 |
| | 1 | 56.7 ± 9.7 |
| | 10 | 37.7 ± 8.2* |
| Rosiglitazone | 10 mpk/day | 27.3 ± 4.7* |

Data are expressed as Mean ± SEM.
n = 8 per group.
*= p < 0.05, compared to that of the vehicle treated group.

The magnitude of weight loss achieved by day 13 did not result in measurable changes in absolute fat mass or percent fat mass at any dose (Table 14). At the 10 nmol/kg dose, there was a significant decrease in absolute lean mass. This decrease was not observed when expressed as percent lean mass. Liver weights were measured during terminal necropsy on day 15 of the study (Table 15). FP1 decreased absolute liver weight and liver weight as a percentage of body weight at the 10 nmol/kg dose. A decrease was observed at the 1 nmol/kg dose, but this did not reach statistical significance for either parameter. Liver fat was measured on a biopsy by NMR (Table 16). FP1 fusion protein decreased hepatic fat content, expressed as a percentage of liver biopsy weight, at 1 and 10 nmol/kg doses. The reduction was significant at the higher dose.

TABLE 14
Body composition after thirteen days of treatment with FP1 q3d in DIO mice

| | Dose | Fat Mass | Lean Mass | Fat Mass | Lean Mass |
|---|---|---|---|---|---|
| Treatment | (nmol/kg) | (g) | (g) | (% of body) | (% of body) |
| Vehicle | NA | 11.6 ± 0.5 | 29.6 ± 0.4 | 23.4 ± 0.8 | 59.6 ± 0.9 |
| FP1 | 0.1 | 12.2 ± 0.4 | 29.7 ± 0.4 | 24.1 ± 0.6 | 58.5 ± 0.6 |
| | 1 | 12.1 ± 0.3 | 28.0 ± 0.4 | 25.1 ± 0.3 | 58.3 ± 0.6 |
| | 10 | 10.6 ± 0.3 | 27.7 ± 0.4* | 23.1 ± 0.3 | 60.6 ± 0.6 |
| Rosiglitazone | 10 mpk/day | 14.9 ± 0.6* | 30.0 ± 0.5 | 27.2 ± 0.4* | 55.0 ± 0.6* |

Data are expressed as Mean ± SEM.
n = 8 per group.
*= p < 0.05, compared to that of the vehicle treated group.

TABLE 15
Liver weight after fifteen days of
treatment with FP1 q3d in DIO mice

| | | Liver | Liver |
|---|---|---|---|
| | Dose | weight | Weight |
| Treatment | (nmol/kg) | (g) | (% of body) |
| Vehicle | NA | 2.6 ± 0.1 | 5.4 ± 0.2 |
| FP1 | 0.1 | 2.9 ± 0.2 | 5.8 ± 0.2 |
| | 1 | 2.2 ± 0.1 | 4.6 ± 0.2 |
| | 10 | 1.9 ± 0.1* | 4.2 ± 0.1* |
| Rosiglitazone | 10 mpk/day | 2.5 ± 0.2 | 4.6 ± 0.2 |

Data are expressed as Mean ± SEM.
n = 8 per group.
*= p < 0.05, compared to that of the vehicle treated group.

TABLE 16
Liver fat content measured after fifteen days
of treatment with FP1 q3d in DIO mice

| Treatment | Dose (nmol/kg) | Fat (%) |
|---|---|---|
| Vehicle | NA | 27.3 ± 2.1 |
| FP1 | 0.1 | 26.0 ± 1.4 |
| | 1 | 22.5 ± 1.4 |
| | 10 | 17.8 ± 1.6* |
| Rosiglitazone | 10 mpk/day | 25.9 ± 0.8 |

Data are expressed as Mean ± SEM.
n = 8 per group.
*= p < 0.05, compared to that of the vehicle treated group.

### Example 11: Effects of FP1 on Blood Glucose Levels and Body Weight in Ob/Ob Mice

The purpose of this experiment was to evaluate the effects of FP1 on body weight and blood glucose levels over eight days of treatment in obese, hyperglycemic, leptin-deficient ob/ob mice.

Male ob/ob mice were weighed and FP1 was administered subcutaneously at 2 mL/kg every three days (q3d) at Day 0, 3 and 6. Mouse and food weights were recorded daily. Glucose was measured daily using a glucometer. At the end of the study, mice were euthanized, and a terminal blood sample was collected.

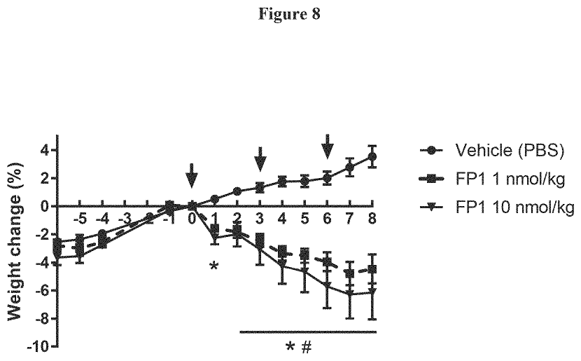

FP1, at the 1 nmol/kg dose, significantly decreased body weight (expressed as a percentage of starting body weight) in ob/ob mice starting at day 2 until day 8, relative to vehicle-treated mice. FP1, at the 10 nmol/kg dose, decreased body weight (expressed as a percentage of starting body weight) in ob/ob mice starting at day 1 until day 8 relative to vehicle-treated mice (Table 17 and FIG. 8).

TABLE 17
Body weight change (% of starting) during
treatment with FP1 q3d in ob/ob mice

| | | |
|---|---|---|
| Treatment | Vehicle | FP1 |

| Day | n/a | 1 nmol/kg | 10 nmol/kg |
|---|---|---|---|
| −6 | −2.5 ± 0.3 | −2.8 ± 0.4 | −3.6 ± 0.5 |
| −5 | −2.4 ± 0.3 | −3.0 ± 0.5 | −3.5 ± 0.5 |
| −4 | −1.9 ± 0.2 | −2.5 ± 0.4 | −2.8 ± 0.3 |
| −1 | −0.3 ± 0.3 | 0.1 ± 0.2 | −0.3 ± 0.2 |
| 0 | 0.0 ± 0.0 | 0.0 ± 0.0 | 0.0 ± 0.0 |
| 1 | 0.5 ± 0.2 | −1.6 ± 0.2 | −2.3 ± 0.5* |
| 2 | 1.1 ± 0.2 | −1.8 ± 0.4* | −2.0 ± 0.9* |
| 3 | 1.3 ± 0.3 | −2.5 ± 0.4* | −3.1 ± 1.1* |
| 4 | 1.8 ± 0.3 | −3.3 ± 0.5* | −4.2 ± 1.3* |
| 5 | 1.8 ± 0.4 | −3.5 ± 0.5* | −4.7 ± 1.5* |
| 6 | 2.0 ± 0.5 | −4.0 ± 0.7* | −5.7 ± 1.6* |
| 7 | 2.8 ± 0.6 | −4.8 ± 0.8* | −6.3 ± 1.7* |
| 8 | 3.5 ± 0.8 | −4.5 ± 1.0* | −6.1 ± 1.9* |

Data are expressed as Mean ± SEM.
n = 8 per group.
*= p < 0.05, compared to that of the vehicle treated group.

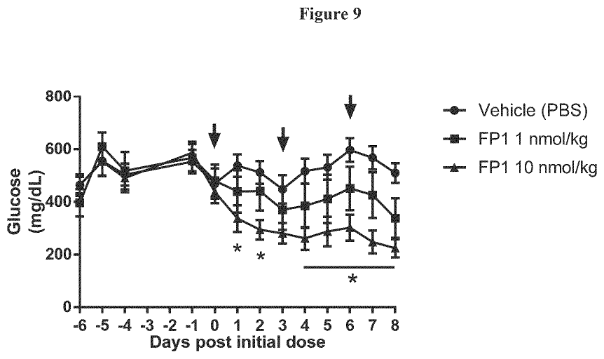

FP1, at the 10 nmol/kg dose, decreased fed blood glucose values in ob/ob mice on study day 1 and 2 and from day 4 until day 8 relative to vehicle-treated mice. A reduction in in blood glucose was observed at 1 nmol/kg; however, this effect did not reach statistical significance (Table 18 and FIG. 9).

TABLE 18
Fed blood glucose during treatment of ob/ob mice with FP1 q3d

| | | |
|---|---|---|
| Treatment | Vehicle | FP1 |

| Day | n/a | 1 nmol/kg | 10 nmol/kg |
|---|---|---|---|
| −6 | 463.6 ± 31.2 | 395.7 ± 52.0 | 463.6 ± 41.0 |
| −5 | 554.9 ± 57.5 | 609.6 ± 53.6 | 552.9 ± 53.0 |
| −4 | 502.9 ± 41.6 | 517.9 ± 71.6 | 490.3 ± 54.4 |
| −1 | 552.1 ± 45.1 | 567.2 ± 51.2 | 586.0 ± 42.6 |
| 0 | 468.8 ± 57.3 | 479.7 ± 61.8 | 437.0 ± 42.7 |
| 1 | 537.0 ± 42.8 | 439.0 ± 80.4 | 336.2 ± 51.0* |
| 2 | 511.7 ± 43.5 | 440.0 ± 74.3 | 293.7 ± 37.6* |
| 3 | 447.8 ± 54.2 | 369.6 ± 75.9 | 279.4 ± 38.8 |
| 4 | 516.3 ± 47.0 | 384.6 ± 84.0 | 261.2 ± 43.7* |
| 5 | 531.0 ± 47.1 | 410.9 ± 92.2 | 286.9 ± 55.8* |
| 6 | 596.1 ± 45.1 | 451.1 ± 82.6 | 301.9 ± 49.3* |
| 7 | 566.7 ± 44.3 | 425.3 ± 86.9 | 246.7 ± 43.5* |
| 8 | 509.9 ± 37.6 | 337.8 ± 75.1 | 223.4 ± 34.1* |

Data are expressed as Mean ± SEM.
n = 8 per group.

### Example 12: Multispecies Pharmacokinetics

Mouse Pharmacokinetics

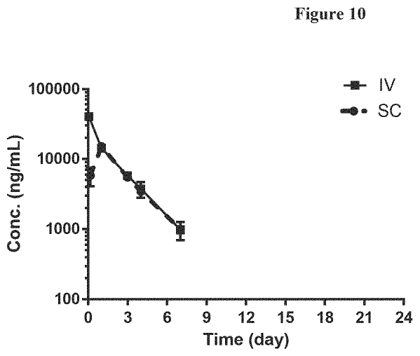

FP1 was administered to female C57Bl/6 mice at a dose of 2 mg/kg IV and SC in PBS, pH 7. Blood samples were collected, serum was processed and drug concentrations were measured up to 7 days following both routes of administration. The concentration of FP1 was determined using an immunoassay method. The serum drug concentration-time profile is summarized in Tables 19 and 20 and illustrated in FIG. 10.

TABLE 19
Serum concentration (nM) of FP1 over time following
a single SC administration in C57Bl/6 female mice

| | FP1 - SC Dose | | | | | | |
|---|---|---|---|---|---|---|---|
| | Animal | Animal | Animal | Animal | Animal | Average | |
| | 56 Result | 57 Result | 58 Result | 60 Result | 63 Result | Result | Std |
| Timepoint | (nM) | (nM) | (nM) | (nM) | (nM) | (nM) | Dev |
| 4 hr | 32.299 | 50.735 | 42.766 | 32.407 | 23.018 | 36.245 | 10.698 |
| 24 hr | 88.822 | 106.418 | 88.648 | 103.841 | 80.346 | 93.615 | 11.093 |
| 72 hr | 33.563 | 38.473 | 32.473 | 33.625 | 32.769 | 34.181 | 2.451 |
| 96 hr | 20.639 | 24.988 | 21.247 | 19.356 | 20.771 | 21.400 | 2.124 |
| Day 7 | 5.399 | 6.919 | 7.234 | 5.994 | 5.637 | 6.237 | 0.803 |

TABLE 20
Serum concentration (nM) of FP1 over time following
a single IV administration in C57Bl/6 female mice

| | FP1 - IV Dose | | | | | | |
|---|---|---|---|---|---|---|---|
| | Animal | Animal | Animal | Animal | Animal | Average | |
| | 52 Result | 53 Result | 65 Result | 66 Result | 70 Result | Result | Std |
| Timepoint | (nM) | (nM) | (nM) | (nM) | (nM) | (nM) | Dev |
| 1 hr | 240.419 | 233.318 | 232.484 | 276.913 | 272.727 | 251.172 | 21.857 |
| 24 hr | 86.823 | 95.774 | 80.201 | 93.153 | 88.853 | 88.961 | 6.027 |
| 72 hr | 33.634 | 37.447 | 33.108 | 41.680 | 34.034 | 35.981 | 3.612 |
| 96 hr | 22.666 | 20.588 | 19.458 | 33.718 | 20.361 | 23.358 | 5.909 |
| Day 7 | 7.401 | 5.606 | 4.896 | 8.556 | 4.205 | 6.133 | 1.803 |

Pharmacokinetic analysis revealed a terminal half-life of 1.67 and 1.57 days for FP1 in C57Bl/6 mice following SC and IV administration, respectively (Table 21). FP1 demonstrated a mean bioavailability of ˜71% following SC administration.

TABLE 21
Mean (±SD) pharmacokinetic parameters of FP1 following
2 mg/kg IV and SC administration in female C57Bl/6 mice

| | | t_{1/2} | CL or CL/F | Vss | Cₘₐₓ | Tₘₐₓ* | AUC₀₋ₗₐₛₜ | AUC_{0-inf} |
|---|---|---|---|---|---|---|---|---|
| Route | | (day) | (ml/day/kg) | (ml/kg) | (ng/ml) | (day) | (day*ng/ml) | (day*ng/ml) |
| SC | Mean | 1.67 | 49.48 | | 14994 | 1 | 38315 | 40734 |
| | (SD) | (0.14) | (4.791) | | (1776) | | (3851) | (4072) |
| IV | Mean | 1.57 | 35.00 | 69.03 | 40231 | 0.04 | 55263 | 57531 |
| | (SD) | (0.19) | (3.13) | (5.8) | (3500) | | (4853) | (5416) |

Note:
*Tmax (median)

Rat Pharmacokinetics

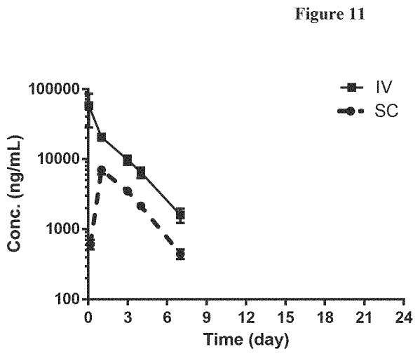

FP1 was administered to female Sprague Dawley rats at a dose of 2 mg/kg IV and SC in PBS, pH 7. Blood samples were collected, serum was processed and drug concentrations were measured up to 7 days following both routes of administration. The concentration of FP1 was determined using an immunoassay method. The serum drug concentration-time profile is summarized in Tables 22 and 23 and illustrated in FIG. 11.

TABLE 22
Serum concentration (nM) of FP1 over time following a
single SC administration in female Sprague Dawley rats.

| | FP1 - Group 3 (SC Dose) | | | | | | |
|---|---|---|---|---|---|---|---|
| | Animal | Animal | Animal | Animal | Animal | Average | |
| | 53 Result | 55 Result | 67 Result | 68 Result | 69 Result | Result | Std |
| Timepoint | (nM) | (nM) | (nM) | (nM) | (nM) | (nM) | Dev |
| 4 hr | 4.766 | 3.500 | 3.932 | 3.546 | 3.250 | 3.799 | 0.593 |
| 24 hr | 45.118 | 53.192 | 39.196 | 39.823 | 40.804 | 43.627 | 5.826 |
| 72 hr | 18.900 | 24.102 | 23.124 | 23.718 | 18.933 | 21.755 | 2.615 |
| 96 hr | 12.193 | 14.333 | 14.185 | 14.669 | 11.256 | 13.327 | 1.511 |
| Day 7 | 2.805 | 2.821 | 2.447 | 3.438 | 2.358 | 2.774 | 0.426 |

TABLE 23
Serum concentration (nM) of FP1 over time following a
single IV administration in female Sprague Dawley rats

| | FP1 - Group 4 (IV Dose) | | | | | | |
|---|---|---|---|---|---|---|---|
| | Animal | Animal | Animal | Animal | Animal | Average | |
| | 51 Result | 52 Result | 57 Result | 64 Result | 66 Result | Result | Std |
| Timepoint | (nM) | (nM) | (nM) | (nM) | (nM) | (nM) | Dev |
| 1 hr | 43.620* | 382.676 | 403.255 | 443.080 | 510.105 | 356.547 | 181.560 |
| 24 hr | 102.665* | 142.661 | 139.066 | 124.528 | 126.425 | 127.069 | 15.728 |
| 72 hr | 46.720 | 60.105 | 67.090 | 59.257 | 70.423 | 60.719 | 9.127 |
| 96 hr | 29.409 | 39.897 | 41.225 | 42.258 | 48.074 | 40.173 | 6.779 |
| Day 7 | 6.976 | 11.251 | 8.913 | 9.540 | 13.006 | 9.937 | 2.298 |

*Repeat analysis confirmed results

Pharmacokinetic analysis revealed a terminal half-life of 1.34 and 1.51 days for FP1 in Sprague Dawley rats following SC and IV administration, respectively (Table 24). FP1 demonstrated a mean bioavailability of ˜23% following SC administration.

TABLE 24
Mean (±SD) pharmacokinetic parameters of FP1 following
2 mg/kg IV and SC administration in Sprague Dawley rats

| | | t_{1/2} | CL or CL/F | Vss | Cₘₐₓ | Tₘₐₓ* | AUC₀₋ₗₐₛₜ | AUC_{0-inf} |
|---|---|---|---|---|---|---|---|---|
| Route | | (day) | (ml/day/kg) | (ml/kg) | (ng/ml) | (day) | (day*ng/ml) | (day*ng/ml) |
| SC | Mean | 1.34 | 100.09 | | 6987 | 1 | 19250 | 20112 |
| | (SD) | (0.04) | (3.97) | | (417) | | (794) | (820) |
| IV | Mean | 1.51 | 24.75 | 53.41 | 59000 | 0.04 | 83028 | 86525 |
| | (SD) | (0.12) | (8.53) | (17.15) | (25031) | | (20126) | (20881) |

Note:
*Tmax (median)

Monkey Pharmacokinetics

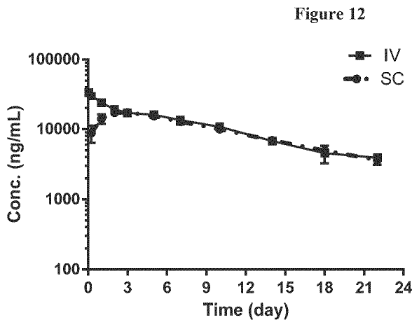

FP1 was administered to naïve male cynomolgus monkeys (Macaca fascicularis) at a dose of 1 mg/kg IV and SC in PBS, pH 7. Blood samples were collected, serum was processed and drug concentrations were measured up to 21 days following both routes of administration, using immunoassay bioanalysis. The serum drug concentration-time profile is summarized in Tables 25 and 26 and illustrated in FIG. 12.

TABLE 25
Serum concentration (nM) of FP1 over time following
a single SC administration in cynomolgus monkeys
as determined by immunoassay

| | | FP1 (SC Dose) | | | | |
|---|---|---|---|---|---|---|
| | | Animal | Animal | Animal | Average | Std |
| Timepoint | | 110 (nM) | 111 (nM) | 112 (nM) | Result (nM) | Dev |
| Predose | | <LLOQ | <LLOQ | <LLOQ | <LLOQ | N/A |
| 6 | hr | 49.304 | 43.784 | 72.110 | 55.066 | 15.017 |
| 24 | hr | 93.368 | 71.958 | 96.863 | 87.396 | 13.483 |
| 48 | hr | 107.689 | 97.509 | 115.144 | 106.781 | 8.853 |
| 72 | hr | 113.601 | 104.190 | 104.449 | 107.414 | 5.360 |
| 120 | hr | 101.490 | 95.049 | 91.717 | 96.085 | 4.968 |
| 168 | hr | 82.167 | 75.435 | 81.569 | 79.724 | 3.726 |
| 240 | hr | 71.033 | 56.732 | 59.266 | 62.344 | 7.631 |
| 336 | hr | 44.380 | 42.758 | 42.571 | 43.236 | 0.995 |
| 432 | hr | 30.911 | 29.445 | 32.839 | 31.065 | 1.702 |
| 528 | hr | 21.277 | 20.404 | 26.427 | 22.703 | 3.255 |

TABLE 26
Serum concentration (nM) of FP1 over time following
a single IV administration in cynomolgus monkeys
as determined by immunoassay

| | | FP1 (IV Dose) | | | | |
|---|---|---|---|---|---|---|
| | | Animal | Animal | Animal | Average | Std |
| Timepoint | | 104 (nM) | 105 (nM) | 106 (nM) | Result (nM) | Dev |
| Predose | | <LLOQ | <LLOQ | <LLOQ | <LLOQ | N/A |
| 1 | hr | 212.661 | 235.168 | 189.000 | 212.276 | 23.087 |
| 6 | hr | 190.315 | 185.331 | 183.575 | 186.407 | 3.497 |
| 24 | hr | 141.743 | 155.943 | 146.487 | 148.058 | 7.229 |
| 48 | hr | 111.765 | 126.105 | 120.744 | 119.538 | 7.246 |
| 72 | hr | 105.076 | 106.955 | 106.441 | 106.157 | 0.971 |
| 120 | hr | 92.591 | 103.882 | 103.757 | 100.077 | 6.483 |
| 168 | hr | 71.368 | 93.055 | 87.706 | 84.043 | 11.298 |
| 240 | hr | 71.554 | 65.093 | 65.685 | 67.444 | 3.572 |
| 336 | hr | 46.184 | 38.961 | 41.696 | 42.280 | 3.647 |
| 432 | hr | 34.589 | 31.266 | 19.492 | 28.449 | 7.933 |
| 528 | hr | 26.885 | 24.154 | 22.422 | 24.487 | 2.250 |

Pharmacokinetic analysis revealed a terminal half-life between 8.5 and 9.2 days for FP1 in cynomolgus monkeys following SC and IV administration, respectively with a mean bioavailability of ˜88% following SC administration (Table 27).

TABLE 27
Mean (±SD) pharmacokinetic parameters of FP1 following
1 mg/kg IV and SC administration in cynomolgus monkeys.

| | | t_{1/2} | CL or CL/F | Vss | Cₘₐₓ | Tₘₐₓ* | AUC₀₋ₗₐₛₜ | AUC_{0-inf} |
|---|---|---|---|---|---|---|---|---|
| Route | | (day) | (ml/day/kg) | (ml/kg) | (ng/ml) | (day) | (day*ng/ml) | (day*ng/ml) |
| SC | Mean | 8.5 | 3.9 | | 17776 | 3 | 211030 | 256202 |
| | (SD) | (1.5) | (0.3) | | (950) | | (11332) | (20192) |
| IV | Mean | 9.2 | 3.4 | 45.6 | 34001 | 0.04 | 239758 | 292206 |
| | (SD) | (0.5) | (0.1) | (1.6) | (3698) | | (6095) | (11516) |

Note:
*Tmax (median)

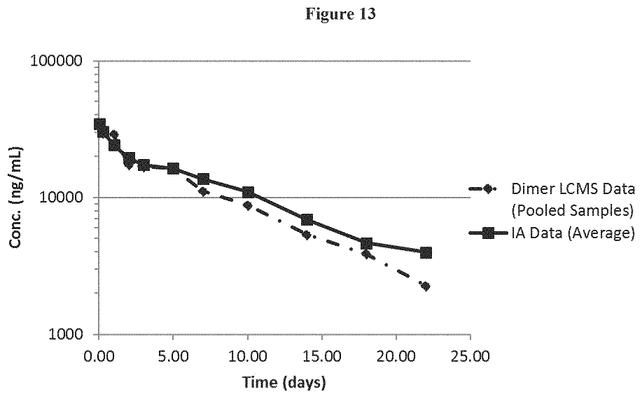

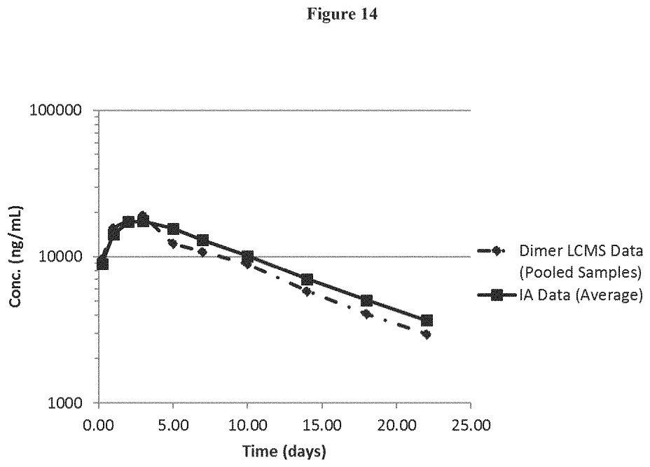

Immuno-affinity capture-LCMS analysis was used to quantitate the concentration of intact dimer present in the serum of cynomolgus monkeys after IV and SC administration (Tables 28 and 29 and FIGS. 13 and 14). Concentrations determined by this method were similar to concentrations determined by the immunoassay (IA), demonstrating that FP1 circulates as an intact dimer, with no detectable metabolic liability in cynomolgus monkeys.

TABLE 28
Serum concentration (ng/mL) of FP1 as an intact dimer over
time following a single IV administration in cynomolgus monkeys
as determined by immuno-affinity capture-LCMS analysis

| Day | Dimer Intact MS Data (Pooled Samples) | IA Data (Average) |
|---|---|---|
| 0.00 | 0 | 0 |
| 0.04 | 33347 | 34537 |
| 0.25 | 29686 | 30328 |
| 1.00 | 28787 | 24089 |
| 2.00 | 17249 | 19449 |
| 3.00 | 16827 | 17272 |
| 5.00 | 16159 | 16282 |
| 7.00 | 11124 | 13674 |
| 10.00 | 8746 | 10973 |
| 14.00 | 5328 | 6879 |
| 18.00 | 3857 | 4629 |
| 22.00 | 2252 | 3984 |

TABLE 29
Serum concentration (ng/mL) of FP1 as an intact dimer over
time following a single SC administration in cynomolgus monkeys
as determined by immuno-affinity capture-LCMS analysis.

| Day | Dimer Intact MS Data (Pooled Samples) | IA Data (Average) |
|---|---|---|
| 0.00 | 0 | 0 |
| 0.25 | 9625 | 8959 |
| 1.00 | 15799 | 14219 |
| 2.00 | 17671 | 17373 |
| 3.00 | 19130 | 17476 |
| 5.00 | 12284 | 15633 |
| 7.00 | 10808 | 12971 |
| 10.00 | 8910 | 10143 |
| 14.00 | 5814 | 7034 |
| 18.00 | 4074 | 5054 |
| 22.00 | 2967 | 3694 |

The concentration of analytes in cynomolgus monkey serum after IV and SC administration was also measured by immuno-affinity capture-trypsin digestion-LC-MS/MS analysis (Tables 30 and 31). Selected tryptic peptides, namely, ALV (ALVLIAFAQYLQQSPFEDHVK), ASL (ASLEDLGWADWVLSPR), and TDT (TDTGVSLQTYDDLLAK), which are located within FP1 near the N-terminus of the HSA region, the N-terminus of GDF15, and the C-terminal of GDF15, respectively. The peptides were monitored as surrogates peptides of FP1. The concentrations of all of the surrogate peptides were similar to each other and the concentrations measured by immunoassay, demonstrating that the GDF15 sequence in FP1 remains intact and linked to the full HSA sequence in vivo.

TABLE 30
Serum concentration (ng/mL) of surrogate peptides representing various regions
of FP1 over time following a single IV administration in cynomolgus monkeys
as determined by immuno-affmity capture-trypsin digestion-LC-MS/MS analysis

| Time point | | FP1 Average (ng/mL) | | | Std Dev | | | IA Data |
|---|---|---|---|---|---|---|---|---|
| Day | hour | ALV | TDT | ASL | ALV | TDT | ASL | (ng/mL) |
| 0.00 | 0 | <LLOQ | <LLOQ | <LLOQ | N/A | N/A | N/A | 0.0 |
| 0.04 | 1 | 32400.0 | 39766.7 | 33333.3 | 2623.0 | 3162.8 | 2722.7 | 34536.9 |
| 0.25 | 6 | 29100.0 | 27166.7 | 30600.0 | 3439.5 | 1006.6 | 2095.2 | 30328.1 |
| 1.00 | 24 | 24366.7 | 23300.0 | 23800.0 | 4215.8 | 3996.2 | 4100.0 | 24088.7 |
| 2.00 | 48 | 19433.3 | 17733.3 | 18700.0 | 1457.2 | 1193.0 | 854.4 | 19448.6 |
| 3.00 | 72 | 18100.0 | 17200.0 | 17166.7 | 360.6 | 871.8 | 1001.7 | 17271.6 |
| 5.00 | 120 | 15966.7 | 14033.3 | 14233.3 | 1001.7 | 642.9 | 1011.6 | 16282.3 |
| 7.00 | 168 | 13733.3 | 11800.0 | 12100.0 | 1115.0 | 600.0 | 300.0 | 13673.6 |
| 10.00 | 240 | 9303.3 | 8570.0 | 8570.0 | 2290.1 | 1682.2 | 1685.9 | 10973.0 |
| 14.00 | 336 | 5860.0 | 5890.0 | 6056.7 | 415.8 | 312.2 | 388.0 | 6878.9 |
| 18.00 | 432 | 4143.3 | 4400.0 | 4226.7 | 374.3 | 52.9 | 571.2 | 4628.6 |
| 22.00 | 528 | 2830.0 | 3256.7 | 2753.3 | 355.4 | 420.0 | 319.0 | 3984.0 |

TABLE 31
Serum concentration (ng/mL) of surrogate peptides representing various regions
of FP1 over time following a single SC administration in cynomolgus monkeys
as determined by immuno-affmity capture-trypsin digestion-LC-MS/MS analysis

| Time point | | FP1 Average (ng/mL) | | | Std Dev | | | IA Data |
|---|---|---|---|---|---|---|---|---|
| Day | hour | ALV | TDT | ASL | ALV | TDT | ASL | (ng/mL) |
| 0.00 | 0 | <LLOQ | <LLOQ | <LLOQ | N/A | N/A | N/A | 0.0 |
| 0.25 | 6 | 9323.3 | 7430.0 | 8123.3 | 1900.3 | 2471.6 | 1954.1 | 8959.1 |
| 1.00 | 24 | 15233.3 | 13390.0 | 14533.3 | 2926.3 | 3222.0 | 2683.9 | 14219.2 |
| 2.00 | 48 | 15366.7 | 14000.0 | 14166.7 | 2579.4 | 1646.2 | 2311.6 | 17373.0 |
| 3.00 | 72 | 17300.0 | 16033.3 | 15333.3 | 1571.6 | 1001.7 | 986.6 | 17476.0 |
| 5.00 | 120 | 15333.3 | 13366.7 | 13666.7 | 1222.0 | 1692.1 | 1501.1 | 15632.9 |
| 7.00 | 168 | 13333.3 | 11633.3 | 11700.0 | 709.5 | 472.6 | 781.0 | 12970.9 |
| 10.00 | 240 | 8496.7 | 7376.7 | 8343.3 | 1475.5 | 189.0 | 1039.1 | 10143.2 |
| 14.00 | 336 | 6046.7 | 6116.7 | 6253.3 | 90.2 | 118.5 | 110.6 | 7034.5 |
| 18.00 | 432 | 4593.3 | 5250.0 | 4636.7 | 802.6 | 1157.5 | 621.7 | 5054.2 |
| 22.00 | 528 | 3056.7 | 3490.0 | 3116.7 | 424.5 | 687.7 | 220.5 | 3693.7 |

Human Plasma Stability Assay

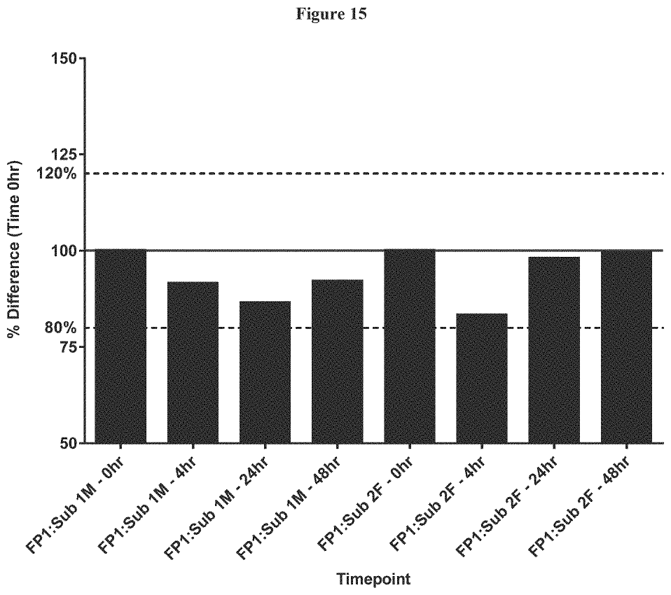

The purpose of this study was to analyze the ex vivo stability of FP1 in human plasma. Fresh, non-frozen human plasma was generated from heparinized blood from two subjects (one male and one female) by centrifugation. FP1 was incubated in this matrix at 37° C. with gentle mixing, for 0, 4, 24 and 48 hours. The concentration of FP1 was determined using an immunoassay method. The average percent difference from the starting concentration (0 hours) ranged from −4.1 to −12.9 and did not increase over time, demonstrating that FP1 is stable in human plasma for up to 48 hours ex vivo (Table 32 and FIG. 15).

TABLE 32
FP1 concentration (μg/mL) after 0, 4, 24, and 48 hours (hr) of ex vivo incubation
in plasma obtained from two human subjects (Sub) as determined by immunoassay

| | Sub 1 | | Sub 2 | | Average | |
|---|---|---|---|---|---|---|
| | Male Conc. | % Diff | Female Conc. | % Diff | Conc. | % Diff |
| Ex Vivo Sample | (ug/mL) | T0 | (ug/mL) | T0 | (ug/mL) | T0 |
| Plasma - T0 hr_FP1 | 11.503 | N/A | 12.649 | N/A | 12.076 | N/A |
| Plasma - T4 hr_FP1 | 10.524 | −8.5 | 10.521 | −16.8 | 10.523 | −12.9 |
| Plasma - T24 hr_FP1 | 9.934 | −13.6 | 12.402 | −2.0 | 11.168 | −7.5 |
| Plasma - T48 hr_FP1 | 10.582 | −8.0 | 12.575 | −0.6 | 11.578 | −4.1 |

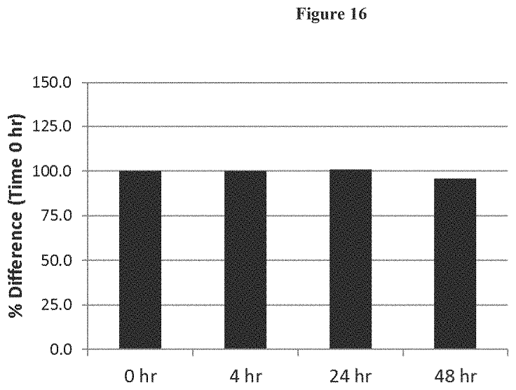

Immuno-affinity capture-LCMS was used to quantitate the concentration of intact dimer present after incubation in human plasma. Concentrations determined by this method were stable over time (0, 4, 24, and 48 hours), demonstrating that FP1 remains an intact dimer in human plasma ex vivo up to 48 hours (Table 33 and FIG. 16).

TABLE 33
Average FP1 concentration (μg/mL) and % difference from starting
concentration as an intact dimer after 0, 4, 24, and 48 hours
(hr) of ex vivo incubation in plasma obtained from two human
subjects as determined by immuno-affinity capture-LCMS analysis

| | Dimer Conc. (ug/mL) | % Difference |
|---|---|---|
| 0 hr | 15.8 | 100.0 |
| 4 hr | 15.8 | 100.1 |
| 24 hr | 15.9 | 100.9 |
| 48 hr | 15.2 | 96.0 |

The Examples 15-19 involve characterization of exemplary fusion protein of the invention, described in Example 5, which has the amino acid sequence of SEQ ID NO: 92 (encoded by nucleotide sequences SEQ ID NO: 95 (codon optimization 1) and SEQ ID NO: 110 (codon optimization 2)). This fusion protein is a fully recombinant protein that exists as a homodimer of a fusion of HSA (C34S) with the deletion variant of the mature human GDF15 (201-308; SEQ ID NO: 8) through a 42-amino acid linker consisting of glycine and serine residues, GS-(GGGGS)₈. The single native free cysteine at position 34 of HSA has been mutated to serine. This particular HSA-GDF15 fusion protein will be referred to as “FP2” in the following examples, for simplicity.

SEQ ID NOs: 92:
DARKSEVAHRFKDLGEENFKALVLIAFAQYLQQSPFEDHVKLVNEVTEF
AKTCVADESAENCDKSLHTLFGDKLCTVATLRETYGEMADCCAKQEPER
NECFLQHKDDNPNLPRLVRPEVDVMCTAFHDNEETFLKKYLYEIARRHP
YFYAPELLFFAKRYKAAFTECCQAADKAACLLPKLDELRDEGKASSAKQ
RLKCASLQKFGERAFKAWAVARLSQRFPKAEFAEVSKLVTDLTKVHTEC
CHGDLLECADDRADLAKYICENQDSISSKLKECCEKPLLEKSHCIAEVE
NDEMPADLPSLAADFVESKDVCKNYAEAKDVFLGMFLYEYARRHPDYSV
VLLLRLAKTYETTLEKCCAAADPHECYAKVFDEFKPLVEEPQNLIKQNC
ELFEQLGEYKFQNALLVRYTKKVPQVSTPTLVEVSRNLGKVGSKCCKHP
EAKRMPCAEDYLSVVLNQLCVLHEKTPVSDRVTKCCTESLVNRRPCFSA
LEVDETYVPKEFNAETFTFHADICTLSEKERQIKKQTALVELVKHKPKA
TKEQLKAVMDDFAAFVEKCCKADDKETCFAEEGKKLVAASQAALGLGSG
GGGSGGGGSGGGGSGGGGSGGGGSGGGGSGGGGSGGGGSDHCPLGPGRC
CRLHTVRASLEDLGWADWVLSPREVQVTMCIGACPSQFRAANMHAQIKT
SLHRLKPDTVPAPCCVPASYNPMVLIQKTDTGVSLQTYDDLLAKDCHCI

### Example 13: The In Vitro Agonist Potency of FP2

The in vitro agonist potency of FP2 was evaluated using a cell-based pAKT assay with SK-N-AS cells stably over-expressing the human GDF15 receptor (GFRAL). GFRAL activity was determined by measuring phospho-AKT (Ser473) level in SK-N-AS human neuroblastoma cells (ATCC) stably transfected to overexpress human GFRAL. Phosphorylation of AKT after treating the GFRAL expressing cells with various concentrations of test article was measured using the Phospho-AKT (Ser473) Assay kit (Cisbio, Bedford, Mass.) according to manufacturer's instructions. Resulting data was used to calculate EC₅₀ values using Prism statistical software (GraphPad Software San Diego). FP2 activated pAKT with a half maximal effective concentration (EC₅₀) of 2.908±0.239 nM (N=3). Native GDF15 served as an assay control and demonstrated agonist activity with an EC₅₀ of 0.153±0.008 nM (N=3).

### Example 14: Effects of FP2 on the Food Intake of C57BI/6 Mice

FP2 was evaluated for its ability to reduce food intake in male C57Bl/6 mice after a single dose. Male C57Bl/6N mice (age 10-12 weeks) obtained from Taconic Biosciences (Hudson, N.Y.) were used in the study. Mice were singly housed in a temperature-controlled room with 12-hour light/dark cycle (6 am/6 pm) and allowed ad libitum access to water and chow. Male C57Bl/6 mice were acclimated for a minimum of 72 hours in the BioDAQ cages; mice were then grouped based on food intake in the last 24 hours into six groups of eight each. Between 4:00-5:00 pm, animals were weighed and dosed with vehicle or compounds via subcutaneous injection. Change in food weight for each cage was recorded continuously by the BioDAQ system, for a period of 48 hours after compound administration. 6×His-FP1 was used as a comparator in this study.

FP2 had significant effects on reducing food intake at 12, 24 and 48 hours after administration at all dose levels tested (Table 34). There was a reduction in percent change in food intake relative to PBS at all time points and all dose levels (Table 35) in mice.

TABLE 34
Effect of a Single Dose of FP2 on Food
Intake over 48 hours in C57Bl/6 Mice
Cumulative Food Intake (g)

| Treatment | 12 hours | 24 hours | 48 hours |
|---|---|---|---|
| PBS | 3.7 ± 0.2 | 4.4 ± 0.2 | 8.8 ± 0.4 |
| FP2, 1 nmol/kg | 2.8 ± 0.4* | 3.0 ± 0.4** | 6.9 ± 0.7* |
| FP2, 4 nmol/kg | 2.4 ± 0.2*** | 3.1 ± 0.2** | 7.0 ± 0.3* |
| FP2, 8 nmol/kg | 1.9 ± 0.2**** | 2.4 ± 0.1**** | 6.2 ± 0.2*** |
| FP2, 16 nmol/kg | 1.8 ± 0.1**** | 2.7 ± 0.1*** | 6.3 ± 0.4** |
| 6xHis-FP1, | 2.3 ± 0.3*** | 2.8 ± 0.4** | 6.6 ± 0.7** |
| 8 nmol/kg | | | |

Data are expressed as Mean ± SEM.
*p ≤ 0.05, versus PBS
**p ≤ 0.01, versus PBS
***p ≤ 0.001, versus PBS
****p ≤ 0.0001, versus PBS, respectively
Statistical analyses used: ANOVA and Dunnett's multiple comparisons test.
n = 8/group, except for 6xHis-FP1 8 nmol/kg (n = 6).

TABLE 35
Effect of a Single Dose of FP2 on Percent Reduction in Food
Intake (Relative to Vehicle) over 48 hours in C57Bl/6 Mice
Percentage of inhibition relative to PBS

| Treatment | 12 hours | 24 hours | 48 hours |
|---|---|---|---|
| PBS | 0.0 ± 7.9 | 0.0 ± 7.7 | 0.0 ± 5.9 |
| FP2, 1 nmol/kg | 22.4 ± 12.5* | 31.8 ± 10.5** | 21.8 ± 8.1* |
| FP2, 4 nmol/kg | 36.6 ± 7.2*** | 30.4 ± 5.8** | 20.2 ± 4.7* |
| FP2, 8 nmol/kg | 47.0 ± 6.2**** | 45.7 ± 3.8**** | 29.7 ± 3.9*** |
| FP2, 16 | 49.2 ± 4.1**** | 38.1 ± 4.1*** | 27.8 ± 5.1** |
| nmol/kg | | | |
| 6xHis-FP1, | 36.9 ± 9.9*** | 36.5 ± 8.8** | 24.6 ± 8.2** |
| 8 nmol/kg | | | |

The anorectic effect of FP2 is expressed as the relative reduction in food intake compared with the respective PBS controls.
Data are expressed as Mean ± SEM.
*p ≤ 0.05, versus PBS
**p ≤ 0.01, versus PBS
***p ≤ 0.001, versus PBS
****p ≤ 0.0001, versus PBS, respectively
Statistical analyses used: ANOVA and Dunnett's multiple comparisons test.
n = 8/group, except for 6xHis-FP1 8 nmol/kg (n = 6).

### Example 15: Effects of FP2 on Food Intake in Sprague Dawley Rats

FP2 was evaluated for its ability to reduce food intake and body weight gain in male Sprague-Dawley rats after a single dose. The animals were obtained from Charles River Labs (Wilmington, Mass.) at 200-225 g body weight and used within one week of delivery. They were housed one per cage on alpha dry bedding and a plastic tube for enrichment in a temperature-controlled room with 12-hour light/dark cycle. They were allowed ad libitum access to water and were fed laboratory rodent diet; Irradiated Certified PicoLab® Rodent Diet 20, 5K75* (supplied from Purina Mills, St. Louis, Mo. via ASAP Quakertown, Pa.). Animal weights were taken and recorded for each rat prior to dosing.

Animals were acclimated for a minimum of 72 hours in the BioDAQ cages; rats were then grouped based on food intake in the last 24 hours into six groups of eight each. Between 4:00-5:00 pm, animals were weighed and dosed with vehicle or compounds via subcutaneous injection. Change in food weight for each cage was recorded continuously by the BioDAQ system, for a period of 48 hours after compound administration. 6×His-FP1 was used as a comparator in this study.

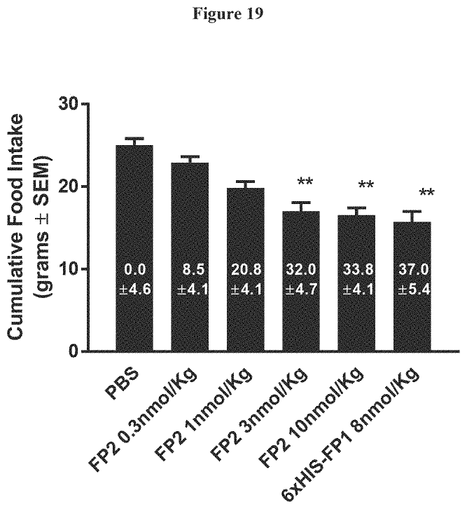

Dose-dependent reductions of food intake were tested after a single dose of FP2. No significant differences in food intake were observed at the dose of 0.3 nmol/kg. Significant effects in reduction of food intake were observed 12 hours but not 24 or 48 hours at 1 nmol/kg. Significant reductions in food intake were observed at all time points for the 3 and 10 nmol/kg dose levels (Table 36, FIG. 19). There was a reduction in percent change in food intake relative to PBS at all time points and all dose levels (Table 37).

TABLE 36
Effect of a single dose of FP2 on food
intake over 48 hours in Sprague Dawley rats.
Cumulative Food Intake (g)

| Treatment | 12 hours | 24 hours | 48 hours |
|---|---|---|---|
| PBS | 21.7 ± 0.6 | 25.0 ± 0.8 | 53.1 ± 1.6 |
| FP2, 0.3 nmol/Kg | 19.5 ± 0.9 | 22.9 ± 0.7 | 48.4 ± 1.5 |
| FP2, 1 nmol/Kg | 17.5 ± 0.9* | 19.8 ± 0.8 | 47.0 ± 2.6 |
| FP2, 3 nmol/Kg | 16.1 ± 1.2** | 17.0 ± 1.0** | 39.2 ± 3.2** |
| FP2, 10 nmol/Kg | 15.8 ± 0.9*** | 16.5 ± 0.9** | 36.5 ± 3.2** |
| 6XHis-FP1 8 nmol/Kg | 15.0 ± 1.4*** | 15.7 ± 1.2** | 37.2 ± 4.5** |

Data are expressed as Mean ± SEM.
*p ≤ 0.05, versus PBS
**p ≤ 0.01, versus PBS
***p ≤ 0.001, versus PBS, respectively
Statistical analyses used: ANOVA and Dunnett's multiple comparisons test.
n = 8/group

TABLE 37
Effect of a single dose of FP2 on percent reduction in food
intake (relative to vehicle) over 48 hours in Sprague Dawley rats.
Percentage of inhibition relative to PBS

| Treatment | 12 hours | 24 hours | 48 hours |
|---|---|---|---|
| PBS | 0.0 ± 4.0 | 0.0 ± 4.6 | 0.0 ± 4.3 |
| FP2 0.3 nmol/kg | 10.0 ± 4.7 | 8.5 ± 4.1 | 8.8 ± 3.9 |
| FP2 1 nmol/kg | 19.3 ± 4.7* | 20.8 ± 4.1 | 11.5 ± 5.5 |
| FP2 3 nmol/kg | 25.9 ± 6.1** | 32.0 ± 4.7** | 26.2 ± 6.5** |
| FP2 10 nmol/kg | 27.4 ± 4.8*** | 33 8 ± 4.1** | 31.2 ± 6.4** |
| 6XHis-FP1 8 nmol/kg | 30.8 ± 6.6*** | 37.0 ± 5.4** | 29.9 ± 8.8** |

The anorectic effect of FP2 is expressed as the relative reduction in food intake compared with the respective PBS controls.
Data are expressed as Mean ± SEM.
*p ≤ 0.05, versus PBS
**p ≤ 0.01, versus PBS
***p ≤ 0.001, versus PBS, versus PBS, respectively
Statistical analyses used: ANOVA and Dunnett's multiple comparisons test.
n = 8/group

### Example 16: Effects of FP2 on Food Intake, Body Weight and Glucose Homeostasis in Diet-Induced Obese (DIO) C57Bl/6 Mice

FP2 was evaluated for its ability to reduce food intake and body weight and improve glucose homeostasis on repeat dosing in male DIO C57Bl/6 mice over a period of 8 days. Male DIO C57Bl/6 mice (age 21 weeks, high fat-fed for 15 weeks) obtained from Taconic Biosciences (Hudson, N.Y.) were used in the study. Mice were singly housed in a temperature-controlled room with 12-hour light/dark cycle (6 am/6 pm) and allowed ad libitum access to water and fed with Research Diet D12492 (Research Diets, New Brunswick, N.J.). Mice were acclimated >1 week in the mouse housing room prior to the experiment. The endpoints of the study were measurements of food intake, body weight, body composition and glycemic endpoints (OGTT, blood glucose). One day prior to dosing, animals were weighed and grouped by body weight (BW). Mice were dosed by subcutaneous injection. Animals dosed with FP2 received this compound on Day 0, Day 3, and Day 6, Day 9 and Day 12. The vehicle group and rosiglitazone group received sterile PBS s.c. on these days as well. Rosliglitazone was provided in the diet at 0.015% w/w ad libitum. BW and food intake were recorded daily, over a period of fifteen days. Blood glucose was measured on Days 0, 7 and 13. An oral glucose tolerance test (OGTT) was performed on Day 14. Insulin levels were measured at selected time points during the OGTT. Mice were euthanized with C02 and terminal blood samples were collected for exposure via cardiac puncture on day 15. A separate PK arm was run with three mice per dose group with a total of 15 mice.

Exposure-Response (E-R) Analysis for FP2 in DIO Mouse

Most animals in the pharmacodynamics (PD) (efficacy) arms had undetectable drug concentrations on the last study day when the pharmacokinetics (PK) samples were obtained, potentially due to immunogenicity. Therefore, the mean PK profiles from the PK arms, instead of individual PK from the PD arms, were used to conduct exposure-response (from day 3, 6 and 9, respectively) for the % weight change from baseline in the PD arms at the corresponding dose level. This method assumes that the PK arms behave similarly to the PD arms in terms of drug exposure.

The Eₘₐₓ model (GraphPad Prism 6, log(agonist) vs. response) was used to correlate exposure with response data (log transformed drug concentrations). Hill Slope was set to be 1. Note that the model fitted EC₁₀ to EC₅₀ values were within two fold amongst day 3, 6 and 9, despite that the Eₘₐₓ estimates were different (Eₘₐₓ=−4.26%, −8.18% and −9.85%, respectively). Some animals on day 9 also showed the loss of drug exposure, due to potential ADA formation and therefore, the E-R parameter estimates based on day 9 data should be interpreted with caution.

The effects of two weeks of exposure of FP2 on food intake, body weight, glucose homeostasis, and liver fat content was assessed in diet induced obese male C57Bl/6 mice. Trough exposure between 1.7 and 3.3 nM FP2 for the 0.3 nmol/kg treatment group, between 7.1 and 14 nM for the 1.0 nmol/kg treatment group, between 20.8 and 41.6 nM for the 3.0 nmol/kg treatment group, and between 28.5 and 112.9 nM FP2 for the 10 nmol/kg treatment group was maintained until day 9 in the PK arm of the study (n=2 or 3, Table 49). After day 9, a decrease in circulating levels was observed in the majority of animals despite continued q3d dosing (Table 49). Consistent with this accelerated clearance, the majority of animals in the PD arm of the study had undetectable circulating levels of FP2 on day 15 (Table 50).

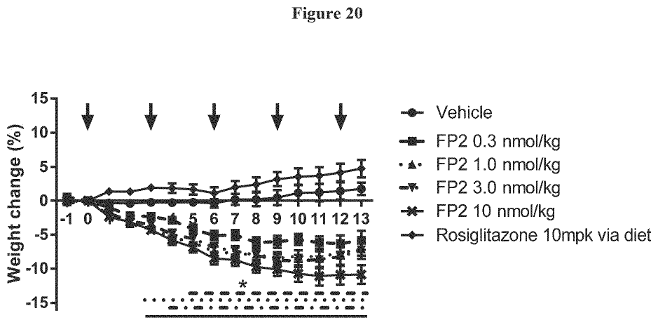

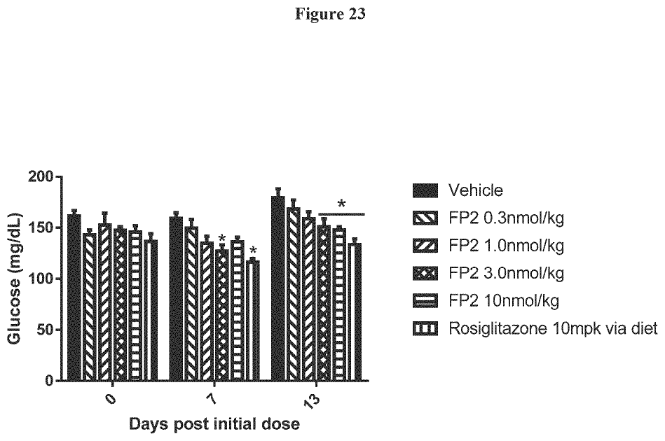

Treatment of DIO mice with FP2 q3d reduced food intake (Table 38), body weight (Table 39, 40 and FIG. 20) and fed blood glucose compared to vehicle treatment (Table 43 and FIG. 23). A significant reduction in food intake was seen on day 2, day 5, and day 8 for 0.3 nmol/kg, from day 1 through day 7 for 1.0 nmol/kg, on day 1, day 2, day 4 through day 6, and day 8 for 3.0 nmol/kg, and on day 1, day 3 through day 6, day 8 and day 9 for 10.0 nmol/kg. Percent body weight changes were significant from day 5 through day 13 for 0.3 nmol/kg, from day 3 through day 13 for 1.0 nmol/kg and 10.0 nmol/kg, and from day 4 through day 13 for 3.0 nmol/kg. Changes in grams of body weight were significant from day 8 for 0.3 nmol/kg, from day 6 for 1.0 nmol/kg, from day 7 for 3.0 nmol/kg and from day 5 for 10.0 nmol/kg. Decreases in fed blood glucose levels were significant on day 7 for the animals in the 3.0 nmol/kg dose level and were significant on day 13 for the animals in the 3.0 and 10.0 nmol/kg dose levels.

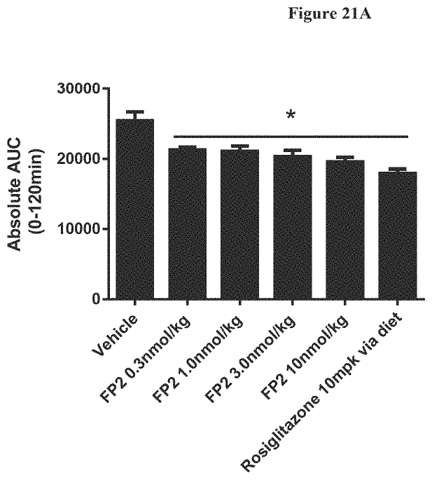

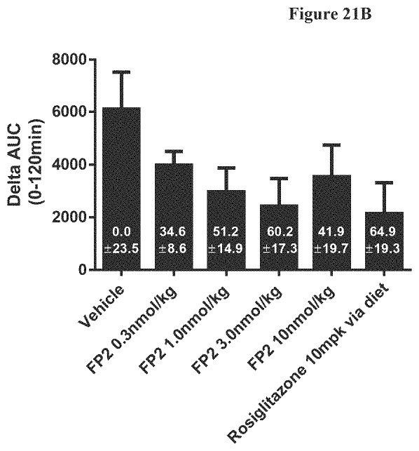

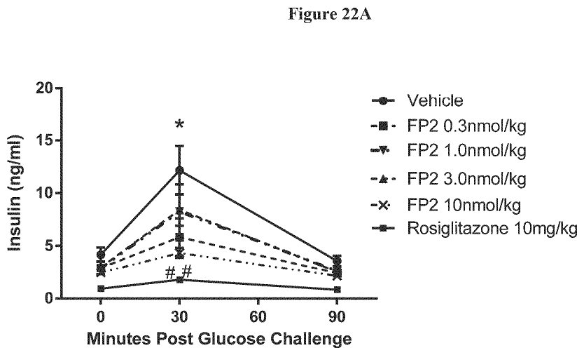

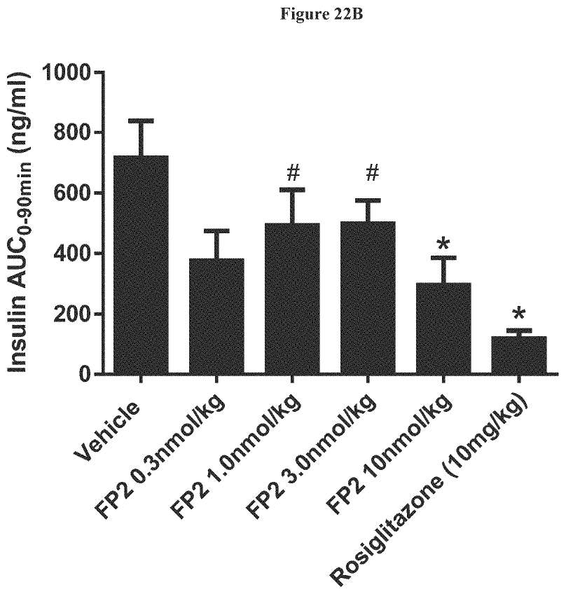

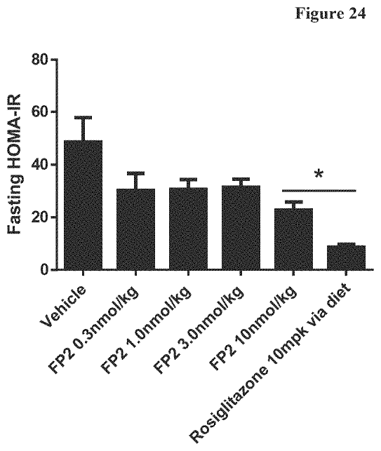

DIO mice treated with FP2 q3d had improved glucose tolerance on day 14 compared to vehicle treatment during an oral glucose challenge (Table 41; FIGS. 21A and 21B). Glucose was significantly lower at 30 minutes for the 0.3 nmol/kg group, at 60 minutes and 120 minutes for the 1.0 nmol/kg group, at 120 minutes for the 3.0 nmol/kg group, and at 30, 90, and 120 minutes for the 10.0 nmol/kg group. Total area under the curve was significant for all dose groups. Insulin levels during the glucose challenge were significantly lower for 0.3 and 10.0 nmol/kg groups at 30 minutes (Table 42; FIGS. 22A and 22B). In addition, compared to vehicle treated animals, there was a significant reduction in the calculated fasted HOMA-IR in DIO mice after 14 days of treatment with FP2 q3d at 10.0 nmol/kg indicative of improved insulin sensitivity (Table 44 and FIG. 24).

Body composition was measured by MRI on day −1 before the start of the study and on day 13 (Table 47 and Table 48). DIO mice treated with FP2 at 1.0 nmol/kg and 10.0 nmol/kg had significant reductions in fat mass on day 13; whereas there were no changes in lean mass for any treatment groups. On day 13, the 10.0 nmol/kg treatment group had a significant increase in percent lean mass and a significant reduction in percent fat mass compared to the vehicle treated group. Changes from day −1 to day 13 were significant for lean mass in the 0.3 nmol/kg, 1.0 nmol/kg, and 10.0 nmol/kg treatment groups and were significant for percent lean mass in the 1.0, 3.0, and 10.0 nmol/kg treatment groups. Changes from day −1 to day 13 were significant for fat mass and percent lean mass in all treatment groups compared to vehicle.

There was no significant difference in endogenous mouse GDF15 serum levels between vehicle treated animals and mice treated with FP2 q3d for 15 days (Table 46).

Conclusion: the results suggest that the higher drug exposure is generally associated with greater % weight change from baseline on a population level across the studied dose groups on day 3, 6 and 9.

Exposure to FP2 over two weeks led to reduced food intake, decreased body weight, decreased blood glucose, improved glucose tolerance and insulin sensitivity in DIO mice. Significant decreases in food intake over multiple days were achieved at 1.0, 3.0, and 10.0 nmol/kg q3d. Body weight was decreased significantly starting three to five days after the initiation of the study. Fed blood glucose on day 13 was significantly decreased after q3d administration of FP2 at 3.0 and 10.0 nmol/kg. Insulin sensitivity represented by significantly decreased fasting HOMA-IR was achieved 14 days after 10.0 nmol/kg FP2 administered q3d. On day 13, a significant increase in percent lean mass and a significant reduction in percent fat mass was observed in DIO mice treated q3d with 10.0 nmol/kg FP2.

TABLE 38
Effect of FP2 on daily food intake (g) over 13 days of treatment.

| Treatment | Vehicle | FP2 (nmol/kg) | | | | Rosiglitazone |
|---|---|---|---|---|---|---|
| Day | N/A | 0.3 | 1.0 | 3.0 | 10.0 | 10 mpk/day |
| 0 | 2.0 ± 0.1 | 1.9 ± 0.1 | 2.2 ± 0.2 | 1.9 ± 0.2 | 1.9 ± 0.2 | 2.2 ± 0.1 |
| 1 | 2.4 ± 0.2 | 2.1 ± 0.1 | 1.6 ± 0.1* | 1.5 ± 0.1* | 1.3 ± 0.1* | 2.7 ± 0.1 |
| 2 | 2.5 ± 0.1 | 1.8 ± 0.1* | 1.7 ± 0.2* | 1.9 ± 0.1* | 2.0 ± 0.1 | 3.0 ± 0.2 |
| 3 | 2.5 ± 0.1 | 2.1 ± 0.1 | 1.7 ± 0.1* | 1.9 ± 0.1 | 1.8 ± 0.1* | 2.8 ± 0.2 |
| 4 | 2.6 ± 0.1 | 2.0 ± 0.1 | 1.8 ± 0.1* | 1.9 ± 0.1* | 1.9 ± 0.1* | 2.9 ± 0.2 |
| 5 | 2.8 ± 0.1 | 2.1 ± 0.2* | 2.2 ± 0.1* | 2.2 ± 0.0* | 1.9 ± 0.1* | 2.7 ± 0.2 |
| 6 | 2.8 ± 0.1 | 2.3 ± 0.2 | 2.1 ± 0.1* | 2.2 ± 0.1* | 2.0 ± 0.1* | 2.8 ± 0.2 |
| 7 | 2.6 ± 0.2 | 2.3 ± 0.1 | 2.0 ± 0.1* | 2.0 ± 0.2 | 2.1 ± 0.1 | 2.9 ± 0.2 |
| 8 | 2.7 ± 0.2 | 2.1 ± 0.1* | 2.2 ± 0.1 | 2.0 ± 0.1* | 2.0 ± 0.1* | 3.1 ± 0.2 |
| 9 | 2.8 ± 0.1 | 2.3 ± 0.1 | 2.3 ± 0.2 | 2.3 ± 0.2\hat{ } | 2.1 ± 0.2* | 3.2 ± 0.1 |
| 10 | 2.6 ± 0.1 | 2.5 ± 0.1 | 2.4 ± 0.2 | 2.3 ± 0.2\hat{ } | 2.1 ± 0.2 | 3.0 ± 0.2 |
| 11 | 2.9 ± 0.1 | 2.5 ± 0.1 | 2.8 ± 0.2 | 2.6 ± 0.4 | 2.6 ± 0.1 | 3.3 ± 0.1 |
| 12 | 2.8 ± 0.1 | 2.7 ± 0.2 | 2.8 ± 0.1 | 2.8 ± 0.2 | 2.9 ± 0.1 | 3.1 ± 0.2 |
| 13 | 2.7 ± 0.1 | 2.5 ± 0.2 | 2.6 ± 0.1 | 2.6 ± 0.1 | 2.2 ± 0.1 | 3.1 ± 0.2 |

Values represent mean ± SEM for data from 8 animals per time per group, except n = 7 when noted by \hat{ }
*p < 0.05, versus vehicle
Statistical analyses used: Two-Way ANOVA RM, Tukey's multiple comparison test

TABLE 39
Effect of FP2 on Percent Body Weight Change Over 13 days of Treatment

| Treatment | Vehicle | FP2 (nmol/kg) | | | | Rosiglitazone |
|---|---|---|---|---|---|---|
| Day | N/A | 0.3 | 1.0 | 3.0 | 10.0 | 10 mpk/day |
| −1 | −0.1 ± 0.4 | 0.5 ± 0.2 | −0.1 ± 0.1 | −0.4 ± 0.2 | 0.0 ± 0.6 | 0.1 ± 0.2 |
| 0 | 0.0 ± 0.0 | 0.0 ± 0.0 | 0.0 ± 0.0 | 0.0 ± 0.0 | 0.0 ± 0.0 | 0.0 ± 0.0 |
| 1 | −0.4 ± 0.5 | −1.0 ± 0.3 | −2.2 ± 0.4 | −1.8 ± 0.6 | −2.4 ± 0.5 | 1.3 ± 0.4 |
| 2 | −0.4 ± 0.3 | −2.2 ± 0.4 | −3.4 ± 0.6 | −3.0 ± 0.6 | −3.1 ± 0.4 | 1.3 ± 0.5 |
| 3 | −0.3 ± 0.4 | −2.3 ± 0.4 | −4.2 ± 0.6* | −3.5 ± 0.6 | −4.3 ± 0.5* | 1.9 ± 0.5 |
| 4 | −0.3 ± 0.5 | −2.9 ± 0.5 | −5.3 ± 0.6* | −4,9 ± 0.7* | −5.8 ± 0.7* | 1.8 ± 0.7 |
| 5 | −0.2 ± 0.4 | −4.2 ± 0.7* | −6.2 ± 0.6* | −5.6 ± 0.7* | −6.8 ± 0.7* | 1.7 ± 0.8 |
| 6 | −0.4 ± 0.6 | −5.1 ± 0.7* | −7.4 ± 0.8* | −6.9 ± 0.7* | −8.5 ± 0.9* | 1.1 ± 0.9 |
| 7 | 0.2 ± 0.8 | −5.1 ± 0.7* | −7.7 ± 0.9* | −7.4 ± 0.7* | −8.7 ± 0.9* | 2.0 ± 1.0 |
| 8 | 0.2 ± 1.0 | −6.1 ± 0.8* | −7.9 ± 0.9* | −8.0 ± 0.8* | −9.7 ± 0.8* | 2.4 ± 1.1 |
| 9 | 0.5 ± 1.1 | −6.0 ± 0.9* | −8.4 ± 0.9* | −8.8 ± 0.9* | −10.1 ± 1.0* | 3.1 ± 1.0 |
| 10 | 1.1 ± 1.2 | −5.7 ± 0.8* | −8.1 ± 0.9* | −8.9 ± 0.9* | −10.7 ± 1.2* | 3.5 ± 1.2 |
| 11 | 1.2 ± 1.3 | −6.1 ± 0.9* | −8.2 ± 0.8* | −8.6 ± 1.3* | −11.1 ± 1.4* | 3.7 ± 1.2 |
| 12 | 1.4 ± 1.3 | −6.3 ± 1.2* | −7.7 ± 0.8* | −8.2 ± 1.4* | −10.9 ± 1.4* | 4.1 ± 1.3 |
| 13 | 1.7 ± 0.9 | −5.8 ± 1.4* | −7.1 ± 1.0* | −7.2 ± 1.4* | −10.9 ± 1.4* | 4.7 ± 1.3 |

Values represent mean ± SEM for data from 8 animals per time per group
*p < 0.05, versus Vehicle
Statistical analyses used: Two-Way ANOVA RM, Tukey's multiple comparison test

TABLE 40
Effect of FP2 on body weight change (g) over 13 days of treatment

| Treatment | Vehicle | FP2 (nmol/kg) | | | | Rosiglitazone |
|---|---|---|---|---|---|---|
| Day | N/A | 0.3 | 1.0 | 3.0 | 10.0 | 10 mpk/day |
| −1 | 44.6 ± 0.6 | 44.5 ± 0.6 | 44.5 ± 0.6 | 44.5 ± 0.6 | 44.5 ± 0.6 | 44.5 ± 0.6 |
| 0 | 44.6 ± 0.6 | 44.3 ± 0.6 | 44.6 ± 0.6 | 44.7 ± 0.6 | 44.6 ± 0.7 | 44.4 ± 0.6 |
| 1 | 44.5 ± 0.7 | 43.8 ± 0.6 | 43.6 ± 0.6 | 43.9 ± 0.7 | 43.5 ± 0.7 | 45.0 ± 0.7 |
| 2 | 44.5 ± 0.7 | 43.3 ± 0.7 | 43.1 ± 0.6 | 43.3 ± 0.7 | 43.2 ± 0.7 | 45.0 ± 0.7 |
| 3 | 44.5 ± 0.7 | 43.2 ± 0.7 | 42.7 ± 0.6 | 43.1 ± 0.7 | 42.6 ± 0.6 | 45.3 ± 0.7 |
| 4 | 44.5 ± 0.7 | 43.0 ± 0.7 | 42.2 ± 0.6 | 42.5 ± 0.7 | 42.0 ± 0.7 | 45.2 ± 0.7 |
| 5 | 44.5 ± 0.7 | 42.4 ± 0.7 | 41.8 ± 0.5 | 42.2 ± 0.7 | 41.5 ± 0.6* | 45.2 ± 0.7 |
| 6 | 44.5 ± 0.7 | 42.0 ± 0.7 | 41.3 ± 0.6* | 41.6 ± 0.7 | 40.7 ± 0.5* | 44.9 ± 0.8 |
| 7 | 44.7 ± 0.7 | 42.0 ± 0.7 | 41.1 ± 0.6* | 41.4 ± 0.7* | 40.6 ± 0.5* | 45.3 ± 0.8 |
| 8 | 44.7 ± 0.7 | 41.6 ± 0.7* | 41.1 ± 0.7* | 41.1 ± 0.7* | 40.2 ± 0.6* | 45.5 ± 0.8 |
| 9 | 44.8 ± 0.7 | 41.6 ± 0.8* | 40.8 ± 0.7* | 40.8 ± 0.8* | 40.0 ± 0.8* | 45.8 ± 0.7 |
| 10 | 45.1 ± 0.7 | 41.8 ± 0.8* | 41.0 ± 0.8* | 40.8 ± 0.8* | 39.8 ± 0.8* | 46.0 ± 0.8 |
| 11 | 45.2 ± 0.8 | 41.6 ± 0.8* | 40.9 ± 0.7* | 40.8 ± 0.9* | 39.6 ± 0.9* | 46.1 ± 0.8 |
| 12 | 45.3 ± 0.7 | 41.5 ± 0.9* | 41.2 ± 0.7* | 41.0 ± 0.9* | 39.7 ± 0.9* | 46.3 ± 0.9 |
| 13 | 45.4 ± 0.7 | 41.7 ± 0.9* | 41.4 ± 0.8* | 41.5 ± 0.9* | 39.7 ± 0.9* | 46.5 ± 0.9 |

Values represent mean ± SEM for data from 8 animals per time per group
*p < 0.05, versus Vehicle
Statistical analyses: Two-Way ANOVA RM, Tukey's multiple comparison test

TABLE 41
Effect of FP2 on blood glucose (mg/dL) levels during an OGTT after 14 days of treatment

| | | | | | | | Total AUC | Δ AUC |
|---|---|---|---|---|---|---|---|---|
| | Dose | Time after Glucose Challenge (min) | | | | | (mg/dL/ | (mg/dL/ |
| Treatment | (nmol/kg) | 0 | 30 | 60 | 90 | 120 | 120 min) | 120 min) |
| Vehicle | NA | 161 ± 10 | 225 ± 17 | 228 ± 14 | 208 ± 18 | 213 ± 19 | 25429 ± 1228 | 6094 ± 1430 |
| FP2 | 0.3 | 144 ± 4 | 163 ± 14* | 209 ± 11 | 180 ± 12 | 171 ± 7 | 21270 ± 399* | 3985 ± 521 |
| | 1.0 | 151 ± 9 | 179 ± 18 | 188 ± 7 | 179 ± 13 | 163 ± 8* | 21096 ± 754* | 2972 ± 907 |
| | 3.0 | 149 ± 8 | 180 ± 18 | 179 ± 11* | 164 ± 6 | 163 ± 9* | 20338 ± 876* | 2426 ± 1058 |
| | 10.0 | 134 ± 6 | 163 ± 7* | 190 ± 13 | 152 ± 7* | 163 ± 8* | 19599 ± 614* | 3539 ± 1198 |
| Rosiglitazone | 10 mpk/day | 132 ± 9 | 152 ± 10* | 162 ± 13* | 138 ± 9* | 159 ± 9* | 17904 ± 632* | 2139 ± 1179 |

Values represent mean ± SEM for data from 8 animals per time per group
*p < 0.05, versus Vehicle
Statistical analyses: Two-Way ANOVA RM, Tukey's multiple comparison test for Glucose Values;
One-Way ANOVA, Tukey's multiple comparison test for AUC

TABLE 42
Effect of FP2 on insulin (ng/mL) levels
during an OGTT after 14 days of treatment

| | Dose | Time after Glucose Challenge (min) | | | Total AUC |
|---|---|---|---|---|---|
| Treatment | (nmol/kg) | 0 | 30 | 90 | (ng/mL/90 min) |
| Vehicle | NA | 4.2 ± 0.7 | 12.2 ± 2.3 | 3.6 ± 0.5 | 716.1 ± 122.0 |
| FP2 | 0.3 | 2.9 ± 0.6 | 5.8 ± 1.8* | 2.4 ± 0.4 | 377.1 ± 97.3 |
| | 1.0 | 2.9 ± 0.4 | 8.2 ± 2.6 | 2.6 ± 0.4 | 491.8 ± 118.2 |
| | 3.0 | 3.0 ± 0.3 | 8.4 ± 1.5 | 2.5 ± 0.3 | 498.6 ± 77.0 |
| | 10.0 | 2.4 ± 0.4 | 4.3 ± 0.5* | 2.1 ± 0.4 | 295.5 ± 32.0* |
| Rosiglitazone | 10 mpk/day | 0.9 ± 0.1 | 1.8 ± 0.2* | 0.8 ± 0.1 | 118.5 ± 9.6* |

Values represent mean ± SEM for data from 8 animals per time per group
*p < 0.05, versus Vehicle
Statistical analyses: Two-Way ANOVA RM, Tukey's multiple comparison test for insulin Values;
One-Way ANOVA, Tukey's multiple comparison test for AUC

TABLE 43
Effect of FP2 on fed blood glucose (mg/dL) levels

| | Dose | Time after start of treatment (days) | | |
|---|---|---|---|---|
| Treatment | (nmol/kg) | 0 | 7 | 13 |
| Vehicle | NA | 162 ± 5 | 159 ± 6 | 180 ± 9 |
| FP2 | 0.3 | 143 ± 5 | 150 ± 9 | 168 ± 9 |
| | 1.0 | 153 ± 12 | 135 ± 7 | 159 ± 7 |
| | 3.0 | 148 ± 3 | 127 ± 6* | 151 ± 8* |
| | 10.0 | 146 ± 6 | 136 ± 4 | 148 ± 3* |
| Rosiglitazone | 10 mpk/day | 137 ± 7 | 117 ± 4* | 134 ± 6* |

Values represent mean ± SEM for data from 8 animals per time per group
*p < 0.05, versus Vehicle
Statistical analyses: Two-Way ANOVA RM, Tukey's multiple comparison test

TABLE 44
Fasted HOMA-IR in DIO mice after 14
days of q3d treatement with FP2

| Treatment | Dose (nmol/kg) | HOMA-IR |
|---|---|---|
| Vehicle | NA | 48.7 ± 9.2 |
| FP2 | 0.1 | 30.1 ± 6.6 |
| | 1.0 | 30.5 ± 3.8 |
| | 3.0 | 31.3 ± 3.2 |
| | 10.0 | 22.7 ± 3.1* |
| Rosiglitazone | 10 mpk/day | 8.7 ± 1.0* |

Values represent mean ± SEM for data from 8 animals per time per group
*p < 0.05, versus Vehicle
Statistical analyses: One-Way ANOVA, Tukey's multiple comparison test

TABLE 45
Liver weight after 15 days of treatment with FP2 q3d in DIO mice

| | | Liver | Liver |
|---|---|---|---|
| | Dose | Weight, | Weight |
| Treatment | (nmol/kg) | (g) | (% of body) |
| Vehicle | NA | 1.9 ± 0.1 | 4.3 ± 0.3 |
| FP2 | 0.1 | 1.7 ± 0.1 | 4.0 ± 0.1 |
| | 1.0 | 1.7 ± 0.0 | 4.2 ± 0.1 |
| | 3.0 | 1.8 ± 0.1 | 4.2 ± 0.1 |
| | 10.0 | 1.7 ± 0.1 | 4.2 ± 0.1 |
| Rosiglitazone | 10 mpk/day | 1.9 ± 0.1 | 4.1 ± 0.2 |

Values represent mean ± SEM for data from 8 animals per time per group
* p < 0.05, versus Vehicle
Statistical analyses: One-Way ANOVA, Tukey's multiple comparison test

TABLE 46
Serum mouse GDF15 (pg/mL) levels after 15
days of treatement with FP2 q3d in DIO mice

| Treatment | Dose (nmol/kg) | mGDF15 |
|---|---|---|
| Vehicle | NA | 258.3 ± 21.4 |
| FP2 | 0.1 | 214.1 ± 10.3 |
| | 1.0 | 191.6 ± 12.7 |
| | 3.0 | 254.5 ± 28.6 |
| | 10.0 | 202.0 ± 10.0 |
| Rosiglitazone | 10 mpk/day | 509.6 ± 26.2* |

Values represent mean ± SEM for data from 8 animals per time per group
*p < 0.05, versus Vehicle
Statistical analyses: One-Way ANOVA, Tukey's multiple comparison test

TABLE 47
Effect of FP2 q3d in DIO mice on body composition (g) measured by MRI

| | Dose | Fat (g) | Fat (g) | Δ Fat | Lean (g) | Lean (g) | Δ Lean |
|---|---|---|---|---|---|---|---|
| Treatment | (nmol/kg) | Day −1 | Day 13 | (g) | Day −1 | Day 13 | (g) |
| Vehicle | NA | 17.4 ± 0.6 | 18.2 ± 0.6 | 0.9 ± 0.3 | 25.4 ± 0.3 | 25.1 ± 0.3 | −0.4 ± 0.2 |
| FP2 | 0.3 | 17.0 ± 0.6 | 15.2 ± 0.8 | −1.9 ± 0.6* | 25.8 ± 0.7 | 24.5 ± 0.5 | −1.2 ± 0.2* |
| | 1.0 | 17.0 ± 0.6 | 14.9 ± 0.6* | −2.1 ± 0.4* | 25.8 ± 0.5 | 24.6 ± 0.4 | −1.2 ± 0.2* |
| | 3.0 | 17.4 ± 0.7 | 15.1 ± 1.1 | −2.3 ± 0.5* | 25.3 ± 0.4 | 24.2 ± 0.4 | −1.1 ± 0.1 |
| | 10.0 | 16.7 ± 0.6 | 13.2 ± 0.8* | −3.6 ± 0.5* | 26.1 ± 0.6 | 24.5 ± 0.6 | −1.7 ± 0.2* |
| Rosiglitazone | 10 mpk/ | 17.2 ± 0.6 | 19.1 ± 0.5 | 1.9 ± 0.6 | 25.5 ± 0.7 | 25.1 ± 0.6 | −0.4 ± 0.2 |
| | day | | | | | | |

Values represent mean ± SEM for data from 8 animals per time per group
*p < 0.05, versus Vehicle
Statistical analyses: One-Way ANOVA, Tukey's multiple comparison test

TABLE 48
Effect of FP2 q3d in DIO mice on body composition (%) measured by MRI

| | Dose | Fat (%) | Fat (%) | Δ Fat | Lean (%) | Lean (%) | Δ Lean |
|---|---|---|---|---|---|---|---|
| Treatment | (nmol/kg) | Day −1 | Day 13 | (%) | Day −1 | Day 13 | (%) |
| Vehicle | NA | 38.1 ± 1.0 | 40.1 ± 1.0 | 2.1 ± 0.3 | 55.9 ± 0.9 | 55.3 ± 0.9 | −0.6 ± 0.3 |
| FP2 | 0.3 | 37.6 ± 1.2 | 36.3 ± 1.3 | −1.3 ± 0.9* | 56.8 ± 1.3 | 59.0 ± 1.4 | 2.2 ± 0.8 |
| | 1.0 | 37.2 ± 1.1 | 35.8 ± 0.9 | −1.4 ± 0.5* | 56.7 ± 1.1 | 59.5 ± 1.0 | 2.8 ± 0.6* |
| | 3.0 | 38.3 ± 1.4 | 36.1 ± 2.0 | −2.3 ± 0.7* | 55.9 ± 1.2 | 58.7 ± 2.0 | 2.8 ± 0.8* |
| | 10.0 | 36.9 ± 1.1 | 33.1 ± 1.5* | −3.8 ± 0.8* | 57.5 ± 1.0 | 61.7 ± 1.4* | 4.2 ± 0.7* |
| Rosiglitazone | 10 mpk/ | 38.0 ± 1.2 | 41.1 ± 0.9 | 3.1 ± 0.9 | 56.2 ± 1.2 | 53.9 ± 0.8 | −2.3 ± 0.8 |
| | day | | | | | | |

Values represent mean ± SEM for data from 8 animals per time per group
*p < 0.05, versus Vehicle
Statistical analyses: One-Way ANOVA, Tukey's multiple comparison test

TABLE 49
FP2 serum exposures (nM) of the PK arm during q3d treatment in DIO mice

| | | Time Post Initial Dose (Time post last dose) | | | | | | |
|---|---|---|---|---|---|---|---|---|
| Treatment | Subject | | | | | Day 14 | Day 15 | Day 17 |
| Group | ID | Day 3 | Day 6 | Day 9 | Day 12 | (48 hr) | (72 hr) | (120 hr) |
| 0.1 | 9 | 1.930 | 2.993 | <LOQ | <LOQ | <LOQ | <LOQ | <LOQ |
| nmol/kg | 10 | 1.853 | 3.369 | 2.817 | <LOQ | <LOQ | <LOQ | <LOQ |
| | 11 | 1.715 | 2.637 | 2.568 | 2.709 | 3.441 | 1.313 | <LOQ |
| 1.0 | 9 | 8.198 | 9.660 | 9.645 | <LOQ | <LOQ | <LOQ | <LOQ |
| nmol/kg | 10 | 7.073 | 10.595 | 8.966 | <LOQ | <LOQ | <LOQ | <LOQ |
| | 11 | 6.689 | 13.967 | 10.802 | <LOQ | <LOQ | <LOQ | <LOQ |
| 3.0 | 9 | 20.802 | 26.329 | 27.863 | <LOQ | <LOQ | <LOQ | <LOQ |
| nmol/kg | 10 | 23.563 | 32.020 | 41.576 | 1.168 | <LOQ | <LOQ | <LOQ |
| | 11 | 21.704 | 30.101 | <LOQ | <LOQ | <LOQ | <LOQ | <LOQ |
| 10.0 | 9 | 28.495 | 33.050 | <LOQ | <LOQ | <LOQ | <LOQ | <LOQ |
| nmol/kg | 10 | 70.779 | 112.898 | <LOQ | <LOQ | <LOQ | <LOQ | <LOQ |
| | 11 | 70.404 | 111.767 | 55.117 | <LOQ | <LOQ | <LOQ | <LOQ |

Data are expressed as concentration for each animal.
<LOQ = below limit of quantitation;
LOQ is 0.494 nM
** Values at Day 3, 6, 9 and 12 are immediately prior to next dose

TABLE 50
Terminal serum exposures (nM) of FP2 after
15 days of q3d treatment in DIO mice

| | |
|---|---|
| Animal | Treatment Group (nmol/kg) |

| ID | 0.1 | 1.0 | 3.0 | 10.0 |
|---|---|---|---|---|
| 1 | 2.567 | <LOQ | <LOQ | <LOQ |
| 2 | <LOQ | <LOQ | <LOQ | 44.149 |
| 3 | <LOQ | <LOQ | <LOQ | <LOQ |
| 4 | 1.144 | 0.678 | <LOQ | <LOQ |
| 5 | <LOQ | <LOQ | <LOQ | <LOQ |
| 6 | 3.440 | <LOQ | <LOQ | <LOQ |
| 7 | 2.727 | <LOQ | <LOQ | <LOQ |
| 8 | <LOQ | <LOQ | <LOQ | <LOQ |

Data are expressed as concentration for each animal.
<LOQ = below limit of quantitation;
LOQ is 0.494 nM

### Example 17: FP2 Multispecies Pharmacokinetics and Immune Response

Mouse Pharmacokinetics

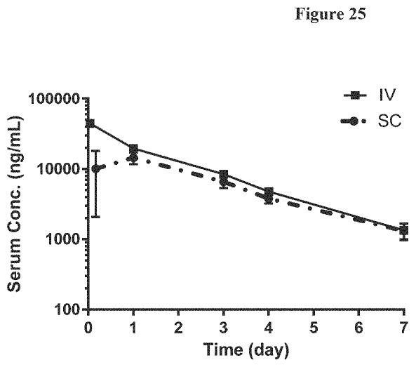

The pharmacokinetic properties of FP2 were evaluated when administered subcutaneously to female C57Bl/6 mice. FP2 was administered subcutaneously (n=5 samples per time point) and intravenously (n=5 samples per time point) to female C57Bl/6 mice (Sage Laboratories, St Louis, Mo.) at a dose level of 2.0 mg/kg in PBS, (pH 7.3-7.5). The collection of sample at the last time point was via a terminal bleed. Blood samples were collected, serum processed and drug concentrations were measured up to 168 hours. The levels of FP2 were measured using an immunoassay method. The drug concentration profiles in plasma are summarized in Table 51 and 52 and illustrated in FIG. 25.

Pharmacokinetic analysis of FP2 in C571Bl/6 mice demonstrated a terminal half-life of ˜1.51 and ˜1.76 days following IV and SC dosing respectively, with a mean bioavailability of ˜61% following SC administration.

TABLE 51
Serum concentration (ng/mL) of FP2 following a
single subcutaneous (SC) dose in C57Bl/6 mice

| | FP2 - SC Dose | | | | | | |
|---|---|---|---|---|---|---|---|
| | Animal | Animal | Animal | Animal | Animal | Average | |
| | 31 Result | 32 Result | 33 Result | 35 Result | 36 Result | Result | Std Dev |
| Timepoint | (ng/mL) | (ng/mL) | (ng/mL) | (ng/mL) | (ng/mL) | (ng/mL) | (ng/mL) |
| 4 hr | 11310.11 | 6323.44 | 23489.21 | 4357.74 | 4835.54 | 10063.21 | 7995.0 |
| 24 hr | 12378.58 | 10896.80 | 17769.03 | 15928.14 | 15404.08 | 14475.33 | 2785.0 |
| 72 hr | 5127.95 | 5569.27 | 6727.37 | 7909.66 | 7550.32 | 6576.91 | 1210.5 |
| 96 hr | 3059.83 | 3350.25 | 4042.04 | 4095.15 | 4431.94 | 3795.84 | 569.0 |
| 168 hr | 784.61 | 1202.66 | 1364.68 | 1557.60 | 1678.02 | 1317.51 | 348.9 |

TABLE 52
Serum concentration (ng/mL) of FP2 following a
single intravenous (IV) dose in C57Bl/6 mice.

| | FP2 - IV Dose | | | | | | |
|---|---|---|---|---|---|---|---|
| | Animal | Animal | Animal | Animal | Animal | Average | Std |
| | 37 Result | 40 Result | 41 Result | 45 Result | 48 Result | Result | Dev |
| Timepoint | (ng/mL) | (ng/mL) | (ng/mL) | (ng/mL) | (ng/mL) | (ng/mL) | (ng/mL) |
| 1 hr | 49573.59 | 42382.59 | 40001.18 | 43085.75 | 46443.59 | 44297.34 | 3742.9 |
| 24 hr | 22173.85 | 19120.39 | 19393.78 | 19798.29 | 17459.96 | 19589.25 | 1696.7 |
| 72 hr | 8334.33 | 7989.10 | 8573.44 | 9086.37 | 8186.68 | 8433.98 | 422.5 |
| 96 hr | 4478.83 | 4699.80 | 4749.93 | 5331.39 | 4579.08 | 4767.81 | 332.3 |
| 168 hr | 1044.24 | 1419.64 | 1393.44 | 1835.63 | 979.49 | 1334.49 | 343.6 |

TABLE 53
Pharmacokinetic parameters of FP2 following 2 mg/kg
IV and 2 mg/kg SC administration in C57Bl/6 mice.

| | | t_{1/2} | CL or CL/F | Vz or Vz/F | Cₘₐₓ | Tₘₐₓ* | AUC₀₋ₗₐₛₜ | AUC_{0-inf} |
|---|---|---|---|---|---|---|---|---|
| Route | | (day) | (ml/day/kg) | (ml/kg) | (ng/ml) | (day) | (day*ng/ml) | (day*ng/ml) |
| SC | Mean | 1.76 | 43 | 108 | 15619 | 1 | 44972 | 48379 |
| | SD | 0.16 | 8 | 20 | 4870 | | 8563 | 9147 |
| IV | Mean | 1.51 | 25 | 55 | 44297 | 0.042 | 76269 | 79253 |
| | SD | 0.18 | 1 | 6 | 3743 | | 3788 | 4051 |

Rat Pharmacokinetics

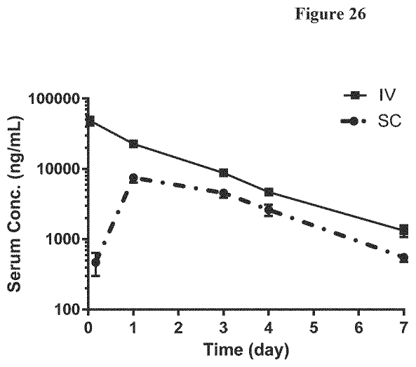

FP2 was administered subcutaneously (n=5 samples per time point) and intravenously (n=5 samples per time point) to female Sprague-Dawley rats (Sage Laboratories, St. Louis, Mo.) at a dose level of 2.0 mg/kg in PBS, (pH 7.3-7.5). The collection of sample at the last time point was via a terminal bleed. Blood samples were collected, serum processed and drug concentrations were measured up to 168 hours. The levels of FP2 were measured using an immunoassay method. The drug concentration profiles in plasma are summarized in Table 54 and 55, and illustrated in FIG. 26. Pharmacokinetic parameters calculated from these data are summarized in Table 56.

Pharmacokinetic analysis of FP2 in Sprague Dawley rats demonstrated a terminal half-life of ˜1.46 and ˜1.37 days following IV and SC dosing respectively, with a mean bioavailability of ˜28% following SC administration.

TABLE 54
Serum concentration (ng/mL) of FP2 following a single
subcutaneous (SC) dose in Sprague-Dawley rats.

| | FP2 - SC Dose | | | | | | |
|---|---|---|---|---|---|---|---|
| | Animal | Animal | Animal | Animal | Animal | Average | Std |
| | 01 Result | 02 Result | 03 Result | 04 Result | 05 Result | Result | Dev |
| Timepoint | (ng/ml) | (ng/ml) | (ng/ml) | (ng/ml) | (ng/ml) | (ng/ml) | (ng/ml) |
| 4 hr | 730.95 | 257.94 | 469.95 | 496.34 | 400.65 | 471.16 | 172.2 |
| 24 hr | 9236.27 | 6658.79 | 7728.92 | 7449.56 | 6502.60 | 7515.23 | 1092.1 |
| 72 hr | 4559.04 | 4464.72 | 5619.82 | 4287.25 | 3844.15 | 4555.00 | 655.6 |
| 96 hr | 2791.97 | 2452.35 | 3387.55 | 2316.06 | 2176.00 | 2624.79 | 483.8 |
| 168 hr | 622.32 | 606.36 | <80.00 | 514.19 | 476.49 | 554.84 | 70.7 |

TABLE 55
Serum concentration (ng/mL) of FP2 following a single
intravenous (IV) dose in Sprague-Dawley rats.

| | FP2 - IV Dose | | | | | | |
|---|---|---|---|---|---|---|---|
| | Animal | Animal | Animal | Animal | Animal | Average | Std |
| | 06 Result | 07 Result | 08 Result | 09 Result | 10 Result | Result | Dev |
| Timepoint | (ng/ml) | (ng/ml) | (ng/ml) | (ng/ml) | (ng/ml) | (ng/ml) | (ng/ml) |
| 1 hr | 60785.80 | 47392.80 | 43046.54 | 46014.25 | 44547.65 | 48357.41 | 7134.5 |
| 24 hr | 25470.36 | 24729.88 | 21157.29 | 20497.32 | 21459.71 | 22662.91 | 2267.1 |
| 72 hr | 8208.46 | 9262.13 | 8866.76 | 8635.93 | 8843.83 | 8763.42 | 384.1 |
| 96 hr | 4433.18 | 4833.22 | 4995.63 | 4630.60 | 4604.13 | 4699.35 | 218.1 |
| 168 hr | 1083.14 | 1469.35 | 1614.61 | 1394.17 | 1053.89 | 1323.03 | 245.7 |

TABLE 56
Pharmacokinetic parameters of FP2 following 2 mg/kg IV
and 2 mg/kg SC administration in Sprague-Dawley Rats.

| | | t_{1/2} | CL or CL/F | Vz or Vz/F | Cₘₐₓ | Tₘₐₓ* | AUC₀₋ₗₐₛₜ | AUC_{0-inf} |
|---|---|---|---|---|---|---|---|---|
| Route | | (day) | (ml/day/kg) | (ml/kg) | (ng/ml) | (day) | (day*ng/ml) | (day*ng/ml) |
| SC | Mean | 1.37 | 84 | 165 | 7515 | 1 | 22614 | 24036 |
| | SD | 0.04 | 10 | 17 | 1092 | | 2509 | 2893 |
| IV | Mean | 1.46 | 23 | 49 | 48357 | 0.042 | 83271 | 86089 |
| | SD | 0.12 | 2 | 6 | 7135 | | 6081 | 5820 |

Monkey Pharmacokinetics

FP2 was administered subcutaneously at 1 mg/kg and intravenously at 1 mg/kg to three male cynomolgus monkeys each in PBS, (pH 7.0-7.6). Blood samples were collected, plasma processed and drug concentrations were measured up to 21 days.

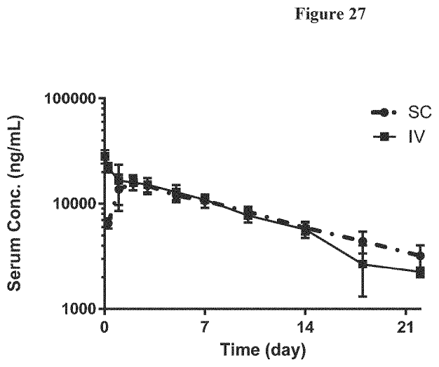

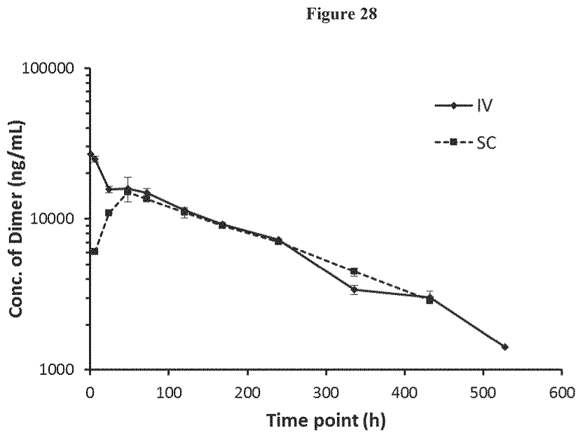

The pharmacokinetics (PK) of FP2 was characterized following administration of a single dose IV (1.0 mg/kg) and SC (1.0 mg/kg) in cynomolgus monkeys. The plasma drug concentration-time profile after SC administration is summarized in Tables 57 and 58 for immunoassay and LCMS analyses respectively and after IV administration in Tables 59 and 60 for immunoassay and LCMS analyses respectively. The immunoassay data is graphed in FIG. 27, and the LCMS data is represented in FIG. 28.

Using results from the immunoassay analysis, the mean NCA-based terminal half-life (t½) for FP2 was ˜7.05 and ˜8.51 days following IV and SC dosing, respectively. The mean PK parameters following IV and SC administration are summarized in Table 61. Using results from the immunoassay bioanalysis, the mean non-compartment model estimated terminal half-life (t½) for FP2 was 7.05 and 8.51 days following IV and SC dosing, respectively. The mean bioavailability (F %) of FP2 was estimated to be ˜98.5% based on AUC₀₋ₗₐₛₜ and estimated to be ˜109.2% based on AUC_{0-inf} in cynomolgus monkeys following SC administration.

TABLE 57
Plasma concentration (ng/mL) of FP2 measured by immunoassay
following a single SC dose in cynomolgus monkeys.

| | Immunoassay | | | | |
|---|---|---|---|---|---|
| Time | Animal | Animal | Animal | SC | Std |
| (hr) | 704 | 705 | 706 | Ave | Dev |
| 0 | <80.0 | <80.0 | <80.0 | <80.0 | N/A |
| 6 | 7365.3 | 6128.5 | 6056.9 | 6516.9 | 735.6 |
| 24 | 19716.8 | 10903.8 | 10554.7 | 13725.1 | 5191.9 |
| 48 | 18191.7 | 14353.7 | 14464.7 | 15670.0 | 2184.5 |
| 72 | 17813.9 | 14207.5 | 12684.0 | 14901.8 | 2634.5 |
| 120 | 13823.9 | 11445.8 | 10590.8 | 11953.5 | 1675.3 |
| 168 | 12359.6 | 10103.8 | 9467.8 | 10643.8 | 1519.7 |
| 240 | 9457.9 | 7642.6 | 8109.1 | 8403.2 | 942.7 |
| 336 | 6796.8 | 5679.8 | 5235.7 | 5904.1 | 804.3 |
| 432 | 5581.3 | 3830.6 | 3746.5 | 4386.1 | 1035.9 |
| 528 | 4126.2 | 2613.4 | 2879.3 | 3206.3 | 807.7 |

N/A = not applicable

TABLE 58
Plasma concentration (ng/mL) of FP2 measured by LCMS
following a single SC dose in cynomolgus monkeys.

| | LC/MS | | | | |
|---|---|---|---|---|---|
| Time | Animal | Animal | Animal | SC | |
| (hr) | 704 | 705 | 706 | Ave | SEM |
| 0 | <1000 | <1000 | <1000 | <1000 | N/A |
| 6 | 6110.0 | 5910 | 6200.0 | 6073.3 | 85.7 |
| 24 | # | 10420.0 | 11300.0 | 10860.0 | 359.3 |
| 48 | 16560.0 | 13510.0 | 14960.0 | 15010.0 | 880.8 |
| 72 | 13690.0 | 13450.0 | 13380.0 | 13506.7 | 93.9 |
| 120 | 11040.0 | # | # | 11040.0 | N/A |
| 168 | # | 8680.0 | 9400.0 | 9040.0 | 293.9 |
| 240 | 6940.0 | 7070.0 | 7140.0 | 7050.0 | 58.6 |
| 336 | 4400.0 | 4580.0 | 4430.0 | 4470.0 | 55.7 |
| 432 | — | 2910.0 | — | 2910.0 | N/A |
| 528 | <1000 | <1000 | <1000 | <1000 | N/A |

— = initial run failed, not enough sample for repeat analysis
# = mislabeled tube, sample excluded from the analysis
N/A = not applicable

TABLE 59
Plasma concentration (ng/mL) of FP2 measured by immunoassay
following a single IV dose in cynomolgus monkeys.

| | Immunoassay | | | | |
|---|---|---|---|---|---|
| Time | Animal | Animal | Animal | IV | Std |
| (hr) | 701 | 702 | 703 | Ave | Dev |
| 0 | <80.0 | <80.0 | <80.0 | <80.0 | N/A |
| 1 | 29938.3 | 31139.0 | 23545.3 | 28207.6 | 4082.0 |
| 6 | 24711.9 | 21173.0 | 20434.4 | 22106.4 | 2286.5 |
| 24 | 19648.4 | 21420.9 | 8662.7 | 16577.3 | 6911.3 |
| 48 | 17790.1 | 17107.0 | 12983.1 | 15960.0 | 2600.7 |
| 72 | 17889.4 | 13871.1 | 13692.4 | 15151.0 | 2373.3 |
| 120 | 15298.6 | 11982.5 | 11361.5 | 12880.9 | 2116.7 |
| 168 | 11752.0 | 10982.8 | 9933.8 | 10889.5 | 912.7 |
| 240 | 8921.1 | 7465.0 | 6733.4 | 7706.5 | 1113.6 |
| 336 | 6572.8 | 5750.5 | 4687.6 | 5670.3 | 945.1 |
| 432 | 1099.6 | 3425.0 | 3458.0 | 2660.9 | 1352.2 |
| 528 | <80.0 | 2069.0 | 2416.2 | 2242.6 | N/A |

N/A = not applicable

TABLE 60
Plasma concentration (ng/mL) of FP2 measured by LCMS
following a single IV dose in cynomolgus monkeys

| | LC/MS | | | | |
|---|---|---|---|---|---|
| Time | Animal | Animal | Animal | IV | |
| (hr) | 701 | 702 | 703 | Ave | SEM |
| 0 | <1000 | <1000 | <1000 | <1000 | N/A |
| 1 | 25340.0 | 28820 | 26220.0 | 26793.3 | 1044.7 |
| 6 | 24610.0 | 26340.0 | 23810.0 | 24920.0 | 746.6 |
| 24 | 18410.0 | 18680.0 | 9810.0 | 15633.3 | 2912.7 |
| 48 | 16290.0 | 17370.0 | 13840.0 | 15833.3 | 1044.3 |
| 72 | 15430.0 | 14920.0 | 14280.0 | 14876.7 | 332.7 |
| 120 | 11180.0 | 11480.0 | 11440.0 | 11366.7 | 94.0 |
| 168 | 9170.0 | # | # | 9170.0 | N/A |
| 240 | 6800.0 | 7380.0 | 7600.0 | 7260.0 | 238.6 |
| 336 | 2870.0 | 3860.0 | 3480.0 | 3403.3 | 288.3 |
| 432 | — | — | 3030.0 | 3030.0 | N/A |
| 528 | 1150.0 | 1680.0 | 1400.0 | 1410.0 | 153.1 |

— = initial run failed, not enough sample for repeat analysis
N/A = not applicable
# = mislabeled tube, sample excluded from the analysis

TABLE 61
Mean (±SD) pharmacokinetic parameters of FP2 following
1 mg/kg IV and SC administration in cynomolgus monkey.

| | | t_{1/2} | CL or CL/F | Vz or Vz/F | Cₘₐₓ | Tₘₐₓ* | AUC₀₋ₗₐₛₜ | AUC_{0-inf} |
|---|---|---|---|---|---|---|---|---|
| Route | | (day) | (ml/day/kg) | (ml/kg) | (ng/ml) | (day) | (day*ng/ml) | (day*ng/ml) |
| SC | Mean | 8.51 | 4.6 | 56 | 16178 | 1.67 | 180792 | 221032 |
| | SD | 1.28 | 0.8 | 7 | 3065 | | 29990 | 44931 |
| IV | Mean | 7.05 | 4.9 | 51 | 28208 | 0.042 | 183468 | 202380 |
| | SD | 1.45 | 0.2 | 12 | 4082 | | 18268 | 9617 |

PK parameters are mean values based on NCA of immunoassay PK data.
*Tmax (median)

Human Plasma Stability Assay

The ex vivo stability of FP2 was examined in fresh heparinized plasma at 37° C. for up to 48 hours. Fresh, non-frozen human plasma was generated from heparinized blood from two subjects (one male and one female) by centrifugation. FP2 was incubated in this matrix at 37° C. with gentle mixing or 0. 4. 24 and 48 hours. The concentration of FP2 was determine using an immunoassay method. An independent immunoaffinity capture followed by LCMS method was used to quantitate the concentration of the intact dimer present in this matrix under the assay conditions.

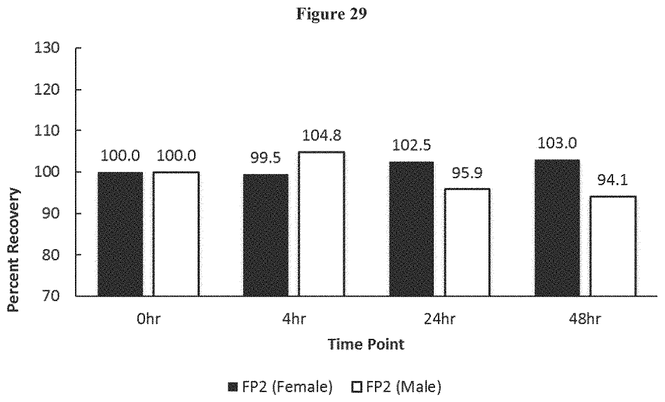

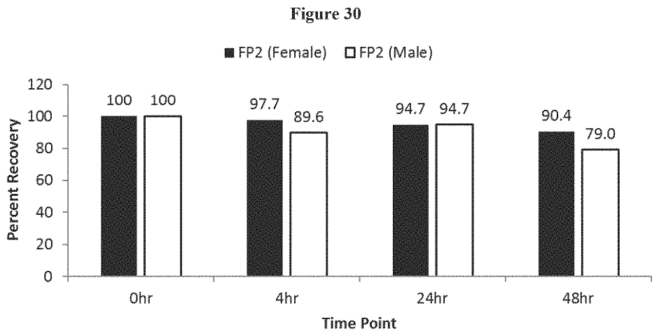

In the immunoassay method, the percent recovery from the starting concentration ranged from 104.8 to 94.1 and did not decrease over time, demonstrating that FP2 is stable in human plasma up to 48 hours ex vivo (FIG. 29 and Table 62). The LCMS method showed that concentrations were stable over time demonstrating that FP2 remains an intact dimer in human plasma up to 48 hours ex vivo (FIG. 30 and Table 63).

TABLE 62
Ex vivo stability of FP2 (Normalized Percent Recovery) over
48 hours in human plasma (ng/ml) measured by immunoassay.

| | | Time | Concentration | Normalized % |
|---|---|---|---|---|
| Compound | Gender | (hr) | (ng/mL) | Recovery |
| FP2 | female | 0 | 9104 | 100.0 |
| | | 4 | 9056 | 99.5 |
| | | 24 | 9332 | 102.5 |
| | | 48 | 9374 | 103.0 |
| | male | 0 | 9473 | 100.0 |
| | | 4 | 9929 | 104.8 |
| | | 24 | 9081 | 95.9 |
| | | 48 | 8912 | 94.1 |

TABLE 63
Ex vivo stability of FP2 (Normalized Percent Recovery) over
48 hours in human plasma (ng/ml) measured by intact LC/MS.

| | | Time | Concentration | Normalized % |
|---|---|---|---|---|
| Compound | Gender | (hr) | (ng/mL) | Recovery |
| FP2 | female | 0 | 11090 | 100.0 |
| | | 4 | 10830 | 97.7 |
| | | 24 | 10500 | 94.7 |
| | | 48 | 10030 | 90.4 |
| | male | 0 | 10760 | 100.0 |
| | | 4 | 9640 | 89.6 |
| | | 24 | 10190 | 94.7 |
| | | 48 | 8500 | 79.0 |

### Example 18: Efficacy of Single Dose FP1 and FP2 in Cynomolgus Monkeys

The effects of FP1 and FP2 on food intake and body weight after a single dose in naïve cynomolgus monkeys were evaluated.

FP1 was administered subcutaneously to a cohort of naïve cynomolgus monkeys at three dose levels; 1, 3 and 10 nmol/kg. A vehicle treated group was also included. The animals were treated in a blinded manner. The study lasted a total of 6 weeks: 2 weeks of baseline food intake measurement and data collection, 4 weeks of data collection after single administration of compound. Plasma drug exposures were measured on days 1, 7, 14, 21, and 28 following dosing.

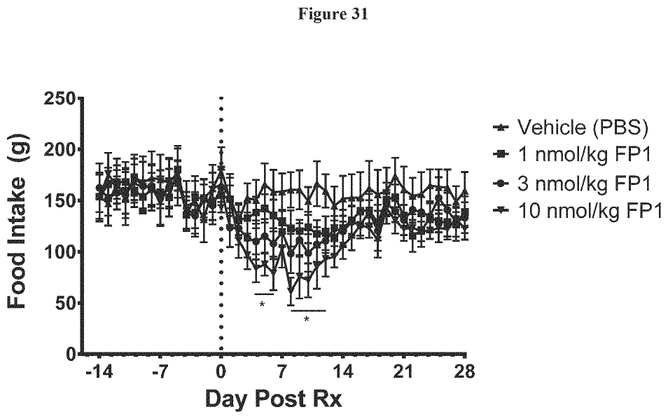

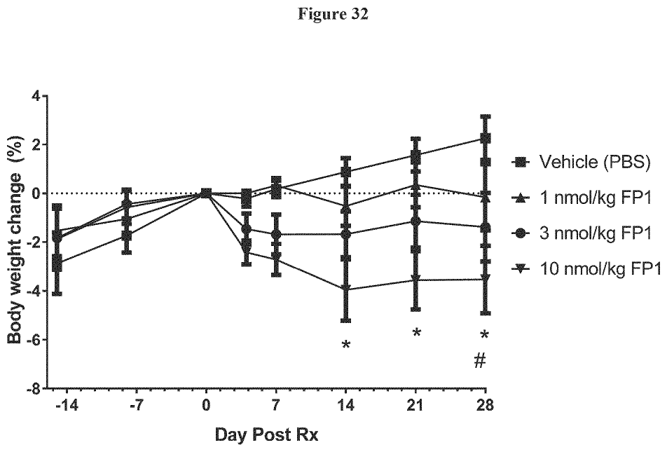

Treatment of cynomolgus monkeys with a single dose of FP1 reduced food intake and body weight compared to vehicle treatment (FIGS. 31-32). A significant reduction in daily food intake was seen on days 4, 5, 6, and 8 through 12 for the 10 nmol/kg dose level (FIG. 31). The weekly average of daily food intake was significantly reduced for during week 2 post administration for the 10 nmol/kg dose level. The 3 nmol/kg dose level had a significant percent reduction from the average weekly food intake prior to dosing on week 2 post administration and the 10 nmol/kg dose level had a significant percent reduction from the average weekly food intake prior to dosing in weeks 1 and 2 post administration. A significant reduction in percent body weight change from day 0 was seen at day 28 for the 3 nmol/kg dose level, and on day 14, 21, and 28 for the 10 nmol/kg dose level (FIG. 32).

FP2 was administered subcutaneously to a cohort of naïve cynomolgus monkeys at three dose levels; 1, 3 and 10 nmol/kg. A vehicle treated group was also included. The animals were treated in a blinded manner. The study lasted a total of 11 weeks: 5 weeks of baseline food intake measurement and data collection, 1 week of treatment and 5 weeks of wash-out phase data collection. Plasma drug exposures were measured on days 1, 7, 14, 21, 28, 35, and 42 following dosing.

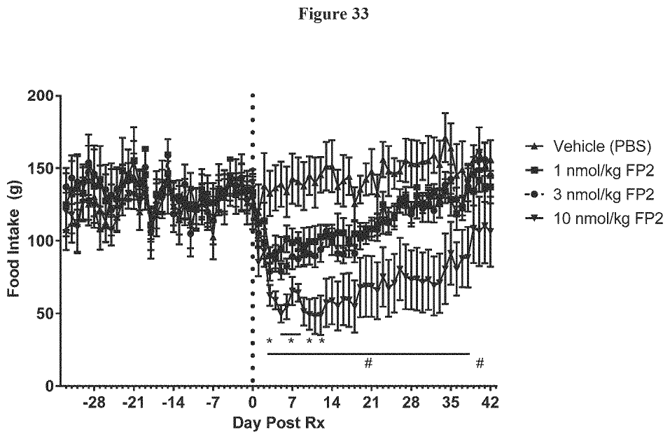

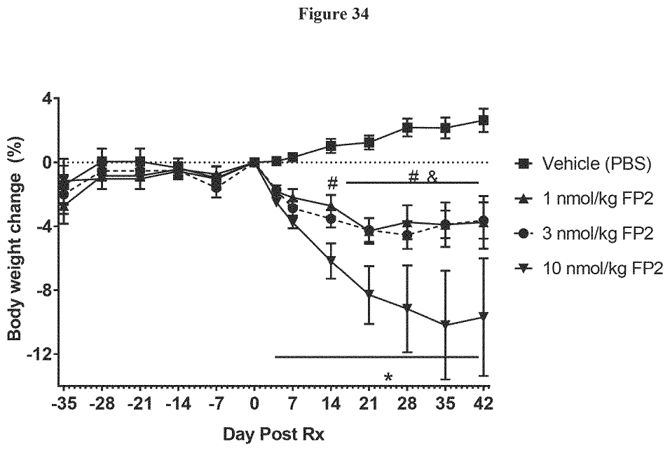

Treatment of cynomolgus monkeys with a single dose of FP2 reduced food intake and body weight compared to vehicle treatment (FIGS. 33-34). A significant reduction in daily food intake was seen on days 3, 5 through 8, 10 and 12 for the 3 nmol/kg dose level and from days 3 through 38 and day 40 for the 10 nmol/kg dose level (FIG. 33). The weekly average of daily food intake was significantly reduced for week 1 post administration for the 3 nmol/kg dose level and significantly reduced for weeks 1 through 6 for the 10 nmol/kg dose level. The 3 nmol/kg dose level had a significant percent reduction from the week prior to dosing in weekly average daily food intake on week 2 post administration and the 10 nmol/kg dose level had a significant percent reduction from the week prior to dosing in weekly average daily food intake on weeks 1 through 6 post administration. A significant reduction in percent body weight change from day 0 was seen from days 21 through 42 for the 1 nmol/kg dose level, from days14 through 42 for the 3 nmol/kg dose level and from days 7 through 42 for the 10 nmol/kg dose level (FIG. 33).

### Example 19: Efficacy of Multiple Dose FP2 in Cynomolgus Monkeys

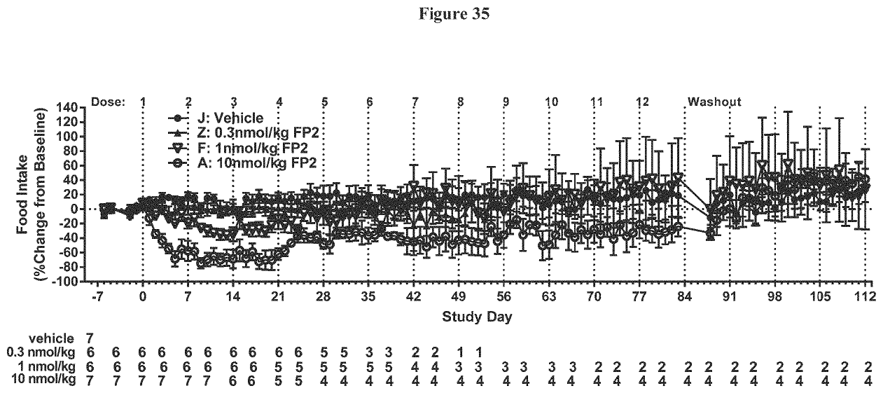

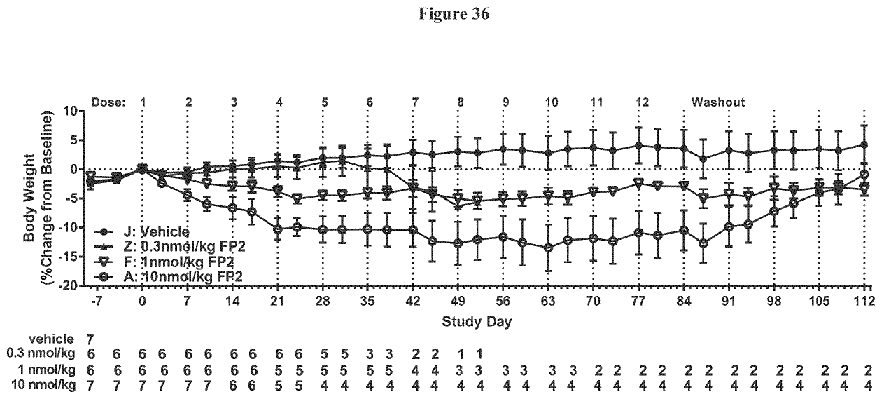

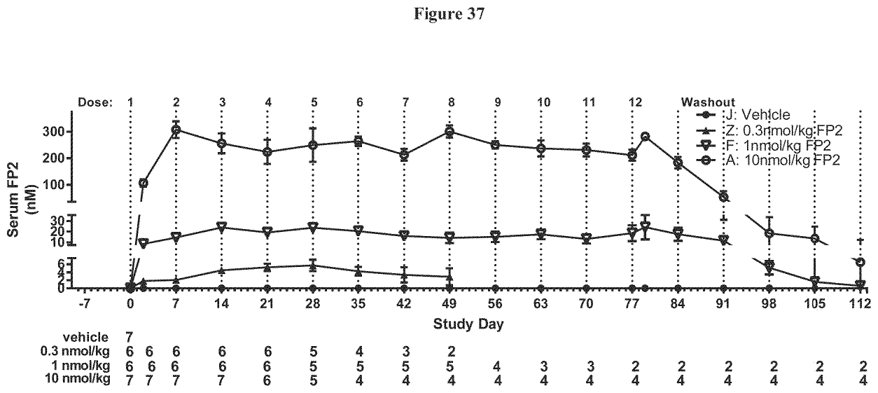

The efficacy of FP2 was evaluated with once-weekly subcutaneous injections to a cohort of naïve spontaneously overweight cynomolgus monkeys (ranging in age from 8-20 years and in body weight from 8.0-11.9 kg) at 3 dose levels: 0.3, 1, and 10 nmol/kg. Food consumption was measured daily, body weight was measured weekly and animals were clinically assessed daily. Treatment of overweight cynomolgus monkeys with 12 weekly doses of FP2 reduced food intake (FIG. 35) and body weight (FIG. 36) compared to vehicle treatment. Circulating FP2 concentration was determined by immunoassay (FIG. 37). Loss of FP2 exposure in some animals at later time points was observed, presumably due to the development of antidrug antibodies (ADA): figures show data collected up to the point prior to loss of exposure (defined as a ≥40% reduction in trough serum drug concentration from the previous measurement for the same animal). No treatment-related adverse effects were noted throughout the study.

### Example 20: Linker Thermal Stability

Thermal stability was investigated for various linkers that connect HSA and GDF15. To evaluate the potential to fragment and aggregate, HSA-GDF15 fusion proteins with various linkers were diluted to 10 mg/ml. After addition of EDTA and Methionine, the samples were incubated under 40° C. for 14 days. Then samples were diluted to the concentration of 1 mg/ml and evaluated under size-exclusion high-performance liquid chromatography (SE-HPLC). Percent of intact protein as well as aggregate and fragment were quantified for these proteins. Table 64 shows that the HSA-GDF15 proteins with linkers that consist of AP repeats are most stable against fragment under thermal stress.

To evaluate the whether these linker affects GDF15 interaction with its receptor, an immunoassay with GFRAL-Fc fusion protein coated on plate and anti-GDF15 or anti-HSA detection was performed, using monoclonal antibodies for GDF15 (Janssen) and HSA (Kerafast, Inc., Boston, Mass.). The assay showed all these linker variants in Table 66 has similar binding to receptor.

TABLE 64
SE-HPLC results after thermal stress for 14 days.

| SEQ | | aggregate | intact | fragment |
|---|---|---|---|---|
| ID NO | Linker | (%) | (%) | (%) |
| 113 | GS(GGGGS)₈ | 3.33 | 84.44 | 12.22 |
| 115 | GA(GGGGA)₈ | 3.51 | 87.98 | 8.5 |
| 117 | (AP)₁₀ | 1.64 | 98.36 | 0 |
| 119 | (AP)₁₂ | 2.36 | 97.64 | 0 |
| 121 | GGS-(EGKSSGSGSESKST)₃-GGS | 1.67 | 85.24 | 13.09 |
| 123 | GS(PGGGS)₈ | 2.96 | 88.12 | 8.91 |
| 125 | GS(AGGGS)₈ | 3.44 | 86.22 | 10.34 |
| 127 | GGS-(EGKSSGSGSESKST)₂-GGS | 1.71 | 91.17 | 7.12 |

### Example 21: Clinical Trial Protocol

A Double-Blind, Placebo-Controlled, Randomized, Single Ascending Dose Study to Investigate the Safety, Tolerability, Pharmacokinetics (incl. Absolute Bioavailability), and Immunogenicity of Subcutaneously Administered FP2 in Overweight, Otherwise Healthy Subjects

Protocol 64739090EDI1001; Phase 1

EudraCT NUMBER: 2018-000324-34

### Abbreviations

ADAs anti-drug antibodies
ALT alanine aminotransferase
Anti-HCV hepatitis C antibody
AST aspartate aminotransferase
AUC area under the curve
BA bioavailability
BLQ below the lowest quantifiable concentration
BMI body mass index
BP blood pressure
BPM Beats per minute
BUN blood urea nitrogen
BW body weight
CNS central nervous system
CRF case report forms (electronic for this study)

### CRU Clinical Research Unit

CV cardiovascular
DCF data clarification form
DG dose group
DIO diet-induced obese

### DRC Data Review Committee

DSM-V Diagnostic and Statistical Manual of Mental Disorders (5th edition)
EC₅₀ half maximal effective concentration
ECG electrocardiogram
eCRF electronic case report form
EDC electronic data capture

### EMA European Medicines Agency

### EU European Union

FcRn neonatal Fc receptor

### FDA US Food and Drug Administration

FIH first-in-human
FSH follicle stimulating hormone

### GCP Good Clinical Practice

### GDF15 Growth Differentiation Factor 15

GFRAL GDNF family receptor-alpha-like, GDF15 receptor
GGT gamma-glutamyl transferase

### GLP Good Laboratory Practices

### HA Health Authority

HbA1c hemoglobin A1c
HBsAg hepatitis B surface antigen
hCG human chorionic gonadotropin
HCV Hepatitis C virus
HDL High-density lipoprotein
HED human equivalent dose
HIV human immunodeficiency virus
HR heart rate
HSA human serum albumin

### IAC Interim Analysis Committee

### IB Investigator's Brochure

ICF informed consent form

### ICH International Conference on Harmonization

### IEC Independent Ethics Committee

### IMP Investigational Medical Product

### IRB Institutional Review Board

IV intravenous
IVRS interactive voice response system
IWRS interactive web response system
LC-MS/MS liquid chromatography/mass spectrometry/mass spectrometry
LDL low-density lipoprotein
LLOQ lower limit of quantification
MABEL minimum anticipated biologic effect level

### MedDRA Medical Dictionary for Regulatory Activities

MRSD maximum recommended starting dose
MRU medical resource utilization
n number (size of a subsample)
N number (total sample size)
NAbs neutralizing antibodies
NAFLD nonalcoholic fatty liver disease
NASH nonalcoholic steatohepatitis
NBE new biological entity
NOAEL no observed adverse effect level
PAD pharmacologically active dose
PAP Papanicolaou smear
PD pharmacodynamic(s)

### PI Principal Investigator

PK pharmacokinetic(s)

### PQC Product Quality Complaint

PRO patient-reported outcome(s) (paper or electronic as appropriate for this study)
PSA prostate specific antigen

### QEWP-5 Questionnaire on Eating and Weight Patterns-5

RBC red blood cell
RET GFRAL-signaling co-receptor
SAD single ascending dose
SAE serious adverse event
SBP systolic blood pressure
SC subcutaneous

### SD Sprague-Dawley

SUSAR suspected unexpected serious adverse reaction
T2DM type 2 diabetes mellitus
TEAE treatment-emergent adverse event
TK toxicokinetics
TSH thyroid stimulating hormone
ULN upper limit of normal

### US United States

### VAS Visual Analog Scale

WCB white blood cell

1. Time and Events Schedule - Screening and Inpatient Period

| Study Period | | | | | | | | |
|---|---|---|---|---|---|---|---|---|
| Parts 1 and 2^{a} | | | | | | | | |
| Screening | Baseline | | Inpatient | | | | | |
| Day | | | | | | | | |
| | | | Day 1 | | | | | |
| Day −28 | | | Predose or Hours Relative to Study Drug Dose | | | | | |
| to −3 | Day −2 | Day −1 | Pre-Doseⁿ | 0 | +0.5 h^{s} | +1 h | +2 h | +4 h |

Screening^{b}

| Informed Consent Form (ICF)^{c} | X | | | |
|---|---|---|---|---|
| ICF for pharmacogenomic sample | X | | | |
| Demographics and Medical History | X | | | |
| Questionnaire on Eating and Weight Patterns-5 | X | | | |
| Inclusion & Exclusion Criteria^{d} | X | X | | |
| Prestudy Therapy | X | X | | |
| Drug, Alcohol and Cotinine Screening^{e} | X | X | | |
| Virus Serology (HBsAg, anti-HCV, HIV)^{e} | X | | | |
| Thyroid Stimulating Hormone (TSH)^{e} | X | | | |
| Follicle Stimulating Hormone (FSH)^{e,f} | X | | | |
| Hemoglobin A1c (HbA1c)^{e} | X | | | |
| Admit to CRU | | X | | |
| Resident in CRU | | X | X | X----------------------------------------------------X |
| Discharge from CRU | | | | |
| Outpatient Visit | X | | | |

Study Drug Administration

| | | |
|---|---|---|
| Randomization^{g} | X | |
| Administer Study Drug^{h} | | X |

Safety Assessments

| Complete Physical Examination | X | | | | | | | |
|---|---|---|---|---|---|---|---|---|
| Brief Physical Examination | | X | | | | | | |
| Height | X | | | | | | | |
| Body Temperature (tympanic) | X | | X | X | | | | |
| Vital Signs (Blood Pressure and Heart Rate)ⁱ | X | | X | X | X | X | X | X |
| Continuous lead II ECG Monitoring^{j} | | | | X-------------X | | | | |
| 12-lead ECG^{k,l} | X | | X^{l} | X | | X | X | X |

Pharmacodynamic Assessments

| Body Weight^{m} | X | X | X |
|---|---|---|---|
| VAS Questionnaires^{o} | | X-----------------------------------------------------X | |
| Standardized Meals for 24-hour food intake | | X | |
| assessment^{o} | | | |

Clinical Laboratory Assessments

| | | |
|---|---|---|
| Pregnancy Test^{p} | X | X |
| Hematology, Chemistry^{e} | X | X |
| Lipid Panel (Total Cholesterol, Triglycerides, HDL | X | X |
| and LDL)^{e} | | |
| Coagulation Panel^{e} | X | X |
| Urinalysis^{e} | X | X |

Exploratory Biomarkers

| | | | |
|---|---|---|---|
| Blood Sample Collection for Exploratory | X | X | X |
| Biomarkers^{q} | | | |

Pharmacokinetic and Immunogenicity Procedures

| | | | | | |
|---|---|---|---|---|---|
| Pharmacokinetic (PK) Blood Sample Collection^{l, q} | X | X^{s} | X | X | X |
| Blood Sample Collection for Anti-Drug Antibodies | X | | | | |
| (ADA)^{q} | | | | | |

Optional Pharmacogenomics Blood Sampling

| | |
|---|---|
| DNA Blood Sample Collection^{t, q} | X |

Ongoing Subject Safety Evaluations

| Adverse Events & Concomitant Medications | X --------------------------------------------------------------- X | | | | |
|---|---|---|---|---|---|
| Reporting^{u} | | | | | |
| Adverse Event (Non-Directive Questions)ⁿ | | X | X | | |
| Local Tolerability Assessment/Local Injection | | | X | X | X |
| Site Evaluation (Part 1)^{v} | | | | | |
| Allergic Reactions/Generalized | | | X | X | X |
| Hypersensitivities^{w} | | | | | |

| | Study Period | | | | | | | | |
|---|---|---|---|---|---|---|---|---|---|
| | Parts 1 and 2a | | | | | | | | |
| | Inpatient | | | | | | | | |
| | Day | | | | | | | | |
| | Day 1 | | | Day 2 | | | Day 3 | Day 4 | Day 5 |
| | Predose or Hours Relative to Study Drug Dose | | | | | | | | |
| | +6 h | +8 h | +12 h | +24 h | +30 h | +36 h | +48 h | +72 h | +96 h |
| Screening^{b} | | | | | | | | | |
| Informed Consent Form (ICF)^{c} | | | | | | | | | |
| ICF for pharmacogenomic sample | | | | | | | | | |
| Demographics and Medical History | | | | | | | | | |
| Questionnaire on Eating and Weight Patterns-5 | | | | | | | | | |
| Inclusion & Exclusion Criteria^{d} | | | | | | | | | |
| Prestudy Therapy | | | | | | | | | |
| Drug, Alcohol and Cotinine Screening^{e} | | | | | | | | | |
| Virus Serology (HBsAg, anti-HCV, HIV)^{e} | | | | | | | | | |
| Thyroid Stimulating Hormone (TSH)^{e} | | | | | | | | | |
| Follicle Stimulating Hormone (FSH)^{e,f} | | | | | | | | | |
| Hemoglobin A1c (HbA1c)^{e} | | | | | | | | | |
| Admit to CRU | | | | | | | | | |
| Resident in CRU | X----------------------------------------------------X | | | | | | | | |
| Discharge from CRU | | | | | | | | | X |
| Outpatient Visit | | | | | | | | | |
| Study Drug Administration | | | | | | | | | |
| Randomization^{g} | | | | | | | | | |
| Administer Study Drug^{h} | | | | | | | | | |
| Safety Assessments | | | | | | | | | |
| Complete Physical Examination | | | | | | | | | |
| Brief Physical Examination | | | | | | | | | X |
| Height | | | | | | | | | |
| Body Temperature (tympanic) | | | | X | | | X | X | X |
| Vital Signs (Blood Pressure and Heart Rate)ⁱ | X | X | X | X | X | X | X | X | X |
| Continuous lead II ECG Monitoring^{j} | | | | | | | | | |
| 12-lead ECG^{k,l} | | X | X | X | | | X | X | X |
| Pharmacodynamic Assessments | | | | | | | | | |
| Body Weight^{m} | | | | X | | | X | X | X |
| VAS Questionnaires^{o} | X-----------------------------------------------------X | | | | | | | | |
| Standardized Meals for 24-hour food intake | | | | | | | X | | |
| assessment^{o} | | | | | | | | | |
| Clinical Laboratory Assessments | | | | | | | | | |
| Pregnancy Test^{p} | | | | | | | | | |
| Hematology, Chemistry^{e} | | | | X | | | X | | X |
| Lipid Panel (Total Cholesterol, Triglycerides, HDL | | | | | | | X | | X |
| and LDL)^{e} | | | | | | | | | |
| Coagulation Panel^{e} | | | | X | | | X | | X |
| Urinalysis^{e} | | | | X | | | X | | X |
| Exploratory Biomarkers | | | | | | | | | |
| Blood Sample Collection for Exploratory | | | | X | | | | | X |
| Biomarkers^{q} | | | | | | | | | |
| Pharmacokinetic and Immunogenicity Procedures | | | | | | | | | |
| Pharmacokinetic (PK) Blood Sample Collection^{l, q} | X | X | X | X | | | X | X | X |
| Blood Sample Collection for Anti-Drug Antibodies | | | | | | | | | |
| (ADA)^{q} | | | | | | | | | |
| Optional Pharmacogenomics Blood Sampling | | | | | | | | | |
| DNA Blood Sample Collection^{t, q} | | | | | | | | | |
| Ongoing Subject Safety Evaluations | | | | | | | | | |
| Adverse Events & Concomitant Medications | X --------------------------------------------------------------- X | | | | | | | | |
| Reporting^{u} | | | | | | | | | |
| Adverse Event (Non-Directive Questions)ⁿ | X | | X | X | X | X | X | X | X |
| Local Tolerability Assessment/Local Injection | X | | X | X | | | X | X | X |
| Site Evaluation (Part 1)^{v} | | | | | | | | | |
| Allergic Reactions/Generalized | X | | X | X | | | X | X | X |
| Hypersensitivities^{w} | | | | | | | | | |

^{a}Part 1 - is double-blinded and will consist of up to 7 dose groups Part 2 - is open-label and will consist of a single dose group.
^{b}Screening - procedures must occur within 4 weeks (28 days) prior to administration of study drug on Day 1.
^{c}Informed Consent - must be obtained prior to initiating any study related procedure.
^{d}Inclusion/Exclusion Criteria - minimum criteria for the availability of documentation supporting the eligibility criteria are described in Section 4 Subject Population Eligibility, and will be confirmed after reviewing baseline assessments prior to dosing.
^{e}See Example 21 Section 9.6.2, Clinical Laboratory Tests, for list of clinical laboratory tests to be obtained; subjects must fast (ie, no food or beverages [except water]) for at least 10 hours before blood is drawn. The baseline clinical laboratory test may be obtained on Day −2 or Day −1, provided results are available for review prior to randomization and dosing on Day 1.
^{f}To be obtained in all female subjects only.
^{g}Randomization will be performed on Day 1 after all of the assessments on Day −2 and Day −1 are performed, reviewed and verified to confirm the subject meets all inclusion and no exclusion criteria (e.g. laboratory results, ECG, etc.).
^{h}Part 1 Dose Escalation/Subcutaneous (SC) Dosing: Subjects will receive a single dose of FP2 or placebo (2 mL maximum volume). Part 2: Subjects will receive a single dose IV infusion FP2 over 30 minutes (constant-rate). All subjects in Parts 1 and 2 will have to fast overnight (at least 10 hours) prior to dosing through 3 hours after dosing. Time 0 for Part 1 is the SC study drug injection time, and for Part 2 is the study drug IV infusion start time.
ⁱVital Signs should be measured after 5 minutes of rest in a supine position and include resting heart rate (HR) and blood pressure (BP). If blood sampling or vital sign measurement is scheduled for the same timepoint as ECG recording, the procedures should be performed in the following order: vital signs, ECG(s), PK, blood draw for safety or exploratory biomarker analysis. Measurements are to be determined with a completely automated blood pressure device. At all timepoints, single blood pressure and HR will be measured and recorded.
^{j}Continuous lead II ECG monitoring will be conducted only in Part 2, and should be started on Day 1 from 30 minutes prior to beginning the IV infusion until 2 hours after the end of the infusion. At the discretion of the investigator, the duration of cardiac monitoring may be extended. k.
^{k}12-lead ECGs: All ECGs, except for the screening ECG, will be measured in triplicate. Subjects should rest for at least 5 minutes in a supine position in a quiet setting without distractions (eg, television, cell phones) and should refrain from talking or moving arms or legs. The triplicate ECGs should be obtained less than 2 minutes apart at each timepoint. When an ECG is performed at the same study timepoint as a PK sample, the PK sample should be taken immediately following the ECG. The 12-Lead ECGs on Day −1 should be time-matched (at the same times) to the 12-Lead ECGs scheduled on Day 1(ie, pre-dose, 1, 2, 4, 8, 12, and 24 hours postdose).
^{m}Body weight: To be obtained prior to the morning meal and after voiding on Days −1, 2, 3, 4 and 5 and pre-dose on Day 1 after voiding. Body weight must be measured in duplicate. Subjects should be weighed on a calibrated scale, while wearing a gown without shoes.
ⁿPre-dose procedures are to be obtained within 30 minutes prior to study drug administration.
^{o}For a detailed description of 24-hour food intake assessment and timing of VAS questionnaires, refer to Example 21 Section 9.3, Pharmacodynamic Evaluations, to the Meals and VAS Questionnaires Time and Events schedule.
^{p}At Screening, a serum pregnancy test is required for all females. Urine pregnancy test can be obtained at all other timepoints.
^{q}Refer to lab manual for sample collection procedures and processing instructions.
^{r}Pharmacokinetic Assessments: All PK blood draws should be performed as close as possible to the scheduled timepoint. When an ECG is performed at the same timepoint, the PK specimen should be taken immediately after the completion of the ECG. For Part 2 (IV infusion) all the collection timepoints are relative to end of infusion. The timing of pharmacokinetic sample collection may be modified (but no additional samples will be collected), if indicated by preliminary PK data from the preceding dose(s).
^{s}Timepoint 0.5 (=t₀ (end of infusion) is only applicable for the IV administration in Part 2.
^{t}The pharmacogenomic (DNA) sample should be collected at the specified time point, however if necessary it may be collected at a later timepoint without constituting a protocol deviation.
^{u}Adverse events and concomitant medications will be recorded starting after the signing of the informed consent until the final study procedure at the end-of-study visit. In addition, adverse events will be queried (using non-directive questions) throughout the study at the timepoints specified.
^{v}Refer to Table 70 in Example 21 Section 9.6.8, Local Injection Site Reaction, for guidelines on reporting toxicity for local injection site reactions.
^{w}Refer to Example 21 Section 9.6.7, Allergic Reactions/General Hypersensitivities, for guidelines on managing allergic and/or hypersensitivity reactions.

1. Time and Events Schedule - Food intake and VAS Questionnaires

| | Clock time^{a} | | | | | | | | | | | |
|---|---|---|---|---|---|---|---|---|---|---|---|---|
| | 0655 | 0700 | 0730 | 0800 | 0900 | 1000 | 1100 | 1155 | 1200 | 1230 | 1300 | 1400 |
| | Hours Relative to First Meal | | | | | | | | | | | |
| | | | | | | | | 4 | | | | |
| | | Time | | 1 | 2 | 3 | 4 | hours | 5 | 5.5 | 6 | 7 |
| | −5 min | 0^{b} | 30 min | hour | hours | hours | hours | 55 min | hours | hours | hours | hours |
| Breakfast ^{c, d, e, f, g} | | X | | | | | | | | | | |
| Lunch ^{c, d, e, f, g} | | | | | | | | | X | | | |
| Snack ^{c, d, e, f, g} | | | | | | | | | | | | |
| Dinner ^{c, d, e, f, g} | | | | | | | | | | | | |
| VAS Questionnaires for | X | | X | X | X | X | X | X | | X | X | X |
| Appetite Ratings on Day −1 | | | | | | | | | | | | |
| and 3^{h} | | | | | | | | | | | | |
| VAS Questionnaires for | X | | X | | | X | | X | | X | X | |
| Appetite Ratings on Day 1, 2, | | | | | | | | | | | | |
| 4 and 5 ^{b, i} | | | | | | | | | | | | |
| VAS Questionnaires for Food | | X | | | | | | | X | | | |
| Palatability Days −1 to 5^{j} | | | | | | | | | | | | |

| | Clock time^{a} | | | | | | | | | |
|---|---|---|---|---|---|---|---|---|---|---|
| | 1500 | 1555 | 1600 | 1630 | 1700 | 1800 | 1900 | 1915 | 1920 | 1950 |
| | Hours Relative to First Meal | | | | | | | | | |
| | | 8 | | | | | | 12 | 12 | 12 |
| | 8 | hours | 9 | 9.5 | 10 | 11 | 12 | hours | hours | hours |
| | hours | 55 min | hours | hours | hours | hours | hours | 15 min | 20 min | 50 min |
| Breakfast ^{c, d, e, f, g} | | | | | | | | | | |
| Lunch ^{c, d, e, f, g} | | | | | | | | | | |
| Snack ^{c, d, e, f, g} | | | X | | | | | | | |
| Dinner ^{c, d, e, f, g} | | | | | | | | | X | |
| VAS Questionnaires for | X | X | | X | X | X | X | X | | X |
| Appetite Ratings on Day −1 | | | | | | | | | | |
| and 3^{h} | | | | | | | | | | |
| VAS Questionnaires for | | X | | X | | | X | X | | X |
| Appetite Ratings on Day 1, 2, | | | | | | | | | | |
| 4 and 5^{b, i} | | | | | | | | | | |
| VAS Questionnaires for Food | | | X | | | | | | X | |
| Palatability Days −1 to 5^{j} | | | | | | | | | | |

^{a}Clock time can be adjusted ±15 minutes if needed.
^{b} Time 0 refers to the beginning of breakfast on Days −1, 2, 3, 4, and 5, and to the beginning of the first meal post-dose on Day 1.
^{c} All subjects should be provided their meals at the same time (±15 minutes) on all days. The timing of the meals has been determined in order to avoid overlap with ECG measurements on Day −1, and it has been calculated assuming that ECGs will be conducted before 7 am, and then either at 0800, 0900, 1100, 1500, and 1900 (if dosing on Day 1 occurs at 0700) or at 1000, 1100, 1300, 1700, and 2100 (if dosing on Day 1 occurs at 0900).
^{d} Breakfast, lunch, snack and dinner should be identical on Days −1 and 3, and should be different from meals administered on the other days.
^{e} On Days −1 and 3, subjects should be visually isolated from one another when consuming the meals to avoid being influenced by other subjects.
^{f} On Days −1 and 3, each meal should be consumed within 30 minutes, and subjects should alert the study personnel if meal is completed prior to 30 minutes. Time of meal start and completion should be recorded.
^{g} On Days −1 and 3, each item of each meal should be weighed before and after consumption, and the amount consumed should be recorded for subsequent calories count.
^{h} VAS questionnaires for Appetite Ratings should be administered right before and at the end of each meal, hourly on Days −1 and 3, and every 3 hours on Days 1, 2, 4 and 5. For details regarding VAS questionnaires for Appetite Ratings refer Example 21 Section 9.3, Pharmacodynamic Evaluations.
ⁱ On Day 1, VAS questionnaires for Appetite Ratings should be administered starting with the first meal of the day (3 hours post-dose)
^{j}VAS questionnaires for Food Palatability should be completed by the subjects after the first bite of food. For details regarding VAS questionnaires for Food Palatability refer to Example 21 Section 9.3, Pharmacodynamic Evaluations.

1. Time and Events Schedule - Outpatient Period

| Parts 1 and 2 | | | | | | | | |
|---|---|---|---|---|---|---|---|---|
| Study Period | | | | | | | | |
| Outpatient Visits^{a} | | | | | | | | |
| Week | | | | | | | | |
| 1 | 2 | 3 | 4 | 6 | 8 | 10 | 12 | |
| Days Relative to Study Drug Dose | | | | | | | | |
| | | | | | | | | End-of- |
| Day 7 | Day 14 | Day 21 | Day 28 | Day 42 | Day 56 | Day 70 | Day 84 | Study^{h} |

Safety Assessments

| | | | | | | | | | |
|---|---|---|---|---|---|---|---|---|---|
| Complete Physical Examination | | | | | | | | X | X |
| Brief Physical Examination | X | X | X | X | X | X | X | | |
| Body Temperature | X | X | X | X | X | X | X | X | X |
| Vital Signs (Blood Pressure and Heart Rate)^{b} | X | X | X | X | X | X | X | X | X |
| 12-lead ECG^{c} | X | X | X | X | X | X | X | X | X |

Pharmacodynamic Assessments

| | | | | | | | | | |
|---|---|---|---|---|---|---|---|---|---|
| Body Weight | X | X | X | X | X | X | X | X | X |

Clinical Laboratory Assessments

| | | | | | | | | | |
|---|---|---|---|---|---|---|---|---|---|
| Pregnancy Test (urine) | | | X | | | X | | X | X |
| Hematology, Chemistry^{d} | X | X | X | X | X | X | X | X | X |
| Lipid Panel (Total Cholesterol, Triglycerides, HDL, and LDL)^{d} | | | | | | | | | X |
| Coagulation Panel^{d} | X | X | X | X | X | X | X | X | X |
| Urinalysis^{d} | X | X | X | X | X | X | X | X | X |

Exploratory Biomarkers

| | | | | |
|---|---|---|---|---|
| Blood Sample Collection for Exploratory Biomarkers^{e} | X | X | X | X |

Pharmacokinetic and Immunogenicity Procedures

| | | | | | | | | |
|---|---|---|---|---|---|---|---|---|
| Pharmacokinetic (PK) Blood Sample Collection^{e,f} | X | X | X | X | X | X | X | X |
| Blood Sample Collection for Anti-Drug Antibodies (ADA)^{e} | | | | X | | X | | X |

Ongoing Subject Safety Evaluations

| Adverse Events & Concomitant Medications Reporting^{g} | X------------------------------------------------------X | | | | | | | | |
|---|---|---|---|---|---|---|---|---|---|
| Adverse Event (Non-Directive Questions)^{g} | X | X | X | X | X | X | X | X | X |

^{a}Outpatient Visits: The visits should be performed in the morning with the subject fasting for at least 10 hours (overnight) prior to study procedures. All reasonable attempts should be made to conduct outpatient visits at the scheduled timepoints (ie, specific day for each visit) but a window within ±1 day is allowed up to the Week 4 (Day 28) visit and a window of ±3 days is allowed for the remaining outpatient visits out to Week 12 (Day 84). All subsequent visits should be scheduled relative to the date of the first study drug dose (Day 1) and not the date of the previous rescheduled visit.
^{b}Vital signs should be measured after 5 minutes of rest in a supine position and include tympanic temperature, resting heart rate (HR), and blood pressure. If blood sampling or vital sign measurement is scheduled for the same timepoint as ECG recording, the procedures should be performed in the following order: vital signs, ECG(s), blood draw. Measurements are to be determined with a completely automated blood pressure device. At all timepoints, single blood pressure and HR will be measured and recorded.
^{c}12-lead ECGs: All ECGs will be measured in triplicate. Subjects should rest for at least 5 minutes in a supine position in a quiet setting without distractions (e.g., television, cell phones) and should refrain from talking or moving arms or legs. The triplicate ECGs should be obtained less than 2 minutes apart at each timepoint. When an ECG is performed at the same study timepoint as a PK sample, the PK sample should be taken immediately following the ECG.
^{d}See Protocol Section 9.6.2, Clinical Laboratory Tests, for list of clinical laboratory tests to be obtained; subjects must fast (ie, no food or beverages [except water]) for at least 10 hours before blood is drawn.
^{e}Refer to lab manual for sample collection procedures, and processing instructions.
^{f}Pharmacokinetic Assessments: All PK blood draws should be performed as close as possible to the scheduled timepoint. When an ECG is performed at the same timepoint, the PK specimen should be taken immediately after the completion of the ECG. For Part 2 (IV infusion) all the collection timepoints are relative to end of infusion. The timing of pharmacokinetic sample collection may be modified (but no additional samples will be collected), if indicated by preliminary PK data from the preceding dose(s).
^{g}Adverse events and concomitant medications will be recorded starting after the signing of the informed consent until the final study procedure at the end-of-study visit. In addition, adverse events will be queried (using non-directive questions) throughout the study at the timepoints specified.
^{h}End-of-Study Visit must occur 7 to 10 days after the Day 84 outpatient visit. For subjects who withdraw early, the end-of-study assessment should occur as soon as possible after study drug administration.

### 1. Introduction

Growth differentiation factor 15 (GDF15) is a circulating protein factor, present as a dimer of 25 kDa in human plasma. Published and internal data support its role in regulation of energy balance primarily affecting energy (ie, food intake).

Subcutaneous (SC) administration of FP2 results in decreased food intake and subsequent body weight (BW) loss in rodents and nonhuman primates. In addition, SC treatment with FP2 results in improved glucose homeostasis and ameliorates insulin resistance in diet-induced obese (DIO) mice, likely due to body weight loss. FP2 exerts its effects by binding to the recently identified GDF15 receptor, GDNF family receptor-alpha-like (GFRAL), which is primarily expressed in the area postrema of the central nervous system (CNS)^{5,17,24,15}. It is hypothesized that FP2 will decrease food intake and subsequently cause body weight loss in obese subjects, which will also lead to an improvement in obesity-associated comorbidities.

1.1. Background

1.1.1. Nonclinical Studies

Pharmacologic Profile

The in vitro agonist potency of FP2 was evaluated using a cell-based pAKT assay with SK-N-AS cells stably over-expressing the human GFRAL receptor. FP2 activated pAKT with a half maximal effective concentration (EC₅₀) of 2.908±0.239 nM (N=3) (see Example 14). Native GDF15 served as an assay control and demonstrated agonist activity with an EC₅₀ of 0.153±0.008 nM (N=3).

FP2 was evaluated for its ability to reduce food intake in multiple species. A single SC administration of FP2 acutely inhibited food intake in male C57Bl/6 mice (see Example 14) and Sprague-Dawley (SD) rats (see Example 15). A single SC administration of FP2 to naïve spontaneously overweight Cynomolgus monkeys led to reduced food intake and subsequent significant body weight loss compared to vehicle-treated animals up to 4 weeks after dosing (Example 18).

Repeated administration of FP2 every 3 days over a period of 2 weeks decreased food intake and body weight, and improved glucose tolerance and insulin sensitivity measured by homeostatic model assessment-insulin resistance in DIO mice (see Example 16). Once-weekly administrations of FP2 to a cohort of naïve spontaneously overweight Cynomolgus monkeys over a period of 12 weeks resulted in significantly reduced food intake and body weight compared to vehicle treatment (see Example 19). Loss of exposure in some animals at later time points was observed, presumably due to the development of anti-drug antibodies (ADAs). No treatment-related adverse effects were noted throughout the study.

Safety Pharmacology

Safety pharmacology endpoints (cardiovascular [CV]-, respiratory- and central nervous system [CNS]-function) were, in accordance with the International Conference on Harmonization (ICH) S6(R1) guideline, assessed as part of the Good Laboratory Practice (GLP) 4-week repeat dose toxicity studies in Cynomolgus monkeys (Study 8372593) and SD rats (Study 8371098). In addition, a standalone CV safety pharmacology study (Study T-2017-044) was conducted in telemetry instrumented Cynomolgus monkeys.

Overall, IV and SC administration of FP2 up to the highest doses had no effects on CV endpoints, core body temperature, respiratory rate, neurological or behavioral endpoints.

Toxicology

Nonclinical safety studies (see Table 65) have been conducted in conformance with GLP, 21 CFR, Part 58 and/or the principles of OECD-GLP in countries that are part of the Organization for Economic Cooperation and Development (OECD) Mutual Acceptance of Data process, and include the appropriate documentation. FP2 test material (Batch No. CVC_PCM01) used for the nonclinical safety studies is considered representative of the clinical test material.

TABLE 65
Overview of Toxicology Studies with FP2

| | | Method of | | |
|---|---|---|---|---|
| | Species | Adminis- | Dura- | Doses |
| Type of Study | and Strain | tration | tion | (mg/kg) |
| Repeat-dose toxicity | SD Rat | SC (biw) | 4 weeks | 0, 1, 10, 100 |
| Repeat-dose toxicity | Cynomolgus | SC/IV (qw) | 4 weeks | 0, 1, 10, 50 |
| | monkey | | | |

Key: biw = twice per week; IV = intravenous; qw = once weekly; SC = subcutaneous; SD = Sprague Dawley.

Repeated administration of FP2 in Cynomolgus monkeys and SD rats was generally well tolerated. There were no mortalities and no overt clinical signs. Some findings (eg, reduction in food intake and body weight) are considered a consequence of the intended mode of action and are not considered adverse effects.

Relevant Species Selection

Since FP2 is a fully recombinant fusion protein with both human GDF15 and HSA domains linked through a short peptide consisting of natural amino acids, the toxicology program is designed primarily following ICH guideline S6(R1), Preclinical Safety Evaluation of Biotechnology-Derived Pharmaceuticals.

In terms of relevant animal species determination, the GDF15 portion of FP2 is the biologically active component whereas the HSA component primarily serves, via its interaction with neonatal Fc receptor (FcRn), to prolong the half-life and thereby to increase the exposure of FP2. The GDF15 receptor (GFRAL) and the GFRAL-signaling co-receptor (RET) have recently been identified^{17,24,5,15}.

In silico amino acid sequence homology analysis of the biologically active component (GDF15), its receptors (GFRAL and RET), its half-life extension component (HSA), and the albumin receptor (FcRn) in different species revealed the highest degree of similarity (95-100%) between human and monkey (ie, Cynomolgus monkey) and reasonably high similarity (78-100%) between human and rat (Table 66).

TABLE 66
Sequence Homology Across Species

| % Sequence Similarity | | | | | | | | |
|---|---|---|---|---|---|---|---|---|
| to Human | | Human | Cyno* | Pig | Dog | Rabbit | Rat** | Mouse |
| Active | GDF15 | 100 | 95.7 | 78.3 | 78.3 | — | 95.7 | 95.7 |
| component | | | | | | | | |
| Receptors | GFRAL | 100 | 100 | 90.9 | 95.5 | 95.5 | 90.9 | 90.9 |
| | RET | 100 | 98.8 | 89.3 | 90.8 | 89.6 | 87.3 | 86.0 |
| Half-life | Albumin | 100 | 97.8 | 87.0 | 93.5 | 95.7 | 93.5 | 93.5 |
| Extension | FcRn(p51) | 100 | 100 | 90.6 | 90.6 | 84.4 | 78.1 | 78.1 |
| | FcRn(b2M) | 100 | 100 | 100 | 100 | 100 | 100 | 80.0 |

Key: Cyno = Cynomolgus monkey; FcRn = neonatal Fc receptor; GFRAL = GDNF family receptor -α like; RET = GFRAL signaling co-receptor,
*non-rodent and
**rodent species selected for toxicity testing.

In vitro binding analyses indicated FP2 binding to recombinant GFRAL fusion proteins of human, rat, and Cynomolgus monkey, with affinity within a 2-fold range between human and Cynomolgus monkey and a 5-fold range between human and rat. In addition, tissue expression analysis of the GFRAL receptor in different species revealed a comparable expression pattern (predominantly in the area postrema in the hindbrain) in rat, monkey and human²⁴.

In vivo pharmacodynamic (PD) studies demonstrated the hypothesized PD effects (eg, reduction in food intake and body weight) of FP2 in both the Cynomolgus monkey and the rat.

However, single-dose PK studies also demonstrate some significant differences in PK between Cynomolgus monkey (T_{1/2}: approximately 7-9 days) and SD rat (T_{1/2}: approximately 1-2 days) due to the lower affinity of human HSA to the rat FcRn receptor but similar affinity of human HSA to Cynomolgus monkey FcRn.

Therefore, the Cynomolgus monkey is considered to be the most relevant/predictive animal species and was selected as the nonrodent species for the first-in-human (FIH)-enabling nonclinical safety studies. The rat was selected as the rodent toxicology species with some limitations in PK.

Pharmacokinetic Profile

The PK and toxicokinetics (TK) of FP2 were characterized in rodents and in lean Cynomolgus monkeys after a single dose and after chronic administration for up to 4 weeks. Median time to reach maximum concentration (Tₘₐₓ) was estimated to be 1 day, 1 day, and 1.67 days for mice, rats and Cynomolgus monkeys, respectively. The clearance (˜25 and 5 mL/day/kg in rodents and monkeys, respectively) and elimination half-life (˜1.5 and 7.1 days in rodents and monkeys, respectively) of FP2 are significantly different between monkeys and rodents, presumably due to differences in affinity of HSA to rodent or monkey FcRn (ie, HSA binds to rodent FcRn at lower affinity than to human FcRn, whereas affinity of HSA to monkeys FcRn is similar to humans). Therefore, monkeys are considered a more predictive species of FP2 PK in humans than rodents. The predicted elimination halflife for a 90-kg person is approximately 12 to 17 days.

FP2 has been shown to be stable as an intact dimer ex vivo in human plasma for up to 48 hours, and in vivo in Cynomolgus monkey after SC and IV administration. It is anticipated that metabolism of the intact FP2 would be via standard proteolytic pathways.

1.1.2. Clinical Studies

This will be the first administration of FP2 in humans; therefore, no clinical experience is available.

Human Pharmacokinetics and Immunogenicity

No human PK studies have been conducted with FP2 to date.

Efficacy/Safety Studies

No clinical studies have been conducted with FP2 to date.

### 2. Objectives and Hypothesis

2.1 Objectives

2.1.1. Part 1: Escalating Single Doses

In overweight (BMI≥25 to ≤29.9 kg/m2), otherwise healthy subjects, after single, escalating SC doses of FP2:

### Primary Objectives

To assess the safety and tolerability of FP2 administered subcutaneously (SC).

### Secondary Objectives

To assess the PK of FP2.
To assess the immunogenicity of FP2 in terms of potential ADA formation, as well as the possible formation of antibodies directed against endogenous GDF15.
To assess pharmacodynamic (PD) endpoints such as body weight and food intake.

### Exploratory Objectives

To evaluate whether administration of FP2 is associated with changes in PD endpoints such as appetite ratings, and food palatability assessed by using visual analogue scale (VAS) questionnaires.
To evaluate whether endogenous levels of GDF15 are associated with PD endpoints.
To evaluate whether PK of FP2 is associated with PD endpoints.

2.1.2. Part 2: Absolute Bioavailability

In overweight (BMI≥25 to ≤29.9 kg/m2), otherwise healthy subjects, after a single intravenous (IV) dose of FP2:

### Primary Objective

To estimate the absolute SC bioavailability of FP2 by administration of a single dose short-term IV infusion over 30 minutes (constant-rate) to age, sex and body-weight matched subjects (matched to those subjects participating in one of the preceding escalating SC dose groups in Part 1).

### Secondary Objective

To assess the safety and tolerability of IV administered FP2

2.2. Hypothesis

No formal statistical hypothesis testing is planned for this study given the primary objective is safety and tolerability. All other analyses will be exploratory.

### 3. Study Design and Rationale

Overview of Study Design

This is the first-in-human (FIH) study for FP2. The study has 2 parts and will be conducted in overweight, otherwise healthy subjects at a single study center. Part 1 is a randomized, double-blind, placebo-controlled study to assess the safety, tolerability, and PK of single ascending SC doses of FP2. Part 2 is an open-label, single-arm study to evaluate the systemic exposure and PK of FP2 administered as a single dose short-term IV infusion over 30 minutes (constant-rate).

A total of up to approximately 62 overweight (BMI≥25 to ≤29.9 kg/m²), otherwise healthy male and female (non-childbearing potential) subjects, are planned to participate in this study (Parts 1 and 2). Up to approximately 56 subjects will be randomly assigned in Part 1 of this study, and approximately 6 subjects will be assigned in Part 2.

Subjects will be screened for eligibility between Day −28 and Day −3. Qualified subjects will be admitted to the Clinical Research Unit (CRU) on Day −2 and will undergo baseline safety assessments. On Day −1 and Day 3, subjects will undergo a 24-hour food intake measurement and will complete VAS questionnaires to assess appetite ratings and food palatability. Subjects will receive study drug on Day 1 and will remain domiciled continuously in the CRU for safety, tolerability, PK/ADA and PD assessments until the morning of Day 5, when upon completing study evaluations, they may be discharged. Subjects will be required to return to the CRU for outpatient visits at Weeks 1 (Day 7), 2 (Day 14), 3 (Day 21), 4 (Day 28), 6 (Day 42), 8 (Day 56), 10 (Day 70), Week 12 (Day 84), and an end-of-study visit (7 to 10 days later). The total study duration for each subject will be up to approximately 17 weeks.

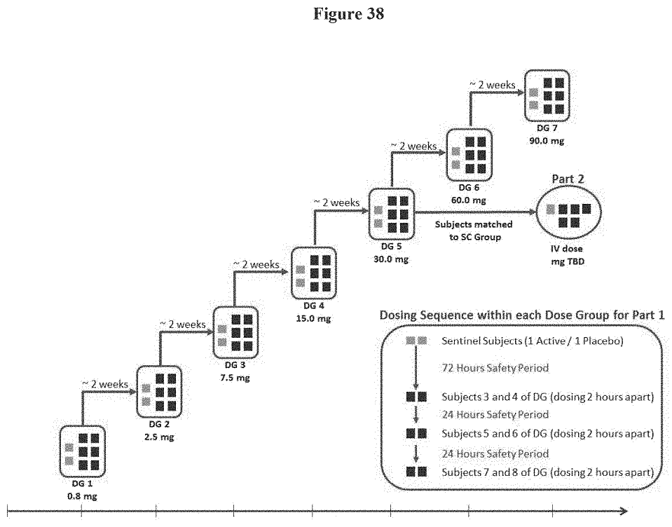

A diagram of the study design is provided in FIG. 38.

3.1.1. Part 1: Single Ascending Dose

Up to 7 dose groups (DGs) of overweight, otherwise healthy subjects (8 subjects per DG) will be studied sequentially. Within each DG, 6 subjects will be randomized to FP2 and 2 subjects will be randomized to matching placebo; thus, the active drug to placebo ratio will be 3:1 for each dose level (see Table 67). Four male subjects and 4 female subjects (randomized 3 active to 1 placebo for each sex group) will be enrolled in the first DG in Part 1 where undiluted study drug is planned to be administered (ie, undiluted formulation of 50 mg/mL) to allow the matching of subjects to the corresponding IV dose group in Part 2 of the study.

The planned dose escalation scheme for FP2 is described in Table 67 below. The dose that will be administered during the study is based on a flat-dose approach calculated for an 80 kg individual as specified in column “FP2 (mg SC)”:

TABLE 67
Planned FP2 Dose Levels in Part 1.

| | | | | Total Number |
|---|---|---|---|---|
| Dose | FP2 | FP2 | Matched Placebo | of Subjects |
| Group | (mg SC) | (mg/kg) | (SC) | per Dose Group |
| 1 | 0.8 mg [N = 6] | 0.01 | Placebo [N = 2] | N = 8 |
| 2 | 2.5 mg [N = 6] | 0.03 | Placebo [N = 2] | N = 8 |
| 3 | 7.5 mg [N = 6] | 0.09 | Placebo [N = 2] | N = 8 |
| 4 | 15 mg [N = 6] | 0.18 | Placebo [N = 2] | N = 8 |
| 5 | 30 mg [N = 6] | 0.36 | Placebo [N = 2] | N = 8 |
| 6 | 60 mg [N = 6] | 0.72 | Placebo [N = 2] | N = 8 |
| 7 | 90 mg [N = 6] | 1.08 | Placebo [N = 2] | N = 8 |

Treatments will be double-blind and randomized at each dose level.

For each dose level, the subjects will be separated into 4 subgroups (n=up to 2/subgroup) and dosed on different days. Two sentinel subjects will be simultaneously dosed first (one placebo, one FP2) on the same day and will complete a 72-hour safety surveillance period before subsequent subjects in the DG may be dosed. Following review of the safety data, up to 2 additional subjects may be dosed (approximately 2 hours apart) per day until all subjects have completed dosing. There will be at least 10 days between the last subject of the preceding group and the first subject of the following DG.

After each completed dose level, preliminary safety and PK data will be reviewed by the Sponsor and Principal Investigator (PI) to decide on the next planned dose level. Each dose-escalation decision will be based on blinded preliminary safety, tolerability and PK data collected in all subjects of a given DG for at least 72 hours post-dose. The minimum number of evaluable subjects (ie, subjects who have completed at minimum the 72-hour post-dose study procedures) required for dose escalation review will be N=7 per DG.

No dose will be administered for which the projected mean serum exposures (Cₘₐₓ or AUC₀₋₄₈ₕᵣ) of FP2 would exceed the lowest No-Observed-Adverse-Effect-Level (NOAEL) exposures from the 1-month GLP toxicology studies in the most relevant species (ie, Cynomolgus monkeys).

3.1.2. Part 2: Absolute Bioavailability

Part 2 is an open-label, single-arm study to evaluate the systemic exposure and PK of administering FP2 as a single dose IV infusion over 30 minutes (constant-rate) to healthy overweight (BMI≥25 to ≤29.9 kg/m²) male and female subjects. The PK data from Part 2 will be used to determine the absolute bioavailability of the SC FP2 dosage form.

Part 2 will enroll 6 overweight (BMI≥25 to ≤29.9 kg/m²), otherwise healthy male (n=3) and female (n=3) subjects. Subjects will be matched for sex, age (±5 yrs), and body weight (±5 kg) to subjects of a SC dose group from Part 1 (probably DG 5; 30 mg, first dose-group in which undiluted FP2 formulation of 50 mg/mL will be used). Part 2 may be initiated before completion of Part 1 of the study after the sponsor and PI have reviewed the blinded preliminary safety and tolerability data from the preceding DGs from Part 1 of the study.

Each eligible subject in Part 2 will receive a single IV dose of FP2 administered as a constant-rate short-term infusion over 30 minutes via an indwelling catheter in a suitable forearm vein. The IV dose for Part 2 will be selected based on the preliminary safety and PK data in Part 1. The selected IV dose will not exceed one third of a dose already assessed as well tolerated in Part 1 to account for expected differences in maximum exposure levels upon IV administration and possibly incomplete bioavailability of the SC formulation. For details see Section 3.5 Dose Selection Part 2 of the Example 21. For safety monitoring, 1 sentinel subject will be dosed first and will complete a 72-hour safety surveillance period before subsequent subjects may be dosed. The remaining 5 subjects will be subdivided into subgroups (dosed at least 24 hours apart), so that no more than 2 subjects will be dosed per day (approximately 2 hours apart).

3.2. Study Design Rationale

3.2.1. General Study Design Considerations

The proposed study is FIH, double blind, randomized, placebo-controlled single ascending dose

(SAD) trial in overweight, otherwise healthy adult subjects to evaluate the safety, tolerability, PK, immunogenicity and PD (ie, food intake, body weight, appetite ratings and food palatability) of FP2, a fully recombinant homodimer of GDF15 fused to HSA.

The study has been designed in agreement with the pertinent regulatory guidelines (EMA Guidance EMEA/CHMP/SWP/28367/07 Rev. 1, 2017; FDA Guidance for Industry, 2005) for first-in-human and other early clinical development studies.

Regarding the anticipated systemic safety, based on the available non-clinical data and pharmacological characteristics, FP2 is not considered to be a “high risk” new biological entity (NBE) according to the criteria outlined in the EMA “Guideline on strategies to identify and mitigate risks for first-in-human clinical trials with investigational medicinal products”⁴.

Important trial design elements such as the determination of a safe starting-dose (based on minimum anticipated biologic effect level [MABEL], pharmacologically active dose [PAD] and NOAEL data), the dose escalation strategy and the definition of stopping criteria, meet current and scientific, medical and ethical standards and requirements (see Sections 3.3, 3.4. 3.5, 3.6 of the Example 21), and are consistent with the designs of other current FIH trials investigating comparable products with similar objectives.

The targeted patient population is well defined and will be carefully selected based on a comprehensive set of applicable inclusion- and exclusion criteria (see Protocol Section 4. Subject Population). All subjects will be monitored with regular safety follow-up over 13 weeks postdose.

The study is designed for and will be conducted in a dedicated CRU under medical monitoring conditions that assure a high probability for the early detection of untoward events and for suitable therapeutic intervention, if required.

3.2.2. Blinding, Control, Study Phase/Periods, Treatment Groups

Part 1

A double-blind, placebo-controlled, randomized study design allows for the best practical assessment of the safety and tolerability profile of FP2 by minimizing potential biases during data collection and evaluation of clinical endpoints. A placebo control will be used for Part 1 to evaluate the frequency and magnitude of changes in clinical endpoints that may occur in the absence of active treatment. Randomization will be used to minimize bias in the assignment of subjects to treatment groups, to increase the likelihood that known and unknown subject attributes (eg, demographic and baseline characteristics) are evenly balanced across treatment groups.

Part 2

Part 2 is an open-label, single-arm study design, that will provide formulation-independent IV PK data on the disposition of FP2 that cannot be otherwise obtained, and will be used to estimate the absolute bioavailability (BA) of the SC FP2 dosage form.

3.2.3. Study Population

The rationale for enrolling overweight, otherwise healthy subjects in the study is as follows:

FP2 will be administered by the SC route and the drug absorption characteristics from SC tissues (ie, rate and extent of absorption) are likely to differ between different subjects (eg, males vs. females) and populations (ie, lean vs. overweight vs. obese subjects). Therefore, the study aims to determine the initial human PK in a relevant population to enable a reliable prediction of repeat-dose PK and dose-selection in a population that will be close to the target population(s) before exposing overweight or obese subjects in longer duration trials.

Subject risks of overweight, otherwise healthy subjects are deemed comparable to those of lean healthy subjects, as screening criteria will exclude subjects with clinically meaningful conditions known to be more prevalent in overweight individuals (eg, T2DM, hypertension);

Enrollment of overweight, otherwise healthy subjects allows a preliminary evaluation of FP2's safety and PD effects (such as food intake, body weight, appetite ratings and food palatability) in a subject population that is close/comparable to the study population anticipated to be enrolled in Phase 2.

3.2.4. Pharmacokinetics and Pharmacodynamics

The timing and duration of PK sampling in this study is based upon the nonclinical PK data, including allometric model predictions. Using this information, a frequent blood sample collection schedule will allow for a full characterization of the PK profile and provide the data required to define key PK parameters needed to support further clinical development.

Twenty-four-hour assessment of food intake (pre-dose and at projected Tₘₐₓ), and body weight monitoring throughout the study, will allow for evaluation of reduction in food intake (ie, decrease in calorie intake, and for possible weight loss, that might occur upon single-dose administration of FP2. Frequent completion of VAS questionnaires will allow for a characterization of changes in appetite behavior that might be associated with reduction in food intake and body weight loss upon treatment with single-dose administration of FP2.

3.2.5. Safety and Tolerability

The majority of findings recorded in the 1-month GLP toxicology studies in rat and monkey with FP2 were considered to be secondary to the significant food intake reduction and body weight losses observed, which was the hypothesized target pharmacology for this class of drug. Overall, FP2 was deemed well tolerated in the toxicology studies and no findings were noted that that would necessitate specialized monitoring.

Therefore, the safety monitoring in this study will consist of serial standard safety assessments such as vital signs (heart rate, systolic and diastolic blood pressure, body temperature), standard clinical laboratory tests (hematology, clinical chemistry, urinalysis, lipids, coagulation), physical exams, the monitoring of treatment-emergent signs and symptoms/adverse events [TEAEs] including allergic reactions/hypersensitivity and local injection site reactions, and the documentation of serial standard 12-lead ECGs. Continuous lead II ECG monitoring will also be conducted in Part 2.

3.2.6. Immunogenicity

As the immunogenic potential of a NBE is part of its overall safety profile, the potential immunogenicity of FP2 will be monitored by serial quantification of ADAs, and screening for antibodies that may be formed against endogenous GDF15.

3.2.9. IV Administration (Part 2)

Study Part 2 is an open-label, single-arm study to evaluate the systemic exposure and PK of administering FP2 as a single dose IV infusion over 30 minutes (constant-rate) to overweight, otherwise healthy subjects, matched for age, sex and body weight to a suitable SC reference group (first dose group in Part 1 where undiluted study drug will be administered). Part 2 will provide formulation-independent IV PK data on the disposition of FP2 that cannot be otherwise obtained, and will be used to estimate the absolute BA of the SC FP2 dosage form. Six subjects receiving IV administrations of FP2 in Part 2 are customary for the estimation of absolute BA and to serve as a benchmark for the assessment of pharmaceutical quality attributes of SC dosage form.

3.3. Dose Selection and Escalation Rationale for Part 1

An indirect response PK/PD model between the PK of FP2 and food intake, integrated with a physiological representation of the relationship between food intake and body weight (BW), was developed for simultaneously characterizing the PK and PD (% change in food intake and BW compared to baseline) of FP2 at 3 studied dose levels in a repeat dose study in overweight Cynomolgus monkeys (see Example 19). In this study, food intake reductions were maximal from week 2 to 3, and showed dose-dependent attenuation with sustained treatment, whereas body weight declined continuously through week 4 (in the 1 nmol/kg group) or week 7 (in the 10 nmol/kg group) and plateaued thereafter. To characterize these observations, a novel physiology-based PK/PD model was developed to describe the treatment-induced changes in both food intake (FI) and consequently body weight (BW) by including terms describing the compensatory changes in food intake and energy expenditure that occur in response to weight loss. BW change was described as a longitudinal effect of food intake change, together with energy expenditure change over time. This PK/PD model was able to describe the food intake and BW trajectories in the 12-week study and provided an exposure-response relationship for FP2 in overweight Cynomolgus monkeys. BW loss-dependent compensatory food intake term in the model allows parameters for drug effect on food intake to remain constant over time for a given exposure. The semi-mechanistic model developed in cynomolgus monkeys enabled further translational modeling based on known relationships between energy intake and BW changes in humans, with the results showing that for a given % reduction in energy intake, humans have a greater % reduction in BW than Cynomolgus monkeys do. The modeling results also support quantitatively the mechanisms of action of FP2 and the BW loss is primarily driven by drug-induced food intake reduction in overweight Cynomolgus monkeys. This modeling approach was used to determine the PAD and efficacious clinical doses/exposures in humans.

Assuming the PK/PD relationship of FP2, as well as physiologically relevant parameters and SC bioavailability are translatable between overweight Cynomolgus monkeys and humans, the preliminarily predicted human once weekly SC dose expected to confer a 20% food intake reduction at Week 12 is approximately 0.08 mg/kg (˜0.5 nmol/kg). Based on published literature^{13,12,11} and model simulations, a dose that provides a 20% reduction in food intake at Week 12 post once-weekly SC dosing may correspond to greater than 10% weight reduction after one year of treatment.

3.3.1. Starting Dose Justification

The starting dose for this study was selected according to pertinent regulatory guidelines^{3,6} for first-in-human studies based on toxicology (NOAEL) and pharmacological data (PAD).

The NOAEL dose in rats and Cynomolgus monkeys for the 1-month toxicity studies was 100 mg/kg for the rat, and 50 mg/kg for the Cynomolgus monkey. The human equivalent doses (HEDs) were calculated by normalization of the doses to body surface area. The maximum recommended starting dose (MRSD) calculation uses a default safety factor of 10 for providing a margin of safety for protection of human subjects receiving the initial clinical dose.⁶ As reflected in the exposure ratio calculations described below, using a safety factor of 10, the MRSD for FP2 was calculated to be 1.6 mg/kg BW. For a person with an 80-kg body weight, the MRSD dose was calculated to be 128 mg.

Based on its pharmacology, FP2 is expected to function like endogenous GDF15 to reduce food intake, which results in loss of body weight. The mechanism of action and the nonclinical safety profile for FP2 suggests that FP2 does not meet the criteria to be considered a high-risk product.

As the repeat-dose Cynomolgus monkey study results showed pharmacological activity (ie, food intake reduction) at much lower doses, the MRSD should be guided by a PAD-based approach. For this purpose, human PK parameters for FP2 were predicted via fixed exponent allometric scaling of model-estimated PK parameters based on body weight after single SC dosing in overweight cynomolgus monkeys.

Based on this, a PK/PD model of FP2 was developed using PK and PD data across 3 studied dose levels in Cynomolgus monkeys, and the model predicted that a single SC dose of 0.05 mg/kg (˜0.3 nmol/kg) will result in approximately 10% maximum food intake reduction, which is considered a meaningful threshold to indicate pharmacological activity. Since the predicted level of food intake reduction associated with 0.05 mg/kg is not regarded critical for the safety of the subjects, and there are no other known safety-critical PD effects, a safety factor of 5 (instead of a default safety factor of 10) is applied to the model-estimated 0.05 mg/kg dose. The choice of this safety factor also considers the within 2-fold in vitro binding affinity of FP2 to recombinant GFRAL fusion proteins of human and Cynomolgus monkey, as well as comparable tissue expression pattern of the GFRAL receptor in monkey and human, and leads to an MRSD of 0.01 mg/kg. As a flat-dosing approach for a body weight of 80 kg will be used in this study, the PAD-based MRSD was calculated to be 0.8 mg (0.01 mg/kg×80 kg). This indicates that the PAD-based MRSD is about 160-fold lower than a NOAEL-based MRSD.

The PAD-based MRSD of 0.01 mg/kg is predicted to yield a maximum serum drug concentration of ˜0.6 nM, which is 13-fold higher than endogenous GDF15 upper normal range levels (ie, ˜0.046 nM or 1.15 ng/mL)², and 5-fold lower than the median endogenous GDF15 level found in pregnant women without complications (ie, ˜3.2 nM or 80,000 pg/mL).²⁰ This concentration value, when corrected for the difference in: 1) human GFRAL receptor binding affinity (11-fold reduced binding affinity of FP2 when compared to endogenous GDF15); and 2) differences in potency from an in vitro functional assay (˜19-fold reduced EC₅₀ values for FP2 vs. endogenous GDF15 in the pAKT functional assay using rhGFRAL-expressing SK-N-AS cells), results in the prediction that the anticipated maximum concentration of FP2 in humans after a single SC dose of 0.01 mg/kg is within 1.2 fold of the endogenous GDF15 normal upper range in lean individuals.

In addition, the Cₘₐₓ from the starting dose of 0.01 mg/kg is expected to be lower than EC₁₀ of the pAKT functional assay readout using rhGFRAL-expressing cells.

3.3.2. Maximum Dose

The NOAEL doses in the 4-week GLP rat or Cynomolgus monkey toxicology studies resulted in mean Cₘₐₓ values of 415 μg/mL (Days 1-4, female and male) and 1,117 μg/mL (Day 22-29, female and male), respectively, and mean AUC values of 883 μg day/mL (Day 1-4, male and female) and 6,341 μg day/mL (Days 22-29, male and female), respectively. No apparent differences in sex delineated mean drug exposures (assessed by Cₘₐₓ and AUC) and other TK parameters were observed between male and female Cynomolgus monkeys. There was a trend that the drug exposures (assessed by Cmax and AUC_{Day1-4} following the first dose) in the female rats were slightly higher than in the male rats. The maximum dose of 1.08 mg/kg BW planned in Part 1 is anticipated to yield mean Cₘₐₓ and AUC exposures that are approximately 97- to 21fold below the mean Cₘₐₓ and AUC exposures, respectively, at the NOAEL dose in the Cynomolgus monkey. As Part 1 of this study progresses, the PK data from each sequential DG will be utilized for simulations that predict systemic exposures of FP2, and further refine these exposure estimates. Subsequent doses may be adjusted based on a review of the emerging safety, tolerability and PK data, but will not exceed the maximum dose planned.

The CV safety study in instrumented Cynomolgus monkeys did not demonstrate any meaningful findings up to the highest SC dose tested of 50 mg/kg, and therefore was not considered to limit the planned maximum exposures summarized above.

3.4. Dose Escalation

The planned dose range in Part 1 will allow characterization of doses that are anticipated to provide safety margins (≥10-fold) from expected repeat-dose exposures of therapeutically effective doses that would be explored in future studies in obese subjects, and will account for potential exposure increases in special populations (eg, subjects with renal and hepatic impairment) and settings (eg, drug-drug interaction studies, thorough QT/QTc study, etc.).

The proposed dose-escalation strategy follows the concept of ˜3-fold dose increments for the first 2 dose-escalation steps up to the 3^{rd} dose level of the study, when the preceding dose levels have been shown to be safe and exposure levels (Cₘₐₓ and AUC_{0-72 hrs}) display approximately dose-proportional linear PK (or less-than-proportional exposure increases). Under the same provisions, the 3^{rd}, 4^{th}, and 5^{th} dose-escalation steps to dose levels 4, 5, and 6 will consist of ˜2-fold dose escalations, while all subsequent dose-escalation steps (if any) would be planned with approximate 50% dose increments (Table 3).

After each completed dose level, preliminary safety and PK data will be reviewed by the Sponsor and PI to decide on the escalation to the next planned dose level. Each dose escalation decision will be based on blinded preliminary safety, tolerability, and PK data collected in all subjects of a given dose group for at least 72 hours post-dose and preliminary PK data obtained for at least 72 hours post-dose. The minimum number of evaluable subjects (ie, subjects who have completed at minimum the 72-hour post-dose study procedures) required for review will be N=7 per dose group. Any additional clinically relevant information from 72 hours post-dose until the review meeting will be communicated by the investigator at the review meeting. With study progress, the cumulative safety data of preceding dose groups will be regularly reviewed as well as part of each dose-escalation decision meeting. The planned doses may be modified, if supported by preliminary PK, safety, and/or tolerability data from preceding dose(s), and may be decreased or repeated, but not increased unless a substantial amendment to the study protocol would be issued and submitted to the competent Health Authority (HA) and Independent Ethics Committee (IEC).

There will be at least 10 days between the last subject of the preceding dose group and the first subject of the following DG.

The NOAEL doses in the 4-week GLP rat or Cynomolgus monkey toxicology studies will be used to guide the targeted upper exposure limit in this study (For details see Example 21, Section 3.3.2).

3.5. Dose Selection Part 2

The dose strength for Part 2 will be selected based on preliminary safety and PK data in Part 1, and will not exceed ⅓ of a dose assessed as well tolerated in Part 1 to provide an about 3-fold safety margin should FP2 SC BA in overweight human subjects be substantially lower than determined in lean Cynomolgus monkeys (absolute BA upon single IV doses of 1.0 mg/kg 99%). As observed Cₘₐₓ values upon IV dosing (50 mg/kg BW) in the 4-week Cynomolgus monkey TK study were only about 2-fold higher than observed with SC administration of the same dose, a 3-fold reduction of a well-tolerated SC dose is expected to provide also adequate margins for a safe maximum exposure (Cₘₐₓ) expected for a constant rate IV infusion of FP2 over 30 minutes. For example, if the 30 mg SC dose administered in Dose Group 5 will be considered to be safe and well tolerated, the IV dose for Part 2 could be selected based on this DG and would be ⅓ of 30 mg or 10 mg. This will be based on the assumption that the absolute BA upon SC dose in overweight subjects is as low as ˜33%, and therefore the 10 mg IV dose strength will ensure that the AUC upon IV administration will not exceed the AUC obtained after SC administration of 30 mg. Since BA is calculated from AUC (not Cₘₐₓ) and 33% BA assumption is conservative, IV dose strength of ⅓ of SC dose is likely to yield Cₘₐₓ (ie, end of infusion) lower than that of 30 mg SC.

3.6. Stopping Criteria

3.6.1. Individual Stopping Criteria—Part 1

As Part 1 of the study involves a single SC administration of FP2 only, individual stopping-criteria for the study drug (such as discontinuation of dosing) are not applicable. However, a randomized subject will not receive study drug if he/she is withdrawn from the study (see Example 21, Section 10.2).

3.6.2. Individual Stopping Criteria—Part 2

The IV infusion will be discontinued in case of medically important AEs, signifying a potential risk to subjects as judged by the Investigator(s). Such important medically AEs include—but are not limited—to the following findings:

Subject has an absolute QT corrected according to Fridericia's formula (QTcF) of ≥500 msec or an increase in QTcF from baseline of >60 msec in 2 successive measurements (15 minutes apart) after the first occurrence.

Subject with tachycardia defined as resting supine heart rate>100 bpm persisting for at least 15 minutes after the first occurrence based on continuous heart rate monitoring.

Subject with bradycardia defined as resting supine heart rate<45 bpm persisting for at least 15 minutes after the first occurrence based on continuous heart rate monitoring.

Subject who developed hypertension defined as resting supine systolic blood pressure (SBP) above 180 mmHg and persisting for at least 15 minutes after the first occurrence.

Subject experiences a severe or serious adverse event and the Investigator believes that for safety reasons it is in the best interest of the subject to discontinue the IV infusion.

Once discontinued for one of those reasons, the study drug administration is to be considered definitely terminated (ie, will not be restarted again. Reasons for study drug discontinuation will be documented).

3.6.3. Study Stopping Criteria

The progression of the study (either within a not yet completed DG or the progression to a higher dose level) will be put on hold if, at any time:
2 subjects are withdrawn from the study (regardless of dose group or period) due to experiencing any medically important or serious adverse event (SAE) assessed by the
Investigator as possibly, probably or very likely related to study drug, or
1 subject experiences an SAE assessed by the Investigator as possibly, probably or very likely related to study drug.

An internal DRC, independent from the clinical study team, may be convened (see Section 11.8). The purpose of the DRC would be to review all unblinded safety data. Upon conclusion of this in-depth safety review, one of the following recommendations would be made:

To continue with the study as planned (ie, there are no significant safety concerns).

To continue with the study by repeating the current dose in more subjects.

To continue with the study at a dose between the current dose and the next planned dose, or at a dose between the current dose and the previous lower dose.

To terminate the study.

### 4. Subject Population

Screening for eligible subjects will be performed within 28 days before administration of the study drug.

For both Parts 1 and 2, at least 2 additional reserve subjects will be admitted on Day −2 of the inpatient period and will undergo all assessments leading up to dosing to ensure that a full dose group is randomized.

Additional subjects may be enrolled if replacements are required; each replacement subject will complete the entire study for the subject they are replacing according to the randomization schedule (see Protocol Section 5, Study Drug Allocation and Blinding). No subject may participate in more than one dose group or part of this study. Reserve subjects may be rescreened for participation in another dose group in the study.

In Part 1, four males and 4 females (randomized 3 active to 1 placebo for each sex group) will be enrolled in the first dose group where undiluted study drug is planned to be administrated (ie, undiluted formulation of 50 mg/mL) to allow the matching of subjects to the corresponding IV dose group in Part 2 of the study.

The inclusion and exclusion criteria for enrolling subjects in this study are described in the following 2 subsections. If there is a question about the inclusion or exclusion criteria below, the Investigator will consult with the appropriate Sponsor representative and resolve any issues before enrolling a subject in the study. Waivers are not allowed.

For a discussion of the statistical considerations of subject selection, refer to Section 11.2 Sample Size Determination of the Example 21.

4.1. Inclusion Criteria

Each potential subject will satisfy all of the following criteria to be enrolled in the study:

Male or female

18 to 45 years of age, inclusive.

Body Mass Index (BMI) between 25.0 and 29.9 kg/m² (inclusive), and body weight≥80 kg.

Healthy on the basis of physical examination, medical history, vital signs, clinical laboratory tests and 12-lead ECG performed at Screening and at baseline (Day −2 and/or Day −1). If any of the results are abnormal, the subject may be included only if the Investigator judges that the abnormalities or deviations from normal are not clinically significant. This determination will be recorded in the subject's source documents and initialed by the Investigator.

Will sign an informed consent form (ICF) indicating that he or she understands the purpose of, and procedures required for, the study and is willing to participate in the study.

Women will be of non-childbearing potential, defined as either:

Postmenopausal

A postmenopausal state is defined as no menses for at least 12 months without an alternative medical cause, and a. follicle stimulating hormone (FSH) level at screening in the postmenopausal range (>40 IU/L or mIU/mL). However, if the subject has had amenorrhea for less than 12 months, then 2 FSH measurements (1 may come from the subject's medical records) are required to confirm postmenopausal state. All women should have a negative serum ß-human chorionic gonadotropin (hCG) pregnancy test at Screening; and a negative urine pregnancy test at admission on Day −2.

Permanently Sterile

Permanent sterilization methods include hysterectomy, bilateral salpingectomy, bilateral tubal occlusion/ligation procedures, and bilateral oophorectomy or otherwise be incapable of pregnancy, as documented by medical records. All women should have a negative serum hCG pregnancy test at Screening; and a negative urine pregnancy test at admission on Day −2.

A resting heart rate (after the subject is supine for 5 minutes) between 50 and 90 beats per minute (bpm). If heart rate is out of range, up to 2 repeated assessments are permitted.

Blood pressure (after the subject is supine for 5 minutes) between 90 and 140 mmHg systolic, inclusive, and no higher than 90 mmHg diastolic. If blood pressure is out of range, up to 2 repeated assessments are permitted.

Men will agree to use condoms (including men who have had vasectomies) even if their partner is pregnant (this is to ensure that the fetus is not exposed to the study drug through vaginal absorption) and to not donate sperm during the study and for 3 months after conclusion of the study. Male subjects should encourage their female partner to use an effective method (eg, prescription oral contraceptives, contraceptive injections, intrauterine device, double barrier method, and contraceptive patch) of contraception in addition to the condom used by the male study subject.

Willing to adhere to the prohibitions and restrictions specified in the study protocol.

Subjects will like and typically eat the food items that will be provided for 24-hr food intake assessment (at least the main entrée and one of the side dishes from the lunch and dinner menus), and will have a habitual meal pattern of 3 main meals per day (breakfast, lunch and dinner).

Will sign a separate informed consent form if he or she agrees to provide an optional DNA sample for research. Refusal to give consent for the optional DNA research sample[s] does not exclude a subject from participation in the study.

4.2. Exclusion Criteria

Any potential subject who meets any of the following criteria will be excluded from participating in the study:

History of, or currently active, significant illness or medical disorders, including (but not limited to) CV disease (including cardiac arrhythmias, myocardial infarction, stroke, peripheral vascular disease), endocrine or metabolic disease (eg, diabetes, hyper/hypothyroidism, severe hypertriglyceridemia [>400 mg/dL]), hematological disease (eg, von Willebrand's disease or other bleeding disorders), respiratory disease, hepatic or gastrointestinal disease, neurological or psychiatric disease, ophthalmologic disorders (including retinal disorders or cataracts), neoplastic disease, skin disorder, renal disorder, or any other illness that the investigator considers should exclude the subject or that could interfere with the interpretation of the study results.

Previous surgical treatment for obesity or recent changes in body weight (>5%) due to dieting, including commercial weight loss programs, or pharmacologic treatment within 6 months of screening.

Lifetime history of any eating disorder or high risk for eating disorder (using the Questionnaire on Eating and Weight Patterns-5 [QEWP-5]²⁵. See Attachment 1.

Lifetime history of malignancy or family history of susceptibility to malignancies defined as same type of cancer in at least 2 close relatives (defined as parents, siblings, children, grandparents, aunts, uncles, nephews, nieces) on the same side of the family, or more than 1 type of cancer in a single person who is a close-relative, or

close relatives who had cancer at a young age (<50 years old), or a close relative with cancers occurring in both of a pair of organs (eg, both kidneys), or more than 1 childhood cancer in siblings, or breast cancer in a male relative, or cancer occurring in many generations (eg, grandfather, father, son) will also be excluded.

History of abnormal or positive results for routine cancer screening tests (eg, prostate specific antigen [PSA] for men, Papanicolaou [PAP] smear or mammogram for women).

Genetic syndromes that predispose to cancer (eg, BRCA1 and BRCA2, Lynch syndrome, familial polyposis syndromes, Li-Fraumeni syndrome, and multiple endocrine neoplasia syndromes).

Aspartate aminotransferase (AST) and alanine aminotransferase (ALT) exceeding the upper limit of normal (ULN) of the clinical laboratory's reference range at Screening or at admission on Day −2.

Total bilirubin exceeding the ULN by 1.5-fold (ie, 1.5×ULN), to account for slightly elevated bilirubin levels in subjects with Gilbert's syndrome (harmless congenital nonhemolytic low grade hyperbilirubinemia due to UGT1A1 polymorphism).

Hemoglobin, hematocrit, or red blood cell count below the lower limit of normal of the clinical laboratory's reference range at Screening or at admission on Day −2.

Abnormal fasting blood glucose (ie, >125 mg/dL or >6.9 mmol/L; matrix plasma from venous blood sample) and/or hemoglobin A1c (HbA_{1c}) (ie, >6.4% [High performance liquid chromatography] or >42 mmol/mol Hb) at Screening or on Day 2. Blood glucose measurements may be repeated during the Screening period in case of suspect of dietary non-compliance with required overnight fasting period.

Serum creatinine above the ULN of the clinical laboratory's reference range at Screening or at admission on Day −2.

Thyroid stimulating hormone (TSH) levels outside normal limits of the clinical laboratory's reference range at Screening.

Taken any disallowed therapies, Pre-study and Concomitant Therapy, up to 30 days before the planned first dose of study drug.

History of drug or alcohol abuse according to Diagnostic and Statistical Manual of Mental Disorders (5^{th} edition) (DSM-V) criteria within 2 years before Screening or positive test result(s) for alcohol or drugs of abuse (including but not limited to barbiturates, opiates, cocaine, cannabinoids, amphetamines and benzodiazepines) at Screening or at admission on Day −2.

Known allergies, hypersensitivity, or intolerance to any of the excipients of FP2 (refer to IB, Section 2.3 Formulation information).

Donated blood or blood products (approximately 450 mL) or lost a significant amount of blood within 2 months before the first administration of study drug.

Received an investigational drug (including investigational vaccines) or used an invasive investigational medical device within one month or within a period less than 10 times the drug's half-life, whichever is longer, before the planned first dose of study drug.

Pregnant, or breast-feeding, or planning to become pregnant while enrolled in this study or within 12 weeks after the last dose of study drug.

Plans to father a child while enrolled in this study or within 12 weeks after the last dose of study drug.

History of hepatitis B surface antigen (HBsAg) or hepatitis C antibody (anti-HCV) positive, or other clinically active liver disease, or tests positive for HBsAg or antiHCV at Screening.

History of human immunodeficiency virus (HIV) antibody positive, or tests positive for HIV at Screening.

Had major surgery, (eg, requiring general anesthesia) within 6 months before Screening, or will not have fully recovered from surgery, or has surgery planned during the time the subject is expected to participate in the study or within 12 weeks after the last dose of study drug administration. Note: Subjects with planned surgical procedures to be conducted under local anesthesia may participate if approved by the investigator.

Subject smokes cigarettes (or equivalent) and/or has used nicotine based products within 3 months prior to study drug administration or having a positive cotinine test at Screening or at admission on Day −2.

Drinks, on average, more than 1,200 mL (ie, 5 cups, combined total volume) of tea/coffee/cocoa/cola/caffeinated beverages (e.g., energy drink) per day.

Subjects who are vegan or vegetarian, have food allergies or food intolerances.

Psychological and/or emotional problems, which would render the informed consent invalid, or limit the ability of the subject to comply with the study requirements.

Subject is unable or unwilling to undergo multiple venipunctures because of poor tolerability or lack of easy access to veins.

Any condition for which, in the opinion of the Investigator, participation would not be in the best interest of the subject (e.g., compromise well-being) or that could prevent, limit, or confound the protocol-specified assessments.

Employee of the Sponsor, Investigator or study site, with direct involvement in the proposed study or other studies under the direction of that investigator or study site, as well as family members of the employees or the Investigator or the Sponsor.

Subject lives in an institution on court or authority order.

Randomized in a previous dose group of this study.

4.3. Prohibitions and Restrictions

Potential subjects will be willing and able to adhere to the following prohibitions and restrictions during the course of the study to be eligible for participation:

Agree to follow all requirements that will be met during the study as noted in the Inclusion and Exclusion Criteria (eg, contraceptive requirements).

Strenuous exercise may affect study specified assessments and safety laboratory results; for this reason, strenuous exercise (eg, long distance running 5 km/day, weight lifting, or any physical activity to which the subject is not accustomed) is to be avoided starting three (3) days before screening, throughout the study, until completion of the end-of-study visit.

Subjects will be instructed to avoid donating blood for at least 3 months after completion (ie, end-of-study visit) of the study.

Alcohol consumption or alcohol-containing products are not permitted beginning at least 24 hours prior to screening and prior to admission to the CRU on Day −2 until the end of the domiciliation period on Day 5, and at least 24 hours prior to all other outpatient clinic visit. During the remaining days of the study, alcohol consumption should be limited to a maximum amount of 24 grams per day in men (ie, 0.5 L of beer/day or 0.25 L of wine/day or 3 glasses [2 cL per glass] of liquor/day), and 12

grams per day in women (ie, 0.25 L of beer/day or 0.125 L of wine/day or 1.5 glasses [2 cL per glass] of liquor/day for females).

Subjects may not consume any food or beverages containing grapefruit juice, Seville oranges (including any orange marmalade), or quinine (eg, tonic water) from 48 hours before Day 1 until Day 5, the end of the domiciliation period.

Subjects will refrain from the use of any methylxanthine-containing products (eg, chocolate bars or beverages, coffee, teas, colas, or energy drinks) from 48 hours before study drug administration on Day 1 until Day 5. On other days between Screening and the follow-up visit, subjects will be instructed not to drink, on average, more than 1,200 mL of tea/coffee/cocoa/cola (5 cups, combined total volume) per day.

Subjects will be instructed to abstain from poppy seed consumption at least 72 hours prior to admission to the CRU as this can interfere with the drug screen.

Smoking cigarettes (or equivalent) and/or the use of nicotine-based products is not allowed from 3 months prior to the study drug administration until completion of the end-of-study visit.

Subjects will not consume any foods or drinks other than those provided by the study site personnel during the in-clinic phase.

Agree to follow the contraceptive requirements as noted in the inclusion criteria. There is no information about the effect of FP2 on sperm or its production in the body, nor is there information about effects on the development of the fetus. It is important that the subject and subject's partners not become pregnant during the study or for up to 3 months after the study. Subjects should inform the Investigator if their partner becomes pregnant during the study or within the 3 months after study completion (ie, end-of-study visit).

### 5. Study Drug Allocation and Blinding

Part 1

All subjects who remain eligible for study participation will be randomized prior to study drug administration on Day 1. A computer-generated randomization schedule will be provided by the Sponsor and retained at the CRU pharmacy.

Within each DG, subjects will be randomly assigned to active treatment (FP2) or placebo based on a computer-generated randomization schedule prepared before the study by or under the supervision of the Sponsor. The randomization will be balanced by using randomly permuted blocks. Within each DG, a total of 6 subjects will receive FP2 and 2 subjects will receive placebo. For the DG in Part 1 that will be matched with Part 2, 4 females and 4 males will be enrolled, with a randomization 3:1 (3 FP2 and 1 placebo) for each sex group.

For each DG, the subjects will be separated into 4 subgroups (n=up to 2/subgroup) and dosed on different days. On Day 1 in each DG, the first subgroup (sentinel group) of 2 subjects will be randomly assigned to either FP2 or placebo in a 1:1 ratio and will be dosed at approximately the same time on the same day to allow assessment of safety and tolerability out to 72 hours. Any AEs reported/observed in the subjects dosed in the first subgroup that may impact the dosing of the remaining subjects in the DG will be communicated to the Sponsor prior to randomization and dosing of additional subjects. The remaining 6 subjects (1 placebo, 5 FP2) will be randomly assigned to either FP2 or placebo in a 5:1 ratio (5 FP2 and 1 placebo). After the sentinel subjects have been dosed, in each DG, the remaining 6 subjects will be dosed over approximately 3 days (at least 24 hours apart) in groups of 2 (dosing approximately 2 hours apart).

An unblinded pharmacist at the CRU will prepare individual subject study drug doses according to the randomization schedule and will apply a blinded label prior to dispensing and mask the injection syringe to avoid accidental unblinding by color of the solution. The study drug administration will be done by study personnel not involved in any safety assessments of the study. The Investigator will be provided with a sealed randomization code for each subject, containing coded details of the study drug. These sealed codes will be kept together in a limited access area that is accessible 24 hours per day. All randomization codes, whether opened or sealed, will be collected after the end of the subject's participation in the study.

Data that may potentially unblind the study drug assignment (ie, study drug serum concentrations, anti-drug antibodies to FP2 data, study drug preparation/accountability data, treatment allocation) will be handled with special care to ensure that the integrity of the blind is maintained and the potential for bias is minimized. This can include making special provisions, such as segregating the data in question from view by the investigators, clinical team, or others as appropriate until the time of database lock and unblinding. Any site or study team access to PK data will be anonymized (ie, only group level data and/or dummy subject numbers assigned if individual subject data).

Under normal circumstances, the blind should not be broken until all subjects have completed the study and the database is finalized. Otherwise, the blind of an individual study subject should be broken only if specific emergency treatment/course of action would be dictated by knowing the treatment status of the subject. In such cases, the Investigator may in an emergency determine the identity of the study drug by opening the sealed code. It is recommended that the Investigator contact the sponsor or its designee if possible to discuss the particular situation, before breaking the blind. Telephone contact with the sponsor or its designee will be available 24 hours per day, 7 days per week. In the event the blind is broken, the sponsor will be informed as soon as possible. The date, time, and reason for the unblinding will be documented in the source document.

Subjects who have had their study drug assignment unblinded should continue to return for scheduled evaluations.

In general, randomization codes will be disclosed fully only when the study is completed and the clinical database is locked. However, for unblinded DRC review, the randomization codes, and if required, the translation of randomization codes into treatment and placebo groups will be disclosed to those authorized.

Part 2

As Part 2 is open-label with a single-treatment, and all subjects will receive the same IV dose of FP2, no randomization or other special provisions for treatment assignment are required.

Subjects will be selected to match individual subjects from the reference SC dose group of study Part 1 for sex, age (±5 years), and body-weight (±5 kg).

Parts 1 and 2

For both Parts 1 and 2, and for each DG, at least 2 additional reserve subjects will be admitted on Day −2 and will undergo all assessments leading up to dosing to ensure that a full dose group is randomized.

Randomization numbers will be sequentially assigned to the eligible subjects starting with 1001 in Part 1 and 3001 in Part 2. Additional subjects may be enrolled as replacements to ensure that in Part 1, at least 7 subjects per dose group complete at a minimum the 72-hour post-dose study procedures, and in Part 2, that 6 subjects complete study procedures for a time equivalent to at least 2 half-lives of FP2 (determined based on the PK data from previous dose groups in Part 1). Replacement subjects will assume the same treatment of the subjects they are replacing and will be assigned a new randomization number which will be equal to the randomization number of the subject being replaced but the first digit replaced with a ‘2’ in Part 1 and a ‘4’ in Part 2. For example, subject 1004 will be replaced by subject 2004 in Part 1 and subject 3006 will be replaced by subject 4006 in Part 2. In Part 2 all subjects will receive FP2 in an open-label fashion.

### 6. Dosage and Administration

6.1 Study Drug

FP2 is supplied as a sterile solution for injection that will be stored at −40° C. and be protected from light. The solution is of brown-lutescent appearance and has a FP2 concentration of 50 mg/mL in 10 mM sodium phosphate, 8% sucrose, and 0.04% polysorbate 20, at a pH 6.5. FP2 is provided frozen in R2 glass vials with a 1.2 mL fill volume (Table 68).

The formulation buffer used in the FP2 formulation will be supplied for this study to also be used as the placebo formulation and as a diluent in the preparation of the initial FP2 SC doses (DG 1 to DG 4). It is a sterile, clear solution consisting of 10 mM sodium phosphate, 8.0% sucrose and 0.04% polysorbate 20. The formulation buffer will be used to prepare the placebo injections. The formulation buffer is provided frozen in R2 glass vials with a 1.2 mL fill volume.

TABLE 68
Description of Study Drugs

| | |
|---|---|
| Active | FP2 |
| Compound: | |
| Dosage | 50 mg/mL solution for SC injection and IV shortterm |
| Forms | infusion. |
| Diluent: | Formulation buffer containing 10 mM sodium |
| | phosphate, 8.0% sucrose and 0.04% polysorbate |
| | 20. |
| Concen- | For the SC administrations, the FP2 formulation will be |
| trations: | reconstituted with an appropriate volume of formulation |
| | buffer to prepare dilutions of 0.5 mg/mL (DG 1), 5.0 |
| | mg/mL (DG 2 and DG 3), and 10.0 mg/mL (DG 4); the |
| | undiluted 50 mg/mL formulation will also be used (DG 5, |
| | DG 6, DG 7). |
| | For the IV administration, the FP2 formulation will be |
| | reconstituted with an appropriate volume of formulation |
| | buffer to prepare a dilution of 10.0 mg/mL. |
| Placebo | Formulation buffer containing 10 mM sodium |
| (Part 2): | phosphate, 8.0% sucrose and 0.04% polysorbate 20. |

Due to the difference in appearance of the FP2 drug product (brown-lutescent solution) and placebo (clear, colourless solution), the unblinded pharmacist will follow the instructions in the Investigational Medicinal Product (IMP) Handling Manual to maintain the study blind and prevent viewing of the product by study personnel and subjects in Part1.

Detailed instructions for dose preparation, dosing procedures, and storage conditions of the study drug will be provided separately to the study site in an additional guidance document.

Part 1 Dosing:

The study drug will be administered by designated, trained and qualified site personnel who are independent of the study team and are not involved in any other aspect of the conduct of the clinical study. Following an overnight fast of at least 10 hours, study drug (FP2 or placebo) will be administered by using 1.0 mL insulin syringes (DG 1, DG 2, and DG 5) or 2.0 mL syringes (DG 3, DG 4, DG 6, and DG 7) as a single SC dose (maximum volume 2 mL) in the right lower quadrant of the abdomen on Day 1. For each dose group, the subjects will be separated into 4 sub-groups (n=up to 2/subgroup) and dosed on different days. Two sentinel subjects will be dosed first (1 placebo, 1 FP2) on the same day at approximately the same time and will complete a 72-hour safety surveillance period before subsequent subjects in the DG may be dosed. Following review of the blinded safety data, up to 2 additional subjects may be dosed per day in a staggered fashion (approximately 2 hours apart, with one subject dosed at ˜7 am and one subject dosed at ˜9 am) until all subjects in the dose group have completed dosing.

Time zero (0) is the time of study drug injection.

All SC injections will be made to the anterior abdominal wall avoiding the 2-inch area (□5 cm) around the umbilicus. Before any injection, the responsible site personnel will inspect/palpate the planned injection site. Injections should not be made in an area of the abdominal wall assessed as abnormal.

A physician experienced and trained in emergency medicine and emergency equipment (including ready-to-use medications for the treatment of anaphylaxis) will be immediately available in the administration room at all times during the administration of study drug.

Part 2 Dosing:

A single dose of FP2 will be administered as a constant-rate infusion over 30 minutes by using an automatic infusion device (Braun Perfusor® Compact S or equivalent device) via an indwelling catheter into a suitable forearm vein via a separate line using an administration set with a filter. Dosing will be performed at approximately the same time each day but in a staggered fashion (one subject per day at ˜7 am and one subject at ˜9 am). Two sentinel subjects will be dosed first, and the next 2 subjects will be dosed at least 24 hours after the 2 sentinel subjects are dosed, followed by the last 2 subjects to be treated at least 24 hours later. One sentinel subject will be dosed first and will complete a 72-hour safety surveillance period before subsequent subjects may be dosed. The remaining 5 subjects will be subdivided into subgroups (dosed at least 24 hours apart), so that no more than 2 subjects will be dosed per day (approximately 2 hours apart).

Time zero (0) is the start time of the study drug IV infusion.

As no placebo comparison is foreseen for the IV administration of FP2 no measures for blinding of the study drug are required.

A physician experienced and trained in emergency medicine and emergency equipment (including ready-to-use medications for the treatment of anaphylaxis) will be immediately available in the administration room at all times during the administration of study drug.

There might be technical issues, requiring temporary discontinuation of the study drug administration (eg, malfunction of indwelling catheter, and need of placement of a new one). In this case, the infusion may be restarted as soon as possible and the time of discontinuation and the respective reason should be documented.

### 7. Treatment Compliance

Study drug will be administered as a SC injection (Part 1) or as an IV infusion (Part 2) by qualified study-site personnel and the details of each administration will be recorded in the electronic data collection system as applicable [Part 1 SC: injection date, injection time, dose volume injected, injection site; Part 2 IV: start and stop times of the IV infusion and volume infused].

### 8. Prestudy and Concomitant Therapy

Pre-study therapies administered up to 30 days before the first dose of study drug will be recorded. Throughout the study, no therapies (prescription or over-the-counter medications, including vaccines, vitamins, mineral supplements, nutritional supplements, herbal supplements [including St. John's Wort, garlic extract and herbal teas]) are allowed within 30 days before the planned first dose of study drug and during the study, except for paracetamol. If it becomes necessary for a subject to receive prescription or nonprescription medications during the study, a subject may be enrolled or continue in the study with agreement and approval by the sponsor (or designee) and the Principal Investigator.

If the administration of any concomitant therapy becomes necessary, it will be reported in the appropriate section of the electronic case report form (eCRF). Recorded information will include a description of the drug, treatment duration, dosing regimen, route of administration, and its indication.

The use of paracetamol is allowed until 3 days before study drug administration. Throughout the study, a maximum of three 500 mg doses of paracetamol per day, and no more than 3 grams per week, will be allowed for the treatment of headache or other pain.

Concomitant therapies will be recorded throughout the study beginning with start of the first dose of study drug to the end-of-study visit. Concomitant therapies should also be recorded beyond the end-of-study visit only in conjunction with new or worsening adverse events and serious adverse events that meet the criteria.

### 9. Study Evaluations

9.1. Study Procedures

9.1.1. Overview

The Time and Events Schedule summarizes the frequency and timing of PK, immunogenicity, PD, exploratory biomarker, pharmacogenomic, and safety measurements applicable to this study.

Timing of the meals and of the VAS questionnaires are specified in the Meals and VAS Questionnaires Time and Events Schedule.

If multiple assessments are scheduled for the same timepoint, and/or if one or more assessments are scheduled for the same time as a meal, it is recommended that procedures be performed in the following sequence: vital signs, ECG, PK, blood draw, VAS questionnaires for appetite ratings, meal, and VAS questionnaires for food palatability (after the first bite of food). Blood collections for PK assessments should be kept as close to the specified time as possible. When an ECG is to be performed at the same timepoint as PK, the PK specimen should be taken immediately after completion of the ECG. Other measurements may be done earlier than specified timepoints if needed. The order of multiple assessments at the same timepoint should be the same throughout the study. Actual dates and times of assessments will be recorded in the source documentation and eCRF.

Vital signs (ie, blood pressure [BP], heart rate [HR]) should be recorded from the opposite arm from which the blood samples are being taken when feasible (except during the time of IV infusion).

Pregnancy testing will be performed in all females at Screening and throughout the study. A serum pregnancy test be performed at Screening and urine pregnancy test will be obtained at all other timepoints in the Time and Events Schedule.

TABLE 69
Volume of Blood to be Collected From Each
Subject (Part 1 and Part 2 of the study)

| | Approximate | No. of | Approximate |
|---|---|---|---|
| | Volume per | Samples | Total Volume |
| | Sample | per | of Blood |
| Type of Sample | (mL) | Subject | (mL)^{a} |
| Safety laboratories | | | |
| Hematology | 3 | 14 | 42 |
| Clinical chemistry | 5 | 14 | 70 |
| Coagulation | 3 | 14 | 42 |
| Lipid panel | 3 | 5 | 15 |
| Serum chemistry^{b} | | | |
| Serology (HIV, hepatitis) | 5 | 1 | 5 |
| Serum β-hCG pregnancy test^{c} | 5 | 1 | 5 |
| TSH | 3 | 1 | 3 |
| FSH^{c} | 3 | 1 | 3 |
| HbAl_{c} | 3 | 1 | 3 |
| Pharmacokinetic samples | 3 | 20 | 60 |
| Immunogenicity | 7.5 | 4 | 30 |
| Exploratory Biomarker | 3 | 9 | 27 |
| samples | | | |
| Pharmacogenomic sample^{d} | 10 | 1 | 10 |
| Approximate Total^{e} | | | 315 |

^{a}Calculated as number of samples multiplied by amount of blood per sample.
^{b}Serum chemistry includes serology (HBsAg, anti-HCV antibody, HIV 1 and 2 antibodies) and serum β-hCG pregnancy tests.
^{c}Only in female subjects.
^{d}A blood sample will be collected only from subjects who have consented to provide an optional DNA sample for research.
^{e}Repeat or unscheduled samples may be taken for safety reasons or technical issues with the samples.
Note:
An indwelling intravenous cannula may be used for blood sample collection. [If a mandarin (obturator) is used, blood loss due to discard is not expected.]

These volumes may be adjusted in the final laboratory manuals as long as the maximum amount of blood drawn from each subject in this study does not exceed 500 mL (allowing for changes in blood collection tube size or availability). Additional blood samples may be collected, if necessary, for additional safety, immunogenicity or PK assessments based on emerging data, but the total blood volume collected from an individual subject during this study will not exceed the amount stated in this study protocol without prior Independent Ethics Committee (IEC) and health authority approvals. Repeat or unscheduled samples may be taken for safety reasons or for technical issues with the samples do not require prior IEC and health authority approvals.

For each subject, the maximum amount of blood drawn in this study will not exceed 500 mL. The total blood volume to be collected from each subject will be approximately 315 mL.

9.1.2. Screening Period (Parts 1 and 2)

Potential subjects will be seen for a Screening visit within 28 days prior to study drug administration on Day 1 to determine subjects' eligibility for study participation. If the subject meets the criteria for enrollment, he/she will be admitted to the CRU on Day −2.

Prior to conducting any study procedure, the PI (or designated study personnel) will review and explain the written ICF to each subject. No study procedures (including fasting for study lab testing) can be performed until after the subject signs the ICF. For Screening visits, all subject reported assessments should be conducted before any tests, procedures, or consultations to discontinue subjects who do not meet any of these entry criteria.

The Investigator (or designated study personnel) will also review and explain the written ICF for optional genetic research samples prior to pharmacogenomic blood sampling.

All adverse events, whether serious or non-serious, will be reported from the time a signed and dated informed consent form is obtained until the final study procedure at the final End-of Study Visit, and also by direct questioning at specific time points. (See the Time & Events Schedule.)

Retesting of abnormal values that could lead to exclusion will be allowed only once. Retesting might take place during an unscheduled visit. If any Screening tests are repeated, test results will meet eligibility requirements and will be available for investigator review prior to admission to the CRU (eg, Day −2).

9.1.3. Inpatient Treatment Period (Parts 1 and 2)

Days −2 and −1 (Baseline)

Qualified subjects will be admitted to the CRU on Day −2 and will undergo baseline safety assessments (Day −2) and baseline ECG collection (time-matched to the Day 1 ECGs) on Day −1 as specified in the Times and Events Schedule. On Day −1 subjects will also undergo a 24-hour food intake measurement and will complete VAS questionnaires to assess appetite ratings and food palatability.

Day 1/ Randomization and Dosing

After confirmation that all enrollment criteria are met, eligible subjects will be randomized on Day 1 just prior to study drug dosing.

The study drug will be administered under the supervision of the PI or his/her designee. Refer to the Time and Events Schedule for study procedure details and timepoints.

For each dose level in Part 1 and in Part 2), the subjects will be separated into subgroups and dosed on different days, so that no more than 2 subjects will be dosed per day. Two sentinel subjects in Part 1 (1 placebo, 1 FP2) and 1 sentinel subject in Part 2 (FP2) will be dosed first) and will complete a 72-hour safety surveillance period before subsequent subjects in the dose group may be dosed. Following review of the safety data, up to 2 additional subjects will be dosed in a staggered fashion (approximately 2 hours apart) per day until all subjects have completed dosing.

Days 3 Through Day 5

On Day 3, measurement of the 24-hour food intake and administration of VAS questionnaires will be repeated.

Subjects will remain domiciled continuously in the CRU for safety, tolerability, PK and PD assessments until the morning of Day 5, when upon completing study evaluations, they may be discharged.

9.1.4. Outpatient Period (Parts 1 and 2)

Subjects will return to the CRU fasted (at least 10 hours) for safety, tolerability, PK, PD, and immunogenicity assessments as detailed in the Time and Events Schedule. Completion of the End-of-study visit constitutes a subject's end of participation in the study. All reasonable attempts should be made to conduct outpatient visits at the scheduled timepoints (ie, specific day for each visit) but a window within ±1 day is allowed up to the Week 4 (Day 28) visit and a window of ±3 days is allowed for the remaining outpatient visits out to the end-of-study visit. All subsequent visits should be scheduled relative to the date of the first study drug dose (Day 1) and not the date of the previous rescheduled visit.

Early Withdrawal

If a subject withdraws from the study for any reason before the end of the outpatient period, then the End-of-Study assessments should be obtained.

9.2. Pharmacokinetics and Immunogenicity

Venous blood samples will be collected over time as specified in the Time and Events Schedules. The PK sampling times may be adjusted based on preliminary PK data from prior dose groups (eg, late sampling times might be omitted if FP2 serum concentrations are below lower limit of quantification[(LLOQ]). The actual date and time of each PK and immunogenicity blood sample (ADA) collection will be recorded on the eCRF in the electronic data collection system. Subjects who terminate study participation prematurely should have final assessment samples collected at the time of termination.

9.2.1. Evaluations

Samples collected for analyses of FP2 serum concentration and antibody to FP2 may additionally be used to evaluate safety or efficacy aspects that address concerns arising during or after the study period, for further characterization of immunogenicity or for the evaluation of relevant biomarkers. Subject confidentiality will be maintained.

9.2.2. Analytical Procedures

Pharmacokinetics

Serum samples will be analyzed to determine concentrations of FP2 using a validated, specific, and sensitive immunoassay method by or under the supervision of the sponsor.

Immunogenicity

The detection and characterization of anti-FP2 antibodies and potential antibody towards endogenous GDF15 in serum will be performed using a validated assay method by or under the supervision of the sponsor. All samples collected for detection of ADAs will be also evaluated for FP2 serum concentration to enable interpretation of the antibody data.

9.2.3. Pharmacokinetic Parameters

Pharmacokinetic parameters of FP2 will be calculated from the serum concentration—time profiles using non-compartmental analyses. Pharmacokinetic parameters following a single administration of FP2 will include, but are not limited to the following:

Cₘₐₓ: Maximum observed serum concentration.

Tₘₐₓ: Time to reach maximum observed serum concentration.

AUC_{inf}: Area under the serum concentration versus time curve from time zero to infinity with extrapolation of the terminal phase.

AUCₗₐₛₜ: Area under the serum concentration versus time curve from time zero to the time corresponding to the last quantifiable concentration.

T_{1/2}: Terminal disposition half-life.

CL: Total systemic clearance (IV only).

CL/F: Apparent total systemic clearance after extravascular administration (SC only).

V_{z}: Volume of distribution based on terminal phase (IV only).

V_{z}/F: Apparent volume of distribution based on terminal phase after extravascular administration (SC only).

F (%): Absolute SC bioavailability to be calculated as the fraction of the administered dose available systemically, expressed as a percentage. Absolute bioavailability is calculated using the following equation.

(%)=______ ______, ××%,

9.2.4. Immunogenicity Assessments

Anti-FP2 antibodies will be evaluated in blood samples collected from all subjects according to the Time and Events Schedule. Additionally, blood samples should also be collected at the end-of-study visit from subjects who are withdrawn from the study. These samples will be tested by the Sponsor or Sponsor's designee.

Blood samples will be screened for antibodies binding to FP2 and the titer of confirmed positive samples will be reported. Other analyses may be performed to further characterize the immunogenicity of FP2.

9.3. Pharmacodynamic Evaluations

Body weight will be measured in duplicate in the morning, prior to breakfast and after voiding as detailed in the Time and Events Schedule. Subjects will be weighed on a calibrated scale, while wearing a gown or light indoor clothing without shoes.

Twenty-four-hour food intake will be assessed on Day −1 and Day 3 by providing 4 meals (breakfast, lunch, snack and dinner) that will be served at the same time on both days. Meals will be standardized between days (identical meals on Day −1 and Day 3). Meals will also be standardized across subjects such that the composition and portion size will be the same for all subjects and will be based on local preferences and guidelines as assessed by the study dietitians. Each item of each meal will be weighed before and after consumption and the grams consumed of each food item will be calculated and recorded. The calories consumed will be estimated based on the grams consumed of each food item, and its nutritional content. The change from Day-1 to Day 3 in 24-hour calories intake will be evaluated. For the days when food intake is not recorded (Day −2, 1, 2, 4, and 5), every meal (including breakfast and snack) should be different than the meals provided on Days −1 and 3.

VAS questionnaires to evaluate appetite ratings (hunger, thirst, nausea and fullness) will be administered hourly during waking hours on Day −1 and Day 3, every 3 hours during waking hours on Days 1, 2, 4 and 5, and right before and at the end of each meal (Days −1 to Day 5). VAS questionnaires to evaluate food palatability (which may indicate possible food aversions) will include questions regarding the pleasantness of the food odor, taste and texture of the meal, and will be completed by the subjects immediately after they have eaten the first bite of the main entrée of each meal (Days −1 to Day 5). For a detailed description of the timepoints at which VAS questionnaires will be administered refer to Time and Event Schedule for Food Intake and VAS Questionnaires.

9.6. Safety Evaluations

9.6.2. Clinical Laboratory Tests

Fasting blood samples for chemistry, hematology, coagulation, lipids and urine sample for urinalysis will be collected. Subjects will fast overnight at least 10 hours prior to all lab sample collections. For times of clinical laboratory assessments refer to the Time and Event schedule. The investigator will review the laboratory results, document this review, and record any clinically relevant changes occurring during the study in the adverse event section of the eCRF. The laboratory reports will be filed with the source documents.

The following tests will be performed by the local laboratory:

Hematology Panel

| | |
|---|---|
| hemoglobin | platelet count |
| hematocrit | percent reticulocytes |

red blood cell (RBC) count
white blood cell (WBC) count with differential
Note: A WBC evaluation may include any abnormal cells, which will then be
reported by the laboratory. A RBC evaluation may include abnormalities in the RBC count, RBC
parameters, or RBC morphology, which will then be reported by the laboratory. In addition, any
other abnormal cells in a blood smear will also be reported.
Coagulation Panel
activated partial thromboplatin time (aPTT) -prothrombin time (PT)
Chemistry Panel

| | |
|---|---|
| sodium | alkaline phosphatase |
| potassium | creatine phosphokinase (CPK) |
| chloride | lactic acid dehydrogenase (LDH) |
| bicarbonate | uric acid |
| blood urea nitrogen (BUN) | calcium |
| creatinine | phosphate |
| glucose | albumin |
| aspartate aminotransferase (AST) | total protein |
| alanine aminotransferase (ALT) | magnesium |
| gamma-glutamyltransferase (GGT) | |
| total bilirubin | |
| direct bilirubin* | |

*Only if the total bilirubin is elevated
Lipid Panel

| | |
|---|---|
| total cholesterol | high density lipoprotein (HDL)-cholesterol |
| triglycerides | low density lipoprotein (LDL)-cholesterol |

Urinalysis

| | |
|---|---|
| Dipstick | Sediment (if dipstick result is |
| specific gravity | abnormal) |
| pH | red blood cells |
| glucose | white blood cells |
| protein | epithelial cells |
| blood | crystals |
| ketones | casts |
| bilirubin | bacteria |
| urobilinogen | |
| nitrite | |
| leukocyte esterase | |

If dipstick result is abnormal, microscopy will be used to measure sediment.

In the microscopic examination, observations other than the presence of WBC, RBC and casts may also be reported by the laboratory.

Other Lab Testing

The following lab testing will be performed at timepoints indicated in the Time and Events Schedules:

Serum and Urine Pregnancy Testing (p-hCG) will be conducted for all females

FSH (females only), TSH and HbA1c

Serology (HIV 1 and 2 antibodies, HBsAg, and anti-HCV antibody

Urine Drug (amphetamine, barbiturates, benzodiazepines, cannabinoids, cocaine, opiates, methadone) and Cotinine Screen

Alcohol breath test

9.6.3. Electrocardiogram

Standard 12-lead ECGs will be collected as specified in the Time and Events Schedule. Each ECG will be printed and stored in the Subject's Medical File at the study site. In addition, ECG data will be transferred electronically to a specialized ECG laboratory (Nabios GmbH, Munich) for centralized analysis according to ICH E14 recommendations. Details of the ECG analysis methodology, the timing of readings and delivery and format of results for dose-escalation meetings will be detailed in a separate ECG Manual.

The 12-lead ECGs will be captured in triplicate less than 2 minutes apart at each time point after subjects have been resting quietly in a supine position for at least 5 minutes, and refraining from talking or moving arms or legs. During the collection of ECGs, subjects should be in a quiet setting without distractions (eg, television, cell phones). If multiple assessments are scheduled for the same timepoint, and/or if one or more assessments are scheduled for the same time as a meal, it is recommended that procedures be performed in the following sequence: vital signs, ECG, PK, blood draw, VAS questionnaires for appetite ratings, meal, and VAS questionnaires for food palatability (after the first bite of food). Blood collections for PK assessments should be kept as close to the specified time as possible. When an ECG is to be performed at the same timepoint as PK, the PK specimen should be taken immediately after completion of the ECG. Other measurements may be done earlier than specified timepoints if needed. The order of multiple assessments at the same timepoint should be the same throughout the study.

ECGs will be done in triplicate at each scheduled time point for more precise QTc interval change assessments. ECGs obtained on Day −1 should be time matched to the ECGs obtained on Day 1. At each time point at which triplicate ECGs are required, 3 individual ECG tracings should be obtained as closely as possible in succession, but no more than 2 minutes apart. The full set of triplicates should be completed in less than 4 minutes.

The average of the triplicate measurements at each time point on Day −1 will serve as each subject's time-matched baseline value for the corresponding parameters on Day 1.

Apart from the off-site central reading process of the ECGs, each ECG should be review after collection by a qualified study site physician for findings of possible clinical safety relevance (eg, QTc interval prolongation). Any pathological findings and respective medical interventions (if any) will be documented in the eCRF.

9.6.4. Continuous Lead II ECG Monitoring (Part 2 Only)

Continuous Lead II ECG monitoring will only be conducted in Part 2 on Day 1 from 30 minutes prior to beginning the IV infusion until 2 hours after completion of the infusion. At the discretion of the investigator (or designee) continuous lead II ECG monitoring may be extended. These data are for real time visual monitoring and any abnormality detected by the ECG monitoring device or by the investigator will be printed out and retained as source data. An unscheduled 12lead ECG measurement should also be performed as soon as possible after an abnormality is identified. Any clinically significant abnormalities will be recorded as adverse events.

9.6.5. Vital Signs

Blood pressure and HR measurements will be assessed in the supine position with a completely automated oscillometric device. Manual techniques will be used only if an automated device is not available. Vital signs should be measured using the opposite arm from which blood samples are being collected (except during the time of IV infusion).

Blood pressure and HR measurements should be preceded by at least 5 minutes of rest in a quiet setting without distractions (eg, television, cell phones). At all timepoints, single BP and HR measurements will be performed and recorded.

The same method for the body temperature measurements (ie, tympanic) will be used for all measurements for all subjects throughout the study duration.

The timepoints of these examinations are specified in the Time and Events Schedule.

9.6.6. Physical Examination

A complete physical examination comprises a routine medical examination including: general appearance, neurological, eyes, ear/nose/throat, thyroid, cardiovascular, respiratory, abdominal/gastrointestinal, hepatic, musculoskeletal, and dermatological.

A brief physical examination includes evaluation of skin, respiratory system, CV system, abdomen (liver, spleen) and CNS.

The timepoints of these examinations are specified in the Time and Events Schedule. A new, clinically significant finding (in the opinion of the investigator) not noted at Screening will be captured as an AE.

9.6.7. Allergic Reactions/General Hypersensitivities

All subjects will be observed and carefully monitored for the development of any allergic reaction during the study and for any infusion reaction during IV study drug administration in Part 2.

A physician will be immediately available at the site at all times during the administration of study drug.

All subjects will be observed carefully for symptoms of an infusion reaction during and after the IV administration as indicated in the Time and Events Schedule. The investigator should use clinical judgment in assessing the intensity of any infusion reactions.

If an infusion reaction is observed, treatment such as oral paracetamol and/or oral/IV antihistamine and/or inhaled ß-agonists and/or IV corticosteroids and/or IV epinephrine may be administered, depending on the nature of the allergic reaction and the severity of symptoms. The following precautions should be applied during the IV administration of study drug in Part 2:

Before an infusion is started, the appropriate personnel, medications (eg, epinephrine, inhaled β-agonists, antihistamines, and corticosteroids), and other requirements such as infusion solutions to treat allergic reactions, including anaphylaxis, will be available.

If a subject has a moderate or severe infusion reaction, the infusion should be terminated immediately and the subject should be treated appropriately according to institutional guidelines.

Subjects discontinuing the study drug due to an infusion reaction will be asked to return for required assessments at all scheduled visits through the End-of-Study visit.

Reactions following study drug administration may occur 1 to 21 days after an infusion or injection and presentation can be variable in signs and symptoms, and not always apparent (including but not limited to myalgia and/or arthralgia with fever and/or rash [that does not represent signs and symptoms of other recognized clinical syndromes], and may be accompanied by other symptoms including pruritus, facial, hand, or lip edema, dysphagia, urticaria, sore throat, and/or headache). Any allergic reaction or hypersensitivity should be recorded as an AE and the type of reaction should be indicated. In the event that a subject experiences a delayed infusion or injection reaction, the subject will be asked to provide additional unscheduled samples (urine, serum, and plasma for inflammatory markers). Every attempt should be made to collect these samples as close as possible to the onset of the adverse event(s) suggestive of a delayed hypersensitivity reaction. These samples will be used to try and understand the etiology of the symptoms. In case a skin reaction (ie, rash) has been observed, the subject will be asked permission to obtain skin biopsies, again to be used to better understand the cause of the rash.

9.6.8. Local Injection Site Reaction (Part 1)

The injection site after SC study drug administration will be evaluated for local injection site reaction at the time points indicated in the Time and Events Schedule.

Any adverse reaction (eg, pain, erythema, and/or induration) should be documented on and should be characterized as described in “Toxicity Grading Scale for Healthy Adult and Adolescent Volunteers Enrolled in Preventive Vaccine Clinical Trials⁷ based on the following 4 parameters: 1) degree of pain, 2) tenderness, 3) erythema/redness, 4) induration/swelling. See details in Table 70 below.

TABLE 70
Toxicity Grading Scale for Infusion and Local Injection Site Reactions

| | | | | Potentially Life |
|---|---|---|---|---|
| Local Reaction to | Mild | Moderate | Severe | Threatening |
| Injectable Product | (Grade 1) | (Grade 2) | (Grade 3) | (Grade 4) |
| Pain | Does not | Repeated use of | Any use of | ER visit or |
| | interfere with | nonnarcotic pain | narcotic pain | hospitalization |
| | activity | reliever >24 hours or | reliever or | |
| | | interferes with | prevents daily | |
| | | activity | activity | |
| Tenderness | Mild | Discomfort with | Significant | ER visit or |
| | discomfort to | movement | discomfort at | hospitalization |
| | touch | | rest | |
| Erythema/Rednessa | 2.5-5 cm | 5.1-10 cm | >10 cm | Necrosis or |
| | | | | exfoliative |
| | | | | dermatitis |
| Induration/Swelling^{b} | 2.5-5 cm and | 5.1-10 cm or | >10 cm or | Necrosis |
| | does not | interferes with | prevents daily | |
| | interfere with | activity | activity | |
| | activity | | | |

ER = emergency room
^{a}In addition to grading the measured local reaction at the greatest single diameter, the measurement should be recorded as a continuous variable.
^{b}Induration/Swelling should be evaluated and graded using the functional scale as well as the actual measurement.

All local injection site reactions captured as AEs with visual findings (eg, redness, induration, swelling,) shall be photographed along with a metric ruler for later assessment.

9.7. Sample Collection and Handling

The actual dates and times of sample collection will be recorded in the eCRF. If blood samples are collected via an indwelling cannula, an appropriate amount (1 mL) of serosanguineous fluid slightly greater than the dead space volume of the lock will be removed from the cannula and discarded before each blood sample is taken. After blood sample collection, the cannula will be flushed with 0.9% sodium chloride, and charged with a volume equal to the dead space volume of the lock. If a mandarin (obturator) is used, blood loss due to discard is not expected. Flushing with Heparin is not allowed.

Refer to the Time and Events Schedule for the timing and frequency of all sample collections.

Instructions for the collection, handling, storage, and shipment of samples are found in the laboratory manual that will be provided. Collection, handling, storage, and shipment of samples will be under the specified, and where applicable, controlled temperature conditions as indicated in the laboratory manual.

10. Subject Completion/Discontinuation of Study Treatment/Withdrawal from the Study

10.1. Completion

A subject will be considered to have completed the study if he or she has completed assessments the end-of-study visit.

10.2. Discontinuation of Study Treatment/Withdrawal from the Study

A subject will be automatically withdrawn from the study for any of the following reasons:

Lost to follow-up

Withdrawal of consent

Death

If a subject withdraws from the study for any reason before the end of the Outpatient Period, then the End-of-Study assessments should be obtained. If the subject is willing to return for any visits up to the End-of-Study Visit, any additional information will be collected and any procedures will be performed as allowed.

If a subject is lost to follow-up, every reasonable effort will be made by the study site personnel to contact the subject and determine the reason for discontinuation/withdrawal. The measures taken to follow-up will be documented.

When a subject withdraws before completing the study, the reason for withdrawal is to be documented in the eCRF and in the source document. Study drug assigned to the withdrawn subject may not be assigned to another subject. Subjects who withdraw may be replaced.

If the PI or Sponsor believes (eg, for safety reasons or tolerability reasons), it is in the best interest of the subject to discontinue further study participation this is permitted, but the subject should still complete the study assessments through the End-of-Study visit.

### 11. Statistical Methods

Statistical analysis will be done by the sponsor or under the authority of the sponsor.

No formal hypothesis testing is planned due to the exploratory and descriptive nature of the study.

11.1. Subject Information

All subjects who receive at least a partial dose of study drug will be included in the descriptive statistics and analyses of safety.

11.2. Sample Size Determination

There are no human PK data available for FP2, therefore, for this initial FIH Phase 1 study, no formal sample size calculation was performed. The number of subjects planned for each dose group is consistent with customary sample sizes in single ascending dose Phase 1 studies of new drugs where the primary objective of the study is safety and tolerability and one of the secondary objectives is to assess the PK profile. In each dose group, 6 subjects will be randomized to FP2 and 2 subjects will be randomized to placebo; thus, the active study drug to placebo ratio will be 3:1 within each dose group. The number of subjects planned is expected to provide sufficient information on the safety, tolerability, and pharmacokinetics of FP2 to permit evaluation of the potential for further clinical development. Two subjects receiving placebo per dose group should be sufficient to allow judgment of safety and tolerability at each dose level, and, if appropriate, placebo subjects will be pooled at the completion of the study for data analyses purposes. If it is not appropriate to pool placebo subjects due to issues such as heterogeneous variance, then alternative statistical methods may be explored (log transformation, etc.) so that all of the data may be used in the analysis.

The sample size of 6 subjects treated with FP2 per dose level is sufficient for estimating the probability of a safety signal (eg, hypersensitivity). Assuming a true risk of a safety signal of ˜10%, 6 subjects receiving active drug allows for detecting at least one event with a probability of ˜47%; while assuming a risk of ˜50%, the probability of detecting at least one event is approximately 98%.

For pharmacokinetic parameters, 6 subjects (3 male; 3 female) receiving IV administrations of FP2 in Part 2 is expected to provide adequate precision for the estimation of absolute bioavailability. The sample size of 6 subjects is expected to provide adequate precision for estimating FP2 AUC upon IV administration and to serve as a benchmark for the assessment of pharmaceutical quality attributes of SC dosage form.

11.3. Pharmacokinetic Analyses

All subjects who receive at least one dose of study drug and have at least one PK and immunogenicity sample collected post-dose will be included in the analyses and reporting of PK data. Subjects will be excluded from the PK analysis if their data do not allow for accurate assessment of the PK (eg, incomplete administration of the study drug; missing information of dosing and sampling times; concentration data not sufficient for PK parameter calculation).

Descriptive statistics (means, median, standard deviations and coefficients of variation) will be used to summarize FP2 serum concentrations at each sampling timepoint and also for the FP2 PK parameters for each DG. All serum concentrations below the lowest quantifiable concentration (BLQ) will be imputed as zero in the summary statistics, and all subjects and samples excluded from the PK analyses will be clearly documented. PK parameters will also be summarized by DG.

Mean and or median serum FP2 concentration time profiles will be plotted over the complete profile after study drug administration. For the mean plots, BLQ values will be set to zero. For individual serum concentration time profiles, BLQ values up to the first measured concentration will be set to zero and after the last measured concentration will be eliminated from the analysis.

Statistical analysis of the PK data will be performed for all subjects receiving at least one FP2 dose. Geometric mean Cₘₐₓ and AUC will be plotted vs. dose to visually assess dose proportionality. Dose-proportionality upon SC administration of FP2 may be examined using linear regression analyses of natural log transformed Cₘₐₓ and AUC data (power model). Additional analyses of the data may be performed as necessary.

Absolute SC bioavailability will be calculated as the fraction of the administered dose available systemically, expressed as a percentage. To examine individual bioavailability, the ratio of individual dose-normalized AUC_{inf} from SC administration vs the geometric mean of dosenormalized AUC_{inf} from the IV administration group will be computed and expressed as a percentage. Partial AUC_{(0-4 weeks)} may be used instead of AUC_{inf}, if deemed appropriate. Absolute SC bioavailability of FP2 will be derived by computing the average of the individual absolute SC bioavailability described above. Ninety-five percent confidence intervals and plots of the individual absolute SC bioavailability may also be explored.

11.4. Immunogenicity Analyses

The incidence of anti-FP2 antibodies will be summarized for all subjects who receive at least 1 dose of FP2 and have appropriate samples for detection of antibodies to FP2 (ie, subjects with at least 1 sample obtained after FP2 dosing).

A listing of subjects who are positive for antibodies to FP2 will be provided. The maximum titers of antibodies to FP2 will be also reported for subjects who are positive for antibodies to FP2.

The incidence of neutralizing antibodies (NAbs) to FP2 will be summarized for subjects who are positive for antibodies to FP2 and have samples evaluable for NAbs to FP2.

11.5. Pharmacodynamic Analyses (Body Weight, Food Intake) and Exploratory

PD Assessments (VAS) and Biomarkers

Pharmacodynamic analyses will be performed on all subjects receiving at least 1 dose of study drug (FP2 or placebo) and with at least 1 PD assessment post-treatment. For each dose, descriptive statistics will be calculated at each time point for each PD endpoint (eg, weight, food intake) and exploratory PD outcomes (VAS) and biomarker parameter. Parameters may be displayed graphically 1) for each subject and 2) as mean values+/−standard deviation (or other appropriate summary measures) by dose vs. planned sampling time for each endpoint for visual assessment of dose related effects. Additional descriptive statistical analyses may include change and percent change from baseline (ie, pre-dose) values of select pharmacodynamic and exploratory biomarkers.

Pharmacodynamic and exploratory biomarkers may be analyzed using a mixed-effect model appropriate for a single ascending dose, sequential panel design. The mixed-effects model may include fixed factors for treatment, visit, treatment by visit interaction, baseline, and baseline by visit interaction, with a random factor for subject. Baseline covariates may also be included in the model (eg, age, gender, weight, etc.). If appropriate, differences (treatment−pooled placebo) in least squares mean change from baseline and 90% confidence intervals for the difference in means may be obtained using the mean squared error from the linear model and referencing a t-distribution. Least squares means and 90% confidence intervals for the PD/biomarker endpoints change from baseline (pre-dose) may also be calculated by treatment group.

Additional exploratory analyses may be performed as necessary. PD parameters such as VAS may be summarized over time within a visit using area under the curve (trapezoidal method) or time weighted average prior to analysis in the linear mixed-effects model described above.

11.6. Pharmacokinetic/Pharmacodynamic Analyses

The relationships between PK and PD data may be explored. Where appropriate, serum drug concentrations and corresponding PD measurements may be plotted to evaluate their relationship. If deemed appropriate, a suitable model may be applied to describe the exposure effect relationship.

The PK/PD relationship may be investigated graphically and if deemed appropriate may be further analyzed using suitable statistical method. PK exposures (Cₘₐₓ and/or AUC_{inf}) versus PD variables (eg, weight, food intake, VAS) may be graphically examined. If the graphical representation of the relationship is deemed reasonable, then these data may be analyzed statistically using a suitable model.

PK exposures (Cₘₐₓ and/or AUC_{inf}) versus safety variables (eg, ECG response) may be graphically explored. If the graphical representation of the relationship is deemed reasonable, then these data may be analyzed statistically using a suitable model.

11.7. Safety Analyses

The reporting of the safety data of all subjects receiving at least 1 dose of FP2 or placebo will include the incidence and type of adverse events, along with absolute values and changes in: blood pressure, heart rate, clinical laboratory data, and 12-lead ECG data from predose to the final post-dose timepoint.

Adverse Events

The verbatim terms used in the eCRF by investigators to identify adverse events will be coded using the Medical Dictionary for Regulatory Activities (MedDRA). Study drug-emergent adverse events are adverse events with onset during the intervention phase or that are a consequence of a pre-existing condition that has worsened since baseline. All reported adverse events will be included in the analysis. For each adverse event, the percentage of subjects who experience at least 1 occurrence of the given event will be summarized by intervention group. In addition, comparisons between intervention groups will be provided if appropriate.

Summaries, listings, datasets, or subject narratives may be provided, as appropriate, for those subjects who die, who discontinue study drug due to an adverse event, or who experience a severe or a serious adverse event.

Clinical Laboratory Tests

Laboratory data will be summarized by type of laboratory test. Reference ranges and markedly abnormal results (specified in the Statistical Analysis Plan) will be used in the summary of laboratory data. Descriptive statistics will be calculated for each laboratory analyte at baseline and for observed values and changes from baseline at each scheduled time point. Changes from baseline results will be presented in pre- versus post-intervention cross-tabulations (with classes for below, within, and above normal ranges). A listing of subjects with any laboratory results outside the reference ranges will be provided. A listing of subjects with any markedly abnormal laboratory results will also be provided.

Electrocardiogram (ECG)

The ECG variables that will be analyzed are HR, PR interval, QRS interval, QT interval, and corrected QT (QTc) interval using the following correction methods: QT corrected according to Bazett's formula (QTcB), QT corrected according to Fridericia's formula (QTcF).^{1,19,14,16}

Descriptive statistics of QTc intervals and changes from baseline will be summarized at each scheduled time point. The percentage of subjects with QTc interval>450 ms, >480 ms, or >500 ms will be summarized, as will the percentage of subjects with QTc interval increases from baseline>30 ms or >60 ms.

All clinically relevant abnormalities in ECG waveform that are changes from the baseline readings will be reported (eg, changes in T-wave morphology or the occurrence of—Uwaves—).

The relationship between JNJ-64379090 serum concentrations and the primary endpoint ΔΔQTc (placebo-corrected change from time-matched baseline QTc) may be quantified using a linear or nonlinear mixed-effects modeling approach, if applicable.

Vital Signs

Descriptive statistics of HR, and blood pressure (systolic and diastolic) (supine) values and changes from baseline will be summarized at each scheduled time point. The percentage of subjects with values beyond clinically important limits will be summarized.

Physical Examination

Descriptive statistics of changes from baseline will be summarized at each scheduled time point.

11.8. Interim Analysis/Data Review Committee

No interim analysis is planned. However, a blinded data review may be performed by the study team after each dose group is complete for the purpose of making a dose-escalation decision.

### 12. Adverse Event Reporting

Timely, accurate, and complete reporting and analysis of safety information from clinical studies are crucial for the protection of subjects, investigators, and the sponsor, and are mandated by regulatory agencies worldwide. The sponsor has established Standard Operating Procedures in conformity with regulatory requirements worldwide to ensure appropriate reporting of safety information; all clinical studies conducted by the sponsor or its affiliates will be conducted in accordance with those procedures.

Method of Detecting Adverse Events and Serious Adverse Events

Care will be taken not to introduce bias when detecting AEs or SAEs. Open-ended and nonleading verbal questioning of the subject is the preferred method to inquire about adverse event occurrence.

Solicited Adverse Events

Solicited AEs are predefined local and systemic events for which the subject is specifically questioned. In this study, local injection site reactions (such as pain or itching) will be solicited as specified in the Time and Events Schedule.

Unsolicited Adverse Events

Unsolicited AEs are all adverse events reported spontaneously (ie, for which the subject is not specifically questioned).

12.1. Definitions

12.1.1. Adverse Event Definitions and Classifications

Adverse Event

An adverse event is any untoward medical occurrence in a clinical study subject administered a medicinal (investigational or non-investigational) product. An adverse event does not necessarily have a causal relationship with the intervention. An adverse event can therefore be any unfavorable and unintended sign (including an abnormal finding), symptom, or disease temporally associated with the use of a medicinal (investigational or non-investigational) product, whether or not related to that medicinal (investigational or non-investigational) product. (Definition per International Conference on Harmonisation [ICH]).

This includes any occurrence that is new in onset or aggravated in severity or frequency from the baseline condition, or abnormal results of diagnostic procedures, including laboratory test abnormalities.

The sponsor collects adverse events starting with the signing of the ICF.

Serious Adverse Event

A serious adverse event based on ICH and EU Guidelines on Pharmacovigilance for Medicinal Products for Human Use is any untoward medical occurrence that at any dose:

Results in death

Is life-threatening

(The subject was at risk of death at the time of the event. It does not refer to an event that hypothetically might have caused death if it were more severe.)

Requires inpatient hospitalization or prolongation of existing hospitalization

Results in persistent or significant disability/incapacity

Is a congenital anomaly/birth defect

Is a suspected transmission of any infectious agent via a medicinal product

Is Medically Important*

*Medical and scientific judgment should be exercised in deciding whether expedited reporting is also appropriate in other situations, such as important medical events that may not be immediately life threatening or result in death or hospitalization but may jeopardize the subject or may require intervention to prevent one of the other outcomes listed in the definition above. These should usually be considered serious.

If a serious and unexpected adverse event occurs for which there is evidence suggesting a causal relationship between the study intervention and the event (eg, death from anaphylaxis), the event will be reported as a serious and unexpected suspected adverse reaction even if it is a component of the study endpoint (eg, all-cause mortality).

Unlisted (Unexpected) Adverse Event/Reference Safety Information

An adverse event is considered unlisted if the nature or severity is not consistent with the applicable product reference safety information. For FP2, the expectedness of an adverse event will be determined by whether or not it is listed in the Investigator's Brochure.

Adverse Event Associated with the Use of the Intervention

An adverse event is considered associated with the use of the intervention if the attribution is possible, probable, or very likely by the definitions listed in Section 12.1.2, Attribution Definitions.

No clinical studies have been conducted with FP2 therefore, no data is available on the effects of FP2 in humans.

12.1.2. Attribution Definitions

Not Related

An adverse event that is not related to the use of the intervention.

Doubtful

An adverse event for which an alternative explanation is more likely, eg, concomitant drug(s), concomitant disease(s), or the relationship in time suggests that a causal relationship is unlikely.

Possible

An adverse event that might be due to the use of the intervention. An alternative explanation, eg, concomitant drug(s), concomitant disease(s), is inconclusive. The relationship in time is reasonable; therefore, the causal relationship cannot be excluded.

Probable

An adverse event that might be due to the use of the intervention. The relationship in time is suggestive (eg, confirmed by dechallenge). An alternative explanation is less likely, eg, concomitant drug(s), concomitant disease(s).

Very Likely

An adverse event that is listed as a possible adverse reaction and cannot be reasonably explained by an alternative explanation, eg, concomitant drug(s), concomitant disease(s). The relationship in time is very suggestive (eg, it is confirmed by dechallenge and rechallenge).

12.1.3. Severity Criteria

An assessment of severity grade will be made using the following general categorical descriptors:

Mild: Awareness of symptoms that are easily tolerated, causing minimal discomfort and not interfering with everyday activities.

Moderate: Sufficient discomfort is present to cause interference with normal activity.

Severe: Extreme distress, causing significant impairment of functioning or incapacitation. Prevents normal everyday activities.

The investigator should use clinical judgment in assessing the severity of events not directly experienced by the subject (eg, laboratory abnormalities).

12.1. Special Reporting Situations

Safety events of interest on a sponsor study intervention that may require expedited reporting or safety evaluation include, but are not limited to:

Overdose of a sponsor study intervention

Suspected abuse/misuse of a sponsor study intervention

Accidental or occupational exposure to a sponsor study intervention

Medication error involving a sponsor product (with or without subject/patient exposure to the sponsor study intervention, e.g., name confusion)

Special reporting situations should be recorded in the eCRF. Any special reporting situation that meets the criteria of a serious adverse event should be recorded on the serious adverse event page of the eCRF.

12.3. Procedures

12.3.1. All Adverse Events

All adverse events and special reporting situations, whether serious or non-serious, will be reported from the time a signed and dated ICF is obtained until completion of the subject's last study-related procedure, which may include contact for follow-up of safety. Serious adverse events, including those spontaneously reported to the investigator by the End-of-Study visit, will be reported using the Serious Adverse Event Form. The sponsor will evaluate any safety information that is spontaneously reported by an investigator beyond the time frame specified in the protocol.

All events that meet the definition of a serious adverse event will be reported as serious adverse events, regardless of whether they are protocol-specific assessments.

All adverse events, regardless of seriousness, severity, or presumed relationship to study intervention, will be recorded using medical terminology in the source document and the eCRF. Whenever possible, diagnoses should be given when signs and symptoms are due to a common etiology (eg, cough, runny nose, sneezing, sore throat, and head congestion should be reported as “upper respiratory infection”). Investigators will record in the eCRF their opinion concerning the relationship of the adverse event to study therapy. All measures required for adverse event management will be recorded in the source document and reported according to sponsor instructions.

### 14. Study Drug Information

14.1. Physical Description of Study Drugs

FP2 supplied for this study is a sterile, brown-yellow solution with a concentration of 50 mg/mL in 10 mM sodium phosphate, 8% sucrose, and 0.04% Polysorbate 20, at a pH 6.5.

FP2 is provided frozen in a glass vial with a 1.2 mL fill volume.

The formulation buffer supplied for this study is a sterile, clear solution consisting of 10 mM sodium phosphate, 8.0% sucrose and 0.04% polysorbate 20. The formulation buffer will be used to prepare the placebo injections and as a diluent in the preparation of the initial FP2 SC doses.

The formulation buffer is provided frozen in a glass vial with a 1.2 mL fill volume.

It will be manufactured and provided under the responsibility of the sponsor. List of excipients is provided in above.

14.2. Packaging

Study drug (FP2 and formulation buffer for dilution of FP2 and as placebo) will be provided as bulk supplies. Study drug will be stored in a locked pharmacy and only transferred to a suitably qualified study team member for administration after preparation for an IV infusion (Part 2) or a blinded preparation for a SC injection (Part 1).

14.3. Labeling

Study drug labels will contain information to meet the applicable regulatory requirements.

14.4. Preparation, Handling, and Storage

All study drug will be stored at controlled temperatures ranging from −30° C. to −40° C. and protected from light.

For initial four planned SC dose-groups, the drug product will be diluted before use to the required concentration (DG 1 0.5 mg/mL; DG 2 and DG 3 5.0 mg/mL; DG 4 10.0 mg/mL) using the formulation buffer. Drug product for IV administration will be diluted to a concentration of 10 mg/mL and administered with in-line filtration.

### REFERENCES

1. Bazett H C (1920). An analysis of the time-relationship of electrocardiograms. Heart. 1920; 7:353-380.
2. Breit S N (2011), Johnen H., Cook A D, Tsai V W W, Mohammad M G, Kuffner T, et al. The TGF-β superfamily cytokine, MIC-1/GDF15: A pleotrophic cytokine with roles in inflammation, cancer and metabolism. Growth Factors. 2011; 29(5):187-195.
3. EMA Guidance. EMEA CHMP SWP 28367 07 Rev. 1 (2017). Guideline on strategies to identify and mitigate risks for first-in-human and early clinical trials with investigational medicinal products. European Medicines Agency; Committee for Medicinal Products for Human Use (CHMP); 20 Jul. 2017.
4. EMA Guidance. EMEA/CHMP/SWP/28367/07 (2007). Guideline on strategies to identify and mitigate risks for first-in-human clinical trials with investigational medicinal products. European Medicines Agency; Committee for Medicinal Products for Human Use (CHMP); 19 Jul. 2007.
5. Emmerson P J (2017), Wang F, Du Y, Liu Q, Pickard R T, Gonciarz M D, et al. The metabolic effects of GDF15 are mediated by the orphan receptor GFRAL. Nat. Med. 2017; (10):1215-1219.
6. FDA Guidance for Industry (2005). Estimating the maximum safe starting dose in initial clinical trials for therapeutics in adult healthy volunteers. US Dep of Health and Human Services. Center for Drug Evaluation and Research (CDER, July 2005).
7. FDA Guidance for Industry (2007). Toxicity Grading Scale for Healthy Adult and Adolescent Volunteers Enrolled in Preventive Vaccine Clinical Trials. US Dep of Health and Human Services. Center for Biologics Evaluation and Research (CBER, September 2007).
8. Fejzo M S (2018), Sazonova O V, Sathirapongsasuti J F, Hallgrímsdóttir I B, 23andMe Research Team, Vacic V, et al. Placenta and appetite genes GDF15 and IGFBP7 are associated with hyperemesis gravidarum. Nature Communications. 2018; 9:1178-1186.
9. Fisher B L (2002) and Schauer P. Medical and surgical options in the treatment of severe obesity. Am J Surg. 2002; 184:9S-16S.
10. Fryar C D (2012), Carroll M D, Ogden C L. Prevalence of overweight, obesity, and extreme obesity among adults: United States, trends 1960-1962 through 2009-2010, 2012. National Center for Health Statistics. Available from http://www.cdc.gov/nchs/data/hestat/obesity_adult_09_10/obesity_adult_09_10.htm Accessed 23 May 2018
11. Gennemark P (2017), Tragardh M, Linden D, Ploj K, Johansson A, Turnbull A, et al. Translational modeling to guide study design and dose choice in obesity exemplified by AZD1979, a melanin-concentrating hormone receptor 1 antagonist. CPT Pharmacometrics Syst Pharmacol. 2017; 6:458-468.
12. Gobel B (2014), Sanghvi A, and Hall K D. Quantifying energy intake changes during obesity pharmacotherapy. Obesity. 2014; 22(10):2105-2108.
13. Hall K D (2011), Sacks G, Chandramohan D, Chow C, Wang Y C, Gortmaker S L, and Swinburn B A. Quantification of the effect of energy imbalance on bodyweight. Lancet. 2011; 378:826-837.
14. Hodges M (1983), Salerno D, Erlien D. Bazett's QT correction reviewed: evidence that a linear QT correction for heart rate is better. J Am Coll Cardiol. 1983; 1:694.
15. Hsu J Y (2017), Crawley S, Chen M, Ayupova D A, Lindhout D A, et al. Non-homeostatic body weight regulation through a brainstem-restricted receptor for GDF15. Nature. 2017; 550(7675):255-259.
16. International Conference on Harmonisation of Technical Requirements for Registration of Pharmaceuticals for Human Use. ICH Harmonized Tripartite Guideline E14: Clinical evaluation of QT/QTc interval prolongation and proarrhythmic potential for non-antiarrhythmic drugs. ICH 12 May 2005.
17. Mullican S E (2017), Lin-Schmidt X, Chin C-N, Chavez J A, Furman J L, et al. GFRAL is the receptor for GDF15 and the ligand promotes weight loss in mice and nonhuman primates. Nat. Med. 2017; (10):1150-1157.
18. Rueda-Clausen C F (2015), Ogunleye A A, and Sharma A M. Health benefits of long-term weight-loss maintenance. Annu. Rev. Nutr. 2015; 35:475-516.
19. Sagie A (1992), Larson M G, Goldberg R J, Bengston J R, Levy D. An improved method for adjusting the QT interval for heart rate (the Framingham Heart Study). Am J Cardiol. 1992; 70:797-801.
20. Sugulle M (2009), Dechend R, Herse F, Weedon-Fekjaer S M, Johnsen G M, et al. Circulating and placental Growth-Differentiation Factor 15 in preeclampsia and in pregnancy complicated by diabetes mellitus. Hypertension. 2009; 54:106-112.
21. The GBD Obesity Collaboration (2013). Global, regional and national prevalence of overweight and obesity in children and adults 1980-2013: a systematic analysis for the Global Burden of Disease Study 2013. Lancet. 2014; 384:766-781.
22. Wolfe B M (2016), Kvach E, and Eckel R H. Treatment of obesity: weight loss and bariatric surgery. Circ Res. 2016; 118:1844-1855.
23. World Health Organization (2012), Blood Donor Selection: Guidelines on Assessing Donor Suitability for Blood Donation. Geneva. Available from: https://www.ncbi.nlm.nih.gov/books/NBK138212/Accessed 21 Jun. 2018.
24. Yang L (2017), Chang C, Sun Z, Madsen D, Zhu H, Padkjaer S, et al. GFRAL is the receptor for GDF15 and is required for the anti-obesity effects of the ligand. Nat. Med. 2017; (10):1158-1166.
25. Yanovski S Z (2015), Marcus M D, Wadden T A, Walsh B T. The Questionnaire on Eating and Weight Patterns5 (QEWP-5): An Updated Screening Instrument for Binge Eating Disorder. Int J Eat Disord. 2015; 48(3): 259-261

### Example 22: Clinical Trial Results

A clinical trial was conducted in accord with the Protocol described in Example 21, with the following exceptions: (1) Part 2 of the study (Absolute Bioavailability) was not conducted; (2) Inclusion Criterion defining the participant age was changed to increase the eligible upper age limit to 55 years; (3) Exclusion Criterion based on the participant having a positive urine cotinine test was modified to allow inclusion of light and intermittent smokers who were willing to abstain from smoking during the in-house period. Dosing was completed for Cohorts 1-6 while dosing for Cohort 7 is ongoing. Forty-eight subjects, split into six cohorts of eight subjects each (Cohorts 1-6), have been exposed to either single doses of FP2 or matching placebo. In each Cohort, 6 subjects were dosed with FP2 and 2 subjects were dosed with matching placebo. Below is a summary of the doses of FP2 (or matching placebo) administered to each Cohort 1-6 (Table 71):

TABLE 71
Cohort 1-6 dosing.

| Cohort | Dose | |
|---|---|---|
| Cohort 1 | 0.8 | mg |
| Cohort 2 | 2.5 | mg |
| Cohort 3 | 7.5 | mg |
| Cohort 4 | 15 | mg |
| Cohort 5 | 30 | mg |
| Cohort 6 | 60 | mg |

Blinded safety and tolerability information are available for all subjects dosed (Cohorts 1-6). Group level summary pharmacokinetic (PK) and pharmacodynamic (PD) results are available for Cohorts 1-5.

Safety and Tolerability: Treatment was generally considered safe and well tolerated.

Pharmacokinetics

Pharmacokinetics data were available for Cohorts 1-5. For increasing doses of FP2 administered, exposures increased in an approximately dose-proportional manner. Tₘₐₓ occurred at Day 6 and the T_{1/2} was approximately 12 days, thus supporting weekly dosing.

TABLE 72
Pharmacokinetics of single ascending doses of FP2.

| | 0.8 mg (n = 6) | 2.5 mg (n = 6) | 7.5 mg (n = 6) | 15 mg (n = 6) | 30 mg (n = 6) |
|---|---|---|---|---|---|
| PK parameters | Mean (SD) | Mean (SD) | Mean (SD) | Mean (SD) | Mean |
| AUC₇₂(μg · hr/ml) | 3.36 (2.94) | 9.71 (5.33) | 27.3 (5.31) | 46.1 (20.2) | 128.9 |
| C₇₂ (μg/ml) | 0.0691 (0.0572) | 0.208 (0.0935) | 0.592 (0.0975) | 1.05 (0.456) | 2.65 |

C₇₂ represents 72% of Cₘₐₓ

Pharmacodynamics

Food intake assessment was conducted at baseline (Day −1) and again on Day 3 after study drug administration. The Tₘₐₓ observed during the first 4 cohorts of this SAD study is at ˜Day 6, and plasma concentration of FP2 at Day 3 is ˜70-80% of the concentration achieved on Day 6.

To assess food intake, four standardized meals (breakfast, lunch, snack, dinner) were provided to the subjects; each subject received the same meals (same food items and same quantities) on both days (i.e., Days −1 and 3). The calories consumed from the 4 meals were summed to obtain the total calories consumed at baseline (Day −1) and on Day 3. Changes in food intake from baseline to Day 3 were used as an exploratory measure of FP2 pharmacodynamic mode of action.

Changes in food intake were variable in both subjects on placebo and on active drug, with changes on Day 3 relative to baseline of approximately ±20% in Cohorts 1 to 4. Nonetheless, food intake decreased by more than 50% (range 55-96%) in 4 out of 6 subjects treated with 30 mg (Cohort 5) of FP2. In comparison, none of the aggregate placebo group (10 subjects) achieved food intake reductions>30%. In this same dose group (Cohort 5, 30 mg) the 2 subjects who showed the greatest decrease in food intake (−85% and −96% reductions in food intake) were also the subjects who reached the highest exposures of FP2). The median value for percent food intake change from baseline to Day 3 post-dose for subjects on either placebo or on FP2 is presented in Table 73.

TABLE 73
Changes in food intake from baseline (Day −1) to Day 3 in subjects
treated with either placebo or with escalating doses of FP2.

| | Median value for % food intake | |
|---|---|---|
| Cohort | change from baseline to Day 3 | |
| (number of subjects) | (5^{th}, 95^{th} percentile) | |
| Placebo (N = 10) | −11.7 | (−28.6, 15.6) |
| Cohort 1 (N = 6) | 2.3 | (−17.7, 14.8) |
| Cohort 2 (N = 6) | 0.7 | (−24.0, 16.1) |
| Cohort 3 (N = 6) | −9.9 | (−26.3, 11.8) |
| Cohort 4 (N = 6) | −10.8 | (−29.8, 5.8) |
| Cohort 5 (N = 6) | −33.7 | (−92.1, 14.8) |
| Cohort 6 (blinded)* (N = 8) | −35.1 | (−68.4, 1.9) |

While the invention has been described in detail, and with reference to specific embodiments thereof, it will be apparent to one of ordinary skill in the art that various changes and modifications can be made therein without departing from the spirit and scope of the invention.

1. A method of decreasing body weight in a subject, comprising administering a composition comprising a fusion protein comprising SEQ ID NO: 92, and at least one pharmaceutically acceptable carrier or diluent, wherein said fusion protein is administered at a dose in the range of 0.8 mg to 90 mg, and wherein the subject's weight is 80 kg or more.

2. The method of claim 1, wherein the subject is overweight.

3. The method of claim 2, wherein the subject has a BMI of 25 kg/m² or more.

4. The method of claim 3, wherein the subject has the BMI in the range of 25 kg/m² to 29.9 kg/m².

5. The method of claim 1, wherein the fusion protein is administered at a dose selected from the group consisting of: 0.8 mg, 2.5 mg, 7.5 mg, 15 mg, 30 mg, 60 mg, and 90 mg.

6-12. (canceled)

13. The method of claim 1, wherein the fusion protein is administered via subcutaneous injection.

14. The method of claim 1 wherein the composition is administered once weekly to the subject.

15. A method of decreasing food intake in a subject, comprising administering a composition comprising a fusion protein comprising SEQ ID NO: 92, and at least one pharmaceutically acceptable carrier or diluent, wherein said fusion protein is administered at a dose in the range of 0.8 mg to 90 mg, and wherein the subject weight is 80 kg or more.

16. The method of claim 15, wherein the subject is overweight.

17. The method of claim 16, wherein the subject has a BMI of 25 kg/m² or more.

18. The method of claim 17, wherein the subject has the BMI in the range of 25 kg/m² to 29.9 kg/m².

19. The method of claim 15, wherein the fusion protein is administered at a dose selected from the group consisting of: 0.8 mg, 2.5 mg, 7.5 mg, 15 mg, 30 mg, 60 mg, and 90 mg.

20-26. (canceled)

27. The method of claim 15, wherein the fusion protein is administered via subcutaneous injection.

28. The method of claim 15 wherein the composition is administered once weekly to the subject.

29. A method of decreasing body weight in a subject, comprising administering a composition comprising a fusion protein comprising SEQ ID NO: 92, and at least one pharmaceutically acceptable carrier or diluent, wherein said composition is administered at a dose in a range of 0.01 mg/kg to 1.08 mg/kg.

30. The method of claim 29, wherein said composition is administered at a dose selected from the group consisting of: 0.01 mg/kg, 0.03 mg/kg, 0.09 mg/kg, 0.18 mg/kg, 0.36 mg/kg, 0.72 mg/kg, and 1.08 mg/kg.

31-37. (canceled)

38. The method of claim 29, wherein the fusion protein is administered via subcutaneous injection.

39. The method of claim 29 wherein the composition is administered once weekly to the subject.

40. A method of decreasing food intake in a subject, comprising administering a composition comprising a fusion protein comprising SEQ ID NO: 92, and at least one pharmaceutically acceptable carrier or diluent, wherein said composition is administered at a dose in a range of 0.01 mg/kg to 1.08 mg/kg.

41. The method of claim 40, wherein said composition is administered at a dose selected from the group consisting of: 0.01 mg/kg, 0.03 mg/kg, 0.09 mg/kg, 0.18 mg/kg, 0.36 mg/kg, 0.72 mg/kg, and 1.08 mg/kg.

42-48. (canceled)

49. The method of claim 41, wherein the fusion protein is administered via subcutaneous injection.

50. The method of claim 41, wherein the composition is administered once weekly to the subject.
